(19) United States
(12) Patent Application Publication
(10) Pub. No.: US 2010/0120747 A1
(43) Pub. Date: May 13, 2010

(54) COMBINATION OF A CHOLINESTERASE INHIBITOR AND A COMPOUND WITH 5-HT6 RECEPTOR AFFINITY

(75) Inventors: Xavier Codony-Soler, Barcelona (ES); Helmut Heinrich Buschmann, San Just Desvern (Barcelona) (ES)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Laboratorios del Dr. Esteve, S.A., Barcelona (ES)

(21) Appl. No.: 12/305,516

(22) PCT Filed: Jun. 22, 2007
(86) PCT No.: PCT/EP07/56234

§ 371 Date: Mar. 26, 2009

(30) Foreign Application Priority Data
Jun. 23, 2006 (EP) 06384012.8

(51) Int. Cl. A61K 31/496; A61K 31/536; A61K 31/404; A61K 31/505; A61K 31/44; A61K 31/4439; A61K 31/437; A61K 31/4525; A61K 31/4965; A61K 31/55; A61P 3/00; A61P 25/00; A61P 1/00

(52) U.S. Cl. 514/217.01; 514/252.13; 514/230.5; 514/254.09; 514/415; 514/275; 514/353; 514/339; 514/303; 514/320; 514/255.03

(57) ABSTRACT

The present invention relates to an active substance combination comprising at least one compound with 5-HT6 receptor affinity, and at least one cholinesterase inhibitor, a medicament comprising said active substance combination, and the use of said active substance combination for the manufacture of a medicament.

### FIELD OF THE INVENTION

The present invention relates to an active substance combination comprising at least one compound with 5-HT₆ receptor affinity, and at least one cholinesterase inhibitor, a medicament comprising said active substance combination, and the use of said active substance combination for the manufacture of a medicament.

### BACKGROUND OF THE INVENTION

Cognitive and/or degenerative brain disorders are characterized clinically by progressive loss of memory, cognition, reasoning, judgement and emotional stability that gradually leads to profound mental deterioration and ultimately death. In an example of such disorders, Alzheimer's disease is a common cause of progressive mental failure (dementia) in aged humans and is believed to represent the fourth most common medical cause of death in the United States. In particular, Alzheimer's disease is associated with degeneration of cholinergic neurons in the basal forebrain that play a fundamental role in cognitive functions, including memory. Cognitive and/or degenerative brain disorders have been observed in varied races and ethnic groups world-wide and presents a major public health problem. These diseases are currently estimated to affect about two to three million individuals in the United States alone and the occurrence will increase world-wide as the human life span increases.

Cognitive and/or degenerative brain disorders are incurable with presently used medications, however, the symptoms of these disorders can be alleviated by using compounds useful for treating cognitive disorders, in particular for treating Alzheimer's disease, such as donepezil, rivastigmine, galantamine and phenserine all of which act as acetylcholinesterase inhibitors.

All acetylcholinesterase inhibitors have serious drawbacks in that they produce undesirable side affects caused by their activity as acetylcholinesterase inhibitors. These undesirable side effects are related to their toxicity caused by their suppression of acetylcholinesterase. Due to the fact that acetylcholinesterase inhibitors, which are administered chronically, have a low therapeutic ratio (i.e. the ratio between toxicity and therapeutic effect) they produce a number of pathologic conditions associated with cholinergic under activity. Therefore due to the chronic nature of treatment for cognitive disorders it has long been desired to provide a medicament which is effective and does not produce the toxic side effects inherent in the use of acetylcholinesterase inhibitors and does not present undesired side effects such as nausea and vomiting and, thus, can be administered for long periods.

### BRIEF DESCRIPTION OF THE INVENTION

It was therefore an object of the present invention to provide a medicament suitable for the prophylaxis and/or treatment of disorders related to acetylcholinesterase, and to 5-HT₆ receptors, which preferably does not show the undesired side effects of the conventional compounds which act as cholinesterase inhibitors, or at least less frequent and/or less pronounced.

In particular, it was an object of the present invention to provide a medicament suitable for the prophylaxis and/or treatment of cognitive disorders, which preferably does not show the undesired side effects of the conventional medicaments for the prophylaxis and/or treatment of cognitive disorders, or at least less frequent and/or less pronounced.

Said object has been achieved by providing an active substance combination comprising

(A) at least one compound with 5-HT₆ receptor affinity, and

(B) at least one cholinesterase inhibitor.

whereby an active substance combination comprising as component (A) the 5-HT6 antagonist SB271046 and as component (B) the cholinesterase inhibitor donepezil hydrochloride is excluded.

Donepezil hydrochloride is sold under the brand name Aricept® as a medication to treat Alzheimer's disease. The respective compound donepezil hydrochloride has the code names BNAG and E-2020, respectively.

Another object of the present invention relates to an active substance combination as defined above for its use as a medicament.

A third object of the invention refers to the use of the combination as defined above for the manufacture of a medicament for simultaneous acetylcholinesterase inhibition and 5-HT6-receptor regulation.

Even another object of the invention relates to the use of the combination as defined above for the manufacture of a medicament for regulation of appetite, for maintenance, increase or reduction of body weight, for prophylaxis and/or treatment of disorders related to food ingestion, preferably for prophylaxis and/or treatment of obesity, anorexia, cachexia, bulimia, diabetes, preferably type II diabetes (non-insulin- dependent diabetes mellitus), or for prophylaxis and/or treatment of gastrointestinal tract disorders, preferably of the irritable bowel syndrome, for prophylaxis and/or treatment of Metabolic Syndrome, Peripheral Nervous System Disorders, Central Nervous System Disorders, arthritis, epilepsy, anxiety, panic, depression, cognitive disorders, memory disorders, cardiovascular diseases, senile dementia processes, such as Alzheimer's, Parkinson's and/or Huntington's Disease, schizophrenia, psychosis, infantile hyperkinesia (ADHD, attention deficit/hyperactivity disorder), pain, hypertensive syndrome, inflammatory diseases, immunologic diseases or for improvement of cognition.

Finally, another object refers to a pharmaceutical formulation, characterized in that it comprises an active substance combination as defined above and optionally one or more pharmacologically acceptable adjuvants.

### DETAILED DESCRIPTION OF THE INVENTION

It has surprisingly been found that the compounds with 5-HT₆ receptor affinity and the compounds which act as cholinesterase inhibitors show a synergistic effect in their pharmacological activities. Consequently, the dose of the corresponding compounds may be reduced in comparison to the dose necessary for an individual administration of said compounds. In particular, the combination of an amount of compound with 5-HT₆ receptor affinity and an amount of cholinesterase inhibitor which both basically do not show any pharmacological activity in these amounts leads to an active substance combination of these compounds which shows a pharmacological activity when these amounts are administered in combination.

According to the invention it has also been found that the action of a acetylcholinesterase inhibitor potentiates the action of the compound with 5-HT₆ receptor affinity, so the combination of a cholinesterase inhibitor and a compound with 5-HT₆ receptor affinity for use in the treatment of disorders that are related to acetylcholinesterase, and to 5-HT₆ receptors may result in a faster onset of action and an increased success rate. The invention therefore resides in the combined action of a cholinesterase inhibitor and a compound with 5-HT₆ receptor affinity, or the dual action of a substance possessing both cholinesterase inhibitor activity and 5-HT₆ receptor affinity, for the treatment of disorders that are related to acetylcholinesterase, and to 5-HT₆ receptors.

Preferably the compounds which are present as component (A) have selective affinity for 5-HT₆ receptors. Thus, these compounds have a higher affinity for the 5-HT₆ receptor than for other 5-HT receptors subtypes and preferably do not substantially bind to other 5-HT receptor subtypes such as 5-HT₁ receptors (e.g. 5-HT_{1A}, 5-HT_{1B}, 5-HT_{1D}, 5-HT_{1E}, 5-HT_{1F}), 5-HT_{2B}, 5-HT_{2C}, 5-HT₂ₐ and 5-HT₇.

More preferably the compounds which are present as component (A) will exhibit an affinity (pKi) for the 5-HT₆ receptor with a value of greater than or equal to about 6, preferably with a value of greater than or equal to about 8, more preferably with a value of greater than or equal to about 9, which is at least 20-fold greater than, preferably at least 30-fold greater than its pKi for the 5-HT_{2c} receptor. Assays that may be used for determining the affinity and selectivity of a 5-HT₆ receptor antagonist and/or a 5-HT₂ receptor antagonist are well known in the art and are also provided in the examples below.

Preferably the compound present as component (A) shows a Kᵢ value for binding to the 5-HT₆-receptor of below or equal to 1 μM, preferably below or equal to 500 nM, more preferably below or equal to 100 nM, even more preferably below or equal to 50 nM, still even more preferably below or equal to 25 nM.

Preferably cell membranes of HEK-293 cells expressing the 5HT₆-human recombinant receptor were used for determining the K. In said membranes the receptor concentration was 2.18 pmol/mg protein and the protein concentration is 9.17 mg/ml. The experimental protocol followed the method of B. L. Roth et al. [B. L. Roth, S. C. Craigo, M. S. Choudhary, A. Uluer, F. J. Monsma, Y. Shen, H. Y. Meltzer, D. R. Sibley: Binding of Typical and Atypical Antipsychotic Agents to 5-Hydroxytryptamine-6 and Hydroxytryptamine-7 Receptors. The Journal of Pharmacology and Experimental Therapeutics, 1994, 268, 1403] which is hereby incorporated by reference and forms part of the disclosure. The exact experimental protocol is also outlined below (see Pharmacological Methods).

In a particular embodiment of the invention, the component (A) exhibit an affinity for the 5-HT₆ receptor acting as agonist thereof. In another particular embodiment, the component (A) exhibit an affinity for the 5-HT₆ receptor acting as antagonist thereof. In still another particular embodiment, the component (A) exhibit an affinity for the 5-HT₆ receptor acting as inverse agonist thereof.

Preferably the compound with 5-HT₆ receptor affinity acts as agonist or inverse agonist of the 5-HT₆ receptor, more preferably the compound with 5-HT₆ receptor affinity acts as agonist of the 5-HT₆ receptor. More preferably the compound with 5-HT₆ receptor affinity acts as selective agonist or selective inverse agonist of the 5-HT₆ receptor, more preferably the compound with 5-HT₆ receptor affinity acts as selective agonist of the 5-HT₆ receptor.

It is possible to classify a compound with 5-HT6 receptor affinity as agonist, inverse agonist or antagonist according to the reference of S. M. Stahl, Essential Psychopharmacology, Neuroscientific basis and practical applications, Ed. Cambridge, 1996, Chapter 3. The respective part of the literature is hereby incorporated by reference and forms part of the disclosure.

An “Agonist” is defined as a compound that binds to a receptor and has an intrinsic effect, and thus, increases the basal activity of a receptor when it contacts the receptor.

An “antagonist” is defined as a compound that competes with an agonist or inverse agonist for binding to a receptor, thereby blocking the action of an agonist or inverse agonist on the receptor. However, an antagonist (also known as a “neutral” antagonist) has no effect on constitutive receptor activity.

An “inverse agonist” is defined as a compound that produces an effect opposite to that of the agonist by occupying the same receptor and, thus, decreases the basal activity of a receptor (i.e., signalling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist.

According to the present invention, the term “cholinesterase inhibitor” denotes acetylcholinesterase inhibitors as well as butyrylcholinesterase inhibitors.

The compound present as component (B) shows an IC₅₀ for the inhibition of acetylcholinesterase in erythrocytes below or equal to 1 μM, preferably below or equal to 500 nM, more preferably below or equal to 300 nM, even more preferably below or equal to 200 nM, still even more preferably below or equal to 100 nM.

Assays that may be used for determining the inhibition of acetylcholinesterase in erythrocytes are well known in the art and are also provided in the following references: J. Med. Chem. 2002, 45, 3684; J. Med. Chem. 2001, 44, 4733 and J. Med. Chem. 2005, 48, 1701. The respective parts of the literature are hereby incorporated by reference and form part of the disclosure.

Preferably the cholinesterase inhibitor according to the present invention is selected from the group consisting of reversible cholinesterase inhibitors and pseudo-reversible cholinesterase inhibitors.

The terms “cholinesterase inhibitor” and “acetylcholinesterase inhibitor” interchangeably refer to a pharmaceutical compound that inhibits the activity of the enzyme acetylcholinesterase (AChE). Cholinesterase inhibitors are generally classified as “reversible,” “pseudo-irreversible” or “slow reversible,” and “irreversible.” “Reversible” cholinesterase inhibitors typically are non-covalent inhibitors. “Pseudo-irreversible,” “pseudo-reversible” or “slow reversible” cholinesterase inhibitors react covalently or noncovalently with AChE with high affinity. Pseudo-irreversible cholinesterase inhibitors typically, but nonexclusively, have a carbamoyl ester linkage and are hydrolysed by AChE, but much more slowly than acetylcholine. Attack by the active centre serine of AChE gives rise to a carbamoylated AChE. The duration of inhibition by the carbamoylating acetylcholinesterase inhibitors can be about 3 to 4 hours. The half-life of such carbamoylating agents, for example, physostigmine, neostigmine, and pyridostigmine, can be about 1 to 2 hours. The distinction between “pseudo-irreversible” and “reversible” cholinesterase inhibitors generally reflects quantitative differences in rates of deacylation of the acyl enzyme. With “pseudo-irreversible” cholinesterase inhibitors, the half-life for hydrolysis of the dimethylcarbamoyl enzyme is about 15 to 30 minutes. “Irreversible” cholinesterase inhibitors are usually organophophorus compounds. With “irreversible” cholinesterase inhibitors, the active enzyme can spontaneously regenerate after several hours or so slowly that the return of AChE activity depends on the synthesis of new enzyme. Acetylcholinesterase inhibitors are well known and discussed in detail in, for example, Goodman and Gilman's The Pharmacological Basis of Therapeutics, Chapter 8, 10.sup.th Ed., Hardman, Limbird and Goodman-Gilman, Eds., McGraw-Hill (2001), hereby incorporated herein by reference.

Preferably as component (A) at least one compound is present which is selected from the group consisting of the benzoxazinone-derived sulfonamide compounds of general formula (Ia)

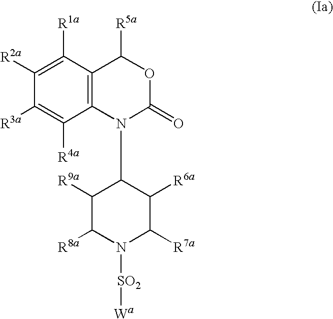

wherein

R^{1a}, R^{2a}, R^{3a} and R^{4a}, independently of one another, each represent a hydrogen atom; halogen; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl- or heteroaryl radical, which may be bonded via an unsubstituted or at least mono-substituted alkylene group and/or which may be condensed with an unsubstituted or at least mono-substituted mono- or bicyclic ringsystem; nitro; cyano; —O—R^{10a}; —O—(C═O)—R^{11a}; —(C═O)—OR^{11a}; —SR^{12a}; —SOR^{12a}; —SO₂R^{12a;}, —NH—SO₂R^{12a}; —SO₂NH₂ or —NR^{13a}R^{14a};

R^{5a} represents a hydrogen atom; an unbranched or branched, saturated or unsaturated, unsubstituted or at least mono-substituted aliphatic radical or a saturated or unsaturated, unsubstituted or at least mono-substituted, optionally at least one heteroatom as ring member containing cycloaliphatic radical;

R^{6a}, R^{7a}, R^{8a}, R^{9a}, independently of one another, each represent a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical; a cyano group or a —C(═O)—OR^{15a} moiety;

W^{a} represents an unbranched or branched, saturated or unsaturated, unsubstituted or at least mono-substituted aliphatic radical;

a saturated or unsaturated, unsubstituted or at least mono-substituted, optionally at least one heteroatom as ring member containing cycloaliphatic radical, which may be bonded via an optionally mono-substituted alkylene group and/or which may be condensed with an unsubstituted or at least mono-substituted mono- or bicyclic ring system;

an unsubstituted or at least mono-substituted aryl or heteroaryl radical, which may be bonded via an unsubstituted or at least mono-substituted alkylene or alkenylene group and/or which may be condensed with an unsubstituted or at least mono-substituted mono- or bicyclic ring system;

a —NR^{16a}R^{17a} moiety, or

a —C(═O)—R^{18a} moiety;

R^{10a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;

R^{11a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;

R^{12a} represents an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;

R^{13a} and R^{14a}, independently of one another, each represent a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;

or R^{13a} and R^{14a} together with the bridging nitrogen atom form a saturated, unsaturated or aromatic heterocyclic ring, which is unsubstituted or at least mono-substituted and/or which may contain at least one further heteroatom as a ring member;

R^{15a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;

R^{16a} represents an unbranched or branched, saturated or unsaturated, unsubstituted or at least mono-substituted aliphatic radical;

R^{17a} represents an unbranched or branched, saturated or unsaturated, unsubstituted or at least mono-substituted aliphatic radical, and

R^{18a} represents an unsubstituted or at least mono-substituted aryl radical;

optionally in form of one of its stereoisomers, preferably enantiomers or diastereomers, its racemate or in form of a mixture of at least two of its stereoisomers, preferably enantiomers or diastereomers, in any mixing ratio, or a physiologically acceptable salt thereof, or a solvate, respectively.

Preferred compounds of general formula (Ia) are those, wherein

R^{1a}, R^{2a}, R^{3a} and R^{4a}, independently of one another, each represent a hydrogen atom; a fluorine atom; a chlorine atom; a bromine atom; a methyl group or a methoxy group;

R^{5a} represents a hydrogen atom;

R^{6a}, R^{7a}, R^{8a} and R^{9a} each represent a hydrogen atom;

W^{a} represents

an alkyl radical selected from the group consisting of methyl; ethyl; n-propyl; isopropyl; n-butyl; sec-butyl; isobutyl and tert-butyl; vinyl (CH₂═CH—); —N(CH₃)₂; 1-naphthyl; benzyl; 2-naphtyl; phenyl; 2-methyl-phenyl; 3-methyl-phenyl; 4-methyl-phenyl; 2-ethyl-phenyl; 3-ethyl-phenyl; 4-ethyl-phenyl; 2-n-propyl-phenyl; 3-n-propyl-phenyl; 4-n-propyl-phenyl; 2-isopropyl-phenyl; 3-isopropyl-phenyl; 4-isopropyl-phenyl; 2-n-butyl-phenyl; 3-n-butyl-phenyl; 4-n-butyl-phenyl; 2-isobutyl-phenyl; 3-isobutyl-phenyl; 4-isobutyl-phenyl; 2-tert-butyl-phenyl; 3-tert-butyl-phenyl; 4-tert-butyl-phenyl; 1,1-dimethylpropyl-phenyl; 2-cyclopentyl-phenyl; 3-cyclopentyl-phenyl; 4-cyclopentyl-phenyl 2-cyclohexyl-phenyl; 3-cyclohexyl-phenyl; 4-cyclohexyl-phenyl; 2-methoxy-phenyl; 3-methoxy-phenyl; 4-methoxy-phenyl; 2-ethoxy-phenyl; 3-ethoxy-phenyl; 4-ethoxy-phenyl; 2-n-propoxy-phenyl; 3-n-propoxy-phenyl; 4-n-propoxy-phenyl; 2-iso-propoxy-phenyl; 3-iso-propoxy-phenyl; 4-isopropoxy-phenyl;2-fluoro-phenyl; 3-fluoro-phenyl; 4-fluoro-phenyl; 2-chloro-phenyl; 3-chloro-phenyl; 4-chloro-phenyl; 2-bromo-phenyl; 3-bromo-phenyl; 4-bromo-phenyl; 2-trifluoromethyl-phenyl; 3-trifluoromethyl-phenyl; 4-trifluoromethyl-phenyl; 2-trifluoromethoxy-phenyl; 3-trifluoromethoxy-phenyl; 4-trifluoromethoxy-phenyl; 2-carboxy-phenyl; 3-carboxy-phenyl; 4-carboxy-phenyl; 2-acetyl-phenyl; 3-acetyl-phenyl; 4-acetyl-phenyl; 2-(C═O)—O—CH₃-phenyl; 3-(C═O)—O—CH₃-phenyl; 4-(C═O)—O—CH₃-phenyl; 2-(CH₂)—(CH₂)—(C═O)—O—CH₃-phenyl; 3-(CH₂)—(CH₂)—(C═O)—O—CH₃-phenyl; 4-(CH₂)—(CH₂)—(C═O)—O—CH₃-phenyl; 2-cyano-phenyl; 3-cyano-phenyl; 4-cyano-phenyl; 2-nitro-phenyl; 3-nitro-phenyl; 4-nitro-phenyl; 4-(4-bromophenoxy)-phenyl; 2-methylsulfonyl-phenyl; 3-methylsulfonyl-phenyl; 4-methylsulfonyl-phenyl; 2-phenyl-phenyl(biphenyl-2-yl); 3-phenyl-phenyl(biphenyl-3-yl); 4-phenyl-phenyl(biphenyl-4-yl); 2-phenoxy-phenyl; 3-phenoxy-phenyl; 4-phenoxy-phenyl; 2,4-dimethyl-phenyl; 3,4-dimethyl-phenyl; 2,4,6-trimethyl-phenyl; 2,3,5,6-tetramethyl-phenyl; pentamethyl-phenyl; 2,5-dimethoxy-phenyl; 3,4-dimethoxy-phenyl; 2,3-dichloro-phenyl; 2,4-dichloro-phenyl; 2,5-dichloro-phenyl; 3,4-dichloro-phenyl; 3,5-dichloro-phenyl; 2,6-dichloro-phenyl; 2,4-difluoro-phenyl; 3,4-difluoro-phenyl; 2,5-difluoro-phenyl; 2,6-difluoro-phenyl; 3-chloro-2-fluoro-phenyl; 3-chloro-4-fluoro-phenyl; 5-chloro-2-fluoro-phenyl; 2,3,4-trichloro-phenyl; 2,4,5-trichloro-phenyl; 2,4,6-trichloro-phenyl; 2,4,5-trifluoro-phenyl; 2,3,4-trifluoro-phenyl-; 2-chloro-4,5-difluoro-phenyl; 2-bromo-4-fluoro-phenyl; 2-bromo-4,6-difluoro-phenyl; 4-chloro-2,5-difluoro-phenyl; 5-chloro-2,4-difluoro-phenyl; 4-bromo-2,5-difluoro-phenyl; 5-bromo-2,4-difluoro-phenyl; pentafluoro-phenyl; 2,4-dinitro-phenyl; 4-chloro-3-nitro-phenyl; 2-methyl-5-nitro-phenyl; 5-bromo-2-methoxy-phenyl; 3-chloro-2-methyl-phenyl; 4-bromo-3-methyl-phenyl; 4-chloro-2,5-dimethyl-phenyl; 4-fluoro-3-methyl-phenyl; 5-fluoro-2-methyl-phenyl; 2-nitro-4-trifluoromethyl-phenyl; 2-methoxy-4-methyl-phenyl; 3,5-d ichloro-2-hydroxy-phenyl; 3,5-dichloro-4-hydroxy-phenyl; 5-chloro-2,4-difluoro-phenyl; 3-chloro-4-(NH)—(C═O)—CH₃-phenyl; 2-chloro-6-methyl-phenyl; 2-chloro-5-trifluoromethyl-phenyl; 2-chloro-5-trifluoromethoxy-phenyl; 4-bromo-2-trifluoromethoxy-phenyl; 4-bromo-2-trifluoromethyl-phenyl; 4-bromo-3-trifluoromethyl-phenyl; 3-carboxy-4-fluoro-phenyl; 3-carboxy-4-chloro-6-fluoro-phenyl; 4-methoxy-2,3,6-trimethyl-phenyl-; or one of the following groups:

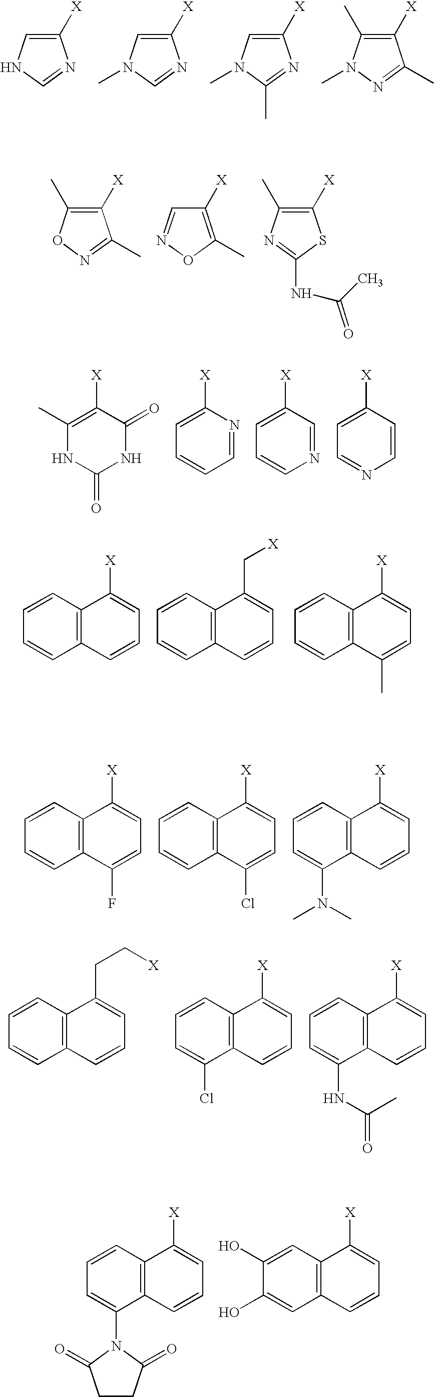

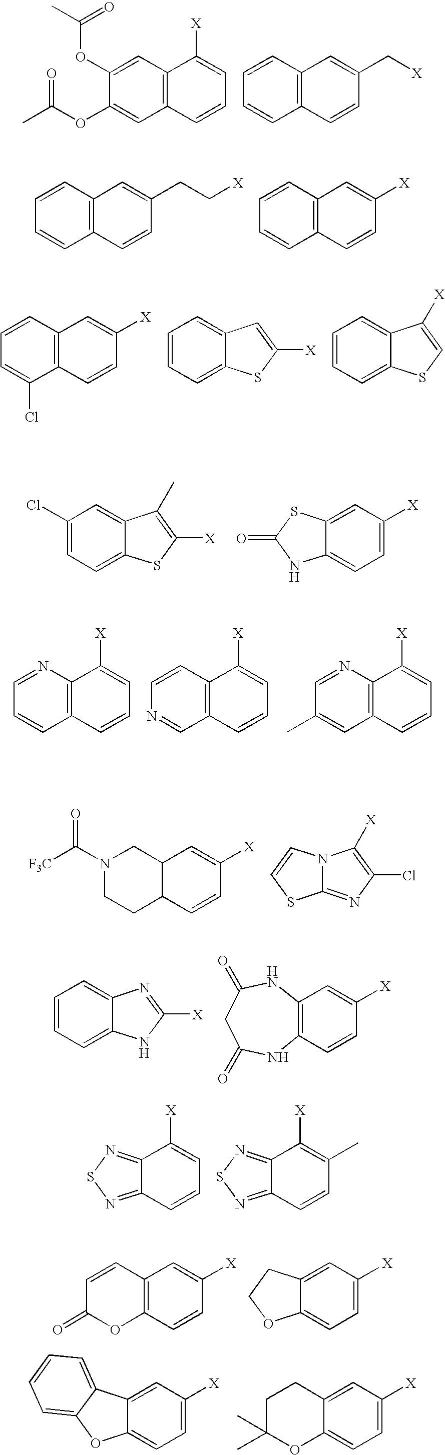

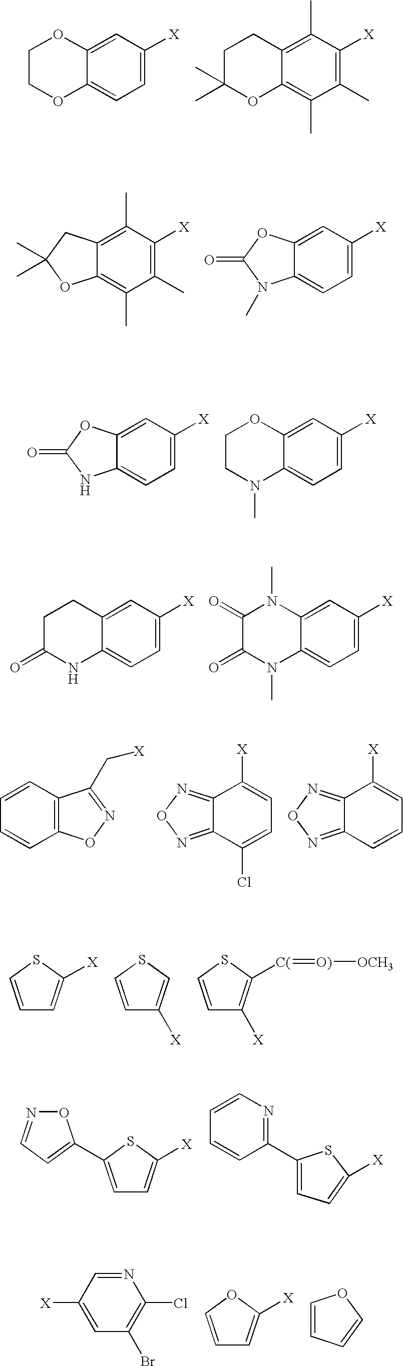

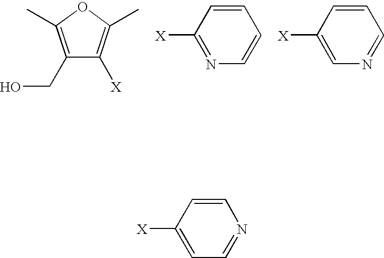

whereby in each case X denotes the position by which the respective substituent W^{a} is bonded to the —SO₂ group of formula (Ia);

optionally in form of one of its stereoisomers, preferably enantiomers or diastereomers, its racemate or in form of a mixture of at least two of its stereoisomers, preferably enantiomers or diastereomers, in any mixing ratio, or a physiologically acceptable salt thereof, or a solvate, respectively.

Particularly preferred compounds of general formula (Ia) are those selected from the group consisting of:

1a 1-[1-(Naphthalene-2-sulfonyl)-piperidin-4-yl]-1,4-dihydro-benzo[d][1,3]oxazin-2-one

1b 1-[1-(Naphthalene-1-sulfonyl)-piperidin-4-yl]-1,4-dihydro-benzo[d][1,3]oxazin-2-one

2 1-[1-(Toluene-4-sulfonyl)-piperidin-4-yl]-1,4-dihydro-benzo[d][1,3]oxazin-2-one

3 1-(1-Phenylmethanesulfonyl-piperidin-4-yl)-1,4-dihydro-benzo[d][1,3]oxazin-2-one

4 1-(1-Benzenesulfonyl-piperidin-4-yl)-6-chloro-1,4-dihydro-benzo[d][1,3]oxazin-2-one

5 6-Chloro-1-[1-(toluene-4-sulfonyl)-piperidin-4-yl]-1,4-dihydro-benzo[d][1,3]oxazin-2-one

6 6-Chloro-1-(1-phenylmethanesulfonyl-piperidin-4-yl)-1,4-dihydro-benzo[d][1,3]oxazin-2-one

7 6-Chloro-1-[1-(naphthalene-1-sulfonyl)-piperidin-4-yl]-1,4-dihydro-benzo[d][1,3]oxazin-2-one

8 6-Chloro-1-[1-(naphthalene-2-sulfonyl)-piperidin-4-yl]-1,4-dihydro-benzo[d][1,3]oxazin-2-one

9 6-Chloro-1-[1-(5-chloro-3-methyl-benzo[b]thiophene-2-sulfonyl)-piperidin-4-yl]-1,4-dihydro-benzo[d][1,3]oxazin-2-one

10 1-[1-(Thiophene-2-sulfonyl)-piperidin-4-yl]-1,4-dihydro-benzo[d][1,3]oxazin-2-one

11 1-[1-(4-Acetyl-benzenesulfonyl)-piperidin-4-yl]-1,4-dihydro-benzo[d][1,3]oxazin-2-one

12 2-[4-(2-Oxo-4H-benzo[d][1,3]oxazin-1-yl)-piperidine-1-sulfonyl]-benzonitrile

13 1-[1-(2,4-Dimethyl-benzenesulfonyl)-piperidin-4-yl]-1,4-dihydro-benzo[d][1,3]oxazin-2-one

14 1-[1-(4-Methoxy-benzenesulfonyl)-piperidin-4-yl]-1,4-dihydro-benzo[d][1,3]oxazin-2-one

15 1-[1-(2-Naphthalen-1-yl-ethanesulfonyl)-piperidin-4-yl]-1,4-dihydro-benzo[d][1,3]oxazin-2-one

16 8-Methyl-1-[1-(thiophene-2-sulfonyl)-piperidin-4-yl]-1,4-dihydro-benzo[d][1,3]oxazin-2-one

17 1-[1-(4-Acetyl-benzenesulfonyl)-piperidin-4-yl]-8-methyl-1,4-dihydro-benzo[d][1,3]oxazin-2-one

18 2-[4-(8-Methyl-2-oxo-4H-benzo[d][1,3]oxazin-1-yl)-piperidine-1-sulfonyl]-benzonitrile

19 1-[1-(2,4-Dimethyl-benzenesulfonyl)-piperidin-4-yl]-8-methyl-1,4-dihydro-benzo[d][1,3]oxazin-2-one

20 1-[1-(4-Methoxy-benzenesulfonyl)-piperidin-4-yl]-8-methyl-1,4-dihydro-benzo[d][1,3]oxazin-2-one

21 8-Methyl-1-[1-(2-naphthalen-1-yl-ethanesulfonyl)-piperidin-4-yl]-1,4-dihydro-benzo[d][1,3]oxazin-2-one

22 4-(8-Methyl-2-oxo-4H-benzo[d][1,3]oxazin-1-yl)-piperidine-1-sulfonic acid dimethylamide

23 2-[4-(2-Oxo-4H-benzo[d][1,3]oxazin-1-yl)-piperidine-1-sulfonyl]-benzoic acid methyl ester

24 1-[1-(3-Trifluoromethyl-benzenesulfonyl)-piperidin-4-yl]-1,4-dihydro-benzo[d][1,3]oxazin-2-one

25 2-[4-(8-Methyl-2-oxo-4H-benzo[d][1,3]oxazin-1-yl)-piperidine-1-sulfonyl]-benzoic acid methyl ester

26 8-Methyl-1-[1-(3-trifluoromethyl-benzenesulfonyl)-piperidin-4-yl]-1,4-dihydro-benzo[d][1,3]oxazin-2-one

27 1-[1-(4-Acetyl-benzenesulfonyl)-piperidin-4-yl]-6-chloro-1,4-dihydro-benzo[d][1,3]oxazin-2-one

28 2-[4-(6-Chloro-2-oxo-4H-benzo[d][1,3]oxazin-1-yl)-piperidine-1-sulfonyl]-benzonitrile

29 6-Chloro-1-[1-(4-methoxy-benzenesulfonyl)-piperidin-4-yl]-1,4-dihydro-benzo[d][1,3]oxazin-2-one

30 2-[4-(6-Chloro-2-oxo-4H-benzo[d][1,3]oxazin-1-yl)-piperidine-1-sulfonyl]-benzoic acid methyl ester

31 6-Chloro-1-[1-(2,4-dimethyl-benzenesulfonyl)-piperidin-4-yl]-1,4-dihydro-benzo[d][1,3]oxazin-2-one

32 6-Chloro-1-[1-(3-trifluoromethyl-benzenesulfonyl)-piperidin-4-yl]-1,4-dihydro-benzo[d][1,3]oxazin-2-one

33 1-[1-(5-Chloro-3-methyl-benzo[b]thiophene-2-sulfonyl)-piperidin-4-yl]-8-methyl-1,4-dihydro-benzo[d][1,3]oxazin-2-one

34 1-{1-[4-(4-Bromo-phenoxy)-benzenesulfonyl]-piperidin-4-yl}-8-methyl-1,4-dihydro-benzo[d][1,3]oxazin-2-one

35 1-[1-(4-Fluoro-benzenesulfonyl)-piperidin-4-yl]-8-methyl-1,4-dihydro-benzo[d][1,3]oxazin-2-one

36 8-Methyl-1-[1-(naphthalene-2-sulfonyl)-piperidin-4-yl]-1,4-dihydro-benzo[d][1,3]oxazin-2-one

37 8-Methyl-1-(1-phenylmethanesulfonyl-piperidin-4-yl)-1,4-dihydro-benzo[d][1,3]oxazin-2-one

38 1-[1-(4-Bromo-benzenesulfonyl)-piperidin-4-yl]-6-chloro-1,4-dihydro-benzo[d][1,3]oxazin-2-one

39 6-Chloro-1-[1-(4-methanesulfonyl-benzenesulfonyl)-piperidin-4-yl]-1,4-dihydro-benzo[d][1,3]oxazin-2-one

40 1-[1-(Butane-1-sulfonyl)-piperidin-4-yl]-6-chloro-1,4-dihydro-benzo[d][1,3]oxazin-2-one

41 1-[1-(4-Bromo-benzenesulfonyl)-piperidin-4-yl]-8-methyl-1,4-dihydro-benzo[d][1,3]oxazin-2-one

42 1-[1-(4-Methanesulfonyl-benzenesulfonyl)-piperidin-4-yl]-8-methyl-1,4-dihydro-benzo[d][1,3]oxazin-2-one

43 1-[1-(Butane-1-sulfonyl)-piperidin-4-yl]-8-methyl-1,4-dihydro-benzo[d][1,3]oxazin-2-one

44 6-Chloro-1-[1-(2-nitro-benzenesulfonyl)-piperidin-4-yl]-1,4-dihydro-benzo[d][1,3]oxazin-2-one

45 6-Chloro-1-[1-(3-nitro-benzenesulfonyl)-piperidin-4-yl]-1,4-dihydro-benzo[d][1,3]oxazin-2-one

46 1-[1-(Biphenyl-4-sulfonyl)-piperidin-4-yl]-6-chloro-1,4-dihydro-benzo[d][1,3]oxazin-2-one

47 8-Methyl-1-[1-(2-nitro-benzenesulfonyl)-piperidin-4-yl]-1,4-dihydro-benzo[d][1,3]oxazin-2-one

48 8-Methyl-1-[1-(3-nitro-benzenesulfonyl)-piperidin-4-yl]-1,4-dihydro-benzo[d][1,3]oxazin-2-one

49 1-[1-(Biphenyl-4-sulfonyl)-piperidin-4-yl]-8-methyl-1,4-dihydro-benzo[d][1,3]oxazin-2-one

50 8-Methyl-1-[1-(4-nitro-benzenesulfonyl)-piperidin-4-yl]-1,4-dihydro-benzo[d][1,3]oxazin-2-one

51 6-Chloro-1-[1-(4-nitro-benzenesulfonyl)-piperidin-4-yl]-1,4-dihydro-benzo[d][1,3]oxazin-2-one

52 1-(1-Ethanesulfonyl-piperidin-4-yl)-1,4-dihydro-benzo[d][1,3]oxazin-2-one

53 1-[1-(Propane-1-sulfonyl)-piperidin-4-yl]-1,4-dihydro-benzo[d][1,3]oxazin-2-one

54 1-[1-(Propane-2-sulfonyl)-piperidin-4-yl]-1,4-dihydro-benzo[d][1,3]oxazin-2-one

55 6-Chloro-1-(1-ethanesulfonyl-piperidin-4-yl)-1,4-dihydro-benzo[d][1,3]oxazin-2-one

56 6-Chloro-1-[1-(propane-1-sulfonyl)-piperidin-4-yl]-1,4-dihydro-benzo[d][1,3]oxazin-2-one

57 6-Chloro-1-[1-(propane-2-sulfonyl)-piperidin-4-yl]-1,4-dihydro-benzo[d][1,3]oxazin-2-one

58 6-Chloro-1-[1-(quinoline-8-sulfonyl)-piperidin-4-yl]-1,4-dihydro-benzo[d][1,3]oxazin-2-one

59 1-[1-(4-Nitro-benzenesulfonyl)-piperidin-4-yl]-1,4-dihydro-benzo[d][1,3]oxazin-2-one

60 6-Methyl-1-[1-(quinoline-8-sulfonyl)-piperidin-4-yl]-1,4-dihydro-benzo[d][1,3]oxazin-2-one

61 6-Methyl-1-[1-(2-naphthalen-1-yl-ethanesulfonyl)-piperidin-4-yl]-1,4-dihydro-benzo[d][1,3]oxazin-2-one

62 6-Methyl-1-[1-(toluene-4-sulfonyl)-piperidin-4-yl]-1,4-dihydro-benzo[d][1,3]oxazin-2-one

63 1-[1-(4-Fluoro-benzenesulfonyl)-piperidin-4-yl]-6-methyl-1,4-dihydro-benzo[d][1,3]oxazin-2-one

64 6-Methyl-1-[1-(naphthalene-1-sulfonyl)-piperidin-4-yl]-1,4-dihydro-benzo[d][1,3]oxazin-2-one

65 6-Methyl-1-[1-(naphthalene-2-sulfonyl)-piperidin-4-yl]-1,4-dihydro-benzo[d][1,3]oxazin-2-one

66 1-[1-(5-Chloro-3-methyl-benzo[b]thiophene-2-sulfonyl)-piperidin-4-yl]-6-methyl-1,4-dihydro-benzo[d][1,3]oxazin-2-one

67 6-Methyl-1-[1-(4-nitro-benzenesulfonyl)-piperidin-4-yl]-1,4-dihydro-benzo[d][1,3]oxazin-2-one

68 1-(1-Benzenesulfonyl-piperidin-4-yl)-6-methyl-1,4-dihydro-benzo[d][1,3]oxazin-2-one

69 1-[1-(4-Chloro-3-nitro-benzenesulfonyl)-piperidin-4-yl]-6-methyl-1,4-dihydro-benzo[d][1,3]oxazin-2-one

70 1-[1-(5-Dimethylamino-naphthalene-1-sulfonyl)-piperidin-4-yl]-6-methyl-1,4-dihydro-benzo[d][1,3]oxazin-2-one

71 1-[1-(4-Chloro-3-nitro-benzenesulfonyl)-piperidin-4-yl]-8-methyl-1,4-dihydro-benzo[d][1,3]oxazin-2-one

72 1-[1-(4-Chloro-3-nitro-benzenesulfonyl)-piperidin-4-yl]-1,4-dihydro-benzo[d][1,3]oxazin-2-one

73 6-Chloro-1-[1-(4-chloro-3-nitro-benzenesulfonyl)-piperidin-4-yl]-1,4-dihydro-benzo[d][1,3]oxazin-2-one

74 6-Chloro-1-[1-(5-dimethylamino-naphthalene-1-sulfonyl)-piperidin-4-yl]-1,4-dihydro-benzo[d][1,3]oxazin-2-one

75 1-[1-(4-Methoxy-2,3,6-trimethyl-benzenesulfonyl)-piperidin-4-yl]-1,4-dihydro-benzo[d][1,3]oxazin-2-one

76 1-[1-(4-Methoxy-2,3,6-trimethyl-benzenesulfonyl)-piperidin-4-yl]-8-methyl-1,4-dihydro-benzo[d][1,3]oxazin-2-one

77 6-Chloro-1-[1-(4-methoxy-2,3,6-trimethyl-benzenesulfonyl)-piperidin-4-yl]-1,4-dihydro-benzo[d][1,3]oxazin-2-one

78 1-[1-(4-Methoxy-2,3,6-trimethyl-benzenesulfonyl)-piperidin-4-yl]-6-methyl-1,4-dihydro-benzo[d][1,3]oxazin-2-one

79 1-[1-(2-Bromo-benzenesulfonyl)-piperidin-4-yl]-1,4-dihydro-benzo[d][1,3]oxazin-2-one

80 1-[1-(2-Bromo-benzenesulfonyl)-piperidin-4-yl]-8-methyl-1,4-dihydro-benzo[d][1,3]oxazin-2-one

81 1-[1-(2-Bromo-benzenesulfonyl)-piperidin-4-yl]-6-chloro-1,4-dihydro-benzo[d][1,3]oxazin-2-one

82 1-[1-(2-Bromo-benzenesulfonyl)-piperidin-4-yl]-6-methyl-1,4-dihydro-benzo[d][1,3]oxazin-2-one

83 6-Chloro-1-[1-(2,3-dichloro-benzenesulfonyl)-piperidin-4-yl]-1,4-dihydro-benzo[d][1,3]oxazin-2-one

84 1-[1-(2,3-Dichloro-benzenesulfonyl)-piperidin-4-yl]-6-methyl-1,4-dihydro-benzo[d][1,3]oxazin-2-one

85 1-[1-(2,4,5-Trichloro-benzenesulfonyl)-piperidin-4-yl]-1,4-dihydro-benzo[d][1,3]oxazin-2-one

86 8-Methyl-1-[1-(2,4,5-trichloro-benzenesulfonyl)-piperidin-4-yl]-1,4-dihydro-benzo[d][1,3]oxazin-2-one

87 6-Chloro-1-[1-(2,4,5-trichloro-benzenesulfonyl)-piperidin-4-yl]-1,4-dihydro-benzo[d][1,3]oxazin-2-one

88 6-Methyl-1-[1-(2,4,5-trichloro-benzenesulfonyl)-piperidin-4-yl]-1,4-dihydro-benzo[d][1,3]oxazin-2-one

89 1-[1-(5-Bromo-2-methoxy-benzenesulfonyl)-piperidin-4-yl]-1,4-dihydro-benzo[d][1,3]oxazin-2-one

90 1-[1-(5-Bromo-2-methoxy-benzenesulfonyl)-piperidin-4-yl]-8-methyl-1,4-dihydro-benzo[d][1,3]oxazin-2-one

91 1-[1-(5-Bromo-2-methoxy-benzenesulfonyl)-piperidin-4-yl]-6-chloro-1,4-dihydro-benzo[d][1,3]oxazin-2-one

92 1-[1-(5-Bromo-2-methoxy-benzenesulfonyl)-piperidin-4-yl]-6-methyl-1,4-dihydro-benzo[d][1,3]oxazin-2-one

93 1-[1-(2,5-Dimethoxy-benzenesulfonyl)-piperidin-4-yl]-1,4-dihydro-benzo[d][1,3]oxazin-2-one

94 1-[1-(2,5-Dimethoxy-benzenesulfonyl)-piperidin-4-yl]-8-methyl-1,4-dihydro-benzo[d][1,3]oxazin-2-one

95 6-Chloro-1-[1-(2,5-dimethoxy-benzenesulfonyl)-piperidin-4-yl]-1,4-dihydro-benzo[d][1,3]oxazin-2-one

96 1-[1-(2,5-Dimethoxy-benzenesulfonyl)-piperidin-4-yl]-6-methyl-1,4-dihydro-benzo[d][1,3]oxazin-2-one

97 1-(1-Pentamethylbenzenesulfonyl-piperidin-4-yl)-1,4-dihydro-benzo[d][1,3]oxazin-2-one

98 8-Methyl-1-(1-pentamethylbenzenesulfonyl-piperidin-4-yl)-1,4-dihydro-benzo[d][1,3]oxazin-2-one

99 6-Chloro-1-(1-pentamethylbenzenesulfonyl-piperidin-4-yl)-1,4-dihydro-benzo[d][1,3]oxazin-2-one

100 6-Methyl-1-(1-pentamethylbenzenesulfonyl-piperidin-4-yl)-1,4-dihydro-benzo[d][1,3]oxazin-2-one

101 1-{1-[2-(2,2,2-Trifluoro-acetyl)-1,2,3,4-tetrahydro-isoquinoline-7-sulfonyl]-piperidin-4-yl}-1,4-dihydro-benzo[d][1,3]oxazin-2-one

102 8-Methyl-1-{1-[2-(2,2,2-trifluoro-acetyl)-1,2,3,4-tetrahydro-isoquinoline-7-sulfonyl]-piperidin-4-yl}-1,4-dihydro-benzo[d][1,3]oxazin-2-one

103 6-Chloro-1-{1-[2-(2,2,2-trifluoro-acetyl)-1,2,3,4-tetrahydro-isoquinoline-7-sulfonyl]-piperidin-4-yl}-1,4-dihydro-benzo[d][1,3]oxazin-2-one

104 6-Methyl-1-{1-[2-(2,2,2-trifluoro-acetyl)-1,2,3,4-tetrahydro-isoquinoline-7-sulfonyl]-piperidin-4-yl}-1,4-dihydro-benzo[d][1,3]oxazin-2-one

105 1-[1-(2-Methyl-5-nitro-benzenesulfonyl)-piperidin-4-yl]-1,4-dihydro-benzo[d][1,3]oxazin-2-one

106 8-Methyl-1-[1-(2-methyl-5-nitro-benzenesulfonyl)-piperidin-4-yl]-1,4-dihydro-benzo[d][1,3]oxazin-2-one

107 6-Chloro-1-[1-(2-methyl-5-nitro-benzenesulfonyl)-piperidin-4-yl]-1,4-dihydro-benzo[d][1,3]oxazin-2-one

108 6-Methyl-1-[1-(2-methyl-5-nitro-benzenesulfonyl)-piperidin-4-yl]-1,4-dihydro-benzo[d][1,3]oxazin-2-one

109 1-[1-(4-Bromo-2,5-difluoro-benzenesulfonyl)-piperidin-4-yl]-1,4-dihydro-benzo[d][1,3]oxazin-2-one

110 1-[1-(4-Bromo-2,5-difluoro-benzenesulfonyl)-piperidin-4-yl]-8-methyl-1,4-dihydro-benzo[d][1,3]oxazin-2-one

111 1-[1-(4-Bromo-2,5-difluoro-benzenesulfonyl)-piperidin-4-yl]-6-chloro-1,4-dihydro-benzo[d][1,3]oxazin-2-one

112 1-[1-(4-Bromo-2,5-difluoro-benzenesulfonyl)-piperidin-4-yl]-6-methyl-1,4-dihydro-benzo[d][1,3]oxazin-2-one

113 1-[1-(4-Chloro-2,5-dimethyl-benzenesulfonyl)-piperidin-4-yl]-1,4-dihydro-benzo[d][1,3]oxazin-2-one

114 1-[1-(4-Chloro-2,5-dimethyl-benzenesulfonyl)-piperidin-4-yl]-8-methyl-1,4-dihydro-benzo[d][1,3]oxazin-2-one

115 6-Chloro-1-[1-(4-chloro-2,5-dimethyl-benzenesulfonyl)-piperidin-4-yl]-1,4-dihydro-benzo[d][1,3]oxazin-2-one

116 1-[1-(4-Chloro-2,5-dimethyl-benzenesulfonyl)-piperidin-4-yl]-6-methyl-1,4-dihydro-benzo[d][1,3]oxazin-2-one

117 1-[1-(4-Methoxy-benzenesulfonyl)-piperidin-4-yl]-6-methyl-1,4-dihydro-benzo[d][1,3]oxazin-2-one

118 1-[1-(4-Isopropyl-benzenesulfonyl)-piperidin-4-yl]-1,4-dihydro-benzo[d][1,3]oxazin-2-one

119 1-[1-(4-Isopropyl-benzenesulfonyl)-piperidin-4-yl]-8-methyl-1,4-dihydro-benzo[d][1,3]oxazin-2-one

120 6-Chloro-1-[1-(4-isopropyl-benzenesulfonyl)-piperidin-4-yl]-1,4-dihydro-benzo[d][1,3]oxazin-2-one

121 1-[1-(4-Isopropyl-benzenesulfonyl)-piperidin-4-yl]-6-methyl-1,4-dihydro-benzo[d][1,3]oxazin-2-one

122 1-[1-(3-Chloro-4-fluoro-benzenesulfonyl)-piperidin-4-yl]-1,4-dihydro-benzo[d][1,3]oxazin-2-one

123 1-[1-(3-Chloro-4-fluoro-benzenesulfonyl)-piperidin-4-yl]-8-methyl-1,4-dihydro-benzo[d][1,3]oxazin-2-one

124 6-Chloro-1-[1-(3-chloro-4-fluoro-benzenesulfonyl)-piperidin-4-yl]-1,4-dihydro-benzo[d][1,3]oxazin-2-one

125 1-[1-(3-Chloro-4-fluoro-benzenesulfonyl)-piperidin-4-yl]-6-methyl-1,4-dihydro-benzo[d][1,3]oxazin-2-one

126 1-[1-(4-Bromo-benzenesulfonyl)-piperidin-4-yl]-6-methyl-1,4-dihydro-benzo[d][1,3]oxazin-2-one

127 6-Methyl-1-[1-(3-nitro-benzenesulfonyl)-piperidin-4-yl]-1,4-dihydro-benzo[d][1,3]oxazin-2-one

128 6-Methyl-1-[1-(3-trifluoromethyl-benzenesulfonyl)-piperidin-4-yl]-1,4-dihydro-benzo[d][1,3]oxazin-2-one

129 1-[1-(4-Trifluoromethoxy-benzenesulfonyl)-piperidin-4-yl]-1,4-dihydro-benzo[d][1,3]oxazin-2-one

130 1-[1-(2-Nitro-4-trifluoromethyl-benzenesulfonyl)-piperidin-4-yl]-1,4-dihydro-benzo[d][1,3]oxazin-2-one

131 1-[1-(3-Fluoro-benzenesulfonyl)-piperidin-4-yl]-1,4-dihydro-benzo[d][1,3]oxazin-2-one

132 1-[1-(2,4-Dichloro-benzenesulfonyl)-piperidin-4-yl]-1,4-dihydro-benzo[d][1,3]oxazin-2-one

133 1-[1-(2,4,6-Trimethyl-benzenesulfonyl)-piperidin-4-yl]-1,4-dihydro-benzo[d][1,3]oxazin-2-one

134 1-[1-(2-Trifluoromethyl-benzenesulfonyl)-piperidin-4-yl]-1,4-dihydro-benzo[d][1,3]oxazin-2-one

135 8-Methyl-1-[1-(4-trifluoromethoxy-benzenesulfonyl)-piperidin-4-yl]-1,4-dihydro-benzo[d][1,3]oxazin-2-one

136 8-Methyl-1-[1-(2-nitro-4-trifluoromethyl-benzenesulfonyl)-piperidin-4-yl]-1,4-dihydro-benzo[d][1,3]oxazin-2-one

137 1-[1-(3-Fluoro-benzenesulfonyl)-piperidin-4-yl]-8-methyl-1,4-dihydro-benzo[d][1,3]oxazin-2-one

138 1-[1-(2,4-Dichloro-benzenesulfonyl)-piperidin-4-yl]-8-methyl-1,4-dihydro-benzo[d][1,3]oxazin-2-one

139 8-Methyl-1-[1-(2,4,6-trimethyl-benzenesulfonyl)-piperidin-4-yl]-1,4-dihydro-benzo[d][1,3]oxazin-2-one

140 8-Methyl-1-[1-(2-trifluoromethyl-benzenesulfonyl)-piperidin-4-yl]-1,4-dihydro-benzo[d][1,3]oxazin-2-one

141 1-[1-(4-Fluoro-benzenesulfonyl)-piperidin-4-yl]-1,4-dihydro-benzo[d][1,3]oxazin-2-one

142 1-[1-(4-Bromo-benzenesulfonyl)-piperidin-4-yl]-1,4-dihydro-benzo[d][1,3]oxazin-2-one

143 1-[1-(3-Nitro-benzenesulfonyl)-piperidin-4-yl]-1,4-dihydro-benzo[d][1,3]oxazin-2-one

144 1-{1-[1-(4-Bromo-phenoxy)-benzenesulfonyl]-piperidin-4-yl}-1,4-dihydro-benzo[d][1,3]oxazin-2-one

145 1-[1-(3-Methoxy-benzenesulfonyl)-piperidin-4-yl]-1,4-dihydro-benzo[d][1,3]oxazin-2-one

146 1-[1-(2-Nitro-benzenesulfonyl)-piperidin-4-yl]-1,4-dihydro-benzo[d][1,3]oxazin-2-one

147 8-Methyl-1-[1-(toluene-4-sulfonyl)-piperidin-4-yl]-1,4-dihydro-benzo[d][1,3]oxazin-2-one

148 1-(1-Benzenesulfonyl-piperidin-4-yl)-8-methyl-1,4-dihydro-benzo[d][1,3]oxazin-2-one

149 1-[1-(3-Methoxy-benzenesulfonyl)-piperidin-4-yl]-8-methyl-1,4-dihydro-benzo[d][1,3]oxazin-2-one

150 1-[1-(2,4-Dimethyl-benzenesulfonyl)-piperidin-4-yl]-6-methyl-1,4-dihydro-benzo[d][1,3]oxazin-2-one

151 1-{1-[4-(4-Bromo-phenoxy)-benzenesulfonyl]-piperidin-4-yl}-6-methyl-1,4-dihydro-benzo[d][1,3]oxazin-2-one

152 6-Methyl-1-[1-(thiophene-2-sulfonyl)-piperidin-4-yl]-1,4-dihydro-benzo[d][1,3]oxazin-2-one

153 1-[1-(Toluene-3-sulfonyl)-piperidin-4-yl]-1,4-dihydro-benzo[d][1,3]oxazin-2-one

154 1-[1-(5-Fluoro-2-methyl-benzenesulfonyl)-piperidin-4-yl]-1,4-dihydro-benzo[d][1,3]oxazin-2-one

155 1-[1-(4-Isopropoxy-benzenesulfonyl)-piperidin-4-yl]-1,4-dihydro-benzo[d][1,3]oxazin-2-one

156 1-[1-(3-Chloro-benzenesulfonyl)-piperidin-4-yl]-1,4-dihydro-benzo[d][1,3]oxazin-2-one

157 1-[1-(3,4-Dimethoxy-benzenesulfonyl)-piperidin-4-yl]-1,4-dihydro-benzo[d][1,3]oxazin-2-one

158 1-(1-Pentafluorobenzenesulfonyl-piperidin-4-yl)-1,4-dihydro-benzo[d][1,3]oxazin-2-one

159 8-Methyl-1-[1-(toluene-3-sulfonyl)-piperidin-4-yl]-1,4-dihydro-benzo[d][1,3]oxazin-2-one

160 1-[1-(5-Fluoro-2-methyl-benzenesulfonyl)-piperidin-4-yl]-8-methyl-1,4-dihydrobenzo[d][1,3]oxazin-2-one

161 1-[1-(4-Isopropoxy-benzenesulfonyl)-piperidin-4-yl]-8-methyl-1,4-dihydro-benzo[d][1,3]oxazin-2-one

162 1-[1-(3-Chloro-benzenesulfonyl)-piperidin-4-yl]-8-methyl-1,4-dihydro-benzo[d][1,3]oxazin-2-one

163 1-[1-(3,4-Dimethoxy-benzenesulfonyl)-piperidin-4-yl]-8-methyl-1,4-dihydro-benzo[d][1,3]oxazin-2-one

164 8-Methyl-1-(1-pentafluorobenzenesulfonyl-piperidin-4-yl)-1,4-dihydro-benzo[d][1,3]oxazin-2-one

165 6-Methyl-1-[1-(toluene-3-sulfonyl)-piperidin-4-yl]-1,4-dihydro-benzo[d][1,3]oxazin-2-one

166 1-[1-(5-Fluoro-2-methyl-benzenesulfonyl)-piperidin-4-yl]-6-methyl-1,4-dihydro-benzo[d][1,3]oxazin-2-one

167 1-[1-(4-Isopropoxy-benzenesulfonyl)-piperidin-4-yl]-6-methyl-1,4-dihydro-benzo[d][1,3]oxazin-2-one

168 1-[1-(3-Chloro-benzenesulfonyl)-piperidin-4-yl]-6-methyl-1,4-dihydro-benzo[d][1,3]oxazin-2-one

169 1-[1-(3,4-Dimethoxy-benzenesulfonyl)-piperidin-4-yl]-6-methyl-1,4-dihydro-benzo[d][1,3]oxazin-2-one

170 6-Methyl-1-(1-pentafluorobenzenesulfonyl-piperidin-4-yl)-1,4-dihydro-benzo[d][1,3]oxazin-2-one

171 6-Methyl-1-[1-(4-trifluoromethoxy-benzenesulfonyl)-piperidin-4-yl]-1,4-dihydro-benzo[d][1,3]oxazin-2-one

172 6-Methyl-1-[1-(2-nitro-4-trifluoromethyl-benzenesulfonyl)-piperidin-4-yl]-1,4-dihydro-benzo[d][1,3]oxazin-2-one

173 1-[1-(3-Fluoro-benzenesulfonyl)-piperidin-4-yl]-6-methyl-1,4-dihydro-benzo[d][1,3]oxazin-2-one

174 1-[1-(2,4-Dichloro-benzenesulfonyl)-piperidin-4-yl]-6-methyl-1,4-dihydro-benzo[d][1,3]oxazin-2-one

175 6-Methyl-1-[1-(2,4,6-trimethyl-benzenesulfonyl)-piperidin-4-yl]-1,4-dihydro-benzo[d][1,3]oxazin-2-one

176 6-Methyl-1-[1-(2-trifluoromethyl-benzenesulfonyl)-piperidin-4-yl]-1,4-dihydro-benzo[d][1,3]oxazin-2-one

177 1-[1-(3-Methoxy-benzenesulfonyl)-piperidin-4-yl]-6-methyl-1,4-dihydro-benzo[d][1,3]oxazin-2-one

178 6-Methyl-1-[1-(2-nitro-benzenesulfonyl)-piperidin-4-yl]-1,4-dihydro-benzo[d][1,3]oxazin-2-one

179 1-[1-(4-Acetyl-benzenesulfonyl)-piperidin-4-yl]-6-methyl-1,4-dihydro-benzo[d][1,3]oxazin-2-one

180 1-[1-(4-Methanesulfonyl-benzenesulfonyl)-piperidin-4-yl]-6-methyl-1,4-dihydro-benzo[d][1,3]oxazin-2-one

181 6-Methyl-1-(1-phenylmethanesulfonyl-piperidin-4-yl)-1,4-dihydro-benzo[d][1,3]oxazin-2-one

182 2-[4-(6-Methyl-2-oxo-4H-benzo[d][1,3]oxazin-1-yl)-piperidine-1-sulfonyl]benzoic acid methyl ester

183 6-Methyl-1-[1-(2-oxo-2H-chromene-6-sulfonyl)-piperidin-4-yl]-1,4-dihydro-benzo[d][1,3]oxazin-2-one

184 6-Chloro-1-[1-(4-fluoro-benzenesulfonyl)-piperidin-4-yl]-1,4-dihydro-benzo[d][1,3]oxazin-2-one

185 6-Chloro-1-[1-(3,5-dichloro-benzenesulfonyl)-piperidin-4-yl]-1,4-dihydro-benzo[d][1,3]oxazin-2-one

186 1-{1-[4-(4-Bromo-phenoxy)-benzenesulfonyl]-piperidin-4-yl}-6-chloro-1,4-dihydro-benzo[d][1,3]oxazin-2-one

187 6-Chloro-1-[1-(thiophene-2-sulfonyl)-piperidin-4-yl]-1,4-dihydro-benzo[d][1,3]oxazin-2-one

188 6-Chloro-1-[1-(3-methoxy-benzenesulfonyl)-piperidin-4-yl]-1,4-dihydro-benzo[d][1,3]oxazin-2-one

189 6-Chloro-1-[1-(2-oxo-2H-chromene-6-sulfonyl)-piperidin-4-yl]-1,4-dihydro-benzo[d][1,3]oxazin-2-one

190 6-Chloro-1-[1-(toluene-3-sulfonyl)-piperidin-4-yl]-1,4-dihydro-benzo[d][1,3]oxazin-2-one

191 6-Chloro-1-[1-(5-fluoro-2-methyl-benzenesulfonyl)-piperidin-4-yl]-1,4-dihydro-benzo[d][1,3]oxazin-2-one

192 6-Chloro-1-[1-(4-isopropoxy-benzenesulfonyl)-piperidin-4-yl]-1,4-dihydro-benzo[d][1,3]oxazin-2-one

193 6-Chloro-1-[1-(3-chloro-benzenesulfonyl)-piperidin-4-yl]-1,4-dihydro-benzo[d][1,3]oxazin-2-one

194 6-Chloro-1-[1-(3,4-dimethoxy-benzenesulfonyl)-piperidin-4-yl]-1,4-dihydro-benzo[d][1,3]oxazin-2-one

195 6-Chloro-1-(1-pentafluorobenzenesulfonyl-piperidin-4-yl)-1,4-dihydro-benzo[d][1,3]oxazin-2-one

196 6-Chloro-1-[1-(4-trifluoromethoxy-benzenesulfonyl)-piperidin-4-yl]-1,4-dihydro-benzo[d][1,3]oxazin-2-one

197 6-Chloro-1-[1-(2-nitro-4-trifluoromethyl-benzenesulfonyl)-piperidin-4-yl]-1,4-dihydro-benzo[d][1,3]oxazin-2-one

198 6-Chloro-1-[1-(3-fluoro-benzenesulfonyl)-piperidin-4-yl]-1,4-dihydro-benzo[d][1,3]oxazin-2-one

199 6-Chloro-1-[1-(2,4-dichloro-benzenesulfonyl)-piperidin-4-yl]-1,4-dihydro-benzo[d][1,3]oxazin-2-one

200 6-Chloro-1-[1-(2,4,6-trimethyl-benzenesulfonyl)-piperidin-4-yl]-1,4-dihydro-benzo[d][1,3]oxazin-2-one

201 6-Chloro-1-[1-(2-trifluoromethyl-benzenesulfonyl)-piperidin-4-yl]-1,4-dihydro-benzo[d][1,3]oxazin-2-one

202 1-[1-(2-Oxo-2H-chromene-6-sulfonyl)-piperidin-4-yl]-1,4-dihydro-benzo[d][1,3]oxazin-2-one

203 1-[1-(3,5-Dichloro-benzenesulfonyl)-piperidin-4-yl]-1,4-dihydro-benzo[d][1,3]oxazin-2-one

204 1-[1-(2,5-Dichloro-benzenesulfonyl)-piperidin-4-yl]-1,4-dihydro-benzo[d][1,3]oxazin-2-one

205 1-[1-(5-Bromo-6-chloro-pyridine-3-sulfonyl)-piperidin-4-yl]-1,4-dihydro-benzo[d][1,3]oxazin-2-one

206 1-[1-(4-Chloro-benzenesulfonyl)-piperidin-4-yl]-1,4-dihydro-benzo[d][1,3]oxazin-2-one

207 1-[1-(2,6-Dichloro-benzenesulfonyl)-piperidin-4-yl]-1,4-dihydro-benzo[d][1,3]oxazin-2-one

208 8-Methyl-1-[1-(2-oxo-2H-chromene-6-sulfonyl)-piperidin-4-yl]-1,4-dihydro-benzo[d][1,3]oxazin-2-one

209 1-[1-(3,5-Dichloro-benzenesulfonyl)-piperidin-4-yl]-8-methyl-1,4-dihydro-benzo[d][1,3]oxazin-2-one

210 1-[1-(2,5-Dichloro-benzenesulfonyl)-piperidin-4-yl]-8-methyl-1,4-dihydro-benzo[d][1,3]oxazin-2-one

211 1-[1-(5-Bromo-6-chloro-pyridine-3-sulfonyl)-piperidin-4-yl]-8-methyl-1,4-dihydro-benzo[d][1,3]oxazin-2-one

212 1-[1-(4-Chloro-benzenesulfonyl)-piperidin-4-yl]-8-methyl-1,4-dihydro-benzo[d][1,3]oxazin-2-one

213 1-[1-(2,6-Dichloro-benzenesulfonyl)-piperidin-4-yl]-8-methyl-1,4-dihydro-benzo[d][1,3]oxazin-2-one

214 1-[1-(Biphenyl-4-sulfonyl)-piperidin-4-yl]-1,4-dihydro-benzo[d][1,3]oxazin-2-one

215 6-Chloro-1-[1-(2,5-dichloro-benzenesulfonyl)-piperidin-4-yl]-1,4-dihydro-benzo[d][1,3]oxazin-2-one

216 1-[1-(5-Bromo-6-chloro-pyridine-3-sulfonyl)-piperidin-4-yl]-6-chloro-1,4-dihydro-benzo[d][1,3]oxazin-2-one

217 6-Chloro-1-[1-(4-chloro-benzenesulfonyl)-piperidin-4-yl]-1,4-dihydro-benzo[d][1,3]oxazin-2-one

218 6-Chloro-1-[1-(2,6-dichloro-benzenesulfonyl)-piperidin-4-yl]-1,4-dihydro-benzo[d][1,3]oxazin-2-one

219 1-[1-(Biphenyl-4-sulfonyl)-piperidin-4-yl]-6-methyl-1,4-dihydro-benzo[d][1,3]oxazin-2-one

220 2-[4-(6-Methyl-2-oxo-4H-benzo[d][1,3]oxazin-1-yl)-piperidine-1-sulfonyl]-benzonitrile

221 1-[1-(2,5-Dichloro-benzenesulfonyl)-piperidin-4-yl]-6-methyl-1,4-dihydro-benzo[d][1,3]oxazin-2-one

222 1-[1-(5-Bromo-6-chloro-pyridine-3-sulfonyl)-piperidin-4-yl]-6-methyl-1,4-dihydro-benzo[d][1,3]oxazin-2-one

223 1-[1-(4-Chloro-benzenesulfonyl)-piperidin-4-yl]-6-methyl-1,4-dihydro-benzo[d][1,3]oxazin-2-one

224 1-[1-(2,6-Dichloro-benzenesulfonyl)-piperidin-4-yl]-6-methyl-1,4-dihydro-benzo[d][1,3]oxazin-2-one

225 1-[1-(3,5-Dichloro-benzenesulfonyl)-piperidin-4-yl]-6-methyl-1,4-dihydro-benzo[d][1,3]oxazin-2-one

226 6-Methyl-1-[1-(1-methyl-1H-imidazole-4-sulfonyl)-piperidin-4-yl]-1,4-dihydro-benzo[d][1,3]oxazin-2-one

227 1-[1-(5-Bromo-2,4-difluoro-benzenesulfonyl)-piperidin-4-yl]-6-methyl-1,4-dihydro-benzo[d][1,3]oxazin-2-one

228 1-[1-(4-Methanesulfonyl-benzenesulfonyl)-piperidin-4-yl]-1,4-dihydro-benzo[d][1,3]oxazin-2-one

229 1-[1-(1-Methyl-1H-imidazole-4-sulfonyl)-piperidin-4-yl]-1,4-dihydro-benzo[d][1,3]oxazin-2-one

230 1-[1-(5-Bromo-2,4-difluoro-benzenesulfonyl)-piperidin-4-yl]-1,4-dihydro-benzo[d][1,3]oxazin-2-one

231 1-[1-(6-Chloro-imidazo[2,1-b]thiazole-5-sulfonyl)-piperidin-4-yl]-1,4-dihydro-benzo[d][1,3]oxazin-2-one

232 1-[1-(4-Ethyl-benzenesulfonyl)-piperidin-4-yl]-1,4-dihydro-benzo[d][1,3]oxazin-2-one

233 1-[1-(Benzo[b]thiophene-3-sulfonyl)-piperidin-4-yl]-1,4-dihydro-benzo[d][1,3]oxazin-2-one

234 1-[1-(6-Chloro-imidazo[2,1-b]thiazole-5-sulfonyl)-piperidin-4-yl]-8-methyl-1,4-dihydro-benzo[d][1,3]oxazin-2-one

235 1-[1-(4-Ethyl-benzenesulfonyl)-piperidin-4-yl]-8-methyl-1,4-dihydro-benzo[d][1,3]oxazin-2-one

236 1-[1-(Benzo[b]thiophene-3-sulfonyl)-piperidin-4-yl]-8-methyl-1,4-dihydro-benzo[d][1,3]oxazin-2-one

237 6-Chloro-1-[1-(6-chloro-imidazo[2,1-b]thiazole-5-sulfonyl)-piperidin-4-yl]-1,4-dihydro-benzo[d][1,3]oxazin-2-one

238 6-Chloro-1-[1-(4-ethyl-benzenesulfonyl)-piperidin-4-yl]-1,4-dihydro-benzo[d][1,3]oxazin-2-one

239 1-[1-(Benzo[b]thiophene-3-sulfonyl)-piperidin-4-yl]-6-chloro-1,4-dihydro-benzo[d][1,3]oxazin-2-one

240 1-[1-(6-Chloro-imidazo[2,1-b]thiazole-5-sulfonyl)-piperidin-4-yl]-6-methyl-1,4-dihydro-benzo[d][1,3]oxazin-2-one

241 1-[1-(4-Ethyl-benzenesulfonyl)-piperidin-4-yl]-6-methyl-1,4-dihydro-benzo[d][1,3]oxazin-2-one

242 1-[1-(Benzo[b]thiophene-3-sulfonyl)-piperidin-4-yl]-6-methyl-1,4-dihydro-benzo[d][1,3]oxazin-2-one

243 1-[1-(7-Chloro-benzo[1,2,5]oxadiazole-4-sulfonyl)-piperidin-4-yl]-1,4-dihydro-benzo[d][1,3]oxazin-2-one

244 1-[1-(2-Methoxy-4-methyl-benzenesulfonyl)-piperidin-4-yl]-1,4-dihydro-benzo[d][1,3]oxazin-2-one

245 3-{4-[4-(2-Oxo-4H-benzo[d][1,3]oxazin-1-yl)-piperidine-1-sulfonyl]-phenyl}-propionic acid methyl ester

246 1-[1-(2,4-Dinitro-benzenesulfonyl)-piperidin-4-yl]-1,4-dihydro-benzo[d][1,3]oxazin-2-one

247 1-[1-(7-Chloro-benzo[1,2,5]oxadiazole-4-sulfonyl)-piperidin-4-yl]-8-methyl-1,4-dihydro-benzo[d][1,3]oxazin-2-one

248 1-[1-(2-Methoxy-4-methyl-benzenesulfonyl)-piperidin-4-yl]-8-methyl-1,4-dihydro-benzo[d][1,3]oxazin-2-one

249 3-{4-[4-(8-Methyl-2-oxo-4H-benzo[d][1,3]oxazin-1-yl)-piperidine-1-sulfonyl]-phenyl}-propionic acid methyl ester

250 1-[1-(2,4-Dinitro-benzenesulfonyl)-piperidin-4-yl]-8-methyl-1,4-dihydro-benzo[d][1,3]oxazin-2-one

251 1-[1-(7-Chloro-benzo[1,2,5]oxadiazole-4-sulfonyl)-piperidin-4-yl]-6-methyl-1,4-dihydro-benzo[d][1,3]oxazin-2-one

252 1-[1-(2-Methoxy-4-methyl-benzenesulfonyl)-piperidin-4-yl]-6-methyl-1,4-dihydro-benzo[d][1,3]oxazin-2-one

253 3-{4-[4-(6-Methyl-2-oxo-4H-benzo[d][1,3]oxazin-1-yl)-piperidine-1-sulfonyl]-phenyl}-propionic acid methyl ester

254 1-[1-(2,4-Dinitro-benzenesulfonyl)-piperidin-4-yl]-6-methyl-1,4-dihydro-benzo[d][1,3]oxazin-2-one

255 6-Chloro-1-[1-(7-chloro-benzo[1,2,5]oxadiazole-4-sulfonyl)-piperidin-4-yl]-1,4-dihydro-benzo[d][1,3]oxazin-2-one

256 6-Chloro-1-[1-(2-methoxy-4-methyl-benzenesulfonyl)-piperidin-4-yl]-1,4-dihydro-benzo[d][1,3]oxazin-2-one

257 3-{4-[4-(6-Chloro-2-oxo-4H-benzo[d][1,3]oxazin-1-yl)-piperidine-1-sulfonyl]-phenyl}-propionic acid methyl ester

258 6-Chloro-1-[1-(2,4-dinitro-benzenesulfonyl)-piperidin-4-yl]-1,4-dihydro-benzo[d][1,3]oxazin-2-one

259 6-Chloro-1-[1-(1-methyl-1H-imidazole-4-sulfonyl)-piperidin-4-yl]-1,4-dihydro-benzo[d][1,3]oxazin-2-one

260 1-[1-(5-Bromo-2,4-difluoro-benzenesulfonyl)-piperidin-4-yl]-6-chloro-1,4-dihydro-benzo[d][1,3]oxazin-2-one

261 8-Methyl-1-[1-(1-methyl-1H-imidazole-4-sulfonyl)-piperidin-4-yl]-1,4-dihydro-benzo[d][1,3]oxazin-2-one

262 1-[1-(5-Bromo-2,4-difluoro-benzenesulfonyl)-piperidin-4-yl]-8-methyl-1,4-dihydro-benzo[d][1,3]oxazin-2-one

263 1-[1-(Benzo[b]thiophene-2-sulfonyl)-piperidin-4-yl]-8-methyl-1,4-dihydro-benzo[d][1,3]oxazin-2-one

264 1-[1-(Benzo[b]thiophene-2-sulfonyl)-piperidin-4-yl]-1,4-dihydro-benzo[d][1,3]oxazin-2-one

265 1-[1-(Benzo[b]thiophene-2-sulfonyl)-piperidin-4-yl]-6-chloro-1,4-dihydro-benzo[d][1,3]oxazin-2-one

266 1-[1-(Benzo[b]thiophene-2-sulfonyl)-piperidin-4-yl]-6-methyl-1,4-dihydro-benzo[d][1,3]oxazin-2-one

267 1-[1-(2,5-Difluoro-benzenesulfonyl)-piperidin-4-yl]-1,4-dihydro-benzo[d][1,3]oxazin-2-one

268 1-[1-(2,5-Difluoro-benzenesulfonyl)-piperidin-4-yl]-8-methyl-1,4-dihydro-benzo[d][1,3]oxazin-2-one

269 6-Chloro-1-[1-(2,5-difluoro-benzenesulfonyl)-piperidin-4-yl]-1,4-dihydro-benzo[d][1,3]oxazin-2-one

270 1-[1-(2,5-Difluoro-benzenesulfonyl)-piperidin-4-yl]-6-methyl-1,4-dihydro-benzo[d][1,3]oxazin-2-one

271 1-[1-(4-Chloro-2,5-difluoro-benzenesulfonyl)-piperidin-4-yl]-1,4-dihydro-benzo[d][1,3]oxazin-2-one

272 1-[1-(4-Chloro-2,5-difluoro-benzenesulfonyl)-piperidin-4-yl]-8-methyl-1,4-dihydro-benzo[d][1,3]oxazin-2-one

273 6-Chloro-1-[1-(4-chloro-2,5-difluoro-benzenesulfonyl)-piperidin-4-yl]-1,4-dihydro-benzo[d][1,3]oxazin-2-one

274 1-[1-(4-Chloro-2,5-difluoro-benzenesulfonyl)-piperidin-4-yl]-6-methyl-1,4-dihydro-benzo[d][1,3]oxazin-2-one

275 1-[1-(2,4,5-Trifluoro-benzenesulfonyl)-piperidin-4-yl]-1,4-dihydro-benzo[d][1,3]oxazin-2-one

276 8-Methyl-1-[1-(2,4,5-trifluoro-benzenesulfonyl)-piperidin-4-yl]-1,4-dihydro-benzo[d][1,3]oxazin-2-one

277 6-Chloro-1-[1-(2,4,5-trifluoro-benzenesulfonyl)-piperidin-4-yl]-1,4-dihydro-benzo[d][1,3]oxazin-2-one

278 6-Methyl-1-[1-(2,4,5-trifluoro-benzenesulfonyl)-piperidin-4-yl]-1,4-dihydro-benzo[d][1,3]oxazin-2-one

279 1-[1-(3,5-Dichloro-2-hydroxy-benzenesulfonyl)-piperidin-4-yl]-1,4-dihydro-benzo[d][1,3]oxazin-2-one

280 1-[1-(2,6-Difluoro-benzenesulfonyl)-piperidin-4-yl]-1,4-dihydro-benzo[d][1,3]oxazin-2-one

281 1-[1-(2,6-Difluoro-benzenesulfonyl)-piperidin-4-yl]-8-methyl-1,4-dihydro-benzo[d][1,3]oxazin-2-one

282 6-Chloro-1-[1-(2,6-difluoro-benzenesulfonyl)-piperidin-4-yl]-1,4-dihydro-benzo[d][1,3]oxazin-2-one

283 1-[1-(2,6-Difluoro-benzenesulfonyl)-piperidin-4-yl]-6-methyl-1,4-dihydro-benzo[d][1,3]oxazin-2-one

284 1-[1-(5-Chloro-2,4-difluoro-benzenesulfonyl)-piperidin-4-yl]-1,4-dihydro-benzo[d][1,3]oxazin-2-one

285 1-[1-(5-Chloro-2,4-difluoro-benzenesulfonyl)-piperidin-4-yl]-8-methyl-1,4-dihydro-benzo[d][1,3]oxazin-2-one

286 6-Chloro-1-[1-(5-chloro-2,4-difluoro-benzenesulfonyl)-piperidin-4-yl]-1,4-dihydro-benzo[d][1,3]oxazin-2-one

287 1-[1-(5-Chloro-2,4-difluoro-benzenesulfonyl)-piperidin-4-yl]-6-methyl-1,4-dihydro-benzo[d][1,3]oxazin-2-one

288 1-[1-(2-Chloro-benzenesulfonyl)-piperidin-4-yl]-1,4-dihydro-benzo[d][1,3]oxazin-2-one

289 1-[1-(2-Chloro-benzenesulfonyl)-piperidin-4-yl]-8-methyl-1,4-dihydro-benzo[d][1,3]oxazin-2-one

290 6-Chloro-1-[1-(2-chloro-benzenesulfonyl)-piperidin-4-yl]-1,4-dihydro-benzo[d][1,3]oxazin-2-one

291 1-[1-(2-Chloro-benzenesulfonyl)-piperidin-4-yl]-6-methyl-1,4-dihydro-benzo[d][1,3]oxazin-2-one

292 6-Chloro-1-[1-(2-naphthalen-1-yl-ethanesulfonyl)-piperidin-4-yl]-1,4-dihydro-benzo[d][1,3]oxazin-2-one

293 6-Bromo-1-[1-(4-bromo-benzenesulfonyl)-piperidin-4-yl]-1,4-dihydro-benzo[d][1,3]oxazin-2-one

294 6-Bromo-1-[1-(toluene-4-sulfonyl)-piperidin-4-yl]-1,4-dihydro-benzo[d][1,3]oxazin-2-one

295 6-Bromo-1-[1-(2,4-dimethyl-benzenesulfonyl)-piperidin-4-yl]-1,4-dihydro-benzo[d][1,3]oxazin-2-one

296 6-Bromo-1-[1-(2-naphthalen-1-yl-ethanesulfonyl)-piperidin-4-yl]-1,4-dihydro-benzo[d][1,3]oxazin-2-one

297 6-Bromo-1-[1-(quinoline-8-sulfonyl)-piperidin-4-yl]-1,4-dihydro-benzo[d][1,3]oxazin-2-one

298 6-Bromo-1-[1-(5-chloro-3-methyl-benzo[b]thiophene-2-sulfonyl)-piperidin-4-yl]-1,4-dihydro-benzo[d][1,3]oxazin-2-one

299 6-Bromo-1-[1-(3-nitro-benzenesulfonyl)-piperidin-4-yl]-1,4-dihydro-benzo[d][1,3]oxazin-2-one

300 6-Bromo-1-[1-(naphthalene-1-sulfonyl)-piperidin-4-yl]-1,4-dihydro-benzo[d][1,3]oxazin-2-one

301 6-Bromo-1-[1-(naphthalene-2-sulfonyl)-piperidin-4-yl]-1,4-dihydro-benzo[d][1,3]oxazin-2-one

302 1-(1-Benzenesulfonyl-piperidin-4-yl)-6-bromo-1,4-dihydro-benzo[d][1,3]oxazin-2-one

303 6-Bromo-1-{1-[1-(4-bromo-phenoxy)-benzenesulfonyl]-piperidin-4-yl}-1,4-dihydro-benzo[d][1,3]oxazin-2-one

304 6-Bromo-1-[1-(thiophene-2-sulfonyl)-piperidin-4-yl]-1,4-dihydro-benzo[d][1,3]oxazin-2-one

305 6-Bromo-1-[1-(2-methyl-5-nitro-benzenesulfonyl)-piperidin-4-yl]-1,4-dihydro-benzo[d][1,3]oxazin-2-one

306 6-Bromo-1-[1-(4-bromo-2,5-difluoro-benzenesulfonyl)-piperidin-4-yl]-1,4-dihydro-benzo[d][1,3]oxazin-2-one

307 6-Bromo-1-[1-(toluene-3-sulfonyl)-piperidin-4-yl]-1,4-dihydro-benzo[d][1,3]oxazin-2-one

308 6-Bromo-1-[1-(5-fluoro-2-methyl-benzenesulfonyl)-piperidin-4-yl]-1,4-dihydro-benzo[d][1,3]oxazin-2-one

309 6-Bromo-1-[1-(4-isopropoxy-benzenesulfonyl)-piperidin-4-yl]-1,4-dihydro-benzo[d][1,3]oxazin-2-one

310 6-Bromo-1-[1-(3-chloro-benzenesulfonyl)-piperidin-4-yl]-1,4-dihydro-benzo[d][1,3]oxazin-2-one

311 6-Bromo-1-[1-(3,4-dimethoxy-benzenesulfonyl)-piperidin-4-yl]-1,4-dihydro-benzo[d][1,3]oxazin-2-one

312 6-Bromo-1-(1-pentafluorobenzenesulfonyl-piperidin-4-yl)-1,4-dihydro-benzo[d][1,3]oxazin-2-one

313 6-Bromo-1-[1-(4-chloro-2,5-dimethyl-benzenesulfonyl)-piperidin-4-yl]-1,4-dihydro-benzo[d][1,3]oxazin-2-one

314 6-Bromo-1-[1-(3-methoxy-benzenesulfonyl)-piperidin-4-yl]-1,4-dihydro-benzo[d][1,3]oxazin-2-one

315 6-Bromo-1-[1-(4-isopropyl-benzenesulfonyl)-piperidin-4-yl]-1,4-dihydro-benzo[d][1,3]oxazin-2-one

316 6-Bromo-1-[1-(4-fluoro-benzenesulfonyl)-piperidin-4-yl]-1,4-dihydro-benzo[d][1,3]oxazin-2-one

317 6-Bromo-1-[1-(3-chloro-4-fluoro-benzenesulfonyl)-piperidin-4-yl]-1,4-dihydro-benzo[d][1,3]oxazin-2-one

318 6-Bromo-1-(1-pentamethylbenzenesulfonyl-piperidin-4-yl)-1,4-dihydro-benzo[d][1,3]oxazin-2-one

319 6-Bromo-1-[1-(2-nitro-benzenesulfonyl)-piperidin-4-yl]-1,4-dihydro-benzo[d][1,3]oxazin-2-one

320 6-Bromo-1-[1-(4-chloro-3-nitro-benzenesulfonyl)-piperidin-4-yl]-1,4-dihydro-benzo[d][1,3]oxazin-2-one

321 6-Bromo-1-[1-(5-dimethylamino-naphthalene-1-sulfonyl)-piperidin-4-yl]-1,4-dihydro-benzo[d][1,3]oxazin-2-one

322 6-Bromo-1-[1-(4-nitro-benzenesulfonyl)-piperidin-4-yl]-1,4-dihydro-benzo[d][1,3]oxazin-2-one

323 1-[1-(4-Acetyl-benzenesulfonyl)-piperidin-4-yl]-6-bromo-1,4-dihydro-benzo[d][1,3]oxazin-2-one

324 6-Bromo-1-[1-(4-methanesulfonyl-benzenesulfonyl)-piperidin-4-yl]-1,4-dihydro-benzo[d][1,3]oxazin-2-one

325 1-[1-(Biphenyl-4-sulfonyl)-piperidin-4-yl]-6-bromo-1,4-dihydro-benzo[d][1,3]oxazin-2-one

326 6-Bromo-1-(1-phenylmethanesulfonyl-piperidin-4-yl)-1,4-dihydro-benzo[d][1,3]oxazin-2-one

327 6-Bromo-1-[1-(2,5-dimethoxy-benzenesulfonyl)-piperidin-4-yl]-1,4-dihydro-benzo[d][1,3]oxazin-2-one

328 6-Bromo-1-{1-[2-(2,2,2-trifluoro-acetyl)-1,2,3,4-tetrahydro-isoquinoline-7-sulfonyl]-piperidin-4-yl}-1,4-dihydro-benzo[d][1,3]oxazin-2-one

329 6-Bromo-1-[1-(2,3-dichloro-benzenesulfonyl)-piperidin-4-yl]-1,4-dihydro-benzo[d][1,3]oxazin-2-one

330 6-Bromo-1-[1-(2,4,5-trichloro-benzenesulfonyl)-piperidin-4-yl]-1,4-dihydro-benzo[d][1,3]oxazin-2-one

331 6-Bromo-1-[1-(5-bromo-2-methoxy-benzenesulfonyl)-piperidin-4-yl]-1,4-dihydro-benzo[d][1,3]oxazin-2-one

332 6-Bromo-1-[1-(4-trifluoromethoxy-benzenesulfonyl)-piperidin-4-yl]-1,4-dihydro-benzo[d][1,3]oxazin-2-one

333 6-Bromo-1-[1-(2-nitro-4-trifluoromethyl-benzenesulfonyl)-piperidin-4-yl]-1,4-dihydro-benzo[d][1,3]oxazin-2-one

334 6-Bromo-1-[1-(3-fluoro-benzenesulfonyl)-piperidin-4-yl]-1,4-dihydro-benzo[d][1,3]oxazin-2-one

335 6-Bromo-1-[1-(2,4-dichloro-benzenesulfonyl)-piperidin-4-yl]-1,4-dihydro-benzo[d][1,3]oxazin-2-one

336 6-Bromo-1-[1-(2,4,6-trimethyl-benzenesulfonyl)-piperidin-4-yl]-1,4-dihydro-benzo[d][1,3]oxazin-2-one

337 6-Bromo-1-[1-(2-trifluoromethyl-benzenesulfonyl)-piperidin-4-yl]-1,4-dihydro-benzo[d][1,3]oxazin-2-one

338 6-Bromo-1-[1-(2-bromo-benzenesulfonyl)-piperidin-4-yl]-1,4-dihydro-benzo[d][1,3]oxazin-2-one

339 6-Bromo-1-[1-(4-methoxy-2,3,6-trimethyl-benzenesulfonyl)-piperidin-4-yl]-1,4-dihydro-benzo[d][1,3]oxazin-2-one

340 1-[1-(3,5-Dichloro-4-hydroxy-benzenesulfonyl)-piperidin-4-yl]-1,4-dihydro-benzo[d][1,3]oxazin-2-one

341 1-[1-(3,5-Dichloro-4-hydroxy-benzenesulfonyl)-piperidin-4-yl]-8-methyl-1,4-dihydro-benzo[d][1,3]oxazin-2-one

342 6-Chloro-1-[1-(3,5-dichloro-4-hydroxy-benzenesulfonyl)-piperidin-4-yl]-1,4-dihydro-benzo[d][1,3]oxazin-2-one

343 1-[1-(3,5-Dichloro-4-hydroxy-benzenesulfonyl)-piperidin-4-yl]-6-methyl-1,4-dihydro-benzo[d][1,3]oxazin-2-one

344 6-Bromo-1-[1-(3,5-dichloro-4-hydroxy-benzenesulfonyl)-piperidin-4-yl]-1,4-dihydro-benzo[d][1,3]oxazin-2-one

345 6-Chloro-1-[1-(3,5-dichloro-2-hydroxy-benzenesulfonyl)-piperidin-4-yl]-1,4-dihydro-benzo[d][1,3]oxazin-2-one

346 6-Bromo-1-[1-(3,5-dichloro-2-hydroxy-benzenesulfonyl)-piperidin-4-yl]-1,4-dihydro-benzo[d][1,3]oxazin-2-one

347 2-[4-(6-Bromo-2-oxo-4H-benzo[d][1,3]oxazin-1-yl)-piperidine-1-sulfonyl]-benzonitrile

348 6-Bromo-1-[1-(4-methoxy-benzenesulfonyl)-piperidin-4-yl]-1,4-dihydro-benzo[d][1,3]oxazin-2-one

349 2-[4-(6-Bromo-2-oxo-4H-benzo[d][1,3]oxazin-1-yl)-piperidine-1-sulfonyl]-benzoic acid methyl ester

350 6-Bromo-1-[1-(3-trifluoromethyl-benzenesulfonyl)-piperidin-4-yl]-1,4-dihydro-benzo[d][1,3]oxazin-2-one

351 6-Bromo-1-[1-(2-oxo-2H-chromene-6-sulfonyl)-piperidin-4-yl]-1,4-dihydro-benzo[d][1,3]oxazin-2-one

352 6-Bromo-1-[1-(3,5-dichloro-benzenesulfonyl)-piperidin-4-yl]-1,4-dihydro-benzo[d][1,3]oxazin-2-one

353 6-Bromo-1-[1-(2,5-dichloro-benzenesulfonyl)-piperidin-4-yl]-1,4-dihydro-benzo[d][1,3]oxazin-2-one

354 6-Bromo-1-[1-(5-bromo-6-chloro-pyridine-3-sulfonyl)-piperidin-4-yl]-1,4-dihydro-benzo[d][1,3]oxazin-2-one

355 6-Bromo-1-[1-(4-chloro-benzenesulfonyl)-piperidin-4-yl]-1,4-dihydro-benzo[d][1,3]oxazin-2-one

356 6-Bromo-1-[1-(2,6-dichloro-benzenesulfonyl)-piperidin-4-yl]-1,4-dihydro-benzo[d][1,3]oxazin-2-one

357 6-Bromo-1-[1-(1-methyl-1H-imidazole-4-sulfonyl)-piperidin-4-yl]-1,4-dihydro-benzo[d][1,3]oxazin-2-one

358 6-Bromo-1-[1-(5-bromo-2,4-difluoro-benzenesulfonyl)-piperidin-4-yl]-1,4-dihydro-benzo[d][1,3]oxazin-2-one

359 6-Bromo-1-[1-(4-ethyl-benzenesulfonyl)-piperidin-4-yl]-1,4-dihydro-benzo[d][1,3]oxazin-2-one

360 6-Bromo-1-[1-(6-chloro-imidazo[2,1-b]thiazole-5-sulfonyl)-piperidin-4-yl]-1,4-dihydro-benzo[d][1,3]oxazin-2-one

361 1-[1-(Benzo[b]thiophene-3-sulfonyl)-piperidin-4-yl]-6-bromo-1,4-dihydro-benzo[d][1,3]oxazin-2-one

362 6-Bromo-1-[1-(7-chloro-benzo[1,2,5]oxadiazole-4-sulfonyl)-piperidin-4-yl]-1,4-dihydro-benzo[d][1,3]oxazin-2-one

363 6-Bromo-1-[1-(2-methoxy-4-methyl-benzenesulfonyl)-piperidin-4-yl]-1,4-dihydro-benzo[d][1,3]oxazin-2-one

364 3-{4-[4-(6-Bromo-2-oxo-4H-benzo[d][1,3]oxazin-1-yl)-piperidine-1-sulfonyl]-phenyl}-propionic acid methyl ester

365 6-Bromo-1-[1-(2,4-dinitro-benzenesulfonyl)-piperidin-4-yl]-1,4-dihydro-benzo[d][1,3]oxazin-2-one

366 1-[1-(Benzo[b]thiophene-2-sulfonyl)-piperidin-4-yl]-6-bromo-1,4-dihydro-benzo[d][1,3]oxazin-2-one

367 6-Bromo-1-[1-(2,5-difluoro-benzenesulfonyl)-piperidin-4-yl]-1,4-dihydro-benzo[d][1,3]oxazin-2-one

368 6-Bromo-1-[1-(4-chloro-2,5-difluoro-benzenesulfonyl)-piperidin-4-yl]-1,4-dihydro-benzo[d][1,3]oxazin-2-one

369 6-Bromo-1-[1-(2,4,5-trifluoro-benzenesulfonyl)-piperidin-4-yl]-1,4-dihydro-benzo[d][1,3]oxazin-2-one

370 6-Bromo-1-[1-(2,6-difluoro-benzenesulfonyl)-piperidin-4-yl]-1,4-dihydro-benzo[d][1,3]oxazin-2-one

371 6-Bromo-1-[1-(5-chloro-2,4-difluoro-benzenesulfonyl)-piperidin-4-yl]-1,4-dihydro-benzo[d][1,3]oxazin-2-one

372 6-Bromo-1-[1-(2-chloro-benzenesulfonyl)-piperidin-4-yl]-1,4-dihydro-benzo[d][1,3]oxazin-2-one

373 6-Bromo-1-[1-(2,3,4-trifluoro-benzenesulfonyl)-piperidin-4-yl]-1,4-dihydro-benzo[d][1,3]oxazin-2-one

374 N-{4-[4-(6-Bromo-2-oxo-4H-benzo[d][1,3]oxazin-1-yl)-piperidine-1-sulfonyl]-2-chloro-phenyl)-acetamide

375 1-[1-(2,3,4-Trifluoro-benzenesulfonyl)-piperidin-4-yl]-1,4-dihydro-benzo[d][1,3]oxazin-2-one

376 8-Methyl-1-[1-(2,3,4-trifluoro-benzenesulfonyl)-piperidin-4-yl]-1,4-dihydro-benzo[d][1,3]oxazin-2-one

377 6-Chloro-1-[1-(2,3,4-trifluoro-benzenesulfonyl)-piperidin-4-yl]-1,4-dihydro-benzo[d][1,3]oxazin-2-one

378 6-Methyl-1-[1-(2,3,4-trifluoro-benzenesulfonyl)-piperidin-4-yl]-1,4-dihydro-benzo[d][1,3]oxazin-2-one

379 N-{2-Chloro-4-[4-(6-methyl-2-oxo-4H-benzo[d][1,3]oxazin-1-yl)-piperidine-1-sulfonyl]-phenyl}-acetamide

380 1-[1-(3,4-Difluoro-benzenesulfonyl)-piperidin-4-yl]-1,4-dihydro-benzo[d][1,3]oxazin-2-one

381 1-[1-(3,4-Difluoro-benzenesulfonyl)-piperidin-4-yl]-8-methyl-1,4-dihydro-benzo[d][1,3]oxazin-2-one

382 6-Chloro-1-[1-(3,4-difluoro-benzenesulfonyl)-piperidin-4-yl]-1,4-dihydro-benzo[d][1,3]oxazin-2-one

383 1-[1-(3,4-Difluoro-benzenesulfonyl)-piperidin-4-yl]-6-methyl-1,4-dihydro-benzo[d][1,3]oxazin-2-one

384 6-Bromo-1-[1-(3,4-difluoro-benzenesulfonyl)-piperidin-4-yl]-1,4-dihydro-benzo[d][1,3]oxazin-2-one

385 N-{2-Chloro-4-[4-(8-methyl-2-oxo-4H-benzo[d][1,3]oxazin-1-yl)-piperidine-1-sulfonyl]-phenyl}-acetamide

386 1-[1-(2-Chloro-4,5-difluoro-benzenesulfonyl)-piperidin-4-yl]-1,4-dihydro-benzo[d][1,3]oxazin-2-one

387 1-[1-(2-Chloro-4,5-difluoro-benzenesulfonyl)-piperidin-4-yl]-8-methyl-1,4-dihydro-benzo[d][1,3]oxazin-2-one

388 6-Chloro-1-[1-(2-chloro-4,5-difluoro-benzenesulfonyl)-piperidin-4-yl]-1,4-dihydro-benzo[d][1,3]oxazin-2-one

389 1-[1-(2-Chloro-4,5-difluoro-benzenesulfonyl)-piperidin-4-yl]-6-methyl-1,4-dihydro-benzo[d][1,3]oxazin-2-one

390 6-Bromo-1-[1-(2-chloro-4,5-difluoro-benzenesulfonyl)-piperidin-4-yl]-1,4-dihydro-benzo[d][1,3]oxazin-2-one

391 N-{2-Chloro-4-[4-(2-oxo-4H-benzo[d][1,3]oxazin-1-yl)-piperidine-1-sulfonyl]-phenyl}-acetamide

392 1-[1-(Benzo[1,2,5]oxadiazole-4-sulfonyl)-piperidin-4-yl]-1,4-dihydro-benzo[d][1,3]oxazin-2-one

393 1-[1-(Benzo[1,2,5]oxadiazole-4-sulfonyl)-piperidin-4-yl]-8-methyl-1,4-dihydro-benzo[d][1,3]oxazin-2-one

394 1-[1-(Benzo[1,2,5]oxadiazole-4-sulfonyl)-piperidin-4-yl]-6-chloro-1,4-dihydro-benzo[d][1,3]oxazin-2-one

395 1-[1-(Benzo[1,2,5]oxadiazole-4-sulfonyl)-piperidin-4-yl]-6-methyl-1,4-dihydro-benzo[d][1,3]oxazin-2-one

396 1-[1-(Benzo[1,2,5]oxadiazole-4-sulfonyl)-piperidin-4-yl]-6-bromo-1,4-dihydro-benzo[d][1,3]oxazin-2-one

397 N-{2-Chloro-4-[4-(6-chloro-2-oxo-4H-benzo[d][1,3]oxazin-1-yl)-piperidine-1-sulfonyl]-phenyl}-acetamide

398 1-[1-(Benzo[1,2,5]thiadiazole-4-sulfonyl)-piperidin-4-yl]-1,4-dihydro-benzo[d][1,3]oxazin-2-one

399 1-[1-(Benzo[1,2,5]thiadiazole-4-sulfonyl)-piperidin-4-yl]-8-methyl-1,4-dihydro-benzo[d][1,3]oxazin-2-one

400 1-[1-(Benzo[1,2,5]thiadiazole-4-sulfonyl)-piperidin-4-yl]-6-chloro-1,4-dihydro-benzo[d][1,3]oxazin-2-one

401 1-[1-(Benzo[1,2,5]thiadiazole-4-sulfonyl)-piperidin-4-yl]-6-methyl-1,4-dihydro-benzo[d][1,3]oxazin-2-one

402 1-[1-(Benzo[1,2,5]thiadiazole-4-sulfonyl)-piperidin-4-yl]-6-bromo-1,4-dihydro-benzo[d][1,3]oxazin-2-one

403 1-(1-Ethanesulfonyl-piperidin-4-yl)-8-methyl-1,4-dihydro-benzo[d][1,3]oxazin-2-one

404 1-[1-(2,4-Difluoro-benzenesulfonyl)-piperidin-4-yl]-1,4-dihydro-benzo[d][1,3]oxazin-2-one

405 1-[1-(2,4-Difluoro-benzenesulfonyl)-piperidin-4-yl]-8-methyl-1,4-dihydro-benzo[d][1,3]oxazin-2-one

406 6-Chloro-1-[1-(2,4-difluoro-benzenesulfonyl)-piperidin-4-yl]-1,4-dihydro-benzo[d][1,3]oxazin-2-one

407 1-[1-(2,4-Difluoro-benzenesulfonyl)-piperidin-4-yl]-6-methyl-1,4-dihydro-benzo[d][1,3]oxazin-2-one

408 6-Bromo-1-[1-(2,4-difluoro-benzenesulfonyl)-piperidin-4-yl]-1,4-dihydro-benzo[d][1,3]oxazin-2-one

409 8-Methyl-1-[1-(propane-2-sulfonyl)-piperidin-4-yl]-1,4-dihydro-benzo[d][1,3]oxazin-2-one

410 1-[1-(3,4-Dichloro-benzenesulfonyl)-piperidin-4-yl]-1,4-dihydro-benzo[d][1,3]oxazin-2-one

411 1-[1-(3,4-Dichloro-benzenesulfonyl)-piperidin-4-yl]-8-methyl-1,4-dihydro-benzo[d][1,3]oxazin-2-one

412 6-Chloro-1-[1-(3,4-dichloro-benzenesulfonyl)-piperidin-4-yl]-1,4-dihydro-benzo[d][1,3]oxazin-2-one

413 1-[1-(3,4-Dichloro-benzenesulfonyl)-piperidin-4-yl]-6-methyl-1,4-dihydro-benzo[d][1,3]oxazin-2-one

414 6-Bromo-1-[1-(3,4-dichloro-benzenesulfonyl)-piperidin-4-yl]-1,4-dihydro-benzo[d][1,3]oxazin-2-one

415 8-Methyl-1-[1-(propane-1-sulfonyl)-piperidin-4-yl]-1,4-dihydro-benzo[d][1,3]oxazin-2-one

416 1-[1-(2-Chloro-6-methyl-benzenesulfonyl)-piperidin-4-yl]-1,4-dihydro-benzo[d][1,3]oxazin-2-one

417 1-[1-(2-Chloro-6-methyl-benzenesulfonyl)-piperidin-4-yl]-8-methyl-1,4-dihydro-benzo[d][1,3]oxazin-2-one

418 6-Chloro-1-[1-(2-chloro-6-methyl-benzenesulfonyl)-piperidin-4-yl]-1,4-dihydro-benzo[d][1,3]oxazin-2-one

419 1-[1-(2-Chloro-6-methyl-benzenesulfonyl)-piperidin-4-yl]-6-methyl-1,4-dihydro-benzo[d][1,3]oxazin-2-one

420 1-[1-(2-Chloro-6-methyl-benzenesulfonyl)-piperidin-4-yl]-6-methyl-1,4-dihydro-benzo[d][1,3]oxazin-2-one

421 8-Methyl-1-[1-(2,3,5,6-tetramethyl-benzenesulfonyl)-piperidin-4-yl]-1,4-dihydro-benzo[d][1,3]oxazin-2-one

422 1-[1-(2,3,4-Trichloro-benzenesulfonyl)-piperidin-4-yl]-1,4-dihydro-benzo[d][1,3]oxazin-2-one

423 8-Methyl-1-[1-(2,3,4-trichloro-benzenesulfonyl)-piperidin-4-yl]-1,4-dihydro-benzo[d][1,3]oxazin-2-one

424 6-Chloro-1-[1-(2,3,4-trichloro-benzenesulfonyl)-piperidin-4-yl]-1,4-dihydro-benzo[d][1,3]oxazin-2-one

425 6-Methyl-1-[1-(2,3,4-trichloro-benzenesulfonyl)-piperidin-4-yl]-1,4-dihydro-benzo[d][1,3]oxazin-2-one

426 6-Bromo-1-[1-(2,3,4-trichloro-benzenesulfonyl)-piperidin-4-yl]-1,4-dihydro-benzo[d][1,3]oxazin-2-one

427 1-[1-(2,3,5,6-Tetramethyl-benzenesulfonyl)-piperidin-4-yl]-1,4-dihydro-benzo[d][1,3]oxazin-2-one

428 1-[1-(Thiophene-3-sulfonyl)-piperidin-4-yl]-1,4-dihydro-benzo[d][1,3]oxazin-2-one

429 8-Methyl-1-[1-(thiophene-3-sulfonyl)-piperidin-4-yl]-1,4-dihydro-benzo[d][1,3]oxazin-2-one

430 6-Chloro-1-[1-(thiophene-3-sulfonyl)-piperidin-4-yl]-1,4-dihydro-benzo[d][1,3]oxazin-2-one

431 6-Methyl-1-[1-(thiophene-3-sulfonyl)-piperidin-4-yl]-1,4-dihydro-benzo[d][1,3]oxazin-2-one

432 6-Bromo-1-[1-(thiophene-3-sulfonyl)-piperidin-4-yl]-1,4-dihydro-benzo[d][1,3]oxazin-2-one

433 6-Chloro-1-[1-(2,3,5,6-tetramethyl-benzenesulfonyl)-piperidin-4-yl]-1,4-dihydro-benzo[d][1,3]oxazin-2-one

434 1-[1-(2,4,6-Trichloro-benzenesulfonyl)-piperidin-4-yl]-1,4-dihydro-benzo[d][1,3]oxazin-2-one

435 8-Methyl-1-[1-(2,4,6-trichloro-benzenesulfonyl)-piperidin-4-yl]-1,4-dihydro-benzo[d][1,3]oxazin-2-one

436 6-Chloro-1-[1-(2,4,6-trichloro-benzenesulfonyl)-piperidin-4-yl]-1,4-dihydro-benzo[d][1,3]oxazin-2-one

437 6-Methyl-1-[1-(2,4,6-trichloro-benzenesulfonyl)-piperidin-4-yl]-1,4-dihydro-benzo[d][1,3]oxazin-2-one

438 6-Bromo-1-[1-(2,4,6-trichloro-benzenesulfonyl)-piperidin-4-yl]-1,4-dihydro-benzo[d][1,3]oxazin-2-one

439 6-Methyl-1-[1-(2,3,5,6-tetramethyl-benzenesulfonyl)-piperidin-4-yl]-1,4-dihydro-benzo[d][1,3]oxazin-2-one

440 1-[1-(2-Bromo-4,6-difluoro-benzenesulfonyl)-piperidin-4-yl]-8-methyl-1,4-dihydro-benzo[d][1,3]oxazin-2-one

441 1-[1-(2-Bromo-4,6-difluoro-benzenesulfonyl)-piperidin-4-yl]-6-chloro-1,4-di hydro-benzo[d][1,3]oxazin-2-one

442 1-[1-(2-Bromo-4,6-difluoro-benzenesulfonyl)-piperidin-4-yl]-6-methyl-1,4-dihydro-benzo[d][1,3]oxazin-2-one

443 6-Bromo-1-[1-(2-bromo-4,6-difluoro-benzenesulfonyl)-piperidin-4-yl]-1,4-dihydro-benzo[d][1,3]oxazin-2-one

444 6-Bromo-1-[1-(2,3,5,6-tetramethyl-benzenesulfonyl)-piperidin-4-yl]-1,4-dihydro-benzo[d][1,3]oxazin-2-one

445 1-[1-(4-Bromo-2-trifluoromethoxy-benzenesulfonyl)-piperidin-4-yl]-1,4-dihydro-benzo[d][1,3]oxazin-2-one

446 1-[1-(4-Bromo-2-trifluoromethoxy-benzenesulfonyl)-piperidin-4-yl]-8-methyl-1,4-dihydro-benzo[d][1,3]oxazin-2-one

447 1-[1-(4-Bromo-2-trifluoromethoxy-benzenesulfonyl)-piperidin-4-yl]-6-chloro-1,4-dihydro-benzo[d][1,3]oxazin-2-one

448 1-[1-(4-Bromo-2-trifluoromethoxy-benzenesulfonyl)-piperidin-4-yl]-6-methyl-1,4-dihydro-benzo[d][1,3]oxazin-2-one

449 6-Bromo-1-[1-(4-bromo-2-trifluoromethoxy-benzenesulfonyl)-piperidin-4-yl]-1,4-dihydro-benzo[d][1,3]oxazin-2-one

450 1-[1-(4-Phenoxy-benzenesulfonyl)-piperidin-4-yl]-1,4-dihydro-benzo[d][1,3]oxazin-2-one

451 1-[1-(3-Bromo-benzenesulfonyl)-piperidin-4-yl]-1,4-dihydro-benzo[d][1,3]oxazin-2-one

452 1-[1-(3-Bromo-benzenesulfonyl)-piperidin-4-yl]-8-methyl-1,4-dihydro-benzo[d][1,3]oxazin-2-one

453 1-[1-(3-Bromo-benzenesulfonyl)-piperidin-4-yl]-6-chloro-1,4-dihydro-benzo[d][1,3]oxazin-2-one

454 1-[1-(3-Bromo-benzenesulfonyl)-piperidin-4-yl]-6-methyl-1,4-dihydro-benzo[d][1,3]oxazin-2-one

455 6-Bromo-1-[1-(3-bromo-benzenesulfonyl)-piperidin-4-yl]-1,4-dihydro-benzo[d][1,3]oxazin-2-one

456 8-Methyl-1-[1-(4-phenoxy-benzenesulfonyl)-piperidin-4-yl]-1,4-dihydro-benzo[d][1,3]oxazin-2-one

457 1-[1-(4-tert-Butyl-benzenesulfonyl)-piperidin-4-yl]-1,4-dihydro-benzo[d][1,3]oxazin-2-one

458 1-[1-(4-tert-Butyl-benzenesulfonyl)-piperidin-4-yl]-8-methyl-1,4-dihydro-benzo[d][1,3]oxazin-2-one

459 1-[1-(4-tert-Butyl-benzenesulfonyl)-piperidin-4-yl]-6-chloro-1,4-dihydro-benzo[d][1,3]oxazin-2-one

460 1-[1-(4-tert-Butyl-benzenesulfonyl)-piperidin-4-yl]-6-methyl-1,4-dihydro-benzo[d][1,3]oxazin-2-one

461 6-Bromo-1-[1-(4-tert-butyl-benzenesulfonyl)-piperidin-4-yl]-1,4-dihydro-benzo[d][1,3]oxazin-2-one

462 6-Chloro-1-[1-(4-phenoxy-benzenesulfonyl)-piperidin-4-yl]-1,4-dihydro-benzo[d][1,3]oxazin-2-one

463 1-[1-(2-Bromo-4,6-difluoro-benzenesulfonyl)-piperidin-4-yl]-1,4-dihydro-benzo[d][1,3]oxazin-2-one

464 1-[1-(2-Methanesulfonyl-benzenesulfonyl)-piperidin-4-yl]-8-methyl-1,4-dihydro-benzo[d][1,3]oxazin-2-one

465 6-Chloro-1-[1-(2-methanesulfonyl-benzenesulfonyl)-piperidin-4-yl]-1,4-dihydro-benzo[d][1,3]oxazin-2-one

466 1-[1-(2-Methanesulfonyl-benzenesulfonyl)-piperidin-4-yl]-6-methyl-1,4-dihydro-benzo[d][1,3]oxazin-2-one

467 6-Bromo-1-[1-(2-methanesulfonyl-benzenesulfonyl)-piperidin-4-yl]-1,4-dihydro-benzo[d][1,3]oxazin-2-one

468 8-Methyl-1-[1-(4-propyl-benzenesulfonyl)-piperidin-4-yl]-1,4-dihydro-benzo[d][1,3]oxazin-2-one

469 6-Chloro-1-[1-(4-propyl-benzenesulfonyl)-piperidin-4-yl]-1,4-dihydro-benzo[d][1,3]oxazin-2-one

470 6-Methyl-1-[1-(4-propyl-benzenesulfonyl)-piperidin-4-yl]-1,4-dihydro-benzo[d][1,3]oxazin-2-one

471 6-Bromo-1-[1-(4-propyl-benzenesulfonyl)-piperidin-4-yl]-1,4-dihydro-benzo[d][1,3]oxazin-2-one

472 1-[1-(3-Chloro-2-methyl-benzenesulfonyl)-piperidin-4-yl]-8-methyl-1,4-dihydro-benzo[d][1,3]oxazin-2-one

473 6-Chloro-1-[1-(3-chloro-2-methyl-benzenesulfonyl)-piperidin-4-yl]-1,4-dihydro-benzo[d][1,3]oxazin-2-one

474 1-[1-(3-Chloro-2-methyl-benzenesulfonyl)-piperidin-4-yl]-6-methyl-1,4-dihydro-benzo[d][1,3]oxazin-2-one

475 6-Bromo-1-[1-(3-chloro-2-methyl-benzenesulfonyl)-piperidin-4-yl]-1,4-dihydro-benzo[d][1,3]oxazin-2-one

476 1-[1-(4-Butyl-benzenesulfonyl)-piperidin-4-yl]-8-methyl-1,4-dihydro-benzo[d][1,3]oxazin-2-one

477 1-[1-(4-Butyl-benzenesulfonyl)-piperidin-4-yl]-6-chloro-1,4-dihydro-benzo[d][1,3]oxazin-2-one

478 1-[1-(4-Butyl-benzenesulfonyl)-piperidin-4-yl]-6-methyl-1,4-dihydro-benzo[d][1,3]oxazin-2-one

479 6-Bromo-1-[1-(4-butyl-benzenesulfonyl)-piperidin-4-yl]-1,4-dihydro-benzo[d][1,3]oxazin-2-one

480 1-[1-(4-Bromo-3-methyl-benzenesulfonyl)-piperidin-4-yl]-8-methyl-1,4-dihydro-benzo[d][1,3]oxazin-2-one

481 1-[1-(4-Bromo-3-methyl-benzenesulfonyl)-piperidin-4-yl]-6-chloro-1,4-dihydro-benzo[d][1,3]oxazin-2-one

482 1-[1-(4-Bromo-3-methyl-benzenesulfonyl)-piperidin-4-yl]-6-methyl-1,4-dihydro-benzo[d][1,3]oxazin-2-one

483 6-Bromo-1-[1-(4-bromo-3-methyl-benzenesulfonyl)-piperidin-4-yl]-1,4-dihydro-benzo[d][1,3]oxazin-2-one

484 1-{1-[4-(1,1-Dimethyl-propyl)-benzenesulfonyl]-piperidin-4-yl}-8-methyl-1,4-dihydro-benzo[d][1,3]oxazin-2-one

485 6-Chloro-1-{1-[1-(1,1-dimethyl-propyl)-benzenesulfonyl]-piperidin-4-yl}-1,4-dihydro-benzo[d][1,3]oxazin-2-one

486 1-{1-[4-(1,1-Dimethyl-propyl)-benzenesulfonyl]-piperidin-4-yl}-6-methyl-1,4-dihydro-benzo[d][1,3]oxazin-2-one

487 6-Bromo-1-{1-[4-(1,1-dimethyl-propyl)-benzenesulfonyl]-piperidin-4-yl}-1,4-dihydro-benzo[d][1,3]oxazin-2-one

488 1-(1-Ethenesulfonyl-piperidin-4-yl)-8-methyl-1,4-dihydro-benzo[d][1,3]oxazin-2-one

489 3-[4-(8-Methyl-2-oxo-4H-benzo[d][1,3]oxazin-1-yl)-piperidine-1-sulfonyl]-benzoic acid

490 3-[4-(6-Methyl-2-oxo-4H-benzo[d][1,3]oxazin-1-yl)-piperidine-1-sulfonyl]-benzoic acid

491 3-[4-(6-Bromo-2-oxo-4H-benzo[d][1,3]oxazin-1-yl)-piperidine-1-sulfonyl]-benzoic acid

492 1-[1-(3-Chloro-2-fluoro-benzenesulfonyl)-piperidin-4-yl]-8-methyl-1,4-dihydro-benzo[d][1,3]oxazin-2-one

493 6-Chloro-1-[1-(3-chloro-2-fluoro-benzenesulfonyl)-piperidin-4-yl]-1,4-dihydro-benzo[d][1,3]oxazin-2-one

494 1-[1-(3-Chloro-2-fluoro-benzenesulfonyl)-piperidin-4-yl]-6-methyl-1,4-dihydro-benzo[d][1,3]oxazin-2-one

495 6-Bromo-1-[1-(3-chloro-2-fluoro-benzenesulfonyl)-piperidin-4-yl]-1,4-dihydro-benzo[d][1,3]oxazin-2-one

496 N-{4-Methyl-5-[4-(8-methyl-2-oxo-4H-benzo[d][1,3]oxazin-1-yl)-piperidine-1-sulfonyl]-thiazol-2-yl}-acetamide

497 N-{5-[4-(6-Chloro-2-oxo-4H-benzo[d][1,3]oxazin-1-yl)-piperidine-1-sulfonyl]-4-methyl-thiazol-2-yl)-acetamide

498 N-{4-Methyl-5-[4-(6-methyl-2-oxo-4H-benzo[d][1,3]oxazin-1-yl)-piperidine-1-sulfonyl]-thiazol-2-yl}-acetamide

499 N-{5-[4-(6-Bromo-2-oxo-4H-benzo[d][1,3]oxazin-1-yl)-piperidine-1-sulfonyl]-4-methyl-thiazol-2-yl)-acetamide

500 1-[1-(2-Bromo-4-fluoro-benzenesulfonyl)-piperidin-4-yl]-8-methyl-1,4-dihydro-benzo[d][1,3]oxazin-2-one

501 1-[1-(2-Bromo-4-fluoro-benzenesulfonyl)-piperidin-4-yl]-6-chloro-1,4-dihydro-benzo[d][1,3]oxazin-2-one

502 1-[1-(2-Bromo-4-fluoro-benzenesulfonyl)-piperidin-4-yl]-6-methyl-1,4-dihydro-benzo[d][1,3]oxazin-2-one

503 6-Bromo-1-[1-(2-bromo-4-fluoro-benzenesulfonyl)-piperidin-4-yl]-1,4-dihydro-benzo[d][1,3]oxazin-2-one

504 1-[1-(5-Chloro-2-fluoro-benzenesulfonyl)-piperidin-4-yl]-8-methyl-1,4-dihydro-benzo[d][1,3]oxazin-2-one

505 6-Chloro-1-[1-(5-chloro-2-fluoro-benzenesulfonyl)-piperidin-4-yl]-1,4-dihydro-benzo[d][1,3]oxazin-2-one

506 1-[1-(5-Chloro-2-fluoro-benzenesulfonyl)-piperidin-4-yl]-6-methyl-1,4-dihydro-benzo[d][1,3]oxazin-2-one

507 6-Bromo-1-[1-(5-chloro-2-fluoro-benzenesulfonyl)-piperidin-4-yl]-1,4-dihydro-benzo[d][1,3]oxazin-2-one

508 1-[1-(4-Bromo-3-trifluoromethyl-benzenesulfonyl)-piperidin-4-yl]-8-methyl-1,4-dihydro-benzo[d][1,3]oxazin-2-one

509 1-[1-(4-Bromo-3-trifluoromethyl-benzenesulfonyl)-piperidin-4-yl]-6-chloro-1,4-dihydro-benzo[d][1,3]oxazin-2-one

510 1-[1-(4-Bromo-3-trifluoromethyl-benzenesulfonyl)-piperidin-4-yl]-6-methyl-1,4-dihydro-benzo[d][1,3]oxazin-2-one

511 6-Bromo-1-[1-(4-bromo-3-trifluoromethyl-benzenesulfonyl)-piperidin-4-yl]-1,4-dihydro-benzo[d][1,3]oxazin-2-one

512 1-[1-(2-Methanesulfonyl-benzenesulfonyl)-piperidin-4-yl]-1,4-dihydro-benzo[d][1,3]oxazin-2-one

513 1-[1-(4-Propyl-benzenesulfonyl)-piperidin-4-yl]-1,4-dihydro-benzo[d][1,3]oxazin-2-one

514 1-[1-(3-Chloro-2-methyl-benzenesulfonyl)-piperidin-4-yl]-1,4-dihydro-benzo[d][1,3]oxazin-2-one

515 1-[1-(4-Butyl-benzenesulfonyl)-piperidin-4-yl]-1,4-dihydro-benzo[d][1,3]oxazin-2-one

516 1-[1-(4-Bromo-3-methyl-benzenesulfonyl)-piperidin-4-yl]-1,4-dihydro-benzo[d][1,3]oxazin-2-one

517 1-{1-[1-(1,1-Dimethyl-propyl)-benzenesulfonyl]-piperidin-4-yl}-1,4-dihydro-benzo[d][1,3]oxazin-2-one

518 N-{4-Methyl-5-[4-(2-oxo-4H-benzo[d][1,3]oxazin-1-yl)-piperidine-1-sulfonyl]-thiazol-2-yl}-acetamide

519 1-[1-(3-Chloro-2-fluoro-benzenesulfonyl)-piperidin-4-yl]-1,4-dihydro-benzo[d][1,3]oxazin-2-one

520 1-[1-(2-Bromo-4-fluoro-benzenesulfonyl)-piperidin-4-yl]-1,4-dihydro-benzo[d][1,3]oxazin-2-one

521 1-[1-(4-Bromo-3-trifluoromethyl-benzenesulfonyl)-piperidin-4-yl]-1,4-dihydro-benzo[d][1,3]oxazin-2-one

522 1-[1-(5-Chloro-2-fluoro-benzenesulfonyl)-piperidin-4-yl]-1,4-dihydro-benzo[d][1,3]oxazin-2-one

523 1-[1-(Isoquinoline-5-sulfonyl)-piperidin-4-yl]-1,4-dihydro-benzo[d][1,3]oxazin-2-one

524 6-Fluoro-1-[1-(2-methanesulfonyl-benzenesulfonyl)-piperidin-4-yl]-1,4-dihydro-benzo[d][1,3]oxazin-2-one

525 6-Fluoro-1-[1-(4-propyl-benzenesulfonyl)-piperidin-4-yl]-1,4-dihydro-benzo[d][1,3]oxazin-2-one

526 1-[1-(3-Chloro-2-methyl-benzenesulfonyl)-piperidin-4-yl]-6-fluoro-1,4-dihydro-benzo[d][1,3]oxazin-2-one

527 1-[1-(4-Butyl-benzenesulfonyl)-piperidin-4-yl]-6-fluoro-1,4-dihydro-benzo[d][1,3]oxazin-2-one

528 1-[1-(4-Bromo-3-methyl-benzenesulfonyl)-piperidin-4-yl]-6-fluoro-1,4-dihydro-benzo[d][1,3]oxazin-2-one

529 1-{1-[1-(1,1-Dimethyl-propyl)-benzenesulfonyl]-piperidin-4-yl}-6-fluoro-1,4-dihydro-benzo[d][1,3]oxazin-2-one

530 N-{5-[4-(6-Fluoro-2-oxo-4H-benzo[d][1,3]oxazin-1-yl)-piperidine-1-sulfonyl]-4-methyl-thiazol-2-yl)-acetamide

531 1-[1-(3-Chloro-2-fluoro-benzenesulfonyl)-piperidin-4-yl]-6-fluoro-1,4-dihydro-benzo[d][1,3]oxazin-2-one

532 1-[1-(2-Bromo-4-fluoro-benzenesulfonyl)-piperidin-4-yl]-6-fluoro-1,4-dihydro-benzo[d][1,3]oxazin-2-one

533 1-[1-(4-Bromo-3-trifluoromethyl-benzenesulfonyl)-piperidin-4-yl]-6-fluoro-1,4-dihydro-benzo[d][1,3]oxazin-2-one

534 1-[1-(5-Chloro-2-fluoro-benzenesulfonyl)-piperidin-4-yl]-6-fluoro-1,4-dihydro-benzo[d][1,3]oxazin-2-one

535 6-Fluoro-1-[1-(isoquinoline-5-sulfonyl)-piperidin-4-yl]-1,4-dihydro-benzo[d][1,3]oxazin-2-one

536 6-Fluoro-1-[1-(quinoline-8-sulfonyl)-piperidin-4-yl]-1,4-dihydro-benzo[d][1,3]oxazin-2-one

537 1-[1-(5-Chloro-3-methyl-benzo[b]thiophene-2-sulfonyl)-piperidin-4-yl]-6-fluoro-1,4-dihydro-benzo[d][1,3]oxazin-2-one

538 6-Fluoro-1-[1-(naphthalene-1-sulfonyl)-piperidin-4-yl]-1,4-dihydro-benzo[d][1,3]oxazin-2-one

539 6-Fluoro-1-[1-(naphthalene-2-sulfonyl)-piperidin-4-yl]-1,4-dihydro-benzo[d][1,3]oxazin-2-one

540 1-[1-(Benzo[b]thiophene-2-sulfonyl)-piperidin-4-yl]-6-fluoro-1,4-dihydro-benzo[d][1,3]oxazin-2-one

541 1-[1-(Benzo[b]thiophene-3-sulfonyl)-piperidin-4-yl]-6-fluoro-1,4-dihydro-benzo[d][1,3]oxazin-2-one

542 8-Methoxy-1-[1-(quinoline-8-sulfonyl)-piperidin-4-yl]-1,4-dihydro-benzo[d][1,3]oxazin-2-one

543 1-[1-(5-Chloro-3-methyl-benzo[b]thiophene-2-sulfonyl)-piperidin-4-yl]-8-methoxy-1,4-dihydro-benzo[d][1,3]oxazin-2-one

544 8-Methoxy-1-[1-(naphthalene-1-sulfonyl)-piperidin-4-yl]-1,4-dihydro-benzo[d][1,3]oxazin-2-one

545 8-Methoxy-1-[1-(naphthalene-2-sulfonyl)-piperidin-4-yl]-1,4-dihydro-benzo[d][1,3]oxazin-2-one

546 1-[1-(Benzo[b]thiophene-2-sulfonyl)-piperidin-4-yl]-8-methoxy-1,4-dihydro-benzo[d][1,3]oxazin-2-one

547 1-[1-(Benzo[b]thiophene-3-sulfonyl)-piperidin-4-yl]-8-methoxy-1,4-dihydro-benzo[d][1,3]oxazin-2-one

548 5-Chloro-1-[1-(2-methanesulfonyl-benzenesulfonyl)-piperidin-4-yl]-1,4-dihydro-benzo[d][1,3]oxazin-2-one

549 5-Chloro-1-[1-(4-propyl-benzenesulfonyl)-piperidin-4-yl]-1,4-dihydro-benzo[d][1,3]oxazin-2-one

550 5-Chloro-1-[1-(3-chloro-2-methyl-benzenesulfonyl)-piperidin-4-yl]-1,4-dihydro-benzo[d][1,3]oxazin-2-one

551 1-[1-(4-Butyl-benzenesulfonyl)-piperidin-4-yl]-5-chloro-1,4-dihydro-benzo[d][1,3]oxazin-2-one

552 1-[1-(4-Bromo-3-methyl-benzenesulfonyl)-piperidin-4-yl]-5-chloro-1,4-dihydro-benzo[d][1,3]oxazin-2-one

553 5-Chloro-1-{1-[1-(1,1-dimethyl-propyl)-benzenesulfonyl]-piperidin-4-yl}-1,4-dihydro-benzo[d][1,3]oxazin-2-one

554 N-{5-[4-(5-Chloro-2-oxo-4H-benzo[d][1,3]oxazin-1-yl)-piperidine-1-sulfonyl]-4-methyl-thiazol-2-yl)-acetamide

555 5-Chloro-1-[1-(3-chloro-2-fluoro-benzenesulfonyl)-piperidin-4-yl]-1,4-dihydro-benzo[d][1,3]oxazin-2-one

556 1-[1-(2-Bromo-4-fluoro-benzenesulfonyl)-piperidin-4-yl]-5-chloro-1,4-dihydro-benzo[d][1,3]oxazin-2-one

557 1-[1-(4-Bromo-3-trifluoromethyl-benzenesulfonyl)-piperidin-4-yl]-5-chloro-1,4-dihydro-benzo[d][1,3]oxazin-2-one

558 5-Chloro-1-[1-(5-chloro-2-fluoro-benzenesulfonyl)-piperidin-4-yl]-1,4-dihydro-benzo[d][1,3]oxazin-2-one

559 5-Chloro-1-[1-(isoquinoline-5-sulfonyl)-piperidin-4-yl]-1,4-dihydro-benzo[d][1,3]oxazin-2-one

560 1-[1-(2-Methanesulfonyl-benzenesulfonyl)-piperidin-4-yl]-8-methoxy-1,4-dihydro-benzo[d][1,3]oxazin-2-one

561 1-[1-(2-Methanesulfonyl-benzenesulfonyl)-piperidin-4-yl]-8-methoxy-1,4-dihydro-benzo[d][1,3]oxazin-2-one

562 1-[1-(3-Chloro-2-methyl-benzenesulfonyl)-piperidin-4-yl]-8-methoxy-1,4-dihydro-benzo[d][1,3]oxazin-2-one

563 1-[1-(4-Butyl-benzenesulfonyl)-piperidin-4-yl]-8-methoxy-1,4-dihydro-benzo[d][1,3]oxazin-2-one

564 1-[1-(4-Bromo-3-methyl-benzenesulfonyl)-piperidin-4-yl]-8-methoxy-1,4-dihydro-benzo[d][1,3]oxazin-2-one

565 1-{1-[1-(1,1-Dimethyl-propyl)-benzenesulfonyl]-piperidin-4-yl}-8-methoxy-1,4-dihydro-benzo[d][1,3]oxazin-2-one

566 N-{5-[4-(8-Methoxy-2-oxo-4H-benzo[d][1,3]oxazin-1-yl)-piperidine-1-sulfonyl]-4-methyl-thiazol-2-yl)-acetamide

567 1-[1-(3-Chloro-2-fluoro-benzenesulfonyl)-piperidin-4-yl]-8-methoxy-1,4-dihydro-benzo[d][1,3]oxazin-2-one

568 1-[1-(2-Bromo-4-fluoro-benzenesulfonyl)-piperidin-4-yl]-8-methoxy-1,4-dihydro-benzo[d][1,3]oxazin-2-one

569 1-[1-(4-Bromo-3-trifluoromethyl-benzenesulfonyl)-piperidin-4-yl]-8-methoxy-1,4-dihydro-benzo[d][1,3]oxazin-2-one

570 1-[1-(5-Chloro-2-fluoro-benzenesulfonyl)-piperidin-4-yl]-8-methoxy-1,4-dihydro-benzo[d][1,3]oxazin-2-one

571 1-[1-(Isoquinoline-5-sulfonyl)-piperidin-4-yl]-8-methoxy-1,4-dihydro-benzo[d][1,3]oxazin-2-one; hydrochloride

572 1-[1-(4-Methyl-naphthalene-1-sulfonyl)-piperidin-4-yl]-1,4-dihydro-benzo[d][1,3]oxazin-2-one

573 6-Chloro-1-[1-(4-methyl-naphthalene-1-sulfonyl)-piperidin-4-yl]-1,4-dihydro-benzo[d][1,3]oxazin-2-one

574 6-Methyl-1-[1-(4-methyl-naphthalene-1-sulfonyl)-piperidin-4-yl]-1,4-dihydro-benzo[d][1,3]oxazin-2-one

575 8-Methyl-1-[1-(4-methyl-naphthalene-1-sulfonyl)-piperidin-4-yl]-1,4-dihydro-benzo[d][1,3]oxazin-2-one

576 6-Fluoro-1-[1-(4-methyl-naphthalene-1-sulfonyl)-piperidin-4-yl]-1,4-dihydro-benzo[d][1,3]oxazin-2-one

577 8-Methoxy-1-[1-(4-methyl-naphthalene-1-sulfonyl)-piperidin-4-yl]-1,4-dihydro-benzo[d][1,3]oxazin-2-one

578 5-Chloro-1-[1-(4-methyl-naphthalene-1-sulfonyl)-piperidin-4-yl]-1,4-dihydro-benzo[d][1,3]oxazin-2-one

579 5-Chloro-1-[1-(naphthalene-1-sulfonyl)-piperidin-4-yl]-1,4-dihydro-benzo[d][1,3]oxazin-2-one

580 5-Chloro-1-[1-(naphthalene-2-sulfonyl)-piperidin-4-yl]-1,4-dihydro-benzo[d][1,3]oxazin-2-one

581 5-Chloro-1-[1-(quinoline-8-sulfonyl)-piperidin-4-yl]-1,4-dihydro-benzo[d][1,3]oxazin-2-one

582 5-Chloro-1-[1-(5-chloro-3-methyl-benzo[b]thiophene-2-sulfonyl)-piperidin-4-yl]-1,4-dihydro-benzo[d][1,3]oxazin-2-one

583 1-[1-(Benzo[b]thiophene-2-sulfonyl)-piperidin-4-yl]-5-chloro-1,4-dihydro-benzo[d][1,3]oxazin-2-one

584 1-[1-(Benzo[b]thiophene-3-sulfonyl)-piperidin-4-yl]-5-chloro-1,4-dihydro-benzo[d][1,3]oxazin-2-one

585 6-Bromo-1-[1-(4-methyl-naphthalene-1-sulfonyl)-piperidin-4-yl]-1,4-dihydro-benzo[d][1,3]oxazin-2-one

586 2-Chloro-4-fluoro-5-[4-(8-methyl-2-oxo-4H-benzo[d][1,3]oxazin-1-yl)-piperidine-1-sulfonyl]-benzoic acid

587 2-Chloro-5-[4-(6-chloro-2-oxo-4H-benzo[d][1,3]oxazin-1-yl)-piperidine-1-sulfonyl]-4-fluoro-benzoic acid

588 2-Chloro-4-fluoro-5-[4-(6-methyl-2-oxo-4H-benzo[d][1,3]oxazin-1-yl)-piperidine-1-sulfonyl]-benzoic acid

589 2-Chloro-4-fluoro-5-[4-(2-oxo-4H-benzo[d][1,3]oxazin-1-yl)-piperidine-1-sulfonyl]-benzoic acid

590 2-Chloro-4-fluoro-5-[4-(8-methoxy-2-oxo-4H-benzo[d][1,3]oxazin-1-yl)-piperidine-1-sulfonyl]-benzoic acid

591 2-Chloro-5-[4-(5-chloro-2-oxo-4H-benzo[d][1,3]oxazin-1-yl)-pi peridine-1-sulfonyl]-4-fluoro-benzoic acid

592 3-[4-(2-Oxo-4H-benzo[d][1,3]oxazin-1-yl)-piperidine-1-sulfonyl]-benzoic acid

593 3-[4-(8-Methoxy-2-oxo-4H-benzo[d][1,3]oxazin-1-yl)-piperidine-1-sulfonyl]-benzoic acid

594 3-[4-(5-Chloro-2-oxo-4H-benzo[d][1,3]oxazin-1-yl)-piperidine-1-sulfonyl]-benzoic acid

595 1-[1-(Isoquinoline-5-sulfonyl)-piperidin-4-yl]-8-methyl-1,4-dihydro-benzo[d][1,3]oxazin-2-one; hydrochloride

596 6-Chloro-1-[1-(isoquinoline-5-sulfonyl)-piperidin-4-yl]-1,4-dihydro-benzo[d][1,3]oxazin-2-one; hydrochloride

597 41-(Isoquinoline-5-sulfonyl)-piperidin-4-yl]-6-methyl-1,4-dihydro-benzo[d][1,3]oxazin-2-one; hydrochloride

598 6,7-Difluoro-1-[1-(quinoline-8-sulfonyl)-piperidin-4-yl]-1,4-dihydro-benzo[d][1,3]oxazin-2-one

599 1-[1-(5-Chloro-3-methyl-benzo[b]thiophene-2-sulfonyl)-piperidin-4-yl]-6,7-difluoro-1,4-dihydro-benzo[d][1,3]oxazin-2-one

600 6,7-Difluoro-1-[1-(naphthalene-1-sulfonyl)-piperidin-4-yl]-1,4-dihydro-benzo[d][1,3]oxazin-2-one

601 6,7-Difluoro-1-[1-(naphthalene-2-sulfonyl)-piperidin-4-yl]-1,4-dihydro-benzo[d][1,3]oxazin-2-one

602 1-[1-(Benzo[b]thiophene-2-sulfonyl)-piperidin-4-yl]-6,7-difluoro-1,4-dihydro-benzo[d][1,3]oxazin-2-one

603 1-[1-(Benzo[b]thiophene-3-sulfonyl)-piperidin-4-yl]-6,7-difluoro-1,4-dihydro-benzo[d][1,3]oxazin-2-one

604 1-[1-(5-Dimethylamino-naphthalene-1-sulfonyl)-piperidin-4-yl]-6,7-difluoro-1,4-dihydro-benzo[d][1,3]oxazin-2-one

605 1-[1-(Biphenyl-4-sulfonyl)-piperidin-4-yl]-6,7-difluoro-1,4-dihydro-benzo[d][1,3]oxazin-2-one

606 1-[1-(Benzo[1,2,5]thiadiazole-4-sulfonyl)-piperidin-4-yl]-6,7-difluoro-1,4-dihydro-benzo[d][1,3]oxazin-2-one

607 1-[1-(Benzo[1,2,5]oxadiazole-4-sulfonyl)-piperidin-4-yl]-6,7-difluoro-1,4-dihydro-benzo[d][1,3]oxazin-2-one

608 1-[1-(7-Chloro-benzo[1,2,5]oxadiazole-4-sulfonyl)-piperidin-4-yl]-6,7-difluoro-1,4-dihydro-benzo[d][1,3]oxazin-2-one

609 6,7-Difluoro-1-[1-(4-methyl-naphthalene-1-sulfonyl)-piperidin-4-yl]-1,4-dihydro-benzo[d][1,3]oxazin-2-one

610 1-[1-(4-Chloro-naphthalene-1-sulfonyl)-piperidin-4-yl]-1,4-dihydro-benzo[d][1,3]oxazin-2-one

611 1-[1-(4-Fluoro-naphthalene-1-sulfonyl)-piperidin-4-yl]-1,4-dihydro-benzo[d][1,3]oxazin-2-one

612 1-[1-(Dibenzofuran-2-sulfonyl)-piperidin-4-yl]-1,4-dihydro-benzo[d][1,3]oxazin-2-one

613 1-[1-(2,3-Dihydro-benzofuran-5-sulfonyl)-piperidin-4-yl]-1,4-dihydro-benzo[d][1,3]oxazin-2-one

614 1-[1-(Biphenyl-2-sulfonyl)-piperidin-4-yl]-1,4-dihydro-benzo[d][1,3]oxazin-2-one

615 1-[1-(5-Isoxazol-5-yl-thiophene-2-sulfonyl)-piperidin-4-yl]-1,4-dihydro-benzo[d][1,3]oxazin-2-one

616 1-[1-(4-Chloro-naphthalene-1-sulfonyl)-piperidin-4-yl]-8-methyl-1,4-dihydro-benzo[d][1,3]oxazin-2-one

617 1-[1-(4-Fluoro-naphthalene-1-sulfonyl)-piperidin-4-yl]-8-methyl-1,4-dihydro-benzo[d][1,3]oxazin-2-one

618 1-[1-(Dibenzofuran-2-sulfonyl)-piperidin-4-yl]-8-methyl-1,4-dihydro-benzo[d][1,3]oxazin-2-one

619 1-[1-(2,3-Dihydro-benzofuran-5-sulfonyl)-piperidin-4-yl]-8-methyl-1,4-dihydro-benzo[d][1,3]oxazin-2-one

620 1-[1-(Biphenyl-2-sulfonyl)-piperidin-4-yl]-8-methyl-1,4-dihydro-benzo[d][1,3]oxazin-2-one

621 1-[1-(5-Isoxazol-5-yl-thiophene-2-sulfonyl)-piperidin-4-yl]-8-methyl-1,4-dihydro-benzo[d][1,3]oxazin-2-one

622 5-Chloro-1-[1-(4-chloro-naphthalene-1-sulfonyl)-piperidin-4-yl]-1,4-dihydro-benzo[d][1,3]oxazin-2-one

623 5-Chloro-1-[1-(4-fluoro-naphthalene-1-sulfonyl)-piperidin-4-yl]-1,4-dihydro-benzo[d][1,3]oxazin-2-one

624 5-Chloro-1-[1-(dibenzofuran-2-sulfonyl)-piperidin-4-yl]-1,4-dihydro-benzo[d][1,3]oxazin-2-one

625 5-Chloro-1-[1-(2,3-dihydro-benzofuran-5-sulfonyl)-piperidin-4-yl]-1,4-dihydro-benzo[d][1,3]oxazin-2-one

626 1-[1-(Biphenyl-2-sulfonyl)-piperidin-4-yl]-5-chloro-1,4-dihydro-benzo[d][1,3]oxazin-2-one

627 5-Chloro-1-[1-(5-isoxazol-5-yl-thiophene-2-sulfonyl)-piperidin-4-yl]-1,4-dihydro-benzo[d][1,3]oxazin-2-one

628 1-[1-(4-Chloro-naphthalene-1-sulfonyl)-piperidin-4-yl]-8-methoxy-1,4-dihydro-benzo[d][1,3]oxazin-2-one

629 1-[1-(4-Fluoro-naphthalene-1-sulfonyl)-piperidin-4-yl]-8-methoxy-1,4-dihydro-benzo[d][1,3]oxazin-2-one

630 1-[1-(Dibenzofuran-2-sulfonyl)-piperidin-4-yl]-8-methoxy-1,4-dihydro-benzo[d][1,3]oxazin-2-one

631 1-[1-(2,3-Dihydro-benzofuran-5-sulfonyl)-piperidin-4-yl]-8-methoxy-1,4-dihydro-benzo[d][1,3]oxazin-2-one

632 1-[1-(Biphenyl-2-sulfonyl)-piperidin-4-yl]-8-methoxy-1,4-dihydro-benzo[d][1,3]oxazin-2-one

633 1-[1-(5-Isoxazol-5-yl-thiophene-2-sulfonyl)-piperidin-4-yl]-8-methoxy-1,4-dihydro-benzo[d][1,3]oxazin-2-one

634 6-Chloro-1-[1-(4-chloro-naphthalene-1-sulfonyl)-piperidin-4-yl]-1,4-dihydro-benzo[d][1,3]oxazin-2-one

635 6-Chloro-1-[1-(4-fluoro-naphthalene-1-sulfonyl)-piperidin-4-yl]-1,4-dihydro-benzo[d][1,3]oxazin-2-one

636 6-Chloro-1-[1-(dibenzofuran-2-sulfonyl)-piperidin-4-yl]-1,4-dihydro-benzo[d][1,3]oxazin-2-one

637 6-Chloro-1-[1-(2,3-dihydro-benzofuran-5-sulfonyl)-piperidin-4-yl]-1,4-dihydro-benzo[d][1,3]oxazin-2-one

638 1-[1-(Biphenyl-2-sulfonyl)-piperidin-4-yl]-6-chloro-1,4-dihydro-benzo[d][1,3]oxazin-2-one

639 6-Chloro-1-[1-(5-isoxazol-5-yl-thiophene-2-sulfonyl)-piperidin-4-yl]-1,4-dihydro-benzo[d][1,3]oxazin-2-one

640 1-[1-(4-Chloro-naphthalene-1-sulfonyl)-piperidin-4-yl]-6-methyl-1,4-dihydro-benzo[d][1,3]oxazin-2-one

641 1-[1-(4-Fluoro-naphthalene-1-sulfonyl)-piperidin-4-yl]-6-methyl-1,4-dihydro-benzo[d][1,3]oxazin-2-one

642 1-[1-(Dibenzofuran-2-sulfonyl)-piperidin-4-yl]-6-methyl-1,4-dihydro-benzo[d][1,3]oxazin-2-one

643 1-[1-(2,3-Dihydro-benzofuran-5-sulfonyl)-piperidin-4-yl]-6-methyl-1,4-dihydro-benzo[d][1,3]oxazin-2-one

644 1-[1-(Biphenyl-2-sulfonyl)-piperidin-4-yl]-6-methyl-1,4-dihydro-benzo[d][1,3]oxazin-2-one

645 1-[1-(5-Isoxazol-5-yl-thiophene-2-sulfonyl)-piperidin-4-yl]-6-methyl-1,4-dihydro-benzo[d][1,3]oxazin-2-one

646 1-[1-(4-Chloro-naphthalene-1-sulfonyl)-piperidin-4-yl]-6,7-difluoro-1,4-dihydro-benzo[d][1,3]oxazin-2-one

647 6,7-Difluoro-1-[1-(4-fluoro-naphthalene-1-sulfonyl)-piperidin-4-yl]-1,4-dihydro-benzo[d][1,3]oxazin-2-one

648 1-[1-(Dibenzofuran-2-sulfonyl)-piperidin-4-yl]-6,7-difluoro-1,4-dihydro-benzo[d][1,3]oxazin-2-one

649 1-[1-(2,3-Dihydro-benzofuran-5-sulfonyl)-piperidin-4-yl]-6,7-difluoro-1,4-dihydro-benzo[d][1,3]oxazin-2-one

650 1-[1-(Biphenyl-2-sulfonyl)-piperidin-4-yl]-6,7-difluoro-1,4-dihydro-benzo[d][1,3]oxazin-2-one

651 6,7-Difluoro-1-[1-(5-isoxazol-5-yl-thiophene-2-sulfonyl)-piperidin-4-yl]-1,4-dihydro-benzo[d][1,3]oxazin-2-one

652 1-[1-(1,2-Dimethyl-1H-imidazole-4-sulfonyl)-piperidin-4-yl]-1,4-dihydro-benzo[d][1,3]oxazin-2-one

653 1-[1-(5-Methyl-benzo[1,2,5]thiadiazole-4-sulfonyl)-piperidin-4-yl]-1,4-dihydro-benzo[d][1,3]oxazin-2-one

654 1-[1-(3,5-Dimethyl-isoxazole-4-sulfonyl)-piperidin-4-yl]-1,4-dihydro-benzo[d][1,3]oxazin-2-one

655 1-[1-(1,2-Dimethyl-1H-imidazole-4-sulfonyl)-piperidin-4-yl]-8-methyl-1,4-dihydro-benzo[d][1,3]oxazin-2-one

656 8-Methyl-1-[1-(5-methyl-benzo[1,2,5]thiadiazole-4-sulfonyl)-piperidin-4-yl]-1,4-dihydro-benzo[d][1,3]oxazin-2-one

657 1-[1-(3,5-Dimethyl-isoxazole-4-sulfonyl)-piperidin-4-yl]-8-methyl-1,4-dihydro-benzo[d][1,3]oxazin-2-one

658 6-Chloro-1-[1-(1,2-dimethyl-1H-imidazole-4-sulfonyl)-piperidin-4-yl]-1,4-dihydro-benzo[d][1,3]oxazin-2-one

659 6-Chloro-1-[1-(5-methyl-benzo[1,2,5]thiadiazole-4-sulfonyl)-piperidin-4-yl]-1,4-dihydro-benzo[d][1,3]oxazin-2-one

660 6-Chloro-1-[1-(3,5-dimethyl-isoxazole-4-sulfonyl)-piperidin-4-yl]-1,4-dihydro-benzo[d][1,3]oxazin-2-one

661 1-[1-(1,2-Dimethyl-1H-imidazole-4-sulfonyl)-piperidin-4-yl]-8-methoxy-1,4-dihydro-benzo[d][1,3]oxazin-2-one

662 8-Methoxy-1-[1-(5-methyl-benzo[1,2,5]thiadiazole-4-sulfonyl)-piperidin-4-yl]-1,4-dihydro-benzo[d][1,3]oxazin-2-one

663 1-[1-(3,5-Dimethyl-isoxazole-4-sulfonyl)-piperidin-4-yl]-8-methoxy-1,4-dihydro-benzo[d][1,3]oxazin-2-one

664 5-Chloro-1-[1-(1,2-dimethyl-1H-imidazole-4-sulfonyl)-piperidin-4-yl]-1,4-dihydro-benzo[d][1,3]oxazin-2-one

665 5-Chloro-1-[1-(5-methyl-benzo[1,2,5]thiadiazole-4-sulfonyl)-piperidin-4-yl]-1,4-dihydro-benzo[d][1,3]oxazin-2-one

666 5-Chloro-1-[1-(3,5-dimethyl-isoxazole-4-sulfonyl)-piperidin-4-yl]-1,4-dihydro-benzo[d][1,3]oxazin-2-one

667 1-[1-(1,2-Dimethyl-1H-imidazole-4-sulfonyl)-piperidin-4-yl]-6-methyl-1,4-dihydro-benzo[d][1,3]oxazin-2-one

668 6-Methyl-1-[1-(5-methyl-benzo[1,2,5]thiadiazole-4-sulfonyl)-piperidin-4-yl]-1,4-dihydro-benzo[d][1,3]oxazin-2-one

669 1-[1-(3,5-Dimethyl-isoxazole-4-sulfonyl)-piperidin-4-yl]-6-methyl-1,4-dihydro-benzo[d][1,3]oxazin-2-one

670 1-[1-(1,2-Dimethyl-1H-imidazole-4-sulfonyl)-piperidin-4-yl]-6-fluoro-1,4-dihydro-benzo[d][1,3]oxazin-2-one

671 6-Fluoro-1-[1-(5-methyl-benzo[1,2,5]thiadiazole-4-sulfonyl)-piperidin-4-yl]-1,4-dihydro-benzo[d][1,3]oxazin-2-one

672 1-[1-(3,5-Dimethyl-isoxazole-4-sulfonyl)-piperidin-4-yl]-6-fluoro-1,4-dihydro-benzo[d][1,3]oxazin-2-one

673 1-[1-(1,2-Dimethyl-1H-imidazole-4-sulfonyl)-piperidin-4-yl]-6,7-difluoro-1,4-dihydro-benzo[d][1,3]oxazin-2-one

674 6,7-Difluoro-1-[1-(5-methyl-benzo[1,2,5]thiadiazole-4-sulfonyl)-piperidin-4-yl]-1,4-dihydro-benzo[d][1,3]oxazin-2-one

675 1-[1-(3,5-Dimethyl-isoxazole-4-sulfonyl)-piperidin-4-yl]-6,7-difluoro-1,4-dihydro-benzo[d][1,3]oxazin-2-one

676 1-[1-(5-Chloro-naphthalene-1-sulfonyl)-piperidin-4-yl]-1,4-dihydro-benzo[d][1,3]oxazin-2-one

677 1-[1-(5-Chloro-naphthalene-2-sulfonyl)-piperidin-4-yl]-1,4-dihydro-benzo[d][1,3]oxazin-2-one

678 N-{5-[4-(2-Oxo-4H-benzo[d][1,3]oxazin-1-yl)-piperidine-1-sulfonyl]-naphthalen-1-yl}-acetamide

679 1-[1-(5-Chloro-naphthalene-1-sulfonyl)-piperidin-4-yl]-8-methyl-1,4-dihydro-benzo[d][1,3]oxazin-2-one

680 1-[1-(5-Chloro-naphthalene-2-sulfonyl)-piperidin-4-yl]-8-methyl-1,4-dihydro-benzo[d][1,3]oxazin-2-one

681 N-{5-[4-(8-Methyl-2-oxo-4H-benzo[d][1,3]oxazin-1-yl)-piperidine-1-sulfonyl]-naphthalen-1-yl}-acetamide

682 5-Chloro-1-[1-(5-chloro-naphthalene-1-sulfonyl)-piperidin-4-yl]-1,4-dihydro-benzo[d][1,3]oxazin-2-one

683 5-Chloro-1-[1-(5-chloro-naphthalene-2-sulfonyl)-piperidin-4-yl]-1,4-dihydro-benzo[d][1,3]oxazin-2-one

684 N-{5-[4-(5-Chloro-2-oxo-4H-benzo[d][1,3]oxazin-1-yl)-piperidine-1-sulfonyl]-naphthalen-1-yl)-acetamide

685 1-[1-(5-Chloro-naphthalene-1-sulfonyl)-piperidin-4-yl]-8-methoxy-1,4-dihydro-benzo[d][1,3]oxazin-2-one

686 1-[1-(5-Chloro-naphthalene-2-sulfonyl)-piperidin-4-yl]-8-methoxy-1,4-dihydro-benzo[d][1,3]oxazin-2-one

687 N-{5-[4-(8-Methoxy-2-oxo-4H-benzo[d][1,3]oxazin-1-yl)-piperidine-1-sulfonyl]-naphthalen-1-yl)-acetamide

688 2,5-Dimethyl-4-[4-(8-methyl-2-oxo-4H-benzo[d][1,3]oxazin-1-yl)-piperidine-1-sulfonyl]-furan-3-carboxylic acid methyl ester

689 8-Methyl-1-[1-(2-oxo-2,3-dihydro-benzothiazole-6-sulfonyl)-piperidin-4-yl]-1,4-dihydro-benzo[d][1,3]oxazin-2-one

690 1-[1-(4-Fluoro-3-methyl-benzenesulfonyl)-piperidin-4-yl]-8-methyl-1,4-dihydro-benzo[d][1,3]oxazin-2-one

691 8-Methyl-1-[1-(2-oxo-2,3-dihydro-benzooxazole-6-sulfonyl)-piperidin-4-yl]-1,4-dihydro-benzo[d][1,3]oxazin-2-one

692 1-[1-(4-Cyclohexyl-benzenesulfonyl)-piperidin-4-yl]-8-methyl-1,4-dihydro-benzo[d][1,3]oxazin-2-one

693 2 ,5-Dimethyl-4-[4-(2-oxo-4H-benzo[d][1,3]oxazin-1-yl)-piperidine-1-sulfonyl]-furan-3-carboxylic acid methyl ester

694 1-[1-(4-Fluoro-3-methyl-benzenesulfonyl)-piperidin-4-yl]-1,4-dihydro-benzo[d][1,3]oxazin-2-one

695 1-[1-(2-Oxo-2,3-dihydro-benzooxazole-6-sulfonyl)-piperidin-4-yl]-1,4-dihydro-benzo[d][1,3]oxazin-2-one

696 1-[1-(4-Cyclohexyl-benzenesulfonyl)-piperidin-4-yl]-1,4-dihydro-benzo[d][1,3]oxazin-2-one

697 2-Fluoro-5-[4-(8-methyl-2-oxo-4H-benzo[d][1,3]oxazin-1-yl)-piperidine-1-sulfonyl]-benzoic acid

698 2-Fluoro-5-[4-(2-oxo-4H-benzo[d][1,3]oxazin-1-yl)-piperidine-1-sulfonyl]-benzoic acid

699 1-[1-(2-Oxo-2,3-dihydro-benzoth iazole-6-sulfonyl)-piperidin-4-yl]-1,4-dihydro-benzo[d][1,3]oxazin-2-one

700 1-[1-(5-Pyridin-2-yl-thiophene-2-sulfonyl)-piperidin-4-yl]-1,4-dihydro-benzo[d][1,3]oxazin-2-one

701 3-[4-(2-Oxo-4H-benzo[d][1,3]oxazin-1-yl)-piperidine-1-sulfonyl]-benzonitrile

702 3-[4-(2-Oxo-4H-benzo[d][1,3]oxazin-1-yl)-pi peridine-1-sulfonyl]-thiophene-2-carboxylic acid methyl ester

703 1-{5-[4-(2-Oxo-4H-benzo[d][1,3]oxazin-1-yl)-piperidine-1-sulfonyl]-naphthalen-1-yl}-pyrrolidine-2,5-dione

704 1-[1-(2-Chloro-5-trifluoromethyl-benzenesulfonyl)-piperidin-4-yl]-1,4-dihydro-benzo[d][1,3]oxazin-2-one

705 1-[1-(3,4-Dimethyl-benzenesulfonyl)-piperidin-4-yl]-1,4-dihydro-benzo[d][1,3]oxazin-2-one

706 8-Methyl-1-[1-(5-pyridin-2-yl-thiophene-2-sulfonyl)-piperidin-4-yl]-1,4-dihydro-benzo[d][1,3]oxazin-2-one

707 3-[4-(8-Methyl-2-oxo-4H-benzo[d][1,3]oxazin-1-yl)-piperidine-1-sulfonyl]-benzonitrile

708 3-[4-(8-Methyl-2-oxo-4H-benzo[d][1,3]oxazin-1-yl)-piperidine-1-sulfonyl]-thiophene-2-carboxylic acid methyl ester

709 1-{5-[4-(8-Methyl-2-oxo-4H-benzo[d][1,3]oxazin-1-yl)-piperidine-1-sulfonyl]-naphthalen-1-yl}-pyrrolidine-2,5-dione

710 1-[1-(2-Chloro-5-trifluoromethyl-benzenesulfonyl)-piperidin-4-yl]-8-methyl-1,4-dihydro-benzo[d][1,3]oxazin-2-one

711 1-[1-(3,4-Dimethyl-benzenesulfonyl)-piperidin-4-yl]-8-methyl-1,4-dihydro-benzo[d][1,3]oxazin-2-one

712 5-Chloro-1-[1-(5-pyridin-2-yl-thiophene-2-sulfonyl)-piperidin-4-yl]-1,4-dihydro-benzo[d][1,3]oxazin-2-one

713 3-[4-(5-Chloro-2-oxo-4H-benzo[d][1,3]oxazin-1-yl)-piperidine-1-sulfonyl]-benzonitrile

714 3-[4-(5-Chloro-2-oxo-4H-benzo[d][1,3]oxazin-1-yl)-piperidine-1-sulfonyl]-thiophene-2-carboxylic acid methyl ester

715 1-{5-[4-(5-Chloro-2-oxo-4H-benzo[d][1,3]oxazin-1-yl)-piperidine-1-sulfonyl]-naphthalen-1-yl}-pyrrolidine-2,5-dione

716 5-Chloro-1-[1-(2-chloro-5-trifluoromethyl-benzenesulfonyl)-piperidin-4-yl]-1,4-dihydro-benzo[d][1,3]oxazin-2-one

717 5-Chloro-1-[1-(3,4-dimethyl-benzenesulfonyl)-piperidin-4-yl]-1,4-dihydro-benzo[d][1,3]oxazin-2-one

718 6-Methyl-1-[1-(5-pyridin-2-yl-thiophene-2-sulfonyl)-piperidin-4-yl]-1,4-dihydro-benzo[d][1,3]oxazin-2-one

719 3-[4-(6-Methyl-2-oxo-4H-benzo[d][1,3]oxazin-1-yl)-piperidine-1-sulfonyl]-benzonitrile

720 3-[4-(6-Methyl-2-oxo-4H-benzo[d][1,3]oxazin-1-yl)-piperidine-1-sulfonyl]-thiophene-2-carboxylic acid methyl ester

721 1-{5-[4-(6-Methyl-2-oxo-4H-benzo[d][1,3]oxazin-1-yl)-piperidine-1-sulfonyl]-naphthalen-1-yl}-pyrrolidine-2,5-dione

722 1-[1-(2-Chloro-5-trifluoromethyl-benzenesulfonyl)-piperidin-4-yl]-6-methyl-1,4-dihydro-benzo[d][1,3]oxazin-2-one

723 1-[1-(3,4-Dimethyl-benzenesulfonyl)-piperidin-4-yl]-6-methyl-1,4-dihydro-benzo[d][1,3]oxazin-2-one

724 6-Chloro-1-[1-(5-pyridin-2-yl-thiophene-2-sulfonyl)-piperidin-4-yl]-1,4-dihydro-benzo[d][1,3]oxazin-2-one

725 3-[4-(6-Chloro-2-oxo-4H-benzo[d][1,3]oxazin-1-yl)-piperidine-1-sulfonyl]-benzonitrile

726 3-[4-(6-Chloro-2-oxo-4H-benzo[d][1,3]oxazin-1-yl)-piperidine-1-sulfonyl]-thiophene-2-carboxylic acid methyl ester

727 1-{5-[4-(6-Chloro-2-oxo-4H-benzo[d][1,3]oxazin-1-yl)-piperidine-1-sulfonyl]-naphthalen-1-yl}-pyrrolidine-2,5-dione

728 6-Chloro-1-[1-(2-chloro-5-trifluoromethyl-benzenesulfonyl)-piperidin-4-yl]-1,4-dihydro-benzo[d][1,3]oxazin-2-one

729 6-Chloro-1-[1-(3,4-dimethyl-benzenesulfonyl)-piperidin-4-yl]-1,4-dihydro-benzo[d][1,3]oxazin-2-one

730 1-[1-(5-Methyl-isoxazole-4-sulfonyl)-piperidin-4-yl]-1,4-dihydro-benzo[d][1,3]oxazin-2-one

731 1-[1-(2,2-Dimethyl-chroman-6-sulfonyl)-piperidin-4-yl]-1,4-dihydro-benzo[d][1,3]oxazin-2-one

732 1-[1-(4-Methyl-3,4-dihydro-2H-benzo[1,4]oxazine-7-sulfonyl)-piperidin-4-yl]-1,4-dihydro-benzo[d][1,3]oxazin-2-one

733 1-[1-(2,3-Dihydro-benzo[1,4]dioxine-6-sulfonyl)-piperidin-4-yl]-1,4-dihydro-benzo[d][1,3]oxazin-2-one

734 1-[1-(1,3,5-Trimethyl-1H-pyrazole-4-sulfonyl)-piperidin-4-yl]-1,4-dihydro-benzo[d][1,3]oxazin-2-one

735 1-[1-(3-Methyl-2-oxo-2,3-dihydro-benzooxazole-6-sulfonyl)-piperidin-4-yl]-1,4-dihydro-benzo[d][1,3]oxazin-2-one

736 8-Methyl-1-[1-(5-methyl-isoxazole-4-sulfonyl)-piperidin-4-yl]-1,4-dihydro-benzo[d][1,3]oxazin-2-one

737 1-[1-(2,2-Dimethyl-chroman-6-sulfonyl)-piperidin-4-yl]-8-methyl-1,4-dihydro-benzo[d][1,3]oxazin-2-one

738 8-Methyl-1-[1-(4-methyl-3,4-dihydro-2H-benzo[1,4]oxazine-7-sulfonyl)-piperidin-4-yl]-1,4-dihydro-benzo[d][1,3]oxazin-2-one

739 1-[1-(2,3-Dihydro-benzo[1,4]dioxine-6-sulfonyl)-piperidin-4-yl]-8-methyl-1,4-dihydro-benzo[d][1,3]oxazin-2-one

740 8-Methyl-1-[1-(1,3,5-trimethyl-1H-pyrazole-4-sulfonyl)-piperidin-4-yl]-1,4-dihydro-benzo[d][1,3]oxazin-2-one

741 8-Methyl-1-[1-(3-methyl-2-oxo-2,3-di hydro-benzooxazole-6-sulfonyl)-piperidin-4-yl]-1,4-dihydro-benzo[d][1,3]oxazin-2-one

742 8-Methoxy-1-[1-(1,3,5-trimethyl-1H-pyrazole-4-sulfonyl)-piperidin-4-yl]-1,4-dihydro-benzo[d][1,3]oxazin-2-one

743 8-Methoxy-1-[1-(3-methyl-2-oxo-2,3-dihydro-benzooxazole-6-sulfonyl)-piperidin-4-yl]-1,4-dihydro-benzo[d][1,3]oxazin-2-one

744 1-[1-(Benzo[d]isoxazol-3-ylmethanesulfonyl)-piperidin-4-yl]-1,4-dihydro-benzo[d][1,3]oxazin-2-one

745 1-[1-(2,2,4,6,7-Pentamethyl-2,3-dihydro-benzofuran-5-sulfonyl)-piperidin-4-yl]-1,4-dihydro-benzo[d][1,3]oxazin-2-one

746 6-Methyl-5-[4-(2-oxo-4H-benzo[d][1,3]oxazin-1-yl)-piperidine-1-sulfonyl]-1H-pyrimidine-2,4-dione

747 1-[1-(3-Methyl-quinoline-8-sulfonyl)-piperidin-4-yl]-1,4-dihydro-benzo[d][1,3]oxazin-2-one

748 1-[1-(2,2,5,7,8-Pentamethyl-chroman-6-sulfonyl)-piperidin-4-yl]-1,4-dihydro-benzo[d][1,3]oxazin-2-one

749 1,4-Dimethyl-644-(2-oxo-4H-benzo[d][1,3]oxazin-1-yl)-piperidine-1-sulfonyl]-1,4-dihydro-quinoxaline-2,3-dione

750 1-[1-(1H-Imidazole-4-sulfonyl)-piperidin-4-yl]-1,4-dihydro-benzo[d][1,3]oxazin-2-one

751 1-[1-(2-Oxo-1,2,3,4-tetrahydro-quinoline-6-sulfonyl)-piperidin-4-yl]-1,4-dihydro-benzo[d][1,3]oxazin-2-one

752 7-[4-(2-Oxo-4H-benzo[d][1,3]oxazin-1-yl)-piperidine-1-sulfonyl]-1,5-dihydro-benzo[b][1,4]diazepine-2,4-dione

753 8-Methyl-1-[1-(3-methyl-quinoline-8-sulfonyl)-piperidin-4-yl]-1,4-dihydro-benzo[d][1,3]oxazin-2-one

754 6-Chloro-1-[1-(3-methyl-quinoline-8-sulfonyl)-piperidin-4-yl]-1,4-dihydro-benzo[d][1,3]oxazin-2-one

755 5-Chloro-1-[1-(3-methyl-quinoline-8-sulfonyl)-piperidin-4-yl]-1,4-dihydro-benzo[d][1,3]oxazin-2-one

756 8-Methoxy-1-[1-(3-methyl-quinoline-8-sulfonyl)-piperidin-4-yl]-1,4-dihydro-benzo[d][1,3]oxazin-2-one

757 1-[1-(Pyridine-2-sulfonyl)-piperidin-4-yl]-1,4-dihydro-benzo[d][1,3]oxazin-2-one

758 1-[1-(6,7-Dihydroxy-naphthalene-1-sulfonyl)-piperidin-4-yl]-1,4-dihydro-benzo[d][1,3]oxazin-2-one

759 Acetic acid 3-acetoxy-5-[4-(2-oxo-4H-benzo[d][1,3]oxazin-1-yl)-piperidine-1-sulfonyl]-naphthalen-2-ylester

760 1-[1-(1H-Benzoimidazole-2-sulfonyl)-piperidin-4-yl]-1,4-dihydro-benzo[d][1,3]oxazin-2-one

761 1-[1-(1H-Benzoimidazole-2-sulfonyl)-piperidin-4-yl]-8-methoxy-1,4-dihydro-benzo[d][1,3]oxazin-2-one

762 1-[1-(1H-Benzoimidazole-2-sulfonyl)-piperidin-4-yl]-5-chloro-1,4-dihydro-benzo[d][1,3]oxazin-2-one

763 1-[1-(2,5-Dimethoxy-benzenesulfonyl)-piperidin-4-yl]-6-fluoro-1,4-dihydro-benzo[d][1,3]oxazin-2-one

764 1-[1-(2,5-Dimethoxy-benzenesulfonyl)-piperidin-4-yl]-8-methoxy-1,4-dihydro-benzo[d][1,3]oxazin-2-one

765 1-[1-(2,5-Dimethoxy-benzenesulfonyl)-piperidin-4-yl]-6,7-difluoro-1,4-dihydro-benzo[d][1,3]oxazin-2-one

766 5-Chloro-1-[1-(2,5-dimethoxy-benzenesulfonyl)-piperidin-4-yl]-1,4-dihydro-benzo[d][1,3]oxazin-2-one

767 1-[1-(5-Dimethylamino-naphthalene-1-sulfonyl)-piperidin-4-yl]-8-methoxy-1,4-dihydro-benzo[d][1,3]oxazin-2-one

768 5-Chloro-1-[1-(5-dimethylamino-naphthalene-1-sulfonyl)-piperidin-4-yl]-1,4-dihydro-benzo[d][1,3]oxazin-2-one

769 6-Chloro-1-[1-(5-chloro-naphthalene-1-sulfonyl)-piperidin-4-yl]-1,4-dihydro-benzo[d][1,3]oxazin-2-one

770 1-[1-(5-Chloro-naphthalene-1-sulfonyl)-piperidin-4-yl]-6-methyl-1,4-dihydro-benzo[d][1,3]oxazin-2-one

771 1-[1-(5-Chloro-naphthalene-1-sulfonyl)-piperidin-4-yl]-6-fluoro-1,4-dihydro-benzo[d][1,3]oxazin-2-one

772 6-Chloro-1-[1-(5-chloro-naphthalene-2-sulfonyl)-piperidin-4-yl]-1,4-dihydro-benzo[d][1,3]oxazin-2-one

773 1-[1-(5-Chloro-naphthalene-2-sulfonyl)-piperidin-4-yl]-6-methyl-1,4-dihydro-benzo[d][1,3]oxazin-2-one

774 1-[1-(5-Chloro-naphthalene-2-sulfonyl)-piperidin-4-yl]-6-fluoro-1,4-dihydro-benzo[d][1,3]oxazin-2-one

775 6-Methyl-1-[1-(3-methyl-quinoline-8-sulfonyl)-piperidin-4-yl]-1,4-dihydro-benzo[d][1,3]oxazin-2-one

776 6-Fluoro-1-[1-(3-methyl-quinoline-8-sulfonyl)-piperidin-4-yl]-1,4-dihydro-benzo[d][1,3]oxazin-2-one

777 6,7-Difluoro-1-[1-(3-methyl-quinoline-8-sulfonyl)-piperidin-4-yl]-1,4-dihydro-benzo[d][1,3]oxazin-2-one

778 6-Chloro-1-[1-(3-methyl-2-oxo-2,3-dihydro-benzooxazole-6-sulfonyl)-piperidin-4-yl]-1,4-dihydro-benzo[d][1,3]oxazin-2-one

779 6-Methyl-1-[1-(3-methyl-2-oxo-2,3-dihydro-benzooxazole-6-sulfonyl)-piperidin-4-yl]-1,4-dihydro-benzo[d][1,3]oxazin-2-one

780 6-Fluoro-1-[1-(3-methyl-2-oxo-2,3-dihydro-benzooxazole-6-sulfonyl)-piperidin-4-yl]-1,4-dihydro-benzo[d][1,3]oxazin-2-one

781 6,7-Difluoro-1-[1-(3-methyl-2-oxo-2,3-dihydro-benzooxazole-6-sulfonyl)-piperidin-4-yl]-1,4-dihydro-benzo[d][1,3]oxazin-2-one

782 5-Chloro-1-[1-(3-methyl-2-oxo-2,3-dihydro-benzooxazole-6-sulfonyl)-piperidin-4-yl]-1,4-dihydro-benzo[d][1,3]oxazin-2-one

783 6-Chloro-1-[1-(4-methyl-3,4-dihydro-2H-benzo[1,4]oxazine-7-sulfonyl)-piperidin-4-yl]-1,4-dihydro-benzo[d][1,3]oxazin-2-one

784 6-Methyl-1-[1-(4-methyl-3,4-dihydro-2H-benzo[1,4]oxazine-7-sulfonyl)-piperidin-4-yl]-1,4-dihydro-benzo[d][1,3]oxazin-2-one

785 6-Fluoro-1-[1-(4-methyl-3,4-dihydro-2H-benzo[1,4]oxazine-7-sulfonyl)-piperidin-4-yl]-1,4-dihydro-benzo[d][1,3]oxazin-2-one

786 8-Methoxy-1-[1-(4-methyl-3,4-dihydro-2H-benzo[1,4]oxazi ne-7-sulfonyl)-piperidin-4-yl]-1,4-dihydro-benzo[d][1,3]oxazin-2-one

787 5-Chloro-1-[1-(4-methyl-3,4-dihydro-2H-benzo[1,4]oxazine-7-sulfonyl)-piperidin-4-yl]-1,4-dihydro-benzo[d][1,3]oxazin-2-on ;

optionally in form of one of its stereoisomers, preferably enantiomers or diastereomers, its racemate or in form of a mixture of at least two of its stereoisomers, preferably enantiomers or diastereomers, in any mixing ratio, or a physiologically acceptable salt thereof, or a solvate, respectively.

The compounds of general formula (Ia) can be prepared according to the disclosure of WO 2005/014045. The respective part of the literature is hereby incorporated by reference and forms part of the present disclosure.

Preferably as component (A) at least one compound is present which is selected from the group consisting of indole-derived sulfonamide compounds of general formula (Ib)

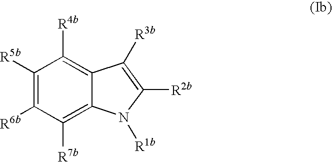

wherein

R^{1b} K represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical,

which may be bonded via a linear or branched alkylene group and/or which may be condensed with an unsubstituted or at least mono-substituted mono- or bicyclic ring system; an unsubstituted or at least mono-substituted aryl or heteroaryl radical, which may be bonded via a linear or branched alkylene group and/or which may be condensed with an unsubstituted or at least mono-substituted mono- or bicyclic ring system; a —(CH₂)_{mb}—NR^{13b}R^{14b} moiety with mb=0, 1, 2, 3, 4 or 5; a —C(═O)—R^{8b} moiety; a —S(═O)₂—R^{9b} moiety; or a —S(═O)₂—C(H)A^{b}B^{b} moiety;

R^{2b} K represents a hydrogen atom; —F; —Cl; —Br; —I; —NO₂; —NH₂; —SH; —OH; —CN; —C(═O)—OH; —O—R^{10b}; —S—R^{11b}; —C(═O)—OR^{12b}; a linear or branched, saturated or unsaturated, unsubstituted or at least mono-substituted, optionally at least one heteroatom as a chain member containing aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R^{3b} represents a hydrogen atom; —F; —Cl; —Br; —I; —NO₂; —CN; —O—R^{10b}; —S—R^{11b}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; a —CH(OC₂H₅)—CH₂—NR^{13b}R^{14b} moiety or a —(CH₂)_{nb}—NR^{13b}R^{14b} moiety with nb=0, 1, 2, 3, 4 or 5; a —S(═O)₂—R^{9b} moiety; a —S(═O)₂—C(H)A^{b}B^{b} moiety; or a —C(═O)—(CH₂)_{pb}—C(═O)—N-D^{b}E^{b} moiety with pb=0, 1, 2, 3, 4 or 5;

R^{4b}, R^{5b}, R^{6b} and R^{7b}, independently of one another, each represent a hydrogen atom; —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{8b}; —S(═O)₂—R^{9b}; —O—R^{19b}; —S—R^{11b}; —C(═O)—OR^{12b}; —N(R^{15b})—S(═O)₂—R^{16b}; —NH—R^{17b}; —NR^{18b}R^{19b}; —C(═O)—NHR^{20b}, —C(═O)—NR^{21b}R^{22b}; —S(═O)₂—NHR^{23b}; —S(═O)₂—NR^{24b}R^{25b}; —O—C(═O)—R^{26b}; —NH—C(═O)—R^{27b}; —NR^{28b}—C(═O)—R^{29b}; NH—C(═O)—O—R^{30b}; NR^{31b}—C(═O)—O—R^{32b}; —S(═O)₂—O—R^{33b}; a halogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene group;

with the proviso that at least one of the substituents R^{b}, R^{5b}, R^{6b} and R^{7b} represents a —N(R^{15b})—S(═O)₂—R^{16b} moiety;

R^{8b}, R^{12b}, R^{17b}, R^{18b}, R^{19b}, R^{20b}, R^{21b}, R^{22b}, R^{23b}, R^{24b}, R^{25b}, R^{26b}, R^{27b}, R^{28b}, R^{29b}, R^{30b}, R^{31b}, R^{32b} and R^{33b}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene, alkenylene or alkinylene group;

R^{9b} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{10b} and R^{11b}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{13b} and R^{14b}, independently of one another, each represent a hydrogen atom; or a linear or branched, saturated or unsaturated, unsubstituted or at least mono-substituted aliphatic radical;

or

R^{13b} and R^{14b} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{15b} represents a hydrogen atom; a linear or branched, saturated or unsaturated, unsubstituted or at least mono-substituted aliphatic radical or a —S(═O)₂—R^{16b} moiety;

R^{16b} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

A^{b} and B^{b} together with the bridging carbon form an unsubstituted or at least mono-substituted, saturated or unsaturated cycloaliphatic ring which may contain at least one further heteroatom as a ring member and/or which may be condensed with an unsubstituted or at least mono-substituted mono- or bicyclic ring system;

D^{b} and E^{b} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

D^{b} and E^{b}, independently of one another, each represent a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ib) are selected from the group consisting of compounds of general formula (Ih)

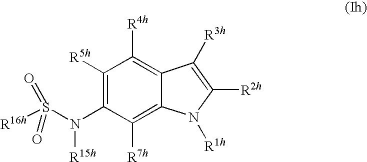

wherein

R^{1h} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₕ—NR^{13h}R^{14h} moiety with mh=0, 1, 2, 3, 4 or 5;

R^{2h} represents a hydrogen atom; —F; —Cl; —Br; —I; —NO₂; —CN; —O—R^{10h}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R^{3h} represents a hydrogen atom; —F; —Cl; —Br; —I; —NO₂; —CN; —O—R^{10h}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₕ—NR^{13h}R^{14h} moiety with nh=0, 1, 2, 3, 4 or 5;

R^{4h}, R^{5h} and R^{7h}, independently of one another, each represent a hydrogen atom; —NO₂; —CN; —O—R^{10h}; —C(═O)—OR^{12h}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{10h} represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{13h} and R^{14h}, independently of one another, each represent a hydrogen atom; or a linear or branched, saturated or unsaturated, unsubstituted or at least mono-substituted aliphatic radical;

or

R^{13h} and R^{14h} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{15h} represents a hydrogen atom; a linear or branched, saturated or unsaturated, unsubstituted or at least mono-substituted aliphatic radical or a —S(═O)₂—R^{16h} moiety;

and R^{16h} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ih) are those, wherein

R^{1h} represents a hydrogen atom or a —(CH₂)ₘₕ—NR^{13h}R^{14h} radical,

R^{2h}, and R^{7h} each represent hydrogen,

R^{3h} represents a hydrogen atom, 1-methyl-piperidin-4-yl or a —(CH₂)ₙₕ—NR^{13h}R^{14h} moiety with nh=0, 1 or 2,

R^{4h} represents chlorine, bromine or a hydrogen atom,

R^{4h} K represents —C(═O)—O—C₂H₅ or a hydrogen atom,

R^{15h} represents hydrogen or a —S(═O)₂—R^{16h} moiety,

R^{13h} and R^{14h}, identical or different, each represent methyl, ethyl, isopropyl or n-propyl, more preferably methyl,

or

R^{13h} and R^{14h}, together with the bridging nitrogen atom form a 5- or 6-membered heterocyclic ring, more preferably form a pyrrolidine ring or a piperidine ring

and

R^{16h} represents an aryl or heteroaryl radical selected from the group consisting of phenyl, naphthyl, thiophenyl, benzo[b]furanyl, benzo[b]thiophenyl and imidazo[2,1-b]thiazoly1 which may be substituted by 1, 2 or 3 substituents selected from the group consisting of chlorine, methyl, phenyl and —O-phenyl and/or which may be bonded via a C₁₋₂ alkylene group,

and mh is 0, 1 or 2,

optionally in form of one of its stereoisomers, preferably enantiomers or diastereomers, a racemate or in form of a mixture of at least two of its stereoisomers, preferably enantiomers and/or diastereomers, in any mixing ratio, or a salt thereof, preferably a corresponding, physiologically acceptable salt thereof, or a corresponding solvate thereof.

Particularly preferred compounds of general formula (Ih) are those selected from the group consisting of:

[788] N-[1-(2-Dimethylaminoethyl)-1H-indol-6-yl]-5-chloro-3-methylbenzo[b]thiophene-2-sulfonamide,

[789] N-[1-(2-Dimethylaminoethyl)-1H-indol-6-yl]-naphthalene-2-sulfonamide,

[790] N-[1-(2-Dimethylaminoethyl)-1H-indol-6-yl]-naphthalene-1-sulfonamide,

[791] N-[1-(2-Dimethylaminoethyl)-1H-indol-6-yl]-6-chloroimidazo[2,1-b]thiazole-5-sulfonamide,

[792] N-[1-(2-Dimethylaminoethyl)-1H-indol-6-yl]-4-phenylbenzenesulfonamide,

[793] N-[1-(2-Dimethylaminoethyl)-1H-indol-6-yl]-2-(naphthalene-1-yl)-ethanesulfonamide,

[794] N-[1-(2-Dimethylaminoethyl)-1H-indol-6-yl]-4-phenoxybenzenesulfonamide,

[795] N-[1-(2-Dimethylaminoethyl)-1H-indol-6-yl]-3,5-dichlorobenzenesulfonamide,

[796] 5-Chloro-3-methyl-N-[1-[2-(pyrrolidin-1-yl)ethyl-1H-indol-6-yl]-benzo[b]thiophene-2-sulfonamide,

[797] N-(1-[2-(Pyrrolidin-1-yl)ethyl]-1H-indol-6-yl]-napthyl-2-sulfonamide,

[798] N-[1-[2-Pyrrolidin-1-yl]ethyl]-1H-indol-6-yl]-naphthalene-1-sulfonamide,

[799] 6-Chloro-N-[1-[2-(pyrrolidin-1-yl)ethyl]-1H-indol-6-yl]-imidazo[2,1-b]thiazole-5-sulfonamide,

[800] 4-Phenyl-N-(1-(2-(pyrrolidin-1-yl)ethyl)-1H-indol-6-yl)-benzenesulfonamide

[801] 2-(Naphth-1-yl)-N-(1-(2-(pyrrolidin-1-yl)ethyl)-1H-indol-6-yl)-ethansulfonamide,

[802] 4-Phenoxy-N-(1-(2-(pyrrolidin-1-yl)ethyl)-1H-indol-6-yl)-benzenesulfonamide

[803] 3,5-Dichloro-N-(1-(2-(pyrrolidin-1-yl)-1H-indol-6-yl)-benzenesulfonamide,

[804] 5-chloro-N-(3-(2-(diethylamino)ethyl)-1H-indol-6-yl)-3-methylbenzo[b]thiophene-2-sulfonamide,

[805] N-(3-(2-(diethylamino)ethyl)-1H-indol-6-yl)naphthalene-2-sulfonamide,

[806] N-(3-(2-(diethylamino)ethyl)-1H-indol-6-yl)naphthalene-1-sulfonamide,

[807] 6-chloro-N-(3-(2-(diethylamino)ethyl)-1H-indol-6-ypimidazo[2,1-b]thiazole-5-sulfonamide,

[808] N-(3-(2-(diethylamino)ethyl)-1H-indol-6-yl)-4-phenylbenzenesulfonamide,

[809] N-(3-(2-(diethylamino)ethyl)-1H-indol-6-yl)-4-phenoxybenzenesulfonamide,

[810] 3,5-dichloro-N-(3-(2-(diethylamino)ethyl)-1H-indol-6-yl)benzenesulfonamide,

[811] 4,5-dichloro-N-(3-(2-(diethylamino)ethyl)-1H-indol-6-ypthiophene-2-sulfonamide,

[812] 5-chloro-N-(3-(2-(diethylamino)ethyl)-1H-indol-6-yl)naphthalene-1-sulfonamide,

[813] 5-chloro-N-(3-(2-(dimethylamino)ethyl)-1H-indol-6-yl)-3-methylbenzo[b]thiophene-2-sulfonamide,

[814] N-(3-(2-(dimethylamino)ethyl)-1H-indol-6-yl)naphthalene-2-sulfonamide,

[815] N-(3-(2-(dimethylamino)ethyl)-1H-indol-6-yl)naphthalene-1-sulfonamide,

[816] 6-chloro-N-(3-(2-(dimethylamino)ethyl)-1H-indol-6-ypimidazo[2,1-b]thiazole-5-sulfonamide,

[817] N-(3-(2-(dimethylamino)ethyl)-1H-indol-6-yl)-4-phenylbenzenesulfonamide,

[818] N-(3-(2-(dimethylamino)ethyl)-1H-indol-6-yl)-2-(naphthalen-1-yl)ethanesulfonamide,

[819] N-(3-(2-(dimethylamino)ethyl)-1H-indol-6-yl)-4-phenoxybenzenesulfonamide,

[820] 3,5-dichloro-N-(3-(2-(dimethylamino)ethyl)-1H-indol-6-yl)benzenesulfonamide,

[821] 4,5-dichloro-N-(3-(2-(dimethylamino)ethyl)-1H-indol-6-ypthiophene-2-sulfonamide,

[822] 5-chloro-N-(3-(2-(dimethylamino)ethyl)-1H-indol-6-yl)naphthalene-1-sulfonamide,

[823] 6-bis(6-chloroimidazo[2,1-b]thiazol-5-ylsulfonyl)amino-3-(2-(dimethylamino)ethyl)-1H-indole,

[824] 6-bis(3,5-dichlorobenzenesulfonyl)amino-3-(2-(dimethylamino)ethyl)-1H-indole,

[825] 6-bis(4,5-dichlorothiophene-2-sulfonyl)amino-3-(2-(dimethylamino)ethyl)-1H-indole,

[826] 6-bis(5-chloro-3-methylbenzo[b]thiophene-2-sulfonyl)amino-3-(2-(dimethylamino)-1-ethoxyethyl)-1H-indole

[827] Ethyl 6-(5-chloro-3-methylbenzo[b]thiophene-2-sulfonamido)-3-(1-methylpiperidin-4-yl)-1H-indole-5-carboxylate,

[828] N-(4-bromo-3-(1-methylpiperidin-4-yl)-1H-indol-6-yl)naphthalene-1-sulfonamide,

[829] N-(7-bromo-3-(2-(dimethylamino)ethyl)-1H-indol-5-yl)benzofuran-2-sulfonamide,

[830] N-(7-methoxy-3-(2-(pyrrolidin-1-yl)ethyl)-1H-indol-5-yl)benzo[c][1,2,5]thiadiazole-4-sulfonamide,

[831] N-(7-methoxy-3-(2-(pyrrolidin-1-yl)ethyl)-1H-indol-5-yl)naphthalene-2-sulfonamide and

[832] 6-chloro-N-(7-methoxy-3-(2-(pyrrolidin-1-yl)ethyl)-1H-indol-5-ypimidazo[2,1-b]thiazole-5-sulfonamide;

and their corresponding salts and solvates.

The compounds of general formula (Ih) can be prepared according to the disclosure of WO 2005/013976 and WO 2006/024535. The respective parts of the literature are hereby incorporated by reference and form part of the present disclosure.

Preferred compounds of general formula (Ib) are selected from the group consisting of compounds of general formula (Ik)

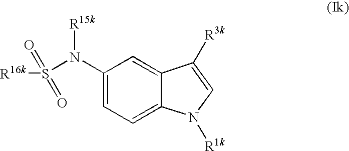

wherein

R^{1k} represents a hydrogen atom; a linear or branched, saturated or unsaturated, unsubstituted or at least mono-substituted aliphatic radical; an unsubstituted or at least mono-substituted phenyl radical or an unsubstituted or at least mono-substituted benzyl radical;

R^{3k} represents a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; or a —(CH₂)ₙₖ—NR^{13k}R^{14k} moiety with nk=0, 1, 2, 3, 4 or 5;

R^{13k} and R^{14k}, independently of one another, each represent a hydrogen atom; or a linear or branched, saturated or unsaturated, unsubstituted or at least mono-substituted aliphatic radical;

or

R^{13k} and R^{14k} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{15k} represents a hydrogen atom; or a linear or branched, saturated or unsaturated, unsubstituted or at least mono-substituted aliphatic radical;

and R^{16k} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ik) are those, wherein

nk represents 0, 1, 2, 3 or 4;

R^{1k} represents hydrogen,

R^{3k} represents a —NR^{13k}R^{14k} moiety or a moiety selected from the group consisting of

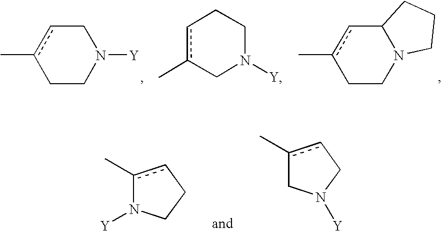

wherein, if present, the dotted line represents an optional chemical bond and Y represents hydrogen, a methyl group or an ethyl group,

R^{15k} represents hydrogen, a methyl group or an ethyl group,

R^{13k} and R^{14k}, identical or different, represent a methyl group, an ethyl group, an n-propyl group, an isopropyl group, an n-butyl group, an isobutyl group, a sec-butyl group, or a tert-butyl group, or

R^{13k} and R^{14k} together with the bridging nitrogen atom form a moiety selected from the group consisting of

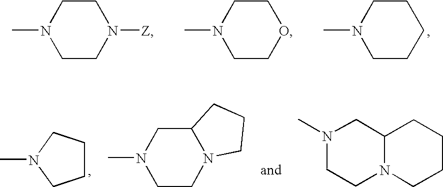

wherein Z represents hydrogen, a methyl group or an ethyl group,

R^{16k} represents a moiety selected from the group consisting of

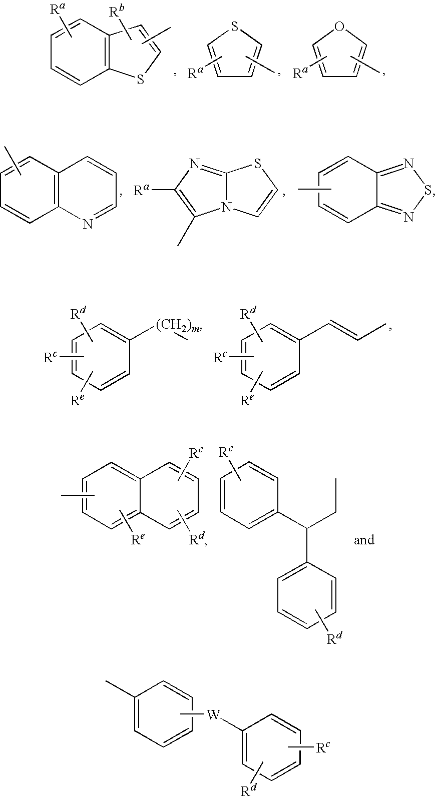

wherein

R^{a} and R^{b} are each independently selected from the group consisting of hydrogen, fluorine, chlorine, bromine, methyl, ethyl, pyridinyl, thiophenyl and furyl,

R^{c}, R^{d} and R^{e} are each independently selected from the group consisting of hydrogen, fluorine, chlorine, bromine, methyl, ethyl, methoxy, ethoxy and —CF₃,

W represents a single chemical bond between the two rings, a CH₂-group, O, S or a NR^{f}-moiety, wherein R^{f} is hydrogen, methyl or ethyl,

m is 0, 1, 2, 3 or 4;

optionally in form of one of its stereoisomers, preferably enantiomers or diastereomers, a racemate or in form of a mixture of at least two of its stereoisomers, preferably enantiomers and/or diastereomers, in any mixing ratio, or a salt thereof, preferably a corresponding, physiologically acceptable salt thereof, or a corresponding solvate thereof.

Particularly preferred compounds of general formula (Ik) are those selected from the group consisting of:

[833] N-[3-(2-diethylaminoethyl)-1H-indol-5-yl]-5-chloro-3-methylbenzo[b]thiophene-2-sulphonamide,

[834] N-[3-(2-diethylaminoethyl)-1H-indol-5-yl]naphthalene-1-sulphonamide,

[835] Hydrochloride N-[3-(2-diethylaminoethyl)-1H-indol-5-yl]naphthalene-1-sulphonamide,

[836] N-[3-(2-diethylaminoethyl)-1H-indol-5-yl]-3,5-dichlorobenzenesulphonamide,

[837] N-[3-(2-diethylaminoethyl)-1H-indol-5-yl]-4-phenylbenzenesulphonamide,

[838] N-[3-(2-diethylaminoethyl)-1H-indol-5-yl]-5-chlorothiophene-2-sulphonamide,

[839] N-[3-(2-dimethylaminoethyl)-1H-indol-5-yl]-5-chloro-3-methylbenzo[b]thiophene-2-sulphonamide,

[840] N-[3-(2-dimethylaminoethyl)-1H-indol-5-yl]naphthalene-1-sulphonamide,

[841] N-[3-(2-dimethylamino-ethyl)-1H-indol-5-yl]-6-chloroimidazo[2,1-b]thiazol-5-sulphonamide,

[842] N-[3-(1-methylpiperidin-4-yl)-1H-indol-5-yl]-5-chloro-3-methylbenzo[b]thiophene-2-sulphonamide,

[843] N-[3-(1-methylpiperidin-4-yl)-1H-indol-5-yl]-5-chloro-3-methylbenzo[b]thiophene-2-sulphonamide hydrochloride,

[844] N-[3-(1-methylpiperidin-4-yl)-1H-indol-5-yl]naphthalene-1-sulphonamide,

[845] N-[3-(1-methylpiperidin-4-yl)-1H-indol-5-yl]naphthalene-1-sulphonamide hydrochloride,

[846] N-[3-(1-methylpiperidin-4-yl)-1H-indol-5-yl]-5-chlorothiophene-2-sulphonamide,

[847] N-[3-(1-methylpiperidin-4-yl)-1H-indol-5-yl]-4-phenylbenzenesulphonamide,

[848] N-[3-(1-methylpiperidin-4-yl)-1H-indol-5-yl]quinoline-8-sulphonamide,

[849] N-[3-(2-diethylaminoethyl)-1H-indol-5-yl]naphthalene-2-sulphonamide,

[850] N-[3-(1-methyl-1,2,3,6-tetrahydropyridin-4-yl)-1H-indol-5-yl]naphthalene-1-sulphonamide,

[851] N-[3-(4-methylpiperazin-1-yl)methyl-1H-indol-5-yl]-5-chloro-3-methylbenzo[b]thiophene-2-sulphonamide,

[852] N-[3-(2-dimethylaminoethyl)-1H-indol-5-yl]-5-(2-pyridipthiophene-2-sulphonamide,

[853] N-[3-(2-dimethylaminoethyl)-1H-indol-5-yl]-2,1,3- benzothiadiazol-4-sulphonamide,

[854] N-[3-(2-dimethylaminoethyl)-1H-indol-5-yl]quinoline-8-sulphonamide,

[855] N-[3-(2-dimethylaminoethyl)-1H-indol-5-yl]-5-chloronaphthalene-2-sulphonamide,

[856] N-[3-(2-dimethylaminoethyl)-1H-indol-5-yl]-4-phenoxybenzenesulphonamide,

[857] N-[3-(2-dimethylaminoethyl)-1H-indol-5-yl]-4-phenylbenzenesulphonamide,

[858] N-[3-(2-diethylaminoethyl)-1H-indol-5-yl]-N-ethyl-naphthalene-2-sulphonamide,

[859] N-{3-[2-(morpholin-4-yl)ethyl]-1H-indol-5-yl}-5-chloro-3-methylbenzo[b]thiophene-2-sulphonamide,

[860] N-{3-[2-(morpholin-4-yl)ethyl]-1H-indol-5-yl}naphthalene-1-sulphonamide,

[861] N-[3-(2-diethylaminoethyl)-1H-indol-5-yl]naphthalene-2-sulphonamide,

[862] N-[3-dimethylaminomethyl-1H-indol-5-yl]-5-chloro-3-methylbenzo[b]thiophene-2-sulphonamide,

[863] N-[3-(2-dipropylaminoethyl)-1H-indol-5-yl]naphthalene-1-sulphonamide,

[864] N-[3-(2-dipropylaminoethyl)-1H-indol-5-yl]-5-chloro-3-methylbenzo[b]thiophene-2-sulphonamide,

[865] N-[3-(2-dibutylaminoethyl)-1H-indol-5-yl]-5-chloro-3-methylbenzo[b]thiophene-2-sulphonamide,

[866] N-[3-(2-dibutylaminoethyl)-1H-indol-5-yl]naphthalene-1-sulphonamide,

[867] N-[3-(2-diethylaminoethyl)-1H-indol-5-yl]-5-chloronaphthalene-1-sulphonamide,

[868] N-[3-(2-diethylaminoethyl)-1H-indol-5-yl]-trans-p-styrenesulphonamide,

[869] N-[3-(4-methylpiperazin-1-yl)methyl-1H-indol-5-yl]-trans-β-styrenesulphonamide,

[870] N-[3-(octahydroindolizin-7-yl)-1H-indol-5-yl]-5-chloro-3-methylbenzo[b]thiophene-2-sulphonamide,

[871] N-[3-(2-diethylaminoethyl)-1H-indol-5-yl]-6-chloroimidazo[2,1-b]thiazol-5-sulphonamide,

[872] N-{3-[2-(morpholin-4-yl)ethyl]-1H-indol-5-yl}naphthalene-2-sulphonamide,

[873] N-[3-(4-methylpiperazin-1-yl)methyl-1H-indol-5-yl]-α-toluenesulphonamide,

[874] N-[3-(3-diethylaminopropyl)-1H-indol-5-yl]naphthalene-2-sulphonamide,

[875] N-[3-(3-diethylaminopropyl)-1H-indol-5-yl]-5-chloro-3-methylbenzo[b]thiophene-2-sulphonamide,

[876] N-{3-[2-(pyrrolidin-1-yl)ethyl]-1H-indol-5-yl}-5-chloro-3-methylbenzo[b]thiophene-2-sulphonamide,

[877] N-{3-[2-(pyrrolidin-1-yl)ethyl]-1H-indol-5-yl}naphthalene-1-sulphonamide,

[878] N-{3-[2-(pyrrolidin-1-yl)ethyl]-1H-indol-5-yl}naphthalene-2-sulphonamide,

[879] N-[3-(2-dipropylaminoethyl)-1H-indol-5-yl]naphthalene-2-sulphonamide,

[880] N-[3-(2-dimethylaminoethyl)-1H-indol-5-yl]-5-chloronaphthalene-1-sulphonamide,

[881] N-[3-(2-dimethylaminoethyl)-1H-indol-5-yl]naphthalene-2-sulphonamide,

[882] N-{3-[2-(morpholin-4-yl)ethyl]-1H-indol-5-yl}quinoline-8-sulphonamide,

[883] N-{3-[2-(morpholin-4-yl)ethyl]-1H-indol-5-yl}-4-phenylbenzenesulphonamide,

[884] N-[3-(4-methylpiperazin-1-yl)ethyl-1H-indol-5-yl]naphthalene-2-sulphonamide and

[885] N-[3-(4-methylpiperazin-1-yl)ethyl-1H-indol-5-yl]-5-chloronaphthalene-1-sulphonamide;

and their corresponding salts and solvates.

The compounds of general formula (Ik) can be prepared according to the disclosure of WO 2004/098588. The respective part of the literature is hereby incorporated by reference and forms part of the present disclosure.

Preferred compounds of general formula (Ib) are selected from the group consisting of compounds of general formula (Im)

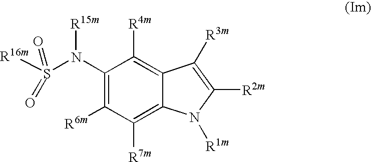

wherein

R^{1m} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₘ—NR^{13m}R^{14m} moiety with mm=0, 1, 2, 3, 4 or 5;

R^{2m} represents a hydrogen atom; —F; —Cl; —Br; —I; —NO₂; —CN; —O—R^{10m}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R^{3m} represents a hydrogen atom; —F; —Cl; —Br; —I; —NO₂; —CN; —O—R^{10m}; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; a linear or branched, saturated or unsaturated, unsubstituted or at least mono-substituted aliphatic radical; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₘ—NR^{13m}R^{14m} moiety with nm=0, 1, 2, 3, 4 or 5;

R^{4m}m, R^{6m} an d R^{7m}, independently of one another, each represent a hydrogen atom; —NO₂; —CN; —O—R^{10m}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{10m} represents a linear or branched, saturated or unsaturated, unsubstituted or at least mono-substituted aliphatic radical; or an unsubstituted or at least mono- substituted aryl or heteroaryl radical, which may be bonded via a linear or branched alkylene group;

R^{13m} and R^{14m}, independently of one another, each represent a hydrogen atom; or a linear or branched, saturated or unsaturated, unsubstituted or at least mono-substituted aliphatic radical;

or

R^{13m} and R^{14m} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{15m} represents a hydrogen atom; a linear or branched, saturated or unsaturated, unsubstituted or at least mono-substituted aliphatic radical or a —S(═O)₂—R^{16m} moiety;

and R^{16m} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Im) are those, wherein

R^{1m} represents a hydrogen atom, ethyl, methyl, n-propyl, n-butyl or a —(CH₂)ₘₘ—NR^{13m}R^{14m} radical,

R^{2m} represents hydrogen or an alkyl radical selected from the group consisting of methyl, ethyl, n-propyl and isopropyl, more preferably hydrogen or methyl,

R^{3m} represents a hydrogen atom; a radical selected from the group consisting of piperidinyl, pyrrolidinyl, (1,2,3,6)-tetrahydropyridinyl and octahydroindolizinyl, which may be subsituted by 1, 2, 3 or 4 radicals selected from the group consisting of methyl, ethyl, n-propyl and n-butyl; or a —(CH₂)ₙₘ—NR^{13m}R^{14m} moiety with nm=0, 1, 2, 3, 4 or 5;

R^{4m} and R^{6m} each represent hydrogen,

R^{7m} represents a hydrogen atom, a chlorine atom, a bromine atom or —O—CH₃,

R^{15m} represents hydrogen,

R^{13m} and R^{14m} identical or different, each represent methyl, ethyl, n-propyl, n-butyl or isopropyl,

or

R^{13m} and R^{14m} together with the bridging nitrogen form a 5- or 6-membered heterocyclic ring, more preferably form a moiety selected from the group consisting of morpholine, piperazine, pyrrolidine and piperidine which may be subsituted by 1, 2, 3 or 4 radicals selected from the group consisting of methyl, ethyl, n-propyl and n-butyl;

R^{16m} represents an aryl or heteroaryl radical selected from the group consisting of phenyl, naphthyl, quinolinyl, benzo[b]furanyl, benzo[b]thiophenyl, benzo[1,2,5]thiadiazolyl, thiophenyl and imidazo[2,1-b]thiazolyl which may be substituted by 1, 2 or 3 substituents selected from the group consisting of fluorine, bromine, chlorine, methyl, phenyl, pyridinyl, nitro, —C(═O)—CH₃, —O—CH₃ and —O-phenyl and/or which may be bonded via a C₁₋₂ alkylene group or a C₂ alkenylene group,

and

mm is 2 or 3,

optionally in form of one of its stereoisomers, preferably enantiomers or diastereomers, its racemate or in form of a mixture of at least two of its stereoisomers, preferably enantiomers or diastereomers, in any mixing ratio, or a salt thereof, preferably a corresponding, physiologically acceptable salt thereof, or a corresponding solvate thereof.

Particularly preferred compounds of general formula (Im) are those selected from the group consisting of

[886] N-[1-(2-dimethylaminoethyl)-1H-indole-5-yl]-5-chloro-3-methylbenzo[b]thiophene-2-sulfonamide,

[887] N-[1-(2-dimethylaminoethyl)-1H-indole-5-yl]-naphthalene-2-sulfonamide,

[888] N-[1-(2-dimethylaminoethyl)-1H-indole-5-yl]-naphthalene-1-sulfonamide,

[889] N-[1-(2-dimethylaminoethyl)-1H-indole-5-yl]-5-chloronaphthalene-1-sulfonamide,

[890] N-[1-(2-dimethylaminoethyl)-1H-indole-5-yl]-benzenesulfonamide,

[891] N-[1-(2-dimethylaminoethyl)-1H-indole-5-yl]-quinoline-8-sulfonamide,

[892] N-[1-(2-dimethylaminoethyl)-1H-indole-5-yl]-4-phenoxybenzenesulfonamide,

[893] N-[1-(2-dimethylaminoethyl)-1H-indole-5-yl]-4-methylbenzenesulfonamide,

[894] N-[1-(2-dimethylaminoethyl)-1H-indole-5-yl]-5-chlorothiophene-2-sulfonamide,

[895] N-[1-(2-dimethylaminoethyl)-1H-indole-5-yl]-benzo[1,2,5]thiadiazole-4-sulfonamide,

[896] N-[1-(2-dimethylaminoethyl)-1H-indole-5-yl]-6-chloroimidazo[2,1-b]thiazole-5-sulfonamide,

[897] N-[1-(2-dimethylaminoethyl)-1H-indole-5-yl]-3,5-dichlorobenzenesulfonamide,

[898] N-[1-(2-dimethylaminoethyl)-1H-indole-5-yl]-3-bromobenzenesulfonamide,

[899] N-[1-(2-dimethylaminoethyl)-1H-indole-5-yl]-3-nitrobenzenesulfonamide,

[900] N-[1-(2-dimethylaminoethyl)-1H-indole-5-yl]-1-phenylmethanesulfonamide,

[901] N-[1-(2-pyrrolidine-1-yl-ethyl)-1H-indole-5-yl]-naphthalene-2-sulfonamide,

[902] N-[1-(2-pyrrolidine-1-yl-ethyl)-1H-indole-5-yl]-naphthalene-1-sulfonamide,

[903] N-[1-(2-pyrrolidine-1-yl-ethyl)-1H-indole-5-yl]-5-chloro-3-methylbenzo[b]thiophene-2-sulfonamide,

[904] trans-N-[1-(2-dimethylaminoethyl)-1H-indole-5-yl]-2-phenylethenesulfonamide,

[905] N-[1-(2-dimethylaminoethyl)-1H-indole-5-yl]-4,5-dichlorothiophene-2-sulfonamide,

[906] N-[1-(2-dimethylaminoethyl)-1H-indole-5-yl]-4-acetylbenzenesulfonamide,

[907] N-[1-(2-dimethylaminoethyl)-1H-indole-5-yl]-4-bromobenzenesulfonamide,

[908] N-[1-(2-dimethylaminoethyl)-1H-indole-5-yl]-4-methoxybenzenesulfonamide,

[909] N-[3-(2-diethylaminoethyl)-1H-indole-5-yl]-5-chloro-3-methylbenzo[b]thiophene-2-sulfonamide,

[910] N-[1-(2-dimethylaminoethyl)-1H-indole-5-yl]-4-nitrobenzenesulfonamide,

[911] N-[1-(2-dimethylaminoethyl)-1H-indole-5-yl]-4-fluorobenzenesulfonamide,

[912] N-[1-(2-diethylaminoethyl)-1H-indole-5-yl]-6-chloroimidazo[2,1-b]thiazole-5-sulfonamide,

[913] N-[1-(2-pyrrolidine-1-yl-ethyl)-1H-indole-5-yl]-]-6-chloroimidazo[2,1-b]thiazole-5-sulfonamide,

[914] N-(1-(2-(diethylamino)ethyl)-1H-indol-5-yl)-naphthalene-2-sulfonamide,

[915] N-(1-(2-(diethylamino)ethyl)-1H-indol-5-yl)-naphthalene-1-sulfonamide,

[916] N-(1-(2-(diethylamino)ethyl)-1H-indol-5-yl)-4-phenylbenzenesulfonamide,

[917] 5-chloro-N-(1-(2-(dimethylamino)ethyl)-2-methyl-1H-indol-5-yl)-3-methylbenzo[b]thiophene-2-sulfonamide,

[918] N-(1-(2-(dimethylamino)ethyl)-2-methyl-1H-indol-5-yl)-naphthalene-2-sulfonamide,

[919] N-(1-(2-(dimethylamino)ethyl)-2-methyl-1H-indol-5-yl)-naphthalene-1-sulfonamide,

[920] 6-chloro-N-(1-(2-(dimethylamino)ethyl)-2-methyl-1H-indol-5-ypimidazo[2,1-b]thiazole-5-sulfonamide,

[921] N-(1-(2-(dimethylamino)ethyl)-2-methyl-1H-indol-5-yl)-4-phenylbenzenesulfonamide,

[922] N-(1-(2-dimethylamino)ethyl)-2-methyl-1H-indol-5-yl)-2-(naphth-1-yl)-ethanesulfonamide,

[923] N-(1-(2-(dimethylamino)ethyl)-2-methyl-1H-indol-5-yl)-4-phenoxy-benzenesulfonamide,

[924] 3,5-dichloro-N-(1-(2-(dimethylamino)ethyl)-2-methyl-1H-indol-5-yl)-benzenesulfonamide,

[925] N-(1-(2-(dimethylamino)ethyl)-2-methyl-1H-indol-5-yl)benzo[b]thiophene-3-sulfonamide,

[926] N-(1-(2-(diethylamino)ethyl)-1H-indol-5-yl)benzo[b]thiophene-3-sulfonamide

[927] N-(1-(2-(dimethylamino)ethyl)-1H-indol-5-yl)benzo[b]thiophene-3-sulfonamide,

[928] 5-chloro-3-methyl-N-(1-(3-(piperidin-1-yl)propyl)-1H-indol-5-yl)benzo[b]thiophene-2-sulfonamide,

[929] N-(1-(3-(piperidin-1-yl)propyl)-1H-indol-5-yl)naphthalene-2-sulfonamide,

[930] N-(1-(3-(piperidin-1-yl)propyl)-1H-indol-5-yl)naphthalene-1-sulfonamide,

[931] 6-chloro-N-(1-(3-piperidin-1-yl)propyl)-1H-indol-5-ypimidazo[2,1-b]thiazole-5-sulfonamide,

[932] 4-phenyl-N-(1-(3-(piperidin-1-yl)propyl)-1H-indol-5-yl)benzenesulfonamide,

[933] 2-(naphth-1-yl)-N-(1-(3-(piperidin-1-yl)propyl)-1H-indol-5-ypethanesulfonamide,

[934] 4-phenoxy-N-(1-(3-(piperidin-1-yl)propyl)-1H-indol-5-yl)benzenesulfonamide,

[935] 3,5-dichloro-N-(1-(3-(piperidin-1-yl)propyl)-1H-indol-5-yl)benzenesulfonylamide,

[936] 4,5-dichloro-N-(1-(3-(piperidin-1-yl)propyl)-1H-indol-5-yl)thiophene-2-sulfonamide and

[937] 5-chloro-N-(1-(3-(piperidin-1-yl)propyl)-1H-indol-5-yl)naphthalene-1-sulfonamide,

[938] N-(3-(2-(diethylamino)ethyl)-7-methoxy-1H-indol-5-yl)naphthalene-2-sulfonamide,

[939] N-(3-(2-(diethylamino)ethyl)-7-methoxy-1H-indol-5-yl)benzo[c][1,2,5]thiadiazole-4-sulfonamide,

[940] 6-chloro-N-(3-(2-(diethylamino)ethyl)-7-methoxy-1H-indol-5-ypimidazo[2,1-b]thiazole-5-sulfonamide,

5-chloro-N-(3-(2-(diethylamino)ethyl)-1H-indol-5-yl)-3-methylbenzo[b]thiophene-2-sulfonamide,

N-(3-(2-(diethylamino)ethyl)-1H-indol-5-yl)naphthalene-1-sulfonamide,

N-(3-(2-(diethylamino)ethyl)-1H-indol-5-yl)naphthalene-1-sulfonamide hydrochloride,

3,5-dichloro-N-(3-(2-(diethylamino)ethyl)-1H-indol-5-yl)benzenesulfonamide,

N-(3-(2-(diethylamino)ethyl)-1H-indol-5-yl)biphenyl-4-sulfonamide,

5-chloro-N-(3-(2-(diethylamino)ethyl)-1H-indol-5-yl)thiophene-2-sulfonamide,

5-chloro-N-(3-(2-(dimethylamino)ethyl)-1H-indol-5-yl)-3-methylbenzo[b]thiophene-2-sulfonamide,

N-(3-(2-(dimethylamino)ethyl)-1H-indol-5-yl)naphthalene-1-sulfonamide,

6-chloro-N-(3-(2-(dimethylamino)ethyl)-1H-indol-5-ypimidazo[2,1-b]thiazole-5-sulfonamide,

5-chloro-3-methyl-N-(3-(1-methylpiperidin-4-yl)-1H-indol-5-yl)benzo[b]thiophene-2-sulfonamide,

5-chloro-3-methyl-N-(3-(1-methylpiperidin-4-yl)-1H-indol-5-yl)benzo[b]thiophene-2-sulfonamide hydrochloride,

N-(3-(1-methylpiperidin-4-yl)-1H-indol-5-yl)naphthalene-1-sulfonamide,

N-(3-(1-methylpiperidin-4-yl)-1H-indol-5-yl)naphthalene-1-sulfonamide hydrochloride,

5-chloro-N-(3-(1-methylpiperidin-4-yl)-1H-indol-5-yl)thiophene-2-sulfonamide,

N-(3-(1-methylpiperidin-4-yl)-1H-indol-5-yl)biphenyl-4-sulfonamide,

N-(3-(1-methylpiperidin-4-yl)-1H-indol-5-yl)quinoline-8-sulfonamide,

N-(3-(2-(diethylamino)ethyl)-1H-indol-5-yl)naphthalene-2-sulfonamide,

N-(3-(1-methyl-1,2,3,6-tetrahydropyridin-4-yl)-1H-indol-5-yl)naphthalene-1-sulfonamide,

5-chloro-3-methyl-N-(3-((4-methylpiperazin-1-yl)methyl)-1H-indol-5-yl)benzo[b]thiophene-2-sulfonamide,

N-(3-(2-(dimethylamino)ethyl)-1H-indol-5-yl)-5-(pyridin-2-yl)thiophene-2-sulfonamide,

N-(3-(2-(dimethylamino)ethyl)-1H-indol-5-yl)benzo[c][1,2,5]thiadiazole-4-sulfonamide,

N-(3-(2-(dimethylamino)ethyl)-1H-indol-5-yl)quinoline-8-sulfonamide,

5-chloro-N-(3-(2-(dimethylamino)ethyl)-1H-indol-5-yl)naphthalene-2-sulfonamide,

N-(3-(2-(dimethylamino)ethyl)-1H-indol-5-yl)-4-phenoxybenzenesulfonamide,

N-(3-(2-(dimethylamino)ethyl)-1H-indol-5-yl)biphenyl-4-sulfonamide,

N-(3-(2-(diethylamino)ethyl)-1-ethyl-1H-indol-5-yl)naphthalene-2-sulfonamide,

5-chloro-3-methyl-N-(3-(2-morpholinoethyl)-1H-indol-5-yl)benzo[b]thiophene-2-sulfonamide,

N-(3-(2-morpholinoethyl)-1H-indol-5-yl)naphthalene-1-sulfonamide,

N-(3-(2-(diethylamino)ethyl)-1-methyl-1H-indol-5-yl)naphthalene-2-sulfonamide,

5-chloro-N-(3-((dimethylamino)methyl)-1H-indol-5-yl)-3-methylbenzo[b]thiophene-2-sulfonamide,

N-(3-(2-(dipropylamino)ethyl)-1H-indol-5-yl)naphthalene-1-sulfonamide,

5-chloro-N-(3-(2-(dipropylamino)ethyl)-1H-indol-5-yl)-3-methylbenzo[b]thiophene-2-sulfonamide,

5-chloro-N-(3-(2-(dibutylamino)ethyl)-1H-indol-5-yl)-3-methylbenzo[b]thiophene-2-sulfonamide,

N-(3-(2-(dibutylamino)ethyl)-1H-indol-5-yl)naphthalene-1-sulfonamide,

5-chloro-N-(3-(2-(diethylamino)ethyl)-1H-indol-5-yl)naphthalene-1-sulfonamide,

N-(3-(2-(diethylamino)ethyl)-1H-indol-5-yl)-2-phenylethenesulfonamide,

N-(3-((4-methylpiperazin-1-yl)methyl)-1H-indol-5-yl)-2-phenylethenesulfonamide,

5-chloro-3-methyl-N-(3-(octahydroindolizin-7-yl)-1H-indol-5-yl)benzo[b]thiophene-2-sulfonamide,

6-chloro-N-(3-(2-(diethylamino)ethyl)-1H-indol-5-yl)-5,7a-dihydroimidazo[2,1-b]thiazole-5-sulfonamide,

N-(3-(2-morpholinoethyl)-1H-indol-5-yl)naphthalene-2-sulfonamide,

N-(3-((4-methylpiperazin-1-yl)methyl)-1H-indol-5-yl)(phenyl)methanesulfonamide,

N-(3-(3-(diethylamino)propyl)-1H-indol-5-yl)naphthalene-2-sulfonamide,

5-chloro-N-(3-(3-(diethylamino)propyl)-1H-indol-5-yl)-3-methylbenzo[b]thiophene-2-sulfonamide,

5-chloro-3-methyl-N-(3-(2-(pyrrolidin-1-yl)ethyl)-1H-indol-5-yl)benzo[b]thiophene-2-sulfonamide,

N-(3-(2-(pyrrolidin-1-yl)ethyl)-1H-indol-5-yl)naphthalene-1-sulfonamide,

N-(3-(2-(pyrrolidin-1-yl)ethyl)-1H-indol-5-yl)naphthalene-2-sulfonamide,

N-(3-(2-(dipropylamino)ethyl)-1H-indol-5-yl)naphthalene-2-sulfonamide,

5-chloro-N-(3-(2-(dimethylamino)ethyl)-1H-indol-5-yl)naphthalene-1-sulfonamide,

N-(3-(2-(dimethylamino)ethyl)-1H-indol-5-yl)naphthalene-2-sulfonamide,

N-(3-(2-morpholinoethyl)-1H-indol-5-yl)quinoline-8-sulfonamide,

N-(3-(2-morpholinoethyl)-1H-indol-5-yl)biphenyl-4-sulfonamide,

N-(3-(2-(4-methylpiperidin-1-yl)ethyl)-1H-indol-5-yl)naphthalene-2-sulfonamide and

5-chloro-N-(3-(2-(4-methylpiperidin-1-yl)ethyl)-1H-indol-5-yl)naphthalene-1-sulfonamide;

and their corresponding salts and solvates.

The compounds of general formula (Im) can be prepared according to the disclosure of WO 2003/042175, WO 2005/013977 and WO 2006/024535. The respective parts of the literature are hereby incorporated by reference and form part of the present disclosure.

Preferred compounds of general formula (Ib) are selected from the group consisting of compounds of general formula (In)

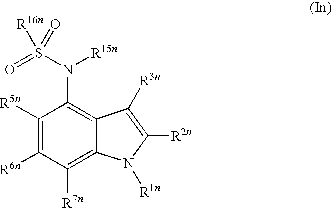

wherein

R¹ⁿ represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₙ—NR¹³ⁿR¹⁴ⁿ moiety with mn=0, 1, 2, 3, 4 or 5;

R²ⁿ represents a hydrogen atom; —F; —Cl; —Br; —I; —NO₂; —CN; —O—R¹⁰ⁿn; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R³ⁿ represents a hydrogen atom; —F; —Cl; —Br; —I; —NO₂; —CN; —O—R¹⁰ⁿ; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₙ—NR¹³ⁿR¹⁴ⁿ moiety with nn=0, 1, 2, 3, 4 or 5;

R⁵ⁿ, R⁶ⁿ and R⁷ⁿ, independently of one another, each represent a hydrogen atom; —NO₂; —CN; —O—R¹⁰ⁿ; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R¹⁰ⁿ represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R¹³ⁿ and R¹⁴ⁿ, independently of one another, each represent a hydrogen atom; or a linear or branched, saturated or unsaturated, unsubstituted or at least mono-substituted aliphatic radical;

or

R¹³ⁿ n and R¹⁴ⁿ together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R¹⁵ⁿ represents a hydrogen atom; a linear or branched, saturated or unsaturated, unsubstituted or at least mono-substituted aliphatic radical or a —S(═O)₂—R¹⁶ⁿ moiety;

R¹⁶ⁿ represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (In) are those, wherein

R¹ⁿ represents a hydrogen atom or a —(CH₂)ₘₙ—NR¹³ⁿR¹⁴ⁿ radical,

R²ⁿ, R⁵ⁿ, R⁶ⁿ and R⁷ⁿ each represent hydrogen,

R³ⁿ represents a hydrogen atom or a —(CH₂)ₙₙ—NR¹³ⁿR¹⁴ⁿ moiety with nn=0, 1 or 2;

R¹⁵ⁿ represents hydrogen,

R¹³ⁿ and R¹⁴ⁿ, identical or different, each represent methyl, ethyl, n-propyl, isopropyl, more preferably methyl,

or

R¹³ⁿ and R¹⁴ⁿ together with the bridging nitrogen form a 5- or 6-membered heterocyclic ring, more preferably form pyrrolidine or piperidine,

and

R¹⁶ⁿ represents an aryl or heteroaryl radical selected from the group consisting of phenyl, naphthyl, benzo[b]thiophenyl and imidazo[2,1-b]thiazolyl which may be substituted by 1, 2 or 3 substituents selected from the group consisting of chlorine, methyl, phenyl and —O-phenyl and/or which may be bonded via a C₁₋₂ alkylene group, and

mn is 1 or 2;

optionally in form of one of its stereoisomers, preferably enantiomers or diastereomers, its racemate or in form of a mixture of at least two of its stereoisomers, preferably enantiomers or diastereomers, in any mixing ratio, or a salt thereof, preferably a corresponding, physiologically acceptable salt thereof, or a corresponding solvate thereof.

Particularly preferred compounds of general formula (In) are those selected from the group consisting of:

[941] N-[1-(2-dimethylaminoethyl)-1H-indole-4-yl]-5-chloro-3-methylbenzo[b]thiophene-2-sulfonamide,

[942] N-[1-(2-dimethylaminoethyl)-1H-indole-4-yl]-naphtalene-2-sulfonamide,

[943] N-[1-(2-dimethylaminoethyl)-1H-indole-4-yl]-naphtalene-1-sulfonamide,

[944] N-[1-(2-dimethylaminoethyl)-1H-indole-4-yl]-4-phenylbenzenesulfonamide,

[945] N-[1-(2-dimethylaminoethyl)-1H-indole-4-yl]-2-(naphtalene-1-yl)-ethanesulfonamide,

[946] N-[1-(2-dimethylaminoethyl)-1H-indole-4-yl]-4-phenoxybenzenesulfonamide,

[947] N-[1-(2-dimethylaminoethyl)-1H-indole-4-yl]-3,5- dichlorobenzenesulfonamide and

[948] 6-chloro-N-[1-(2-dimethylaminoethyl)-1H-indol-4-yl]-imidazo[2,1-b]thiazole-5-sulfonamide

[949] N-(3-(2-(dimethylamino)ethyl)-1H-indol-4-yl)-4-biphenylsulfonamide,

[950] N-(3-(2-(d imethylamino)ethyl)-1H-indol-4-yl)-4-phenoxybenzenesulfonamide,

[951] 3,5-dichloro-N-(3-(2-(dimethylamino)ethyl)-1H-indol-4-yl)benzenesulfonamide,

[952] 5-chloro-N-(3-(2-(dimethylamino)ethyl)-1H-indol-4-yl)-3-methylbenzo[b]thiophene-2-sulfonamide,

[953] N-(3-(2-(dimethylamino)ethyl)-1H-indol-4-yl)naphthalene-1-sulfonamide,

[954] 5-chloro-N-(3-(2-(dimethylamino)ethyl)-1H-indol-4-yl)naphthalene-2-sulfonamide,

[955] N-(3-(2-(dimethylamino)ethyl)-1H-indol-4-yl)naphthalene-2-sulfonamide,

[956] 6-chloro-N-(3-(2-(dimethylamino)ethyl)-1H-indol-4-ypimidazo[2,1-b]thiazole-5-sulfonamide,

[957] N-(3-(2-(dimethylamino)ethyl)-1H-indol-4-yl)-2-(naphthalen-1-yl)ethanesulfonamide,

and their corresponding salts and solvates.

The compounds of general formula (In) can be prepared according to the disclosure of WO 2005/13978 and WO 2006/024535. The respective parts of the literature are hereby incorporated by reference and form part of the present disclosure.

Preferred compounds of general formula (Ib) are selected from the group consisting of compounds of general formula (Io)

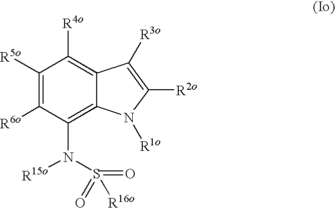

wherein

R^{1o} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₒ—NR^{13o}R^{14o} moiety with mo=0, 1, 2, 3, 4 or 5;

R^{2o} represents a hydrogen atom; —F; —Cl; —Br; —I; —NO₂; —CN; —O—R^{10o}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R^{3o} represents a hydrogen atom; —F; —Cl; —Br; —I; —NO₂; —CN; —O—R^{10o}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; a —CH(OC₂H₅)—CH₂—NR^{13o}R^{14o} moiety or a —(CH₂)ₙₒ—NR^{13o}R^{14o} moiety with no=0, 1, 2, 3, 4 or 5;

R^{4o}, R^{5o} and R^{6o}, independently of one another, each represent a hydrogen atom; —NO₂; —CN; —O—R^{10o}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{10o} represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{13o} and R^{14o}, independently of one another, each represent a hydrogen atom; or a linear or branched, saturated or unsaturated, unsubstituted or at least mono-substituted aliphatic radical;

or

R^{13o} and R^{14o} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{15o} represents a hydrogen atom; a linear or branched, saturated or unsaturated, unsubstituted or at least mono-substituted aliphatic radical or a —S(═O)₂—R^{16o} moiety;

and R^{16o} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Io) are those, wherein

R¹ is —(CH₂)ₘₒ—NR^{13o}R^{14o} radical,

R^{2o}, R^{4o} and R^{6o} each represent hydrogen,

R^{3o} represents a hydrogen atom, a —CH(OC₂H₅)—CH₂—NR^{13o}R^{14o} moiety or a —(CH₂)ₙₒ—NR^{13o}R^{14o} moiety with no=0, 1 or 2,

R^{5o} represents a hydrogen atom, chlorine or bromine,

R^{15o} represents hydrogen or a —S(═O)₂—R1^{6o} moiety,

R^{13o} and R^{14o}, identical or different, each represent methyl, ethyl, n-propyl or isopropyl, more preferably methyl,

or

R^{13o} and R^{14o} together with the bridging nitrogen atom form a 5- or 6-membered heterocyclic ring, more preferably form a pyrrolidine or piperidine ring,

R^{16o} represents an aryl or heteroaryl radical selected from the group consisting of phenyl, naphthyl, benzo[b]thiophenyl and imidazo[2,1-b]thiazolyl which may be substituted by 1, 2 or 3 substituents selected from the group consisting of chlorine, methyl and phenyl and/or which may be bonded via a C₁-₂ alkylene group,

and

no is 1 or 2;

optionally in form of one of its stereoisomers, preferably enantiomers or diastereomers, its racemate or in form of a mixture of at least two of its stereoisomers, preferably enantiomers or diastereomers, in any mixing ratio, or a salt thereof, preferably a corresponding, physiologically acceptable salt thereof, or a corresponding solvate thereof.

Particularly preferred compounds of general formula (Io) are those selected from the group consisting of:
N-[1-(2-dimethylaminoethyl)-1H-indole-7-yl]-naphtalene-1-sulfonamide,
N-[1-(2-dimethylaminoethyl)-1H-indole-7-yl]-5-chloro-3-methylbenzo[b]thiophene-2-sulfonamide,
N-[1-(2-dimethylaminoethyl)-1H-indole-7-yl]-4-phenylbenzenesulfonamide and
N-[1-(2-dimethylaminoethyl)-1H-indole-7-yl]-6-chloroimidazo[2,1-b]thiazole-5-sulfonamide
5-chloro-3-methyl-N-(1-(2-(pyrrolidin-1-yl)ethyl)-1H-indol-7-yl)-benzo[b]thiophen-2-sulfonamide,
N-(1-(2-(pyrrolidin-1-yl)ethyl)-1H-indol-7-yl)naphthalene-1-sulfonamide,
6-chloro-N-(1-(2-(pyrroldin-1-yl)ethyl)-1H-indol-7-yl)imidazo[2,1-b]thiazole-5-sulfonamide and
2-(naphth-1-yl)-N-(1-(2-(pyrrolidin-1-yl)ethyl)-1H-indol-7-yl)ethansulfonamide
5-chloro-N-(3-(2-(dimethylamino)-1-ethoxyethyl)-1H-indol-7-yl)-3-methylbenzo[b]thiophene-2-sulfonamide,
5-chloro-N-(3-(2-(dimethylamino)ethyl)-1H-indol-7-yl)-3-methylbenzo[b]thiophene-2-sulfonamide,
7-bis(5-chloro-3-methylbenzo[b]thiophene-2-sulfonyl)amino-3-(2-(diethylamino)-1-ethoxyethyl)-1H-indole,
5-chloro-N-(3-(2-(diethylamino)-1-ethoxyethyl)-1H-indol-7-yl)-3-methylbenzo[b]thiophene-2-sulfonamide,
7-bis(5-chloro-3-methylbenzo[b]thiophene-2-sulfonyl)amino-3-(2-(dimethylamino)ethyl)-1H-indole,
7-bis(5-chloro-3-methylbenzo[b]thiophene-2-sulfonyl)amino-3-(2-(diethylamino)ethyl)-1H-indole,
5-chloro-N-(3-(2-(diethylamino)ethyl)-1H-indol-7-yl)-3-methylbenzo[b]thiophene-2-sulfonamide,
7-bis(6-chloroimidazo[2,1-b]thiazol-5-ylsulfonyl)amino-3-(2-(dimethylamino)ethyl)-1H-indole,
N-(5-bromo-3-(2-(pyrrolidin-1-yl)ethyl)-1H-indol-7-yl)-6-chloroimidazo[2,1-b]thiazole-5-sulfonamide,

and their corresponding salts and solvates.

The compounds of general formula (Io) can be prepared according to the disclosure of WO 2005/13979 and WO 2006/024535. The respective parts of the literature are hereby incorporated by reference and form part of the present disclosure.

Preferred compounds of general formula (Ib) are selected from the group consisting of compounds of general formula (Ip)

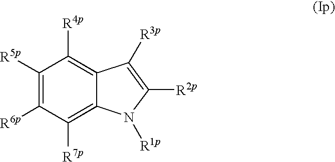

wherein

R^{1p} represents a —S(═O)₂—R^{9p} moiety or a —S(═O)₂—C(H)A^{p}B^{p} moiety;

R^{2p} represents a hydrogen atom; —F; —Cl; —Br; —I; —NO₂; —OH; —CN; —O—R^{10p}; —S—R^{11p}; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system;

R^{3p} represents a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; or a —(CH₂)ₙₚ—NR^{13p}R^{14p} moiety with np=0, 1, 2, 3, 4 or 5;

R^{4p}, R^{5p}, R^{6p} and R^{7p}, independently of one another, each represent a hydrogen atom; —NO₂; —NH₂; —OH; —CN; —C(═O)—R^{8p}; —O—R^{10p}; —S—R^{11p}; —NH—R^{17p}; —NR^{18p}R^{19p}; atom; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched alkylene group;

R^{8p} represents a hydrogen atom or a linear or branched, saturated or unsaturated, unsubstituted or at least mono-substituted aliphatic radical;

R^{8p}, R^{17p}, R^{18p} and R^{19p}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene, alkenylene or alkinylene group;

R^{9p} represents a linear or branched, saturated or unsaturated, unsubstituted or at least mono-substituted aliphatic radical;

R^{10p} and R^{11p}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{13p} and R^{14p}, independently of one another, each represent a hydrogen atom; or a linear or branched, saturated or unsaturated, unsubstituted or at least mono-substituted aliphatic radical;

or

R^{13p} and R^{14p} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

A^{p} and B^{p} together with the bridging carbon form an unsubstituted or at least mono-substituted, saturated or unsaturated cycloaliphatic ring which may contain at least one further heteroatom as a ring member and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ip) are those, wherein

R^{1p} represents a —S(═O)₂—C(H)A^{p}B^{p} moiety;

R^{1p}, R^{3p}, R^{4p} and R^{6p} each represent hydrogen,

R^{3p} represents a —(CH₂)ₙₚ—NR^{13p}R^{14p} moiety or an unsaturated, optionally at least one nitrogen atom as a ring member containing 5- or 6-membered cycloaliphatic radical, which may be substituted by a methyl group and/or which may be condensed with a 5-membered cycloaliphatic ring, more preferably R^{3p} represents a —(CH₂)ₙₚ—NR^{13p}R^{14p} moiety or a moiety selected from the group consisting of

R^{5p} represents H, fluorine, chlorine, nitro or a —NH₂ group,

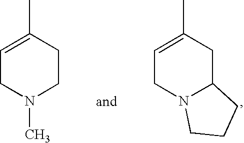

R^{13p} and R^{14p}, identical or different, each represent methyl, ethyl, n-propyl or isopropyl, more preferably methyl,

or

R^{13p} and R^{14p} together with the bridging nitrogen atom form a 5- or 6-membered heterocyclic ring, more preferably form a pyrrolidine or piperidine ring,

A^{p} and B^{p} together with the carbon atom to which they are bonded form a saturated or unsaturated C₃-C₈ cycloaliphatic ring, more preferably form a cyclohexyl ring,

and

np is 0, 1 or 2;

optionally in form of one of their stereoisomers, preferably enantiomers or diastereomers, their racemate or in form of a mixture of at least two of their stereoisomers, preferably enantiomers or diastereomers, in any mixing ratio, or a salt thereof, preferably a corresponding physiologically acceptable salt thereof or a corresponding solvate thereof.

Particularly preferred compounds of general formula (Ip) are those selected from the group consisting of
1-Cyclohexanesulfonyl-3-(1-methyl-1,2,3,6-tetrahydropyridine-4-yl)-5-nitro-1H-indole,
5-chloro-1-cyclohexanesulfonyl-3-(1-methyl-1,2,3,6-tetrahydropyridine-4-yl)-1H-indole,
5-Amino-1-cyclohexanesulfonyl-3-(1-methyl-1,2,3,6-tetrahydropyridine-4-yl)-1H-indole and
1-Cyclohexanesulfonyl-5-fluoro-3-(1,2,3,5,8,8a-hexahydro-indolizine-7-yl)-1H-indole hydrochloride

and their corresponding salts and solvates.

The compounds of general formula (Ip) can be prepared according to the disclosure of WO 2005/013974. The respective part of the literature is hereby incorporated by reference and forms part of the present disclosure.

Preferred compounds of general formula (Ib) are selected from the group consisting of compounds of general formula (Iq)

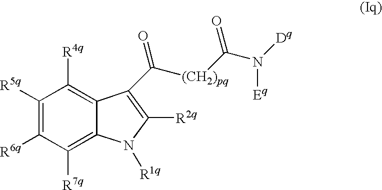

wherein

R^{1q} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical,

which may be bonded via a linear or branched alkylene group and/or which may be condensed with an unsubstituted or at least mono-substituted mono- or bicyclic ring system; an unsubstituted or at least mono-substituted aryl or heteroaryl radical, which may be bonded via a linear or branched alkylene group and/or which may be condensed with an unsubstituted or at least mono-substituted mono- or bicyclic ring system; a —C(═O)—R^{8q} moiety; a —S(═O)₂—R^{9q} moiety;

R^{2q} represents a hydrogen atom; —F; —Cl; —Br; —I; —NO₂; —NH₂; —SH; —OH; —CN; a linear or branched, saturated or unsaturated, unsubstituted or at least mono-substituted, optionally at least one heteroatom as a chain member containing aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched alkylene group and/or which may be condensed with an unsubstituted or at least mono-substituted mono- or bicyclic ring system;

R^{4q}, R^{5q}, R^{6q} and R^{7q}, independently of one another, each represent a hydrogen atom; —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{8q}; —S(═O)₂—R^{9q}; —O—R^{10q}; —S—R^{11q}; —C(═O)—OR^{12q}; —N(R^{15q})—S(═O)₂—R^{16q}; —NH—R^{17q}; —NR^{18q}R^{19q}; —C(═O)—NHR^{20q}, —C(═O)—NR^{21q}R^{22q}; —S(═O)₂—NHR^{23q}; —S(═O)₂—NR^{24q}R^{25q}; —O—C(═O)—R^{26q}; —NH—C(═O)—R^{27q}; —NR^{28q}—C(═O)—R^{29q}; NH—C(═O)—O—R^{30q}; NR^{31q}—C(═O)—O—R^{32q}; —S(═O)₂—O—R^{33q}; a halogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene group;

with the proviso that at least one of the substituents R^{4q}, R^{5q}, R^{6q} and R^{7q} represents a —N(R^{15q})—S(═O)₂—R^{16q} moiety;

R^{8q}, R^{12q}, R^{17q}, R^{18q}, R^{19q}, R^{20q}, R^{21q}, R^{22q}, R^{23q}, R^{24q}, R^{25q}, R^{26q}, R^{27q}, R^{28q}, R^{29q}, R^{30q}, R^{31q}, R^{32q} and R^{33q}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene, alkenylene or alkinylene group;

R^{9q}represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{10q} and R^{11q}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;

R^{15q} represents a hydrogen atom; a linear or branched, saturated or unsaturated, unsubstituted or at least mono-substituted aliphatic radical or a —S(═O)₂—R^{16q} moiety;

R^{16q} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

D^{q} and E^{q} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

or

D^{q} and E^{q}, independently of one another, each represent a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Iq) are those, wherein

pq is 0,

R^{1q} represents a hydrogen atom,

R^{2q} represents a hydrogen atom,

D^{q} and E^{q}, identical or different, represent an alkyl radical selected from the group consisting of methyl, ethyl, n-propyl, isopropyl, n-butyl, sec-butyl, isobutyl and tert-butyl,

one of the substituents R^{4q}, R^{5q}, R^{6q}and R^{7q} represents an —N(R^{15q})—S(═S)—R^{16q}-moiety while the other three of these substituents each represent a hydrogen atom,

R^{15q} represents a hydrogen atom,

R^{16q} represents an aryl or heteroaryl radical selected from the group consisting of phenyl, 1-naphthyl, 2-naphthyl, pyrazolyl, thiophenyl (thiophenyl), benzo[b]-thiophenyl, benzo[b]furanyl, quinolinyl, isoquinolinyl, imidazo[2,1-b]thiazolyl, 2-oxo-2,3-dihydro-benzooxazolyl and 2-oxo-2,3-dihydrobenzo[d]thiazolyl, whereby said aryl or heteroaryl radical may be bonded via a —(CH₂)_{1, 2 or 3}— group and/or may be substituted by 1, 2, 3, 4 or 5 substituents independently selected from the group consisting of methyl, ethyl, n-propyl, isopropyl, n-butyl, sec-butyl, isobutyl, tert-butyl, F, Cl, Br, I, —CN, —CF₃, —CF₂H, CFH₂, —C(═O)—O—CH₃, C(═O)—O—CH₂—CH₃, cyclopropyl, cyclobutyl, cyclopentyl, cyclohexyl, phenyl, phenoxy and benzyl;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Particularly preferred compounds of general formula (Iq) are those selected from the group consisting of:
2-[5-(5-chloro-3-methyl-benzo[b]thiophene-2-sulfonylamino)-1H-indol-3-yl]-N,N-diethyl-2-oxoacetamide,
N,N-Diethyl-2-[5-(naphthalene-2-sulfonylamino)-1H-indol-3-yl]-2-oxo-acetamide,
N,N-Diethyl-2-[5-(naphthalene-1-sulfonylamino)-1H-indol-3-yl]-2-oxo-acetamide,
2-[5-(Biphenyl-4-sulfonylamino)-1H-indol-3-yl]-N,N-diethyl-2-oxo-acetamide,
N,N-Diethyl-2-oxo-2-[5-(quinoline-8-sulfonylamino)-1H-indol-3-yl]-acetamide,
N,N-Dimethyl-2-[5-(naphthalene-2-sulfonylamino)-1H-indol-3-yl]-2-oxo-acetamide,
N,N-Dimethyl-2-[5-(naphthalene-1-sulfonylamino)-1H-indol-3-yl]-2-oxo-acetamide,
2-[5-(5-chloro-3-methyl-benzo[b]thiophene-2-sulfonylamino)-1H-indol-3-yl]-N,N-dimethyl-2-oxo-acetamide,
2-[5-(6-chloro-imidazo[2,1-b]thiazole-5-sulfonylamino)-1H-indol-3-yl]-N,N-diethyl-2-oxo-acetamide,
2-[5-(6-chloro-imidazo[2,1-b]thiazole-5-sulfonylamino)-1H-indol-3-yl]-N,N-dimethyl-2-oxo-acetamide,
N,N-Dimethyl-2-[4-(naphthalene-1-sulfonylamino)-1H-indol-3-yl]-2-oxo-acetamide,
2-[4-(5-chloro-3-methyl-benzo[b]thiophene-2-sulfonylamino)-1H-indole-3-yl]-N,N-dimethyl-2-oxo-acetamide,
2-[4-(6-chloro-imidazo[2,1-b]thiazole-5-sulfonylamino)-1H-indol-3-yl]-N,N-dimethyl-2-oxo-acetamide,
N,N-Dimethyl-2-[5-[(4-fluoro-3-methyl-phenyl)-1-sulfonylamino]-1H-indol-3-yl]-2-oxo-acetamide,
5-(3-Dimethylaminooxalyl-1H-indol-5-ylsulfamoyl)-3-methyl-benzofuran-2-carboxylic acid ethyl ester,
2-[5-(Biphenyl-4-sulfonylamino)-1H-indol-3-yl]-N,N-dimethyl-2-oxo-acetamide,
N,N-Dimethyl-2-oxo-2-[5-(2-oxo-2,3-dihydro-benzoxazole-6-sulfonylamino)-1H-indol-3-yl]acetamide,
N,N-Dimethyl-2-oxo-2-[5-(2-oxo-2,3-dihydrobenzo[d]thiazole-6-sulfonamido)-1H-indol-3-yl]acetamide,
2-[5-[(4-Cyclohexyl-phenyl)-1-sulfonylamino]-1H-indol-3-yl]-N,N-dimethyl-2-oxo-acetamide,
N,N-Dimethyl-2-[5-[(4-phenoxy-phenyl)-1-sulfonylamino]-1H-indol-3-yl]-2-oxo-acetamide,
2-(5-(5-chloro-3-methylbenzo[b]thiophene-2-sulfonamido)-2-methyl-1H-indol-3-yl)-N,N-dimethyl-2-oxoacetamide,
2-(5-(6-chloroimidazo[2,1-b]thiazole-5-sulfonamido)-2-methyl-1H-indol-3-yl)-N,N-dimethyl-2-oxoacetamide,
2-(6-(5-chloro-3-methylbenzo[b]thiophene-2-sulfonamido)-1H-indol-3-yl)-N,N-dimethyl-2-oxoacetamide,
N,N-dimethyl-2-(6-(naphthalene-3-sulfonamido)-1H-indol-3-yl)-2-oxoacetamide
2-(6-(biphenyl-4-sulfonamido)-1H-indol-3-yl)-N,N-dimethyl-2-oxoacetamide,
N,N-dimethyl-2-(6-(naphthalene-1-sulfonamido)-1H-indol-3-yl)-2-oxoacetamide,
N,N-dimethyl-2-(6-(2-(naphthalen-1-yl)ethylsulfonamido)-1H-indol-3-yl)-2-oxoacetamide,
N,N-dimethyl-2-oxo-2-(6-(4-phenoxyphenylsulfonamido)-1H-indol-3-yl)acetamide,
2-(6-(3,4-dichlorothiophene-2-sulfonamido)-1H-indol-3-yl)-N,N-dimethyl-2-oxoacetamide,
2-(6-(3,5-dichlorophenylsulfonamido)-1H-indol-3-yl)-N,N-dimethyl-2-oxoacetamide,
2-(6-(1-chloronaphthalene-6-sulfonamido)-1H-indol-3-yl)-N,N-dimethyl-2-oxoacetamide,
2-(6-(6-chloroimidazo[2,1-b]thiazole-5-sulfonamido)-1H-indol-3-yl)-N,N-dimethyl-2-oxoacetamide,
N,N-diethyl-2-(2-methyl-5-(5-methyl-1-phenyl-1H-pyrazole-4-sulfonamido)-1H-indol-3-yl)-2-oxoacetamide and
N,N-diethyl-2-(2-methyl-5-(1,3,5-trimethyl-1H-pyrazole-4-sulfonamido)-1H-indol-3-yl)-2-oxoacetamide;

and their corresponding salts and solvates.

The compounds of general formula (Iq) can be prepared according to the disclosure of WO 2006/015867. The respective part of the literature is hereby incorporated by reference and forms part of the present disclosure.

Preferably as component (A) at least one compound is present which is selected from the group consisting of indazolyl- and (2,3)-dihydro-indolyl-derived sulfonamide compounds of general formula (Ic)

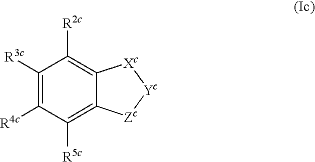

wherein

X^{c}—Y^{c} from left to right represents CR^{1c}═N and Z^{c} is N[(CH₂)_{nc}R^{6c}]

or

X^{c}—Y^{c} from left to right represents CR^{7c}═N, Z^{c} is NH, R^{7c} represents the following moiety

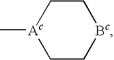

A^{c} represents CH or N and B^{c} represents NR^{8c}, O or S;

X^{c}—Y^{c} from left to right represents C[(CH₂)_{nc}R^{9c}]═N and Z^{c} is NR^{10c}

or

X^{c}—Y^{c} represents CH₂—CH₂ and Z^{c} is N[(CH₂)_{nc}R^{11c}];

nc is 0, 1, 2, 3 or 4;

R^{1c} represents a hydrogen atom; NO₂; —NH₂; —SH; —OH; —CN; —C(═O)—R^{12c}; —OR^{13c}; —SR^{14c}; —F; —Cl, —Br; —I; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{2c}, R^{3c}, R^{4c} and R^{5c}, independently of one another, each represent a hydrogen atom; —NO₂; —NH₂; —SH; —OH; —CN; —C(═O)—H; —C(═O)—R^{12c}; —OR^{13c}; —SR^{14c}; —N(R^{15c})—S(═O)₂—R^{16c}; —NH—R^{17c}; —NR^{18c}R^{19c}; —F; —Cl, —Br; —I; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

with the proviso that at least one of the substituents R^{2c}, R^{3c}, R^{4c} and R^{5c} represents a —N(R^{15c})—S(═O)₂—R^{16c} moiety;

R^{6c}, R^{9c} and R^{11c}, independently of one another, each represent a —NR^{20c}R^{21c} radical

or

a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{8c} represents —C(═O)—R^{22c}; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{10c} represents a linear or branched, saturated or unsaturated, unsubstituted or at least mono-substituted aliphatic radical; or a —S(═O)₂—R^{23c} moiety;

R^{12c}, R^{13c}, R^{14 c}, R^{17c}, R^{18c} and R^{19c}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{15c} represents a hydrogen atom; a linear or branched, saturated or unsaturated, unsubstituted or at least mono-substituted aliphatic radical; or a —S(═O)₂—R^{24c} moiety;

R^{16c} and R^{24c}, independently of one another, each represent an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{20c} and R^{21c}, independently of one another, each represent a hydrogen atom; or a linear or branched, saturated or unsaturated, unsubstituted or at least mono-substituted aliphatic radical;

or R^{20c} and R^{21c} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{22c} represents a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

and

R^{23c} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ic) are selected from the group consisting of compounds of general formula (Ir)

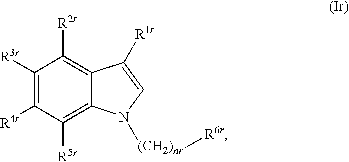

wherein

nr is 0, 1 or 2;

R^{1r} represents a hydrogen atom or an alkyl radical selected from the group consisting of methyl, ethyl, n-propyl, isopropyl, n-butyl, sec-butyl, isobutyl and tert-butyl;

R^{2r}, R^{3r}, R^{4r} and R⁵r, independent from one another, each represent a hydrogen atom; —NO₂; —NH₂; —SH; —OH; —CN; —N(R^{15r})—S(═O)₂—R^{16r}; F; Cl; Br; I; or an alkyl radical selected from the group consisting of methyl, ethyl, n-propyl, isopropyl, n-butyl, sec-butyl, isobutyl and tert-butyl;

with the proviso that at least one of the substituents R^{2r}, R^{3r}, R^{4r} and R^{5r} represents a —N(R^{15r})—S(═O)₂—R^{16r} moiety;

R^{6r} represents a —NR^{20r}R^{21r} radical;

R^{15r} represents a hydrogen atom or an alkyl radical selected from the group consisting of methyl, ethyl, n-propyl, isopropyl, n-butyl, sec-butyl, isobutyl and tert-butyl

R^{16r} represents an aryl or heteroaryl radical selected from the group consisting of phenyl, naphthyl, benzo[b]furanyl, benzo[b]thiophenyl and imidazo[2,1-b]thiazolyl, which may be substituted with 1, 2 or 3 substituent(s) independently selected from the group consisting of methyl, ethyl, n-propyl, isopropyl, n-butyl, sec-butyl, isobutyl, tert-butyl, F, Cl, Br, I, —CN, phenyl, phenoxy and benzyl

and

R^{20r} and R^{21r}, independent from one another, each represent an alkyl radical selected from the group consisting of methyl, ethyl, n-propyl, isopropyl, n-butyl, sec-butyl, isobutyl and tert-butyl;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Particularly preferred compounds of general formula (Ir) are those selected from the group consisting of:
N-(1-(2-(Dimethylamino)ethyl)-1H-indazol-6-yl)napthalene-2-sulfonamide and
5-chloro-N-(1-(2-(dimethylamino)ethyl)-1H-indazol-6-yl)-3-methylbenzo[b]thiophene-2-sulfonamide;

optionally in form of a physiologically acceptable salt thereof, or a corresponding solvate thereof.

Preferred compounds of general formula (Ic) are selected from the group consisting of compounds of general formula (Is)

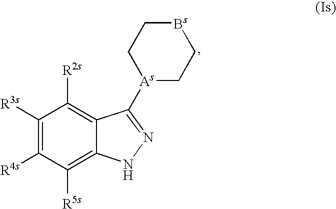

wherein

A^{s} represents CH and Bₛ represents NR^{8s}

or

A^{s} represents N and B^{s} represents NR^{8s}

or

A^{s} represents N and B^{s} represents O

or

A^{s} represents N and B^{s} represents S;

R^{2s}, R^{3s}, R^{4s} and R^{5s}, independent from one another, each represent a hydrogen atom; —NO₂; —NH₂; —SH; —OH; —CN; —N(R^{15s})—S(═O)₂—R^{16s}; F; Cl; Br; I; or an alkyl radical selected from the group consisting of methyl, ethyl, n-propyl, isopropyl, n-butyl, sec-butyl, isobutyl and tert-butyl;

with the proviso that at least one of the substituents R^{2s}, R^{3s}, R^{4s} and R^{5s} represents a —N(R^{15s})—S(═O)₂—R^{16s} moiety;

R^{8s} represents an alkyl radical selected from the group consisting of methyl, ethyl, n-propyl, isopropyl, n-butyl, sec-butyl, isobutyl and tert-butyl;

R^{15s} represents a hydrogen atom or an alkyl radical selected from the group consisting of methyl, ethyl, n-propyl, isopropyl, n-butyl, sec-butyl, isobutyl and tert-butyl

and

R^{16s} represents an aryl or heteroaryl radical selected from the group consisting of phenyl, naphthyl, benzo[b]furanyl, benzo[b]thiophenyl and imidazo[2,1-b]thiazolyl, which may be substituted with 1, 2 or 3 substituent(s) independently selected from the group consisting of methyl, ethyl, n-propyl, isopropyl, n-butyl, sec-butyl, isobutyl, tert-butyl, F, Cl, Br, I, —CN, phenyl, phenoxy and benzyl;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Particularly preferred compounds of general formula (Is) are those selected from the group consisting of:
Naphthalene-2-sulfonic acid[3-(1-methyl-piperidin-4-yl)-1H-indazol-5-yl]-amide,
5-chloro-3-methyl-benzo[b]thiophene-2-sulfonic acid[3-(1-methyl-piperidin-4-yl)-1H-indazol-5-yl]-amide,
Naphthalene-1-sulfonic acid[3-(1-methyl-piperidin-4-yl)-1H-indazol-5-yl]-amide,
4-Phenylbenzene-4-sulfonic acid[3-(1-methyl-piperidin-4-yl)-1H-indazol-5-yl]-amide,
N-[3-(1-Methyl-piperidin-4-yl)-1H-indazol-5-yl]-4-phenoxy-benzenesulfonamide and
N-[3-(1-Methyl-piperidin-4-yl)-1H-indazol-5-yl]benzenesulfonamide;

optionally in form of a physiologically acceptable salt thereof, or a corresponding solvate thereof.

Preferred compounds of general formula (Ic) are selected from the group consisting of compounds of general formula (It)

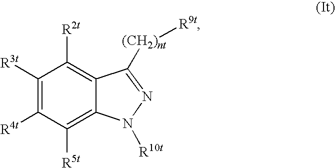

wherein

nt is 0, 1 or 2;

R^{2t}, R^{3t}, R^{4t} and R^{5t}, independent from one another, each represent a hydrogen atom; —NO₂; —NH₂; —SH; —OH; —CN; —N(R^{15t})—S(═O)₂—R^{16t}; F; Cl; Br; I; or an alkyl radical selected from the group consisting of methyl, ethyl, n-propyl, isopropyl, n-butyl, sec-butyl, isobutyl and tert-butyl;

with the proviso that at least one of the substituents R^{2t}, R^{3t}, R^{4t} and R^{5t} represents a —N(R^{15t})—S(═O)₂—R^{16t} moiety;

R^{9t} represents a —NR^{20t}R^{21t} radical;

R^{10t} represents an alkyl radical selected from the group consisting of methyl, ethyl, n-propyl, isopropyl, n-butyl, sec-butyl, isobutyl and tert-butyl or a —S(═O)₂—R^{23t} moiety;

R^{15t} represents a hydrogen atom or an alkyl radical selected from the group consisting of methyl, ethyl, n-propyl, isopropyl, n-butyl, sec-butyl, isobutyl and tert-butyl

R^{16t} represents an aryl or heteroaryl radical selected from the group consisting of phenyl, naphthyl, benzo[b]furanyl, benzo[b]thiophenyl and imidazo[2,1-b]thiazolyl, which may be substituted with 1, 2 or 3 substituent(s) independently selected from the group consisting of methyl, ethyl, n-propyl, isopropyl, n-butyl, sec-butyl, isobutyl, tert-butyl, F, Cl, Br, I, —CN, phenyl, phenoxy and benzyl.

R^{20t} and R^{21t}, independent from one another, each represent an alkyl radical selected from the group consisting of methyl, ethyl, n-propyl, isopropyl, n-butyl, sec-butyl,

isobutyl and tert-butyl;

or

R^{20t} and R^{21t} together with the bridging nitrogen atom form an unsubstituted moiety selected from the group consisting of

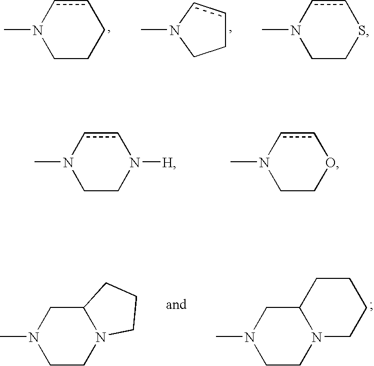

wherein, if present, the dotted line represents an optional chemical bond;

and

R^{23t} represents an alkyl radical selected from the group consisting of methyl, ethyl, n-propyl, isopropyl, n-butyl, sec-butyl, isobutyl and tert-butyl

or an aryl or heteroaryl radical selected from the group consisting of phenyl, naphthyl, benzo[b]furanyl, benzo[b]thiophenyl and imidazo[2,1-b]thiazolyl, which may be substituted with 1, 2 or 3 substituent(s) independently selected from the group consisting of methyl, ethyl, n-propyl, isopropyl, n-butyl, sec-butyl, isobutyl, tert-butyl, F, Cl, Br, I, —CN, phenyl, phenoxy and benzyl;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ic) are selected from the group consisting of compounds of general formula (Iu)

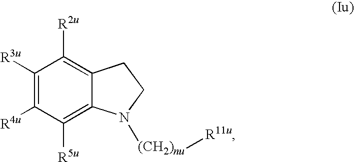

wherein

nu is 0, 1 or 2;

R^{2u}, R^{3u}, R^{4u} and R^{5u}, independent from one another, each represent a hydrogen atom; —NO₂; —NH₂; —SH; —OH; —CN; —C(═O)—H; —C(═O)—R^{12u}; —OR^{13u}; —SR^{14u}; —N(R^{15u})—S(═O)₂—R^{16u}; —NH—R^{17u}; —NR^{18u}R^{19u}; F; Cl; Br; I; or an alkyl radical selected from the group consisting of methyl, ethyl, n-propyl, isopropyl, n-butyl, sec-butyl, isobutyl and tert-butyl which may be substituted with 1, 2 or 3 substituent(s) independently selected from the group consisting of F, Cl, Br, —OH, —NH₂, —SH, —O—CH₃, —O—C₂H₅, —NO₂, —CN and —S—CH₃;

with the proviso that at least one of the substituents R^{2u}, R^{3u}, R^{4u} and R^{5u} represents a —N(R^{15u})—S(═O)₂—R^{16u} moiety;

R^{11u} represents a —NR^{20u}R^{21u} radical

or

a (hetero)cycloaliphatic radical selected from the group consisting of

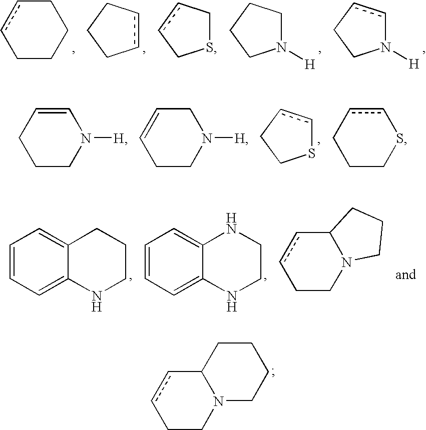

whereby each of these afore mentioned cyclic moieties may be substituted with 1, 2 or 3 substituent(s) independently selected from the group consisting of oxo (═O), thioxo (═S), methyl, ethyl, n-propyl, isopropyl, n-butyl, sec-butyl, isobutyl, tert-butyl, —O—CH₃, —O—C₂H₅, —S—CH₃, —S—C₂H₅, —C(═O)—OH, —C(═O)—O—CH₃, F, Cl, Br, I, —CN, —OCF₃, —SCF₃, —OH, —SH, —NH₂, —NH—CH₃, —NH—C₂H₅, —N(CH₃)₂, —N(C₂H₅)₂, —NO₂, —CHO, —CF₂H and —CFH₂ in any position including the —NH groups and is not bonded via a nitrogen atom and, if present, the dotted line represents an optional chemical bond;

R^{12u}, R^{13u}, R^{14u}, R^{17u}, R^{18u} and R^{19u}, independent from one another, each represent an alkyl radical selected from the group consisting of methyl, ethyl, n-propyl, isopropyl, n-butyl, sec-butyl, isobutyl and tert-butyl; a (hetero)cycloaliphatic radical selected from the group consisting of cyclopentyl, cyclohexyl, pyrrolidinyl, piperidinyl, morpholinyl, thiomorpholinyl and piperazinyl, which may be bonded via a —(CH₂)_{1, 2 or 3}— group and which may be substituted with 1, 2 or 3 substituent(s) independently selected from the group consisting of oxo (═O), thioxo (═S), methyl, ethyl, —O—CH₃, —O—C₂H₅, —S—CH₃, —C(═O)—OH, —C(═O)—O—CH₃, —F, Cl, Br, I, —CN, —OCF₃, —SCF₃, —OH, —SH, —NH₂, —NH—CH₃, —NH—C₂H₅, —N(CH₃)₂ and —N(C₂H₅)₂; or an aryl or heteroaryl radical selected from the group consisting of phenyl, naphthyl, pyridinyl, furyl(furanyl), thiophenyl(thiophenyl) and pyrrolyl, which may be bonded via a —(CH₂)_{1, 2 or 3}— group and which may be substituted with 1, 2 or 3 substituent(s) independently selected from the group consisting of —CF₃, methyl, ethyl, —O—CH₃, —O—C₂H₅, —O—CH₂—CH₂—CH₃, —S—CH₂H₅, —C(═O)—OH, —O(═O)—O—CH₃, —C(═O)—O—CH₂—CH₃, F, Cl, Br, I, —CN, —OCF₃, —SCF₃, —OH, —SH, —NH₂, —NH—CH₃, —NH—C₂H₅, —N(CH₃)₂ and —N(C₂H₅)₂;

R^{15u} represents a hydrogen atom; or an alkyl radical selected from the group consisting of methyl, ethyl, n-propyl, isopropyl, n-butyl, sec-butyl, isobutyl and tert-butyl which may be substituted with 1, 2 or 3 substituent(s) independently selected from the group consisting of F, Cl, Br, —OH, —NH₂, —SH, —O—CH₃, —O—C₂H₅, —NO₂, —CN and —S—CH₃;

R^{16u} represents an aryl or heteroaryl radical selected from the group consisting of phenyl, naphthyl, pyridinyl, furyl(furanyl), thiophenyl(thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, indolyl, isoindolyl, pyrimidinyl, pyrazinyl, quinolinyl, isoquinolinyl, benzo[b]furanyl, benzo[b]thiophenyl, benzothiadiazolyl, benzoxadiazolyl, benzoxazolyl, benzthiazolyl, benzisoxazolyl, benzisothiazolyl and imidazo[2,1-b]thiazolyl, which may be bonded via a —(CH₂)_{1, 2 or 3}— group and which may be substituted with 1, 2 or 3 substituent(s) independently selected from the group consisting of —CF₃, methyl, ethyl, n-propyl, isopropyl, n-butyl, sec-butyl, isobutyl, tert-butyl, —O—CH₃, —O—C₂H₅, —S—CH₃, —S—C₂H₅, F, Cl, Br, I, —CN, —OCF₃, —SCF₃, —OH, —SH, —NH₂, —NH—CH₃, —NH—C₂H₅, —N(CH₃)₂, —N(C₂H₅)₂, —NO₂, phenyl, phenoxy and benzyl;

and

R^{20u} and R^{21u}, independent from one another, each represent a hydrogen atom; or an alkyl radical selected from the group consisting of methyl, ethyl, n-propyl, isopropyl, n-butyl, sec-butyl, isobutyl and tert-butyl;

or

R^{20u} and R^{21u} together with the bridging nitrogen atom form a moiety selected from the group consisting of

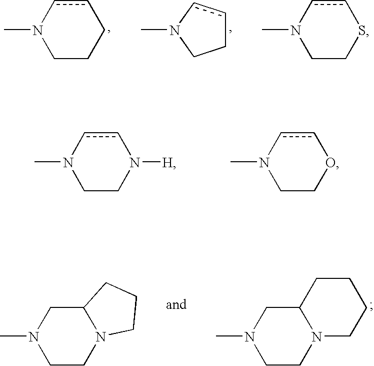

whereby each of these afore mentioned cyclic moieties may be substituted with 1, 2 or 3 substituent(s) independently selected from the group consisting of oxo (═O), thioxo (═S), methyl, ethyl, —O—CH₃, —O—C₂H₅, —S—CH₃, —S—C₂H₅, —C(═O)—OH, —C(═O)—O—CH₃, —C(═O)—O—CH₂—CH₃, F, Cl, Br, I, —CN, —OCF₃, —SCF₃, —OH, —SH, —NH₂, —NH—CH₃, —NH—C₂H₅, —N(CH₃)₂ and —N(C₂H₅)₂ in any position including the —NH groups; and, if present, the dotted line represents an optional chemical bond;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

Particularly preferred compounds of general formula (Iu) are those selected from the group consisting of:
N-[1-(2-Dimethylamino)ethyl)-2,3-dihydro-1H-indol-6-yl]-6-chloro-imidazo[2,1-b]thiazol-5-sulfonamide;

optionally in form of a physiologically acceptable salt thereof, or a corresponding solvate thereof.

The compounds of general formulae Ic, Ir, Is, It or Iu given above are prepared by a process, wherein at least one compound of general formula II,

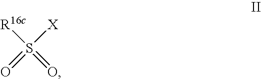

wherein R^{16c} has the meaning given above and X represents a leaving group, preferably a halogen atom, more preferably a chlorine atom, is reacted with at least one compound of general formula III,

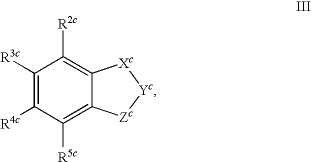

wherein X^{c}, Y^{c}, Z^{c} and R^{2c} to R^{5c} have the meaning given above with the proviso that at least one of the substituents R^{2c}, R^{3c}, R^{4c} and R^{5c}, represents a —NH₂ group, in a suitable reaction medium, preferably in the presence of at least one base, to yield a compound of general formula I, wherein X^{c}, Y^{c}, Z^{c} and R^{2c} to R^{5c} have the meaning given above with the proviso that at least one of the substituents R^{2c}, R^{3c}, R_{4c} and R^{5c} represents a —N(H)—S(═O)₂—R^{16c} group and R^{16c} has the meaning given above, which is optionally purified and/or isolated,

and optionally said compound of general formula I, wherein X^{c}, Y^{c}, Z^{c} and R^{2c} to R^{5c} have the meaning given above with the proviso that at least one of the substituents R^{2c}, R^{3c}, R^{4c} and R^{5c} represents a —N(H)—S(═O)₂—R^{16c} group and R^{16c} has the meaning given above, is reacted with at least one compound of general formula R^{15c}—X, wherein R^{15c} has the meaning given above and X represents a halogen atom, preferably a chlorine atom, in a suitable reaction medium, in the presence of at least one base, preferably at least one base selected from the group consisting of metal hydroxides, metal carbonates, metal alkoxides, preferably sodium methoxide or potassium tert-butoxid, metal hydrides and organometallic compounds, preferably n-butyllithium and tert-butyllithium,

or with at least one compound of general formula X—S(═O)₂—R^{24c}, wherein R^{24c} has the meaning given above and X represents a leaving group, preferably a halogen atom, more preferably a chlorine atom, in a suitable reaction medium, preferably in the presence of at least one base,

to yield a compound of general formula I, wherein X^{c}, Y^{c}, Z^{c} and R^{2c} to R^{5c} have the meaning given above with the proviso that at least one of the substituents R^{2c}, R^{3c}, R^{4c} and Rc⁵ represents a —N(R^{15c})—S(═O)₂—R^{16c} group and R^{15c} and R^{16c} have the meaning given above, which is optionally purified and/or isolated.

Suitable reaction media for the reaction between compounds of general formulae II and III include organic solvents, such as dialkyl ether, preferably diethyl ether, or a cyclic ether, preferably tetrahydrofuran or dioxane; or a halogenated hydrocarbon, preferably dichloromethane or chloroform; an alcohol, preferably methanol or ethanol; a dipolar aprotic solvent, preferably acetonitrile, pyridine or dimethylformamide, or any other suitable reaction medium. Of course, mixtures of at least two classes of solvents or of at least two solvents of one class may also be used.

The reaction between compounds of general formulae II and III is preferably carried out in the presence of at least one suitable base, for example, an inorganic base such as a hydroxide or a carbonate of an alkali metal and/or an organic base, preferably triethylamine or pyridine.

The reaction between compounds of general formulae II and III is preferably carried out at a temperature between −10° C. and ambient temperature, i.e. approximately 25° C. and the reaction time is preferably between 5 minutes and 24 hours.

Suitable reaction media for the reaction between compounds of general formula I, wherein X^{c}, Y^{c}, Z^{c} and R^{2c} to R^{5c} have the meaning given above with the proviso that at least one of the substituents R^{2c}, R^{3c}, R^{4c} and R^{5c} represents a —N(H)—S(═O)₂—R^{16c} group and R^{16c} has the meaning given above and compounds of general formula R^{15c}—X are dialkyl ether, preferably diethyl ether, or a cyclic ether, preferably tetrahydrofuran or dioxane, a hydrocarbon, preferably toluene, an alcohol, preferably methanol or ethanol, a dipolar aprotic solvent, preferably acetonitrile, pyridine or dimethylformamide, or any other suitable reaction medium. Of course, mixtures of at least two classes of solvents or of at least two solvents of one class may also be used.

The afore mentioned reaction is preferably carried out at a temperature between −10° C. and ambient temperature, i.e. approximately 25° C. and the reaction time is preferably 1 and 24 hours.

Suitable reaction media for the reaction between compounds of general formula I, wherein X^{c}, Y^{c}, Z^{c} and R^{2c} to R^{5c} have the meaning given above with the proviso that at least one of the substituents R^{2c}, R^{3c}, R^{4c} and R^{5c} represents a —N(H)—S(═O)₂—R^{16c} group and R^{16c} has the meaning given above, and compounds of general formula X—S(═O)₂—R^{24c} include organic solvents, such as dialkyl ether, preferably diethyl ether, or a cyclic ether, preferably tetrahydrofuran or dioxane; or a halogenated hydrocarbon, preferably dichloromethane or chloroform; an alcohol, preferably methanol or ethanol; a dipolar aprotic solvent, preferably acetonitrile, pyridine or dimethylformamide, or any other suitable reaction medium. Of course, mixtures of at least two classes of solvents or of at least two solvents of one class may also be used.

The afore mentioned reaction is preferably carried out in the presence of at least one suitable base, for example, an inorganic base such as a hydroxide or a carbonate of an alkali metal and/or an organic base, preferably triethylamine or pyridine.

The afore mentioned reaction is preferably carried out at a temperature between −10° C. and ambient temperature, i.e. approximately 25° C. and the reaction time is preferably between 5 minutes and 24 hours.

Those skilled in the art understand that the process described above can also be applied to the synthesis of compounds of general formula Ir, Is, It and Iu given above.

The compounds of general formula Ic, Ir, Is, It or Iu given above may be purified and/or isolated according to methods well known to those skilled in the art. Preferably, the compounds of general formula Ic, Ir, Is, It or Iu may be isolated by evaporating the reaction medium, addition of water and adjusting the pH value to obtain the compound in form of a solid that can be isolated by filtration, or by extraction with a solvent that is not miscible with water such as chloroform and purification by chromatography or recrystallisation from a suitable solvent.

The compounds of general formula II are commercially available or may be prepared according to methods well known in the art, for example, analogous to the methods described in the bibliography of E. E. Gilbert, Synthesis, 1969, 1, 3. The respective part of the literature description cited above is hereby incorporated by reference and forms part of the disclosure.

The compounds of general formula III are commercially available or may also be prepared according to standard methods known in the prior art, for example by methods similar to those described in the literature: Savitskaya, N. V. et al. Synthesis of 5-amino-3-(b-aminoethyl)indazole. Zhurnal Obshchei Khimii (1961), 31 1924-1926; Zhang, Han-Cheng et al. Discovery and Optimization of a Novel Series of Thrombin Receptor (PAR-1) Antagonists: Potent, Selective Peptide Mimetics Based on Indole and Indazole Templates. Journal of Medicinal Chemistry (2001), 44(7), 1021-1024; Ono, Shinichiro et al. Preparation of piperidine derivatives as muscarinic receptors stimulator for treatment of schizophrenia. WO 2004069828 A1; Wrzeciono, U. et al. Synthesis and antiinflammatory activity of some indazole derivatives. Part 36. Azoles. Pharmazie (1993), 48(8); 582-584; Filla, S. A. et al. Preparation o 3-(1-methylpiperidin-4-yl)-1H-indoles and 3-(1-methylpiperidin-4-yl)4-aza-1H-indoles as 5-HT1F agonist. WO 2000/487; Dumas, J. et al. Preparation of bicyclic (hetero)aryl- and pyridine-containing diaryl ureas as Raf kinase and angiogenesis inhibitors useful in the treatment of cancer and other disorders. WO 200478748; Mueller, S. G. et al. Preparation of ethynylpyridines and related compounds as melanin-concentrating hormone receptor (MCH-1) antagonist for the treatment of metabolic disorders. WO 200439780; Maeno, K. Preparation of aminoalkylindazole derivatives as 5-HT2c receptor agonists. WO 98/30548; Zhao, E.-C. et al. Synthesis of dialkylaminoalkyl derivatives of indazole. Zhurnal Obshchei Khimii (1959), 29, 1012-1020. Ham, P. et al. Preparation of N-heteroaryl-4′-oxadiazolylbiphenylcarboxamides as 5HT1D antagonists. WO 9532967A1; Stenkamp, D. et al. Preparation of arylamides as melanin concentrating hormone (MCH) receptor antagonists. WO 200403974. The respective parts of the literature are hereby incorporated by reference and form part of the disclosure.

The compounds of general formula Ic, Ir, Is, It or Iu given above may be purified and/or isolated according to methods well known to those skilled in the art. Preferably, the compounds of general formula Ic, Ir, Is, It or Iu may be isolated by evaporating the reaction medium, addition of water and then adjusting the pH value to obtain the compound in form of a solid that can be isolated by filtration, or by extraction with a solvent that is not miscible with water such as chloroform and purified by chromatography or recrystallisation from a suitable solvent.

During some synthetic reactions described above or while preparing the compounds of general formulae Ic, Ir, Is, It, Iu, II and II the protection of sensitive or reactive groups may be necessary and/or desirable. This can be performed by using conventional protective groups like those described in Protective groups in Organic Chemistry, ed. J. F. W. McOmie, Plenum Press, 1973; T. W. Greene & P. G. M. Wuts and Protective Groups in Organic Chemistry, John Wiley & sons, 1991. The respective parts of the description is hereby incorporated by reference and forms part of the disclosure. The protective groups may be eliminated when convenient by means well-known to those skilled in the art.

If the substituted indazolyl sulfonamide or 2,3-dihydro-indolyl sulfonamide compounds of general formula Ic are obtained in form of a mixture of stereoisomers, particularly enantiomers or diastereomers, said mixtures may be separated by standard procedures known to those skilled in the art, e.g. chromatographic methods or crystallization with chiral reagents.

The substituted indazolyl sulfonamide or 2,3-dihydro-indolyl sulfonamide compounds of general formula Ic and in each case stereoisomers thereof may be obtained in form of a corresponding salt according to methods well known to those skilled in the art, e.g. by reacting said compound with at least one inorganic and/or organic acid, preferably in a suitable reaction medium. Suitable reaction media include, for example, any of the ones given above. Suitable inorganic acids include but are not limited to hydrochloric acid, hydrobromic acid, phosphoric acid, sulfuric acid, nitric acid, suitable organic acids include but are not limited to citric acid, maleic acid, fumaric acid, tartaric acid, or derivatives thereof, p-toluenesulfonic acid, methanesulfonic acid or camphersulfonic acid.

Preferably as component (A) at least one compound is present which is selected from the group consisting of phenyl-piperazine-derived compounds of general formula (Id)

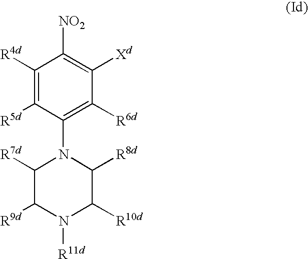

wherein

X^{d} represents a —NR^{1d}R^{2d} moiety or a —OR^{3d} moiety;

R^{1d} represents a linear or branched, saturated or unsaturated, unsubstituted or at least mono-substituted aliphatic radical;

an unsubstituted or at least mono-substituted radical selected from the group consisting of adamantyl, bicyclo[2.2.1]heptyl and bicyclo[3.1.1]heptyl, which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);

a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);

or a —C(═O)—R^{12d} moiety;

R^{2d} represents a hydrogen atom or a linear or branched, saturated or unsaturated, unsubstituted or at least mono-substituted aliphatic radical;

or

R^{1d} and R^{2d} together with the bridging nitrogen form an optionally at least mono-substituted, saturated, unsaturated or aromatic heterocyclic ring which may contain at least one further heteroatom as a ring member and/or which may be condensed with an unsubstituted or at least mono-substituted mono- or bicyclic ring system;

R^{3d} represents or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

R^{4d}, R^{5d} and R^{6d}, independently of one another, each represent a hydrogen atom or a halogen atom;

or

R^{4d} and R^{6d} together with the bridging carbon atoms form an unsubstituted 5- or 6-membered heterocyclic ring which contains 1, 2 or 3 heteroatom(s) independently selected from the group consisting of nitrogen, oxygen and sulfur as ring member(s) and which together with the phenyl ring which it is fused with forms a 9- or 10-membered bicyclic aromatic ring system;

R^{7d} and R^{8d}, independently of one another, each represent a hydrogen atom or a linear or branched, saturated or unsaturated, unsubstituted or at least mono-substituted aliphatic radical;

R^{9d} and R^{10d}, independently of one another, each represent a hydrogen atom or a linear or branched, saturated or unsaturated, unsubstituted or at least mono-substituted aliphatic radical;

R^{11d} represents a hydrogen atom; or a linear or branched, saturated or unsaturated, unsubstituted or at least mono-substituted aliphatic radical, which may contain 1, 2 or 3 heteroatom(s) independently selected from the group consisting of nitrogen, oxygen and sulfur as chain member(s);

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

-a-C(═O)—R^{13d} moiety or a —S(═O)₂—R^{14d} moiety;

R_{12d} represents a linear or branched, saturated or unsaturated, unsubstituted or at least mono-substituted aliphatic radical;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

and

R^{13d} and R^{14d}, independently of one another, each represent a linear or branched, saturated or unsaturated, unsubstituted or at least mono-substituted aliphatic radical; or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Id) are those, wherein

X^{d} represents a —NR^{1d}R^{2d} moiety or a —OR^{3d} moiety;

R^{1d} represents an alkyl radical selected from the group consisting of —CH₂—CH₂—OH and —CH₂—CH₂—CH₂—OH;

an unsubstituted adamantyl radical;

an unsubstituted phenyl or pyrrolyl radical;

an unsubstituted napthyl radical which is bonded via an alkylene group selected form the group consisting of —CH₂—, —CH(CH₃)—, —CH₂—CH₂—, —CH₂—CH₂—CH₂— and —CH₂—CH₂—O—;

a phenyl radical which may be substituted with 1, 2 or 3 substituent(s) independently selected from the group consisting of methyl, ethyl, tert-butyl, methoxy, F and Cl and said phenyl radical is bonded via an alkylene group selected form the group consisting of —CH₂—, —CH(CH₃)—, —CH(Phenyl)-, —CH₂—CH₂—, —CH₂—CH₂—CH₂— and —CH₂—CH₂—O—;

a heteroaryl radical selected from the group consisting of pyridinyl, furanyl and pyrrolyl, whereby said pyridinyl, furanyl or pyrrolyl radical may be substituted with 1, 2 or 3 substituent(s) independently selected from the group consisting of methyl, ethyl, tert-butyl, methoxy, F and Cl and said pyridinyl, furanyl or pyrrolyl radical is bonded via an alkylene group selected form the group consisting of —CH₂—, —CH(CH₃)—, —CH₂—CH₂—, —CH₂—CH₂—CH₂— and —CH₂—CH₂—O—;

or a —C(═O)—R^{12d} moiety;

R^{2d} represents a hydrogen atom or a methyl radical;

or

R^{1d} and R^{2d} together with the bridging nitrogen atom form a moiety selected from the group consisting of:

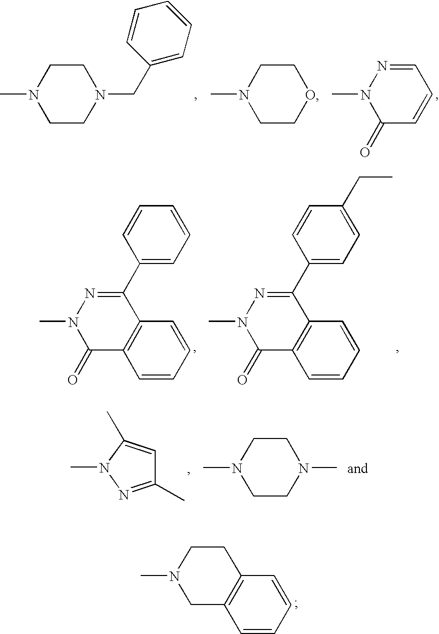

R^{3d} represents an unsubstituted phenyl radical;

R^{4d}, R^{5d} and R^{6d}, identical or different, each represent a hydrogen atom or a fluorine atom;

or

R^{4d} and R^{5d} together with the bridging carbon atoms form the following moiety,

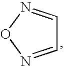

which together with the phenyl ring which it is fused with forms the following substituted bicyclic aromatic ring system

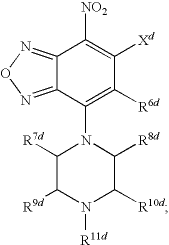

R^{7d} and R^{8d} each represent a hydrogen atom;

R^{9d} and R^{10d}, identical or different, each represent a hydrogen atom or a methyl radical;

R^{11d} represents a hydrogen atom;

an alkyl radical selected from the group consisting of methyl, n-butyl and —CH₂—CH₂—OH;

an unsubstituted phenyl or pyridinyl radical whereby said phenyl or pyridinyl radical may be bonded via a —(CH₂)— group;

a —C(═O)—R^{12d} moiety or a —S(═O)₂—R^{13d} moiety;

R^{12d} represents a phenyl or a thiophenyl radical whereby said phenyl or thiophenyl radical may be substituted with 1, 2 or 3 substituent(s) selected from the group consisting of methyl and chlorine;

R^{13d} represents a methyl radical or a phenyl or a thiophenyl radical whereby said phenyl or thiophenyl radical may be substituted with 1, 2 or 3 substituent(s) selected from the group consisting of methyl and chlorine

and

R^{14d} represents a methyl radical or a phenyl radical which may be substituted with 1, 2 or 3 methyl radical(s);

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

Particularly preferred compounds of general formula (Id) are those selected from the group consisting of:
[5-(4-Methyl-piperazin-1-yl)-2-nitro-phenyl]-(2-pyrrol-1-yl-ethyl)-amine,
(4-Fluoro-benzyl)-[5-(4-methyl-piperazin-1-yl)-2-nitro-phenyl]-amine,
N-[5-(4-Methyl-piperazin-1-yl)-2-nitro-phenyl]-benzamide,
N-Methyl-N-[5-(4-methyl-piperazin-1-yl)-2-nitro-phenyl]-benzamide,
[5-(4-Methyl-piperazin-1-yl)-2-nitro-phenyl]-pyrrol-1-yl-amine,
2-[5-(4-Methyl-piperazin-1-yl)-2-nitro-phenyl]-2H-pyridazin-3-one,
1-Methyl-4-(4-nitro-3-phenoxy-phenyl)-piperazine,
Benzyl-[5-(4-butyl-piperazin-1-yl)-2-nitro-phenyl]-amine,
Benzyl-[2-nitro-5-(4-pyridin-2-yl-piperazin-1-yl)-phenyl]-amine and
Benzyl-[2-nitro-5-(4-phenyl-2-yl-piperazin-1-yl)-phenyl]-amine;
Furan-2-ylmethy-[5-(4-methyl-piperazin-1-yl)-2-nitro-phenyl]-amine,
2-[4-(4-nitro-3-phenethylamino-phenyl)-piperazin-1-yl]-ethanol,
(2-nitro-5-piperazin-1-yl-phenyl)-(2-o-tolyloxy-ethyl)-amine
2-[4-(3-Benzylamino-4-nitro-phenyl)-piperazin-1-yl]-ethanol,
4-[5-(4-Methyl-piperazin-1-yl)-2-nitro-phenyl]-morpholine,
2-[5-(4-Methyl-piperazin-1-yl)-2-nitro-phenyl]-4-phenyl-2H-phthalazin-1-one,
[2-(4-chloro-phenoxy)-ethyl]-[5-(3,5-dimethyl-piperazin-1-yl)-2-nitro-phenyl]-amine,
2-[4-[3-(Benzhydryl-amino)-4-nitro-phenyl]-piperazin-1-yl]-ethanol,
4-[4-Fluoro-5-(4-methyl-piperazin-1-yl)-2-nitro-phenyl]-morpholine,
2-[2-nitro-5-[4-(toluene-4-sulfonyl)-piperazin-1-yl]-phenyl]-1,2,3,4-tetrahydro-isoquinoline,
1-[3-(3,5-Dimethyl-pyrazol-1-yl)-4-nitro-phenyl]-4-methyl-piperazine,
Benzyl-(2-nitro-5-piperazin-1-yl-phenyl)-amine,
[5-(4-Methyl-piperazin-1-yl)-2-nitro-phenyl]-phenethyl-amine,
[5-(4-Methyl-piperazin-1-yl)-2-nitro-phenyl]-pyridin-3-ylmethyl-amine,
(3-chloro-phenyl)-[4-[3-[(furanyl-2-ylmethyl)-amino]-4-nitro-phenyl]-piperazin-1-yl]-methanone,
(2-nitro-5-piperazin-1-yl-phenyl)-pyridin-3-ylmethyl-amine,
1-Benzyl-4-(2-nitro-5-piperazin-1-yl-phenyl)-piperazine,
Furan-2-ylmethyl-[5-(4-methanesulfonyl-piperazin-1-yl)-2-nitro-phenyl]-amine,
Benzhydryl-[5-(4-methyl-piperazin-1-yl)-2-nitro-phenyl]-amine,
(2-nitro-5-piperazin-1-yl-phenyl)-(2-phenoxy-ethyl)amine,
2-[5-(4-Methyl-piperazin-1-yl)-2-nitro-phenyl]-4-phenyl-2H-phthalazin-1-one,
1-[3-(3,5-Dimethyl-pyrazol-1-yl)-4-nitro-phenyl]-piperazine,
(2-nitro-5-piperazin-1-yl-phenyl)-phenethyl-amine,
[5-(4-Benzenesulfonyl-piperazin-1-yl)-2-nitro-phenyl]-furan-2-ylmethyl-amine,
[2-(3,4-Dimethoxy-phenyl)-ethyl]-(2-nitro-5-piperazin-1-yl-phenyl)-amine,
[4-[3-[(Furan-2-ylmethyl)-amino]-4-nitro-phenyl]-piperazin-1-yl]-m-tolyl-methanone,
[4-[3-[(Furan-2-ylmethyl)-amino]-4-nitro-phenyl]-piperazin-1-yl]-phenyl-methanone,
[4-[3-(3,5-Dimethyl-pyrazol-1-yl)-4-nitro-phenyl]-piperazin-1-yl]-thiophen-2-yl-methanone,
4-(4-Ethyl-phenyl)-2-[5-[4-methyl-piperazin-1-yl)-2-nitro-phenyl]-2H-phthalalazin-1-one,
3-[7-(4-Methyl-piperazin-1-yl)-4-nitro-benzo[1,2,5]oxadiazol-5-ylamino]-propan-1-ol,
3-[4-nitro-7-(4-phenyl-piperazin-1-yl)-benzo[1,2,5]oxadiazol-5-ylamino]-ethan-1-ol,
3-[4-nitro-7-(4-pyridin-piperazin-1-yl)-benzo[1,2,5]oxadiazol-5-ylamino]-propan-1-ol,
[2-(3,4-Dimethoxy-phenyl)-ethyl]-[5-(4-methyl-piperazin-1-yl)-2-nitro-phenyl]-amine,
[2-(3,4-Dimethyl-phenyl)-ethyl]-[5-(4-methyl-piperazin-1-yl)-2-nitro-phenyl]-amine,
[2-(4-tert-Butyl-phenoxy)-ethyl]-[5-(4-methyl-piperazin-1-yl)-2-nitro-phenyl]-amine,
[2-(4-Methoxy-phenoxy)-ethyl]-[5-(4-methyl-piperazin-1-yl)-2-nitro-phenyl]-amine,
[5-(4-Methyl-piperazin-1-yl)-2-nitro-phenyl]-(2-m-tolyloxy-ethyl)-amine,
[2-(4-chloro-phenoxy)-ethyl]-[5-(4-methyl-piperazin-1-yl)-2-nitro-phenyl]-amine,
(2-nitro-5-piperazin-1-yl-phenyl)-(1-phenyl-ethyl)-amine,
[2-(3-Methoxy-phenoxy)-ethyl]-[5-(4-methyl-piperazin-1-yl)-2-nitro-phenyl]-amine,
[2-(2-Methoxy-phenoxy)-ethyl]-[5-(4-methyl-piperazin-1-yl)-2-nitro-phenyl]-amine,
(4-chloro-benzyl)-(2-nitro-5-piperazin-1-yl-phenyl)-amine,
Benzyl-[5-(4-benzyl-piperazin-1-yl)-2-nitro-phenyl]-amine,
Benzyl-[5-(4-methyl-piperazin-1-yl)-2-nitro-phenyl]-amine,
[5-(4-Methyl-piperazin-1-yl)-2-nitro-phenyl]-(2-o-tolyloxy-ethyl)-amine,
(4-chloro-benzyl)-[5-(4-methyl-piperazin-1-yl)-2-nitro-phenyl]-amine,
Furan-2-ylmethyl-(2-nitro-5-piperazin-1-yl-phenyl)amine,
[2-(4-chloro-phenoxy)-ethyl]-(2-nitro-5-piperazin-1-yl-phenyl)-amine,
[5-(4-Methyl-piperazin-1-yl)-2-nitro-phenyl]-(1-phenyl-ethyl)-amine
1-[4-(3-Benzylamino-4-nitro-phenyl)-piperazin-1-yl]-ethanone,
2-[4-[3-(4-Methylpiperazin-1-yl)-4-nitrophenyl]piperazin-1-yl]ethanol,
2-[4-[3-[2-(Naphthalen-2-yloxy)ethylamino]-4-nitrophenyl]piperazin-1-yl]ethanol,
2-[4-(3-{[1-(1-Adamantyl)ethyl]amino}-4-nitrophenyl)piperazin-1-yl]ethanol and
2-[4-[3-(3,4-Dimethoxyphenethylamino)-4-nitrophenyl]piperazin-1-yl]ethanol;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof.

The compounds of general formula Id are prepared by a process, wherein at least one nitrobenzene compound of general formula II,

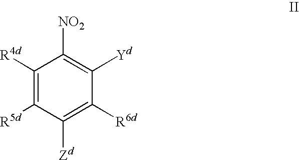

wherein R^{4d} to R^{6d} have any of the above given meanings, Y^{d} represents a chlorine atom, and Z^{d} represents a bromine or iodine atom; is reacted with at least one compound of general formula III,

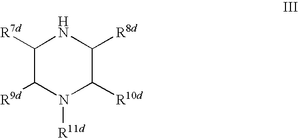

wherein R^{7d} to R^{11d} have any of the above given meanings in a suitable reaction medium, preferably in at least an organic solvent, more preferably in at least an organic solvent selected from the group consisting of tetrahydrofuran, toluene and dioxane, preferably in the presence of at least one catalyst, more preferably in the presence of at least a palladium source, even more preferably in the presence of PdCl₂(dppf) wherein dppf is 1,1-bis(diphenylphosphino)-ferrocene, and/or at least one auxiliary agent, preferably 1,1-bis(diphenylphosphino)-ferrocene, and/or at least one base, preferably sodium tert-pentoxide, to yield a compound of general formula IV,

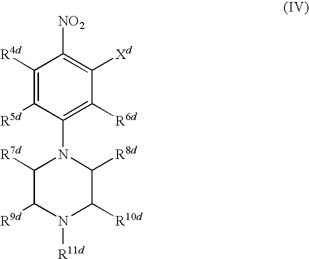

wherein R^{4d} to R^{11d} have any of the above given meanings and Y^{d} represents a chlorine atom; which is optionally purified and/or isolated, and the compound of general formula IV is reacted with at least one compound of general formula V,

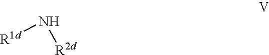

wherein R^{1d} and R^{2d} have any of the above given meanings, in a suitable reaction medium, preferably in at least an organic solvent, more preferably in at least an organic solvent selected from the group consisting of toluene or dimethoxyethane, preferably in the presence of at least one catalyst, more preferably in the presence of at least a palladium source, even more preferably in the presence of at least a palladium source selected from the group consisting of Pd(OAc)₂, wherein OAc is acetate, and Pd₂dba₃, wherein dba is dibenzylidene acetone, and/or at least one auxiliary agent, preferably (biph)P(tBu)₂, wherein biph is biphenyl and tBu is tert-butyl, and/or at least one base, preferably at least one base selected from the group consisting of K₃PO₄ and sodium tert-pentoxide to yield a compound of general formula VI,

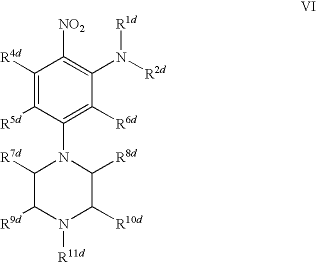

wherein R^{1d}, R^{2d} and R^{4d} to R^{11d} have any of the above given meanings which is optionally purified and/or isolated.

The compounds of general formula Id are prepared by a process, wherein at least one nitrobenzene compound of general formula VII,

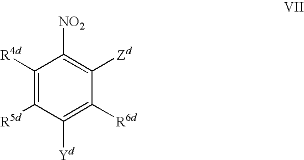

wherein R^{4d} to R^{6d} have any of the above given meanings, Z^{d} represents a bromine or iodine atom, and Y^{d} represents a chlorine atom, is reacted with at least one compound of general formula V,

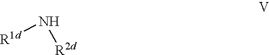

wherein R^{1d} and R^{2d} have any of the above given meanings in a suitable reaction medium, preferably in at least an organic solvent, more preferably in at least an organic solvent selected from the group consisting of toluene and dioxane, preferably in the presence of at least one catalyst, more preferably in the presence of at least a palladium and/or copper source, even more preferably in the presence of at least a palladium and/or copper source selected from the group consisting of Pd(OAc)₂, wherein OAc is acetate, Pd₂dba₃, wherein dba is dibenzylidene acetone, and copper(I)iodide, and/or at least one auxiliary agent, preferably at least one auxiliary agent selected from the group consisting of 4,5-bis(diphenylphosphino)-9,9-dimethylxanthene (Xantphos), 1,1-bis(diphenylphosphino-ferrocene and P(tBu)₃, wherein tBu is tert-Butyl, and/or at least one base, preferably at least one base selected from the group consisting of K₃PO₄, Cs₂CO₃ and trans-1,2-diamino-methylcyclohexane, to yield a compound of general formula VIII,

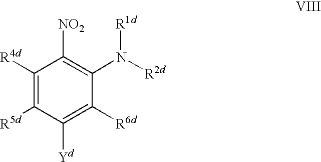

wherein R^{1d}, R^{2d} and R^{4d} to R^{6d} have any of the above given meanings and Y^{d} represents a chlorine atom; which is optionally purified and/or isolated, and the compound of general formula VIII is reacted with at least one compound of general formula III,

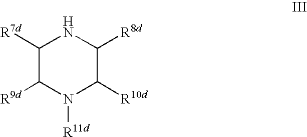

wherein R^{7d} to R^{11d} have any of the above given meanings, in a suitable reaction medium, preferably in at least an organic solvent, more preferably in at least an organic solvent selected from the group consisting of toluene, tetrahydrofuran and dimethoxyethane, preferably in the presence of at least one catalyst, more preferably in the presence of at least a palladium source, even more preferably in the presence of at least a palladium source selected from the group consisting of Pd(OAc)₂, wherein OAc is acetate, and Pd₂dba₃, wherein dba is dibenzylidene acetone, and/or at least one auxiliary agent, preferably (biph)P(tBu)₂, wherein biph is biphenyl and tBu is tert-butyl, and/or at least one base, preferably at least one base selected from the group consisting of K₃PO₄ or sodium tert-pentoxide, to yield a compound of general formula VI,

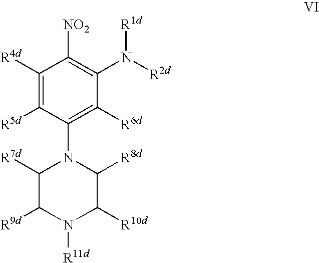

wherein R^{1d}, R^{2d} and R^{4d} to R^{11d} have any of the above given meanings, which is optionally purified and/or isolated.

The compounds of general formula Id are prepared by a process, wherein at least one nitrobenzene compound of general formula II,

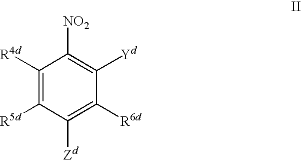

wherein R^{4d} to R^{6d} have any of the above given meanings, Y^{d} represents a chlorine atom, and Z^{d} represents a bromine or iodine atom; is reacted with at least one compound of general formula III,

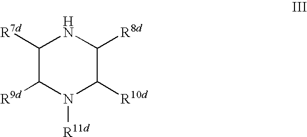

wherein R^{7d} to R^{11d} have any of the above given meanings, in a suitable reaction medium, preferably in at least an organic solvent, more preferably in at least an organic solvent selected from the group consisting of tetrahydrofuran or dioxane, preferably in the presence of at least one catalyst, more preferably in the presence of at least a palladium source, even more preferably in the presence of PdCl₂(dppf), wherein dppf is 1,1-bis(diphenylphosphino)-ferrocene, and/or at least one auxiliary agent, preferably 1,1-bis(diphenylphosphino)-ferrocene, and/or at least one base, preferably sodium tert-pentoxide, to yield a compound of general formula IV,

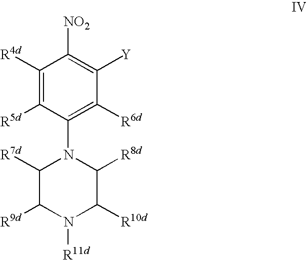

wherein R^{4d} to R^{11d} have any of the above given meanings and Y^{d} represents a chlorine atom; which is optionally purified and/or isolated, and the compound of general formula IV is reacted with at least one compound of general formula IX,

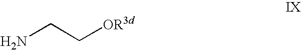

wherein R^{3d} has any of the above given meanings, in a suitable reaction medium, preferably in at least an organic solvent, more preferably in at least an organic solvent selected from the group consisting of toluene or dimethoxyethane, preferably in the presence of at least one catalyst, more preferably in the presence of at least a palladium source, even more preferably in the presence of at least a palladium source selected from the group consisting of Pd(OAc)₂, wherein OAc is acetate, and Pd₂dba₃, wherein dba is dibenzylidene acetone, and/or at least one auxiliary agent, preferably (biph)P(tBu)₂, wherein biph is biphenyl and tBu is tert-butyl, and/or at least one base, preferably at least one base selected from the group consisting of K₃PO₄ and sodium tert-pentoxide to yield a compound of general formula X,

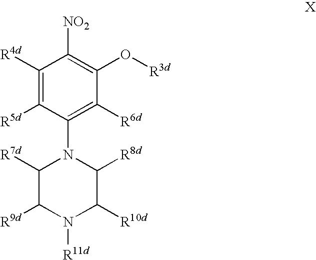

wherein R^{3d} to R^{11d} have any of the above given meanings, which is optionally purified and/or isolated.

Suitable reaction media include organic solvents, such as dialkyl ether, preferably diethyl ether and dimethoxyethane, or a cyclic ether, preferably tetrahydrofuran or dioxane; or a halogenated hydrocarbon, preferably dichloromethane or chloroform; an alcohol, preferably methanol or ethanol; an aprotic solvent, preferably acetonitrile, toluene, pyridine or dimethylformamide, or any other suitable reaction medium. Of course, mixtures of at least two classes of solvents or of at least two solvents of one class may also be used.

All of above mentioned reactions are preferably carried out in an oven-dried vial. The catalyst, the auxiliary agent, the base and the compound of general formula II, IV, VII or VIII are added in each case and the vial is subsequently evacuated and purged with argon. The organic solvent and the compound of general formula III, V or IX are added and the reaction is carried out in a sealed vial at a temperature between 100° C. and 110° C., preferably at 100° C. in case of tetrahydrofuran or toluene as the organic solvent and at 110° C. in case of dimethoxyethane and dioxane as the organic solvent.

Suitable reaction conditions for carrying out the reaction between compounds of general formula II, IV, VII or VIII and compounds of general formula III, V or IX are described in the references of J. F. Hartwig et al., J. Am. Chem. Soc. 1996, 118, 7217-7218; S. L. Buchwald et al., J. Am. Chem. Soc. 2002, 124, 6043-6048; S. L. Buchwald et al. J. Am. Chem. Soc. 2002, 124, 7241-7424 and S. L. Buchwald et al., J. Am. Chem. Soc. 2002, 124, 11684-11688. The respective part of the description is hereby incorporated by reference and forms part of the present disclosure.

The compounds of general formulas IV, VI, VIII and X given above may be purified and/or isolated according to methods well known to those skilled in the art.

The compounds of general formulas IV, VI, VIII and X may be isolated by evaporating the reaction medium, addition of water and adjusting the pH value to obtain the compound in form of a solid that can be isolated by filtration, or by extraction with a solvent that is not miscible with water such as chloroform and purification by chromatography or recrystallisation from a suitable solvent.

Preferably, the compounds of general formula IV, VI, VIII and X may be obtained by filtration of the reaction mixture and subsequent separation of the reaction mixture on a TLC plate. Alternatively, the compounds of general formula I may be isolated by addition of water and methanol to the reaction mixture, evaporating the reaction mixture and purifying the residue by preparative HPLC.

The compounds of general formula II and VII are commercially available or may be prepared according to methods well known in the art, for example, analogous to the methods described in the bibliography of A. McKillop et al., Tetrahedron 1987, 43, 1753. The respective part of the literature description cited above is hereby incorporated by reference and forms part of the disclosure.

The compounds of general formula III, V and IX are commercially available or may be prepared according to methods well known in the art.

During some synthetic reactions described above or while preparing the compounds of general formulas III, V, VI, IX or X the protection of sensitive or reactive groups may be necessary and/or desirable. This can be performed by using conventional protective groups like those described in Protective groups in Organic Chemistry, ed. J. F. W. McOmie, Plenum Press, 1973; T. W. Greene & P. G. M. Wuts and Protective Groups in Organic Chemistry, John Wiley & sons, 1991. The respective parts of the description is hereby incorporated by reference and forms part of the disclosure. The protective groups may be eliminated when convenient by means well-known to those skilled in the art.

If the nitro-substituted phenyl-piperazine compounds of general formula Id are obtained in form of a mixture of stereoisomers, particularly enantiomers or diastereomers, said mixtures may be separated by standard procedures known to those skilled in the art, e.g. chromatographic methods or crystallization with chiral reagents.

The nitro-substituted phenyl-piperazine compounds of general formula Id and in each case stereoisomers thereof may be obtained in form of a corresponding salt according to methods well known to those skilled in the art, e.g. by reacting said compound with at least one inorganic and/or organic acid, preferably in a suitable reaction medium. Suitable reaction media include, for example, any of the ones given above.

Preferably as component (A) at least one compound is present which is selected from the group consisting of phenyl-piperazine-derived compounds of general formula (Ie)

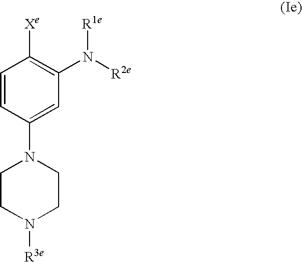

wherein

X^{e} represents —CN, —C(═O)—OH, —C(═O)—OR^{4e}, —O—R^{5e}, —NH₂, —NR^{6e}—C(═O)—R^{7e}, —NH—S(═O)₂—R^{8e} or —NH—R^{9e};

R^{1e} represents a hydrogen atom;

a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);

R^{2e} represents a hydrogen atom or a —C(═O)—R^{10e} moiety;

or

R₁ₑ and R^{2e} together with the bridging nitrogen form a nitro (NO₂)— group or

an unsubstituted or at least mono-substituted 5- or 6-membered heteroaryl radical which may be condensed with an unsubstituted or at least mono-substituted mono- or bicyclic ring system;

R^{3e} represents a linear or branched, saturated or unsaturated, unsubstituted or at least mono-substituted aliphatic radical;

R^{4e} represents a linear or branched, saturated or unsaturated, unsubstituted or at least mono-substituted aliphatic radical;

R^{5e} represents a linear or branched, saturated or unsaturated, unsubstituted or at least mono-substituted aliphatic radical;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

R^{6e} represents a hydrogen atom or

an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

R^{7e} represents a linear or branched, saturated or unsaturated, unsubstituted or at least mono-substituted aliphatic radical;

R₈ₑ represents a linear or branched, saturated or unsaturated, unsubstituted or at least mono-substituted aliphatic radical;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

R^{9e} represents a linear or branched, saturated or unsaturated, unsubstituted or at least mono-substituted aliphatic radical;

or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

and

R^{10e} represents a linear or branched, saturated or unsaturated, unsubstituted or at least mono-substituted aliphatic radical;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Preferred compounds of general formula (Ie) are those, wherein

X^{e} represents —CN, —C(═O)—OH, —C(═O)—OR^{4e}, —O—R^{5e}, —NH₂, —NR^{6e}—C(═O)—R^{7e}, —NH—S(═O)₂—R^{8e} or —NH—R^{9e};

R^{1e} represents

a hydrogen atom; or

an aryl or heteroaryl radical selected from the group consisting of phenyl, naphthyl, furyl(furanyl) and thiophenyl(thiophenyl), whereby said aryl or heteroaryl radical is bonded via a —(CH₂)—, —(CH₂)—(CH₂)—, —(CH₂)—(CH₂)—(CH₂)—, —O—(CH₂)—(CH₂)—, or —(CH₂)— (CH₂)—O— group and/or may be unsubstituted or substituted with 1, 2 or 3 substituent(s) independently selected from the group consisting of methyl, ethyl, n-propyl, isopropyl, n-butyl, tert-butyl, sec-butyl, isobutyl, —O—CH₃, —O—C₂H₅, —O—CH₂—CH₂—CH₃, —O—CH(CH₃)₂, —O—C(CH₃)₃, F, Cl, Br, —CN, —CF₃, —OCF₃, —OH and —SH.

R² represents a hydrogen atom or a —C(═O)—R¹⁰ moiety;

R^{1e} and R^{2e} together with the bridging nitrogen atom form a nitro group or moiety selected from the group consisting of

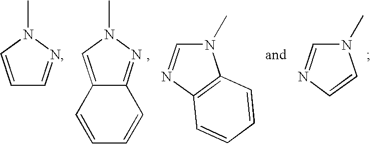

whereby each of these afore mentioned cyclic moieties may be unsubstituted or substituted with 1, 2 or 3 substituent(s) independently selected from the group consisting of methyl, ethyl, n-propyl, isopropyl, n-butyl, tert-butyl, sec-butyl, isobutyl, F, Cl, Br, I, —CN and —CF₃,

R^{3e} represents a methyl or ethyl radical;

R^{4e} represents a methyl or ethyl radical;

R^{5e} represents

an alkyl radical selected from the group consisting of methyl, ethyl, n-propyl, isopropyl, n-butyl, sec-butyl, isobutyl and tert-butyl;

or an aryl or heteroaryl radical selected from the group consisting of phenyl, naphthyl, furyl(furanyl) and thiophenyl(thiophenyl), whereby said aryl or heteroaryl radical is bonded via a —(CH₂)—, —(CH₂)—(CH₂)— or —(CH₂)—(CH₂)—(CH₂)— group and/or may be unsubstituted or substituted with 1, 2 or 3 substituent(s) independently selected from the group consisting of methyl, ethyl, n-propyl, isopropyl, n-butyl, tert-butyl, sec-butyl, isobutyl, —O—CH₃, —O—C₂H₅, —O—CH₂—CH₂—CH₃, —O—CH(CH₃)₂, —O—C(CH₃)₃, F, Cl, Br, —CN, —CF₃, —OCF₃, —OH and —SH;

R^{6e} represents a hydrogen atom, or

a phenyl radical, whereby said phenyl radical may be bonded via a —(CH₂)— group and/or may be unsubstituted or substituted with 1, 2 or 3 substituent(s) independently selected from the group consisting of methyl, ethyl, n-propyl, isopropyl, n-butyl, tert-butyl, sec-butyl, isobutyl, —O—CH₃, —O—C₂H₅, F, Cl, Br, —CF₃, —OCF₃, —OH and —SH;

R^{7e} represents a methyl or ethyl radical;

R^{8e} represents

an alkyl radical selected from the group consisting of methyl, ethyl, n-propyl, isopropyl, n-butyl, sec-butyl, isobutyl and tert-butyl; or

an aryl radical selected from the group consisting of phenyl and naphthyl, whereby said aryl radical may be bonded via a —(CH₂)—, or —(CH₂)—(CH₂)— group and/or may be unsubstituted or substituted with 1, 2 or 3 substituent(s) independently selected from the group consisting of methyl, ethyl, n-propyl, isopropyl, n-butyl, tert-butyl, sec-butyl, isobutyl, —O—CH₃, —O—C₂H₅, —O—CH₂—CH₂—CH₃, —O—CH(CH₃)₂, —O—C(CH₃)₃, F, Cl, Br, —CN, —CF₃, —OCF₃, —OH and —SH,

R^{9e} represents

an alkyl radical selected from the group consisting of methyl, ethyl, n-propyl, isopropyl, n-butyl, sec-butyl, isobutyl and tert-butyl;

or an aryl radical selected from the group consisting of phenyl and naphthyl, whereby said aryl radical may be bonded via a —(CH₂)—, —(CH₂)—(CH₂)— or —(CH₂)—(CH₂)—(CH₂)— group and/or may be unsubstituted or substituted with 1, 2 or 3 substituent(s) independently selected from the group consisting of methyl, ethyl, n-propyl, isopropyl, n-butyl, tert-butyl, sec-butyl, isobutyl, —O—CH₃, —O—C₂H₅, —O—CH₂—CH₂—CH₃, —O—CH(CH₃)₂, —O—C(CH₃)₃, F, Cl, Br, —CN, —CF₃, —OCF₃, —OH and —SH;

R^{10e} represents a methyl or ethyl radical;

optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

Particularly preferred compounds of general formula (Ie) are those selected from the group consisting of:
4-(4-Methyl-piperazin-1-yl)-2-phenethylamino-benzoic acid,
2-[(Furan-2-ylmethyl)-amino]-4-(4-methyl-piperazin-1-yl)-benzonitrile,
2-[(Furan-2-ylmethyl)-amino]-4-(4-methyl-piperazin-1-yl)-benzoic acid,
2-Benzylamino-4-(4-methyl-piperazin-1-yl)-benzoic acid methyl ester,
2-Benzylamino-4-(4-methyl-piperazin-1-yl)-benzonitrile,
4-(4-Methyl-piperazin-1-yl)-2-phenethylamino-benzoic acid methyl ester,
2-[(Furan-2-ylmethyl)-amino]-4-(4-methyl-piperazin-1-yl)-benzoic acid methyl ester,
2-Benzylamino-4-(4-methyl-piperazin-1-yl)-benzoic acid,
[2-Benzyloxy-5-(4-methyl-piperazin-1-yl)-phenyl]-phenethyl-amine,
[2-Benzyloxy-5-(4-methyl-piperazin-1-yl)-phenyl]-furan-2-yl-methyl amine,
Benzyl-[2-methoxy-5-(4-methyl-piperazin-1-yl)-phenyl]-amine,
[2-Methoxy-5-(4-methyl-piperazin-1-yl)-phenyl]-phenethyl-amine,
Furan-2-ylmethy-[2-methoxy-5-(4-methyl-piperazin-1-yl)-phenyl]-amine,
Benzyl-[2-benzyloxy-5-(4-methyl-piperazin-1-yl)-phenyl]-amine,
N-[2-Acetyl-(2-phenoxyethyl)-amino]-4-(4-methyl-piperazin-1-yl)-phenyl]-acetamide,
N-[4-(4-Methyl-piperazin-1-yl)-2-(2-phenoxy-ethylamino)-phenyl]-acetamide,
N-[2-(Acetyl-amino)-4-(4-methyl-piperazin-1-yl)-phenyl]-N-benzyl-acetamide,
N-[2-(3,5-Dimethyl-pyrazol-1-yl)-4-(4-methyl-piperazin-1-yl)-phenyl]-acetamide,
N-[2-(Acetyl-furan-2-ylmethyl-amino)-4-(4-methyl-piperazin-1-yl)-phenyl]-acetamide,
N-[2-Benzylamino-4-(4-methyl-piperazin-1-yl)-phenyl]-acetamide,
N-[2-[Furan-2-ylmethyl)-amino]-4-(4-methyl-piperazin-1-yl)-phenyl]-acetamide,
N-[2-Amino-5-(4-methyl-piperazin-1-yl)-phenyl]-N-furan-2-ylmethyl-acetamide,
N-[2-Amino-4-(4-methyl-piperazin-1-yl)-phenyl]-N-benzyl-acetamide,
N-[2-Benzylamino-4-(4-methyl-piperazin-1-yl)-phenyl]-benzenesulfonamide,
N-[2-Benzylamino-4-(4-methyl-piperazin-1-yl)-phenyl]-methansulfonamide,
2-Benzyloxy-5-(4-methyl-piperazin-1-yl)-phenylamine,
Benzyl-[4-(4-methyl-piperazin-1-yl)-2-nitro-phenyl]-amine and
2-Cyano-(5-piperazin-1-yl-methyl)-2-phenoxy-ethylamine

optionally in form of one of their stereoisomers, preferably enantiomers or diasteromers, a racemate or in form of a mixture of at least two stereoisomers, preferably enantiomers and/or diastereomers, in any mixing ratio, or a physiologically acceptable salt thereof, or a corresponding solvate thereof.

The compounds of general formula Ie are prepared by a process, wherein at least one substituted benzene compound of general formula II,

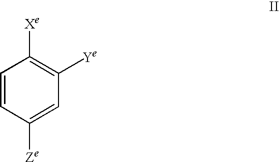

wherein X^{e} represents —CN, —C(═O)—OR^{4e}, —O—R^{5e} or —NO₂, R^{4e} and R^{5e} have any of the above given meanings, Y^{e} represents a chlorine atom, and Z^{e} represents a bromine or iodine atom; is reacted with at least one piperazine compound of general formula III,

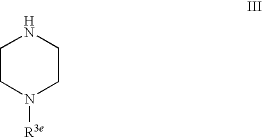

wherein R^{3e} has any of the above given meanings, in a suitable reaction medium, preferably in at least one organic solvent, more preferably in at least one organic solvent selected from the group consisting of tetrahydrofuran, toluene or dioxane, preferably in the presence of at least one catalyst, more preferably in the presence of at least a palladium source, even more preferably in the presence of at least one palladium source selected from the group consisting of Pd(OAc)₂, wherein OAc is acetate, and PdCl₂(dppf), wherein dppf is 1,1-bis(diphenylphosphino)-ferrocene, and/or at least one auxiliary agent, preferably at least one auxiliary agent selected from the group consisting of 1,1-bis(diphenylphosphino)-ferrocene and 2,2′-bis(diphenylphosphino)-1′1-binaphthyl (BINAP), optionally in form of its enantiomers or a racemate, and/or at least one base, preferably at least one base selected from the group consisting of sodium tert-pentoxide and Cs₂CO₃ to yield a compound of general formula IV,

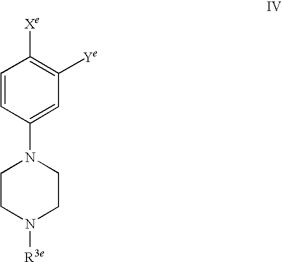

wherein X^{e} represents —CN, —C(═)—OR^{4e}, —O—R^{5e} or —NO₂, R^{3e}, R^{4e} and R^{5e} have and of the above given meanings and Yₑ presents a chlorine atom; which is optionally purified and/or isolated, and the compound of general formula IV is reacted with at least one compound of general formula V,

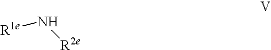

wherein R^{1e} and R^{2e} have any of the above given meanings or one of them represents a protecting group, preferably a —C(═O)—O—C(CH₃)₃ group, in a suitable reaction medium, preferably in at least one organic solvent, more preferably in at least one organic solvent selected from the group consisting of toluene, dioxane and dimethoxyethane, preferably in the presence of at least one catalyst, more preferably in the presence of at least a palladium source, even more preferably in the presence of at least a palladium source selected from the group consisting of Pd(OAc)₂, wherein OAc is acetate, and Pd₂dba₃, wherein dba is dibenzylidene acetone, and/or at least one auxiliary agent, preferably at least one auxiliary agent selected from the group consisting of (biph)P(tBu)₂, wherein biph is biphenyl and tBu is tert-butyl, and 4,5-bis(diphenylphosphino)-9,9-dimethylxanthene (Xantphos), and and/or at least one base, preferably at least one base selected from the group consisting of K₃PO₄, Cs₂CO₃ and sodium tert-pentoxide to yield a compound of general formula VI,

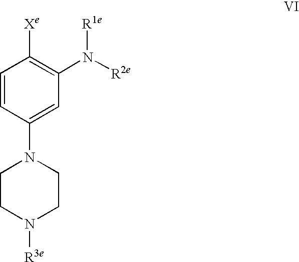

wherein X^{e} represents —CN, —C(═O)—OR^{4e}, —O—R^{5e} or —NO₂, R^{1e}, R^{2e} have any of the above given meanings or one of them represents a protecting group, preferably —C(═O)—O—C(CH₃)₃ and R^{3e}, R^{4e} and R^{5e} have any of the above given meanings, said compound of general formula VI is being optionally purified and/or isolated,

or at least one substituted benzene compound of general formula IIa,

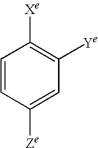

wherein X^{e} represents —CN, —C(═O)—OR^{4e}, —O—R₅ₑ or —NO₂, R^{4e} and R^{5e} have any of the above given meanings, Z^{e} represents a chlorine atom, Y^{e} represents a bromine or iodine atom, is reacted with at least one compound of general formula V,

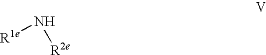

wherein R^{1e} and R^{2e} have any of the above given meanings or one of them represents a protecting group, preferably a —C(═O)—O—C(CH₃)₃ group in a suitable reaction medium, preferably in at least one organic solvent, more preferably in at least one organic solvent selected from the group consisting of toluene, dimethoxyethane and dioxane, preferably in the presence of at least one catalyst, more preferably in the presence of at least a palladium and/or copper source, even more preferably in the presence of at least a palladium and/or copper source selected from the group consisting of Pd(OAc)₂, wherein OAc is acetate, Pd₂dba₃, wherein dba is dibenzylidene acetone, and copper(I)iodide, and/or at least one auxiliary agent, preferably at least an auxiliary agent selected from the group consisting of 4,5-bis(diphenylphosphino)-9,9-dimethylxanthene (Xantphos), 1,1-bis(diphenylphosphino-ferrocene and P(tBu)₃ wherein tBu is tert-Butyl, and/or at least one base, preferably at least one base selected from the group consisting of K₃PO₄, Cs₂CO₃ and trans-1,2-diamino-methylcyclohexane to yield a compound of general formula VII,

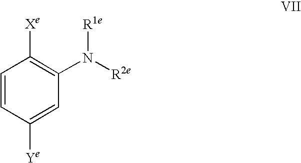

wherein X^{e} represents —CN, —C(═O)—OR^{4e}, —O—R^{5e} or —NO₂, R^{1e} and R^{2e} have any of the above given meanings or one of them represents a protecting group, preferably a —C(═O)—O—C(CH₃)₃ group, R^{4e} and R^{5e} have any of the above given meanings, and Y represents a chlorine atom; said compound of general formula being optionally purified and/or isolated, and the compound of general formula VII is reacted with at least one compound of general formula III,

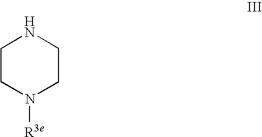

wherein R^{3e} has any of the above given meanings, in a suitable reaction medium, preferably in at least one organic solvent, more preferably in at least one organic solvent selected from the group consisting of tetrahydrofuran, toluene or dioxane, preferably in the presence of at least one catalyst, more preferably in the presence of at least a palladium source, even more preferably in the presence of at least one palladium source selected from the group consisting of Pd(OAc)₂, wherein OAc is acetate, and PdCl₂(dppf), wherein dppf is 1,1-bis(diphenylphosphino)-ferrocene, and/or at least one auxiliary agent, preferably at least one auxiliary agent selected from the group consisting of 1,1-bis(diphenylphosphino)-ferrocene and 2,2′-bis(diphenylphosphino)-1′1-binaphthyl (BINAP), optionally in form of its enantiomers or a racemate, and/or at least one base, preferably at least one base selected from the group consisting of sodium tert-pentoxide and Cs₂CO₃, to yield a compound of general formula VI,

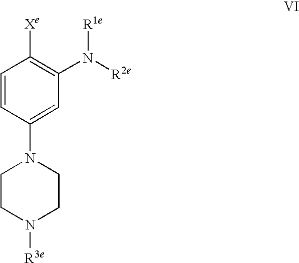

wherein X^{e} represents —CN, —C(═O)—OR^{4e}, —O—R^{5e} or —NO₂, R^{1e} and R^{2e} have any of the above given meanings or one of them represents a protecting group, preferably a —C(═O)—O—C(CH₃)₃ group, and R^{3e}, R^{4e} and R^{5e} have any of the above given meanings, and said compound of general formula VI is optionally purified and/or isolated,

or

at least one substituted benzene compound of general formula VIII,

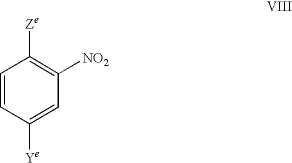

wherein Z^{e} represents bromine or iodine and Y^{e} represents chlorine, is reacted with at least one compound of general formula IX,

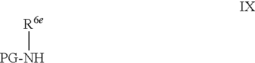

wherein R^{6e} has any of the above given meanings and PG represents a protecting group, preferably a —C(═O)—O—C(CH₃)₃ group, in a suitable reaction medium, preferably in at least one organic solvent, more preferably in at least one organic solvent selected from the group consisting of toluene and dioxane, preferably in the presence of at least one catalyst, more preferably in the presence of at least a palladium and/or copper source, even more preferably in the presence of at least a palladium and/or copper source selected from the group consisting of Pd(OAc)₂ wherein OAc is acetate, Pd₂dba₃ wherein dba is dibenzylidene acetone and copper(I)iodide, and/or at least one auxiliary agent, preferably at least an auxiliary agent selected from the group consisting of 4,5-bis(diphenylphosphino)-9,9-dimethylxanthene (Xantphos), 1,1-bis(diphenylphosphino-ferrocene and P(tBu)₃ wherein tBu is tert-Butyl, and/or at least one base, preferably at least one base selected from the group consisting of K₃PO₄, Cs₂CO₃ and trans-1,2-diamino-methylcyclohexane to yield a compound of general formula XI,

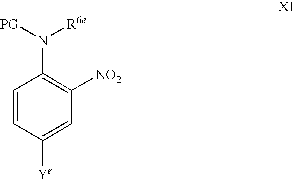

wherein R^{6e} has any of the above given meanings, PG represents a protecting group, preferably a —C(═O)—O—C(CH₃)₃ group and Y^{e} represents chlorine; which is optionally purified and/or isolated, and the compound of general formula XI reacted with at least one compound of general formula III,

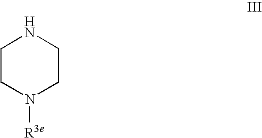

wherein R^{3e} has any of the above given meanings, in a suitable reaction medium, preferably in at least one organic solvent, more preferably in at least one organic solvent selected from the group consisting of tetrahydrofuran, toluene or dioxane, preferably in the presence of at least one catalyst, more preferably in the presence of at least a palladium source, even more preferably in the presence of at least one palladium source selected from the group consisting of Pd(OAc)₂, wherein OAc is acetate, and PdCl₂(dppf), wherein dppf is 1,1-bis(diphenylphosphino)-ferrocene, and/or at least one auxiliary agent, preferably at least one auxiliary agent selected from the group consisting of 1,1-bis(diphenylphosphino)-ferrocene and 2,2′-bis(diphenylphosphino)-1′1-binaphthyl (BINAP), optionally in form of its enantiomers or a racemate, and/or at least one base, preferably at least one base selected from the group consisting of sodium tert-pentoxide and Cs₂CO₃, to yield a compound of general formula XII,

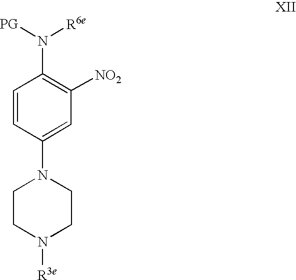

wherein R^{3e} and R^{6e} have any of the above given meanings and PG represents a protecting group, preferably a —C(═O)—O—C(CH₃)₃ group, which is optionally purified and/or isolated, and the compound of general formula XII is reacted with at least one acid in a suitable reaction medium to yield a compound of general formula XIII,

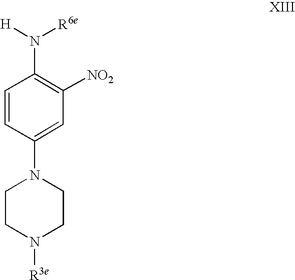

wherein R^{3e} and R^{6e} have any of the above given meanings, which is optionally purified and/or isolated, and the compound of general formula is reacted with hydrogen in the presence of at least one catalyst, preferably in the presence of at least one palladium source, more preferably in the presence of palladium on charcoal, in a suitable reaction medium, preferably in at least one organic solvent, more preferably in an organic solvent selected from the group consisting of dioxane, tetrahydrofuran and diethyl ether, to yield a compound of general formula XIV,

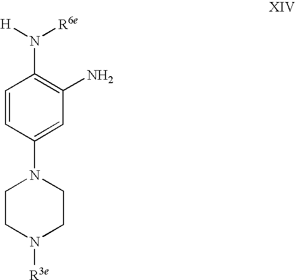

wherein R^{3e} and R^{6e} have any of the above given meanings, which is optionally purified and/or isolated, and the compound of general formula XIV is reacted with at least one compound of general formula R^{7e}—C(═O)—O—C(═O)—R^{7e}, wherein R^{7e} any of the above given meanings, and/or at least one compound of general formula R^{10e}—C(═O)—O—C(═O)—R^{10e}, wherein R^{10e} has any of the above given meanings, optionally in the presence of at least one base, preferably in the presence of at least one organic base, more preferably in the presence of at least an organic base selected from the group consisting of pyridine, triethylamine and diisopropylethylamine, in a suitable reaction medium, preferably in at least one organic solvent, more preferably in at least one organic solvent selected from the group consisting of dioxane, tetrahydrofuran and diethyl ether, to yield a compound of general formula I, wherein Xₑ represents —NR^{6e}—C(═O)R^{7e}, R^{1e} represents a hydrogen atom, R^{2e} represents a hydrogen atom or a —C(═O)—R^{10e}-moiety and R^{3e}, R₆ₑ, R^{7e} and R^{10e} have any of the above given meanings, which is optionally purified and/or isolated,

and/or at least one compound of general formula VI, wherein X^{e} represents —CN, —C(═O)—OR^{4e} or —O—R^{5e}, R^{1e} and R^{2e} have any of the above given meanings or one of them represents a protecting group, preferably a —C(═O)—O—C(CH₃)₃— group, R^{3e}, R^{4e} and R^{5e} have any of the above given meanings, is reacted with at least one acid, preferably at least one acid selected from the group consisting of sulfuric acid, hydrochloric acid and acetic acid, in a suitable reaction medium, preferably in at least one organic solvent, more preferably in at least one organic solvent selected from the group consisting of dioxane and tetrahydrofuran, to yield a compound of general formula I, wherein X^{e} represents —CN, —C(═O)—OR^{4e} or —O—R^{5e}, R^{1e} and R^{3e} to R^{5e} have any of the above given meanings and R^{2e} represents hydrogen, which is optionally purified and/or isolated,

and optionally at least one compound of general formula I, wherein X^{e} represents —CN, —C(═O)—OR^{4e} or —O—R^{5e}, R₁ₑ and R^{3e} to R^{5e} have any of the above given meanings and R^{2e} represents hydrogen, is reacted with hydrogen in the presence of at least one catalyst, preferably in the presence of at least one palladium source, more preferably in the presence of palladium on charcoal, in a suitable reaction medium, preferably in at least one organic solvent, more preferably in an organic solvent selected from the group consisting of dioxane, tetrahydrofuran and diethyl ether, to yield a compound of general formula I, wherein Xₑ represents —CN, —C(═O)—OR^{4e} or —O—R^{5e}, R^{3e} to R^{5e} have any of the above given meanings and R^{1e} and R^{2e} each represent hydrogen,

at least one compound of general formula VI, wherein X^{e} represents —C(═O)—OR^{4e}, R^{1e} and R^{2e} have any of the above given meanings or one of them represents a protecting group, preferably a —C(═O)—O—C(CH₃)₃— group, R^{3e} and R^{4e} have any of the above given meanings, is reacted with at least one base, preferably at least one metal hydroxide, more preferably at least one metal hydroxide selected from the group consisting of lithium hydroxide and potassium hydroxide, in a suitable reaction medium, preferably in a mixture of at least one organic solvent and water, more preferably in a mixture of at least one organic solvent selected from the group consisting of dioxane, ethanol and methanol and water, to yield a compound of general formula XV, wherein X^{e} represents —C(═O)—OH, R^{1e} and R^{2e} have any of the above given meanings or one of them represents a protecting group, preferably a —C(═O)—O—C(CH₃)₃— group, R^{3e} has any of the above given meanings, which is optionally purified and/or isolated and at least one compound of general formula XV is reacted with at least one acid, preferably at least one acid selected from the group consisting of sulfuric acid, hydrochloric acid and acetic acid, in a suitable reaction medium, preferably in at least one organic solvent, more preferably in at least one organic solvent selected from the group consisting of dioxane and tetrahydrofuran, to yield a compound of general formula I, wherein X^{e} represents —C(═O)—OH, R^{1e} and R^{3e} have any of the above given meanings and R^{2e} represents hydrogen, which is optionally purified and/or isolated,

and/or

at least one compound of general formula VI, wherein X^{e} represents —NO₂, R^{1e} and R^{2e} have any of the above given meanings or one of them represents a protecting group, preferably a —C(═O)—O—C(CH₃)₃— group and R^{3e} has any of the above given meanings, is reacted with hydrogen in the presence of at least one catalyst, preferably in the presence of at least one palladium source, more preferably in the presence of palladium on charcoal, in a suitable reaction medium, preferably in at least one organic solvent, more preferably in an organic solvent selected from the group consisting of dioxane, tetrahydrofuran and diethyl ether, to yield a compound of general formula XVI, wherein X^{e} represents —NH₂, R^{1e} and R^{2e} have any of the above given meanings or one of them represents a protecting group, preferably a —C(═O)—O—C(CH₃)₃— group, and R^{3e} has any of the above given meanings, which is optionally purified and/or isolated, and at least one compound of general formula XVI, is reacted with at least one acid, preferably at least one acid selected from the group consisting of sulfuric acid, hydrochloric acid and acetic acid, in a suitable reaction medium, preferably in at least one organic solvent, more preferably in at least one organic solvent selected from the group consisting of dioxane and tetrahydrofuran, to yield a compound of general formula I, wherein X^{e} represents —NH₂, R^{1e} and R^{3e} have any of the above given meanings and R^{2e} represents hydrogen, which is optionally purified and/or isolated,

and optionally at least one compound of general formula I, wherein X^{e} represents —NH₂, R^{1e} and R^{3e} have any of the above given meanings and R^{2e} represents hydrogen, is reacted with at least one compound of general formula R^{7e}—C(═O)—O—C(═O)—R^{7e} and/or at least one compound of general formula R^{10e}—C(═O)—O—C(═O)—R^{10e}, wherein R^{7e} and R^{10e} have any of the above given meanings optionally in the presence of at least one base, preferably in the presence of at least one organic base, more preferably in the presence of at least an organic base selected from the group consisting of pyridine, triethylamine and diisopropylethylamine, in a suitable reaction medium, preferably in at least one organic solvent, more preferably in at least one organic solvent selected from the group consisting of dioxane, tetrahydrofuran and diethyl ether, to yield a compound of general formula I, wherein X^{e} represents —NH—C(═O)—R^{7e} and R^{1e} to R^{3e} have any of the above given meanings, which is optionally purified and/or isolated,

and/or optionally at least one compound of general formula I, wherein X^{e} represents —NH₂ and R^{1e} and R^{3e} have any of the above given meanings and R^{2e} e represents hydrogen, is reacted with at least one compound of general formula R^{8e}—S(═O)—W, wherein R^{8e} has any of the above given meanings and W represents a halogen atom, preferably a chlorine atom, optionally in the presence of at least one base, preferably in the presence of at least one organic base, more preferably in the presence of an organic base selected from the group consisting of pyridine, triethylamine and diisopropylethylamine, in a suitable reaction medium, preferably in at least one organic solvent, more preferably in at least one organic solvent selected from the group consisting of dioxane, tetrahydrofuran and diethyl ether, to yield a compound of general formula I, wherein X^{e} represents —NH—S(═O)₂—R^{8e} and R^{1e}, R^{3e} and R^{8e} have any of the above given meanings and R^{2e} represents hydrogen, which is optionally purified and/or isolated.

Suitable reaction media include organic solvents, such as dialkyl ether, preferably diethyl ether and dimethoxyethane, or a cyclic ether, preferably tetrahydrofuran or dioxane; or a halogenated hydrocarbon, preferably dichloromethane or chloroform; an alcohol, preferably methanol or ethanol; an aprotic solvent, preferably acetonitrile, pyridine, toluene or dimethylformamide, or any other suitable reaction medium. Of course, mixtures of at least two classes of solvents or of at least two solvents of one class may also be used.

If the above mentioned reactions are carried out in an oven-dried vial, the catalyst, the auxiliary agent, the base and the compound of general formula II, IIa, IV, VII, VIII or XI are added in each case and the vial is subsequently evacuated and purged with argon. The organic solvent and the compound of general formula III, V and IX are added and the reaction is carried out in a sealed vial at a temperature between 100° C. and 110° C., preferably at 100° C. in case of tetrahydrofuran or toluene as the organic solvent and at 110° C. in case of dimethoxyethane and dioxane as the organic solvent.

Suitable reaction conditions for carrying out the reaction between compounds of general formula II, IIa, IV, VII, VIII or XI and compounds of general formula III, V and IX are described in the references of J. F. Hartwig et al., J. Am. Chem. Soc. 1996, 118, 7217-7218; S. L. Buchwald et al., J. Org. Chem. 2000, 65, 1144-1157; S. L. Buchwald et al., J. Am. Chem. Soc. 2002, 124, 6043-6048; S. L. Buchwald et al. J. Am. Chem. Soc. 2002, 124, 7241-7424 and S. L. Buchwald et al., J. Am. Chem. Soc. 2002, 124, 11684-11688. The respective part of the description is hereby incorporated by reference and forms part of the present disclosure.

The compounds of general formula I, IV, VI, VII, XI, XII, XIII, XIV, XV and XVI may be isolated by evaporating the reaction medium, addition of water and adjusting the pH value to obtain the compound in form of a solid that can be isolated by filtration, or by extraction with a solvent that is not miscible with water such as chloroform and purification by chromatography or recrystallisation from a suitable solvent.

Preferably, the compounds of general formula I, IV, VI, VII, XI, XII, XIII, XIV, XV and XVI may be obtained by filtration of the reaction mixture and subsequent separation of the reaction mixture on a TLC plate. Alternatively, the compounds of general formula I, IV, VI, VII, XI, XII, XIII, XIV, XV and XVI may be isolated by addition of water and methanol to the reaction mixture, evaporating the reaction mixture and purifying the residue by preparative HPLC.

The compounds of general formula II, IIa, VIII and IX are commercially available or may be prepared according to methods well known in the art, for example, analogous to the methods described in the bibliography of A. McKillop et al., Tetrahedron 1987, 43, 1753. The respective part of the literature description cited above is hereby incorporated by reference and forms part of the disclosure.

During some synthetic reactions described above or while preparing the compounds of general formulas II, IIa, VIII and IX the protection of sensitive or reactive groups may be necessary and/or desirable. This can be performed by using conventional protective groups like those described in Protective groups in Organic Chemistry, ed. J. F. W. McOmie, Plenum Press, 1973; T. W. Greene & P. G. M. Wuts and Protective Groups in Organic Chemistry, John Wiley & sons, 1991. The respective parts of the description is hereby incorporated by reference and forms part of the disclosure. The protective groups may be eliminated when convenient by means well-known to those skilled in the art.

If the substituted phenyl-piperazine compounds of general formula Ie are obtained in form of a mixture of stereoisomers, particularly enantiomers or diastereomers, said mixtures may be separated by standard procedures known to those skilled in the art, e.g. chromatographic methods or crystallization with chiral reagents.

The substituted phenyl-piperazine compounds of general formula Ie and in each case stereoisomers thereof may be obtained in form of a corresponding salt according to methods well known to those skilled in the art, e.g. by reacting said compound with at least one inorganic and/or organic acid, preferably in a suitable reaction medium. Suitable reaction media include, for example, any of the ones given above.

Preferably as component (A) at least one compound is present which is selected from the group consisting of tetrahydroisoquinoline-derived sulfonamide compounds of general formula (If)

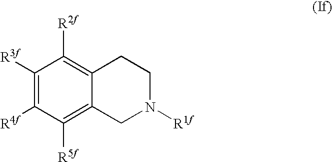

wherein

R^{1f} represents a hydrogen atom; a —C(═O)—OR^{37f} moiety;

a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;

R^{2f}, R^{3f}, R^{4f} and R^{5f}, independently of one another, each represent a hydrogen atom; F, Cl, Br, I, —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{8f}; —S(═O)—R^{7f}; —S(═O)₂—R^{7f}; —OR^{8f}; —SR^{9f}; —C(═O)—OR^{10f}; —N(R^{11f})—S(═O)₂—R^{12f}; —NR^{13f}R^{14f}; —NH—R^{15f}; —C(═O)—NR^{16f}R^{17f}; C(═O)—NHR^{18f};

a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;

with the proviso that at least one of the substituents R^{2f}, R^{3f}, R^{4f} and R^{5f} represents a —N(R^{11f})—(═O)₂—R^{12f} moiety;

R^{6f}, R^{7f}, R^{8f}, R^{9f}, R^{10f}, R^{13f}, R^{14f}, R^{15f}, R^{16f}, R^{17f} and R^{18f},

independently of one another, each represent a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;

R_{11f} represents a hydrogen atom or a linear or branched, saturated or unsaturated, unsubstituted or at least mono-substituted aliphatic radical;

R^{12f} represents a phenyl radical of general formula (Af),

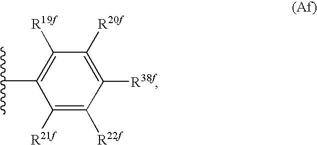

wherein

R^{19f}, R^{20f}, R^{21f}, R^{22f} and R^{38f}, independently of one another, each represent a hydrogen atom; F, Cl, Br, I, —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{23f}; —S(═O)—R^{24f}; —S(═O)₂—R^{24f}; —OR^{25f}; —SR^{26f}; —C(═O)—OR^{27f}; —N(R^{28f})—S(═O)₂—R^{29f}; —NH—S(═O)₂—R^{30f}; —NR^{31f}R^{32f}; —NH—R^{33f}; —C(═O)—NHR^{34f}; —C(═O)—NR^{35f}R^{36f}; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;

an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;

or a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;

R^{23f}, R^{27f}, R^{28f}, R^{29f} and R^{30f}, independently of one another, each represent a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;

R^{24f}, R^{26f}, R^{31f}, R^{32f} and R^{33f}, each represent a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be condensed with an unsubstituted or at least mono-substituted saturated or unsaturated, but not aromatic, mono- or bicyclic ring system;

R^{25f}, R^{34f}, R^{35f} and R^{36f}, represents a linear or branched, saturated or unsaturated, unsubstituted or at least mono-substituted aliphatic radical;

and R^{37f} represents a linear or branched, saturated or unsaturated, unsubstituted or at least mono-substituted aliphatic radical;

a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;

or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Preferred compounds of general formula (If) are those, wherein

R^{1f} represents a hydrogen atom; a —C(═O)—OR^{37f} moiety; a radical selected from the group consisting of methyl, ethyl, n-propyl, isopropyl, n-butyl, sec-butyl, isobutyl, tert-butyl, n-pentyl, n-hexyl, —CH₂—NH₂, —CH₂—NH—CH₃, —CH₂—N(CH₃)₂, —CH₂—N(C₂H₅)₂, —CH₂—NH—C₂H₅, —CH₂—CH₂—NH₂, —CH₂—CH₂—NH—CH₃, —CH₂—CH₂—N(CH₃)₂, —CH₂—CH₂—N(C₂H₅)₂, —CH₂—CH₂—NH—C₂H₅, —CH₂—CH₂—CH₂—NH—CH₃, —CH₂—CH₂—CH₂—N(CH₃)₂, —CH₂—CH₂—CH₂—N(C₂H₅)₂ and —CH₂—CH₂—CH₂—NH—C₂H₅; or a (hetero)cycloaliphatic radical selected from the group consisting of imidazolidinyl, aziridinyl, azetidinyl, pyrrolidinyl, piperidinyl, morpholinyl, thiomorpholinyl, piperazinyl, pyrazolidinyl and azepanyl, which may be bonded via a —(CH₂)_{1, 2 or 3}— group and which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl and isobutyl;

R^{2f}, R^{3f}, R^{4f} and R^{5f}, independently of one another, each represent a hydrogen atom; F, Cl, Br, I, —NO₂; —O—CH₃; —O—C₂H₅; —O—CF₃; —O—CFH₂; —O—CF₂H; —O—CH₂—CF₃; —O—CF₂—CF₃; —S—CH₃; —S—C₂H₅; —S—CF₃; —S—CFH₂; —S—CF₂H; —S—CH₂—CF₃; —S—CF₂—CF₃; —N(R^{11f})—S(═O)₂—R^{12f}; or a radical selected from the group consisting of methyl, ethyl, n-propyl, isopropyl, tert-butyl, —CF₃, —CFH₂, —CF₂H, —CH₂—CF₃ and —CF₂—CF₃;

with the proviso that at least one of the substituents R^{2f}, R^{3f}, R^{4f} and R^{5f} represents a —N(R^{11f})—S(═O)₂—R^{12f} moiety;

R^{11f} represents a hydrogen atom, —S(═O)₂—R^{12f} or an alkyl radical selected from the group consisting of methyl, ethyl, n-propyl, isopropyl, n-butyl, sec-butyl, isobutyl and tert-butyl;

R^{12f} represents a phenyl radical of general formula (Af),

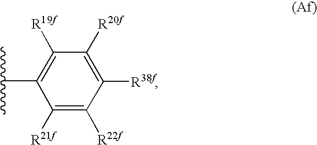

wherein

R^{19f}, R^{20f}, R^{21f}, R^{22f} and R^{38f}, independently of one another, each represent a hydrogen atom; F, Cl, Br, I, —NO₂; —NH₂; —SH; —OH; —CN; —C(═O)—OH; —C(═O)—H; —C(═O)—CH₃; —C(═O)—C₂H₅; —O—CH₃; —O—C₂H₅; —O—CF₃; —O—CFH₂; —O—CF₂H; —O—CH₂—CF₃; —O—CF₂—CF₃; —S—CH₃; —S—C₂H₅; —S—CF₃; —S—CFH₂; —S—CF₂H; —S—CH₂—CF₃; —S—CF₂—CF₃; —C(═O)—OCH₃; —C(═O)—OC₂H₅; methyl, ethyl, n-propyl, isopropyl, n-butyl, isobutyl, sec-butyl, tert-butyl, —CF₃, —CF₂H, —CFH₂, —CH₂—CF₃ and —CF₂—CF₃;

a radical selected from the group consisting of naphthyl, [1,3]-benzodioxolyl, [1,4]-benzodioxanyl, benzo[b]furanyl, benzo[b]thiophenyl, benzo[2,1,3]thiadiazolyl, [1,2,3]-benzothiadiazolyl, [2,1,3]-benzoxadiazolyl, [1,2,3]-benzoxadiazolyl, benzoxazolyl, benzothiazolyl, benzisoxazolyl, benzisothiazolyl and imidazo[2,1-b]thiazolyl, which may be unsubstituted or optionally substituted with 1, 2, 3, 4 or 5 substituent(s) independently selected from the group consisting of methyl, ethyl, n-propyl, isopropyl, n-butyl, tert-butyl, sec-butyl, isobutyl, n-pentyl, —O—CH₃, —O—C₂H₅, F, Cl, Br, I, —CN, —CF₃, —OCF₃, —SCF₃, —CF₂H and —CFH₂;

or a radical selected from the group consisting of pyridinyl, pyrrolyl, imidazolyl, pyrazolyl, triazolyl, pyridazinyl, pyrimidinyl and pyrazinyl, which may be unsubstituted or optionally substituted with 1, 2, 3, 4 or 5 substituent(s) independently selected from the group consisting of F, Cl, Br, I, —NO₂; methyl, ethyl, n-propyl, isopropyl, n-butyl, isobutyl, sec-butyl, tert-butyl, —CF₃, —CF₂H, —CFH₂, —CH₂—CF₃ and —CF₂—CF₃;

and R^{37f} represents a radical selected from the group consisting of methyl, ethyl, n-propyl, isopropyl, n-butyl, sec-butyl, isobutyl, tert-butyl, n-pentyl, n-hexyl, fluorenyl, fluorenylmethyl, phenyl, benzyl and naphthyl;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

Particularly preferred compounds of general formula (If) are those selected from the group consisting of:
N-(1,2,3,4-tetrahydroisoquinolin-6-yl)naphthalene-1-sulfonamide hydrochloride,
2,2-dimethyl-6-(N-methylnaphthalene-1-sulfonamido)-1,2,3,4-tetrahydroisoquinolinium iodide,
N-(2-methyl-1,2,3,4-tetrahydroisoquinolin-6-yl)naphthalene-1-sulfonamide hydrochloride,
5-chloro-3-methyl-N-(1,2,3,4-tetrahydroisoquinolin-6-yl)benzo[b]thiophene-2-sulfonamide hydrochloride,
5-chloro-3-methyl-N-(2-methyl-1,2,3,4-tetrahydroisoquinolin-6-yl)benzo[b]thiophene-2-sulfonamide hydrochloride,
4-methyl-N-(1,2,3,4-tetrahydroisoquinolin-6-yl)naphthalene-1-sulfonamide hydrochloride,
4-methyl-N-(2-methyl-1,2,3,4-tetrahydroisoquinolin-6-yl)naphthalene-1-sulfonamide hydrochloride,
N-(1,2,3,4-tetrahydroisoquinolin-6-yl)naphthalene-2-sulfonamide hydrochloride,
N-(2-methyl-1,2,3,4-tetrahydroisoquinolin-6-yl)naphthalene-2-sulfonamide hydrochloride,
6-Chloro-N-(1,2,3,4-tetrahydroisoquinolin-6-yl)imidazo[2,1-b]thiazole-5-sulfonamide hydrochloride,
2-methoxy-5-methyl-N-(1,2,3,4-tetrahydroisoquinolin-6-yl)benzenesulfonamide hydrochloride,
N-(1,2,3,4-tetrahydroisoquinolin-6-yl)pyridine-3-sulfonamide dihydrochloride,
6-(naphthalene-1-sulfonylamino)-3,4-dihydro-1H-isoquinoline-2-carboxylic acid tert-butyl ester,
6-(5-chloro-3-methyl-benzo[b]thiophene-2-sulfonylamino)-3,4-dihydro-1H-isoquinoline-2-carboxylic acid tert-butyl ester,
6-(4-methyl-naphthalene-1-sulfonylamino)-3,4-dihydro-1H-isoquinoline-2-carboxylic acid tert-butyl ester,
6-(naphthalene-2-sulfonylamino)-3,4-dihydro-1H-isoquinoline-2-carboxylic acid tert-butyl ester,
6-(2-methoxy-5-methyl-benzenesulfonylamino)-3,4-dihydro-1H-isoquinoline-2-carboxylic acid tert-butyl ester and

6-(pyridine-3-sulfonylamino)-3,4-dihydro-1H-isoquinoline-2carboxylic acid tert-butyl ester;

optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

The compounds of general formula If are prepared by a process, wherein at least one compound of general formula IV,

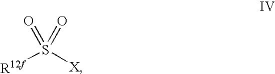

wherein R^{12f} has the meaning given above and X represents a leaving group, preferably a halogen atom, particularly preferably a chlorine atom, is reacted with at least one compound of general formula V,

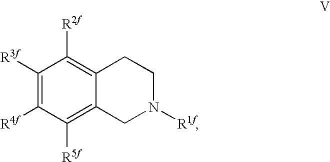

wherein R^{1f} to R^{5f} have the meaning given above, with the proviso that at least one substituent of the group consisting of R^{2f}, R^{3f}, R^{4f} and R^{5f} represents a —N(H)(R^{11f}) moiety, wherein R^{11f} has the meaning given above, or a protected derivative thereof, in a reaction medium, preferably in a reaction medium selected from the group consisting of pyridine, chloroform, dichloromethane, tetrahydrofurane and mixtures thereof, preferably in the presence of at least one base, more preferably in the presence of at least one base selected from the group consisting of triethylamine, diisopropylethylamine and diethylisopropylamine, preferably at a temperature between 0° C. and 30° C.

If the substituted tetrahydroisoquinoline compounds of general formula If are obtained in form of a mixture of stereoisomers, particularly enantiomers or diastereomers, said mixtures may be separated by standard procedures known to those skilled in the art, e.g. chromatographic methods or crystallization with chiral reagents.

The substituted tetrahydroisoquinoline compounds of general formula If and in each case stereoisomers thereof may be obtained in form of a corresponding salt according to methods well known to those skilled in the art, e.g. by reacting said compound with at least one inorganic and/or organic acid, preferably in a suitable reaction medium. Suitable reaction media include, for example, any of the ones given above.

Compounds of general formula IV are in most cases commercially available or may be prepared by processes known to those skilled in the art.

Compounds of general formula V are in most cases commercially available or may be prepared by processes known to those skilled in the art.

In particular, 1,2,3,4-tetrahydroisoquinoline compounds with an amino group in position 5 can be prepared starting from 5-nitro-1,2,3,4-tetrahydroisoquinoline compounds. A process for the preparation of the latter compounds is described in K. V. Rao et al., Journal of Heterocyclic Chemistry, 1973, 10, 213 to 215.

In particular, 1,2,3,4-tetrahydroisoquinoline compounds with an amino group in position 6 are commercially available or can be prepared starting from 6-nitro-1,2,3,4-tetrahydroisoquinoline compounds. A process for the preparation of the latter compounds is described in G. J. Quallich, Journal of Organic Chemistry, 1998, 63, 4116 to 4119.

1,2,3,4-tetrahydroisoquinoline compounds with a nitro group in position 6 or 8 may be prepared by established procedures described in M. Tercel, Journal of Medicinal Chemistry, 1996, 39, 1084 to 1094.

In particular, 1,2,3,4-tetrahydroisoquinoline compounds with an amino group in position 7 are commercially available or can be prepared starting from 7-nitro-1,2,3,4-tetrahydroisoquinoline compounds. A process for the preparation of the latter compounds is described in J. F. Ajao et al., Journal of Heterocyclic Chemistry, 1985, 22, 329 to 331.

The N-methyl-8-amino-substituted 1,2,3,4-tetrahydroisoquinoline compounds were prepared by bromination and nitration of the corresponding 1,2,3,4-tetrahydroisoquinolines followed by two-step standard reduction conditions as described in M. Rey, Helvetica Chimica Acta, 1985, 66, 1828 to 1834.

If any of the substituents in any of the above defined formulae represents or comprises a (hetero)cycloaliphatic radical, preferably a C₃₋₉ cycloalkyl radical or a C₄₋₉ cycloalkenyl radical, or a heterocyclic ring, preferably a 3- to 8-membered heterocyclic ring, said (hetero)cycloaliphatic radical, heterocyclic ring, C₃₋₉ cycloalkyl radical or C₄₋₉ cycloalkenyl radical may—if not defined otherwise—be unsubstituted or substituted by one or more substituents, preferably unsubstituted or optionally substituted with 1, 2, 3, 4 or 5 substituent(s). Said substituent(s) may preferably be selected independently from the group consisting of oxo (═O), thioxo (═S), C₁₋₅-alkyl, —O—C₁₋₅-alkyl, —S—C₁₋₅-alkyl, —C(═O)—OH, —C(═O)—C₁₋₅-alkyl, —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenoxy and benzyl; whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₃—CH₃—CH₃, —C(═O)—O—CH(CH₃)₂, —C(═O)—O—C(CH₃)₃, —C(═O)—CH₃, —C(═O)—C₂H₅, —C(═O)—CH₃—CH₃—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N—(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, —S(═O)₂-phenyl, phenyl, phenoxy and benzyl; whereby in each occurrence said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

If any of the substituents in any of the above defined formulae represents or comprises a cycloaliphatic radical, C₃₋₉ cycloalkyl radical or C₄₋₉ cycloalkenyl radical which contains one or more, preferably 1, 2 or 3 heteroatom(s) as ring member(s), unless defined otherwise, each of these heteroatom(s) may preferably be selected independently from the group consisting of N, O and S.

If any of the substituents in any of the above defined formulae represents or comprises a heterocyclic ring which contains at least one further, preferably 1 or 2 further heteroatom(s) as ring member(s), unless defined otherwise, each of these heteroatom(s) may preferably be selected independently from the group consisting of N, O and S.

Suitable saturated or unsaturated, optionally at least one heteroatom as ring member containing cycloaliphatic radicals, C₃₋₉ cycloalkyl radicals or C₄₋₉ cycloalkenyl radicals may preferably be selected from the group consisting of cyclopropyl, cyclobutyl, cyclopentyl, cyclohexyl, cycloheptyl, cyclooctyl, cyclononyl, cyclopentenyl, cyclohexenyl, cycloheptenyl, cyclooctenyl, pyrrolidinyl, piperidinyl, piperazinyl, homopiperazinyl, morpholinyl, aziridinyl, azetidinyl, imidazolidinyl, thiomorpholinyl, pyrazolidinyl, tetrahydrofuranyl, tetrahydrothiophenyl, azepanyl, diazepanyl, azocanyl, (2,5)-dihydrofuranyl, (2,5)-dihydrothiophenyl, (2,3)-dihydrofuranyl, (2,3)-dihydrofuranyl, (2,5)-dihydro-1H-pyrrolyl, (2,3)-dihydro-1H-pyrrolyl, tetrahydrothiopyranyl, tetrahydropyranyl, (3,4)-dihydro-2H-pyranyl, (3,4)-dihydro-2H-thiopyranyl, (1,2,3,6)-tetrahydropyridinyl, (1,2,3,4)-tetrahydropyridinyl, (1,2,5,6)-tetrahydropyridinyl, [1,3]-oxazinanyl, hexahydropyrimidinyl, (5,6)-dihydro-4H-pyrimidinyl, oxazolidinyl, (1,3)-dioxanyl, (1,4)-dioxanyl and (1,3)-dioxolanyl.

Suitable saturated or unsaturated heterocyclic rings which may be condensed with an unsubstituted or at least mono-substituted mono- or bicyclic ring system may preferably be selected from the group consisting of pyrrolidinyl, piperidinyl, piperazinyl, homopiperazinyl, morpholinyl, aziridinyl, azetidinyl, imidazolidinyl, thiomorpholinyl, pyrazolidinyl, azepanyl, diazepanyl, azocanyl, (1,2,3,6)-tetrahydropyridinyl, (1,2,3,4)-tetrahydropyridinyl, (1,2,5,6)-tetrahydropyridinyl, hexahydropyrimidinyl, (5,6)-dihydro-4H-pyrimidinyl, pyridazin-3(2H)-on-yl, phthalazin-1(2H)-on-yl, indolinyl, isoindolinyl, decahydronaphthyl, (1,2,3,4)-tetrahydroquinolinyl, (1,2,3,4)-tetrahydroisoquinolinyl, (1,2,3,4)-tetrahydronaphthyl, octahydro-cyclopenta[c]pyrrolyl, (1,3,4,7,9a)-hexahydro-2H-quinolizinyl, (1,2,3,5,6,8a)-hexahydro-indolizinyl, decahydroquinolinyl, octahydropyrrolo[1,2-a]pyrazinyl, octahydro-1H-pyrido[1,2-a]pyrazinyl, dodecahydrocarbazolyl, 9H-carbazolyl, decahydroisoquinolinyl and (2,3)-dihydro-1H-benzo[de]isoquinolinyl.

Suitable aromatic heterocyclic rings which may be condensed with an unsubstituted or at least mono-substituted mono- or bicyclic ring system may preferably be selected from the group consisting of imidazolyl, pyrazolyl, triazolyl, (4,5,6,7)-tetrahydro-2H-indazolyl, indazolyl and benzimidazolyl.

Suitable saturated or unsaturated, optionally at least one heteroatom as ring member containing cycloaliphatic radicals, C₃₋₉ cycloalkyl radicals or C₄₋₉ cycloalkenyl radicals which are condensed with an unsubstituted or at least mono-substituted mono- or bicyclic ring system may preferably be selected from the group consisting of indolinyl, isoindolinyl, decahydronaphthyl, (1,2,3,4)-tetrahydroquinolinyl, (1,2,3,4)-tetrahydroisoquinolinyl, (1,2,3,4)-tetrahydronaphthyl, octahydro-cyclopenta[c]pyrrolyl, (1,3,4,7,9a)-hexahydro-2H-quinolizinyl, (1,2,3,5,6,8a)-hexahydro-indolizinyl, decahydroquinolinyl, dodecahydrocarbazolyl, 9H-carbazolyl, decahydroisoquinolinyl, (6,7)-dihydro-4H-thieno[3,2-c]pyridinyl, (2,3)-dihydro-1H-benzo[de]isoquinolinyl, octahydropyrrolo[1,2-a]pyrazinyl, octahydro-1H-pyrido[1,2-a]pyrazinyl, (1,2,3,7,8,8a)-hexahydroindolizinyl, (2,6,7,8,9,9a)-hexahydro-1H-quinolizinyl, octahydroindolizinyl, octahydro-1H-quinolizinyl, (1,2,3,5,8,8a)-hexahydroindolizinyl, (4,6,7,8,9,9a)-hexahydro-1H-quinolizinyl, fluorenyl and (1,2,3,4)-tetrahydroquinoxalinyl.

If any of the substituents in any of the above defined formulae represents an alkylene group, preferably an C₁₋₆ alkylene group, an alkenylene group, preferably an C₂₋₆ alkenylene group or an alkinylene group, preferably an C₂₋₆ alkinylene group, which may be substituted, said alkylene group, C₂₋₆ alkylene group, alkenylene group, C₂₋₆ alkenylene group, alkinylene group or C₂₋₆ alkinylene group may be unsubstituted or substituted by one or more substituents, preferably unsubstituted or optionally substituted with 1, 2 or 3 substituent(s). Said substituent(s) may preferably be selected independently from the group consisting of —O—C₁₋₅-alkyl, —S—C₁₋₅-alkyl, —F, Cl, Br, I, —CN, —CF₃, —OCF₃, —SCF₃, —OH, —SH, —NH₂, —NH(C₁₋₅-alkyl) and —N(C₁₋₅-alkyl)₂, whereby in each occurrence C₁₋₅-alkyl may be linear or branched. An alkenylene group comprises at least one carbon-carbon double bond, an alkinylene group comprises at least one carbon-carbon triple bond.

Suitable alkylene groups include —(CH₂)—, —CH(CH₃)—, —CH(phenyl), —(CH₂)₂—, —(CH₂)₃—, —(CH₂)₄—, —(CH₂)₅ and —(CH₂)₆—, suitable alkenylene groups include —CH═CH—, —CH₂—CH═CH— and —CH═CH—CH₂— and suitable alkinylene groups include —C≡C—, —CH₂—C≡C— and —C≡C—CH₂—.

If any of the substituents in any of the above defined formulae represents or comprises an aryl radical, including a 6-membered aryl radical such as phenyl or a 10-membered aryl radical such as naphthyl or a 14-membered aryl radical such as anthracenyl, said aryl radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of C₁₋₅-alkyl, —O—C₁₋₅-alkyl, —S—C₁₋₅-alkyl, —C(═O)—OH, —C(═O)—C₁₋₅-alkyl, —O(═O)—O—C₁₋₅-alkyl, —O—C(═O)—C₁₋₅-alkyl, F, Cl, Br, I, —CN, —CF₃, —OCF₃, —SCF₃, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —O(═O)—N(C₁₋₅-alkyl)₂, —S(═O)₂—C₁₋₅-alkyl, —S(═O)₂-phenyl, —C₁₋₅-alkylene-C(═O)—OH, —C₁₋₅-alkylene—C(═O)—O—C₁₋₅-alkyl, —NH—C(═O)—C₁₋₅-alkyl, —NH—S(═O)₂-C₁₋₅-alkyl, pyrrolidinyl, piperdinyl, morpholinyl, oxazolyl, isoxazolyl, pyridinyl, cyclopropyl, cyclobutyl, cyclopentyl, cyclohexyl, thiophenyl, furanyl, pyrrolidin-2,5-dionyl, phenyl, phenoxy and benzyl; whereby in each occurrence C₁₋₅-alkyl may be linear or branched and whereby said cyclic substituent(s) may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₃—CH₃—CH₃, —C(═O)—O—CH(CH₃)₂, —C(═O)—O—C(CH₃)₃, —C(═O)—CH₃, —C(═O)—C₂H₅, —C(═O)—CH₃—CH₃—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

Preferred aryl radicals, which may optionally be at least mono-substituted, are phenyl and naphthyl.

Suitable aryl radicals, which are condensed with an unsubstituted or at least mono-substituted saturated or unsaturated mono- or bicyclic ring system, may preferably be selected from the group consisting of indolinyl, isoindolinyl, (1,2,3,4)-tetrahydroquinolinyl, (1,2,3,4)-tetrahydroisoquinolinyl, (1,2,3,4)-tetrahydronaphthyl, (1,2,3,4)-tetrahydroquinoxalinyl, benzo[d]oxazol-2(3H)-onyl, benzo[d]thiazol-2(3H)-onyl, 2,3-dihydrobenzo[b][1,4]dioxinyl, benzo[d][1,3]dioxolyl, 3,4-dihydro-2H-benzo[b][1,4]oxazinyl, isochromanyl, chromanyl, 2,3-dihydrobenzofuranyl and 1H-benzo[b][1,4]diazepine-2,4(3H,5H)-dionyl.

If any of the substituents in any of the above defined formulae represents or comprises a heteroaryl radical, including a monocyclic 5- or 6-membered heteroaryl radical or a bi- or tricyclic 8-, 9-, 10-, 11-, 12-, 13- or 14 membered heteroaryl radical, said heteroaryl radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of C₁₋₅-alkyl, —O—C₁₋₅-alkyl, —S—C₁₋₅-alkyl, —C(═O)—OH, —C(═O)—C₁₋₅-alkyl, —O(═O)—O—C₁₋₅-alkyl, —O—C(═O)—C₁₋₅-alkyl, F, Cl, Br, I, —CN, —CF₃, —CF₃, —OCF₃, —SCF₃, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —O(═O)—N(C₁₋₅-alkyl)₂, —S(═O)₂—C₁₋₅-alkyl, —S(═O)₂-phenyl, —C₁₋₅-alkylene-C(═O)—OH, —C₁₋₅-alkylene-C(═O)—O—C₁₋₅-alkyl, —NH—C(═O)—C₁₋₅-alkyl, —NH—S(═O)₂—C₁₋₅-alkyl, pyrrolidinyl, piperdinyl, morpholinyl, oxazolyl, isoxazolyl, pyridinyl, cyclopropyl, cyclobutyl, cyclopentyl, cyclohexyl, thiophenyl, furanyl, pyrrolidin-2,5-dionyl, phenyl, phenoxy and benzyl; whereby in each occurrence C₁₋₅-alkyl may be linear or branched and whereby said cyclic substituent(s) may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected independently from the group consisting of methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—C₂H₅, —C(═O)—O—CH₃—CH₃—CH₃, —C(═O)—O—CH(CH₃)₂, —C(═O)—O—C(CH₃)₃, —C(═O)—CH₃, —C(═O)—C₂H₅, —C(═O)—CH₃—CH₃—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂ and —S(═O)₂—CH₃.

The heteroatom(s), which are present as ring member(s) in the heteroaryl radical, may, unless defined otherwise, independently be selected from the group consisting of nitrogen, oxygen and sulphur. Preferably the heteroaryl radical comprises 1, 2, 3 or 4 heteroatom(s).

Suitable bi- or tricyclic heteroaryl radicals, which may optionally be at least mono-substituted, may preferably be selected from the group consisting of indolyl, isoindolyl, quinolinyl, isoquinolinyl, benzo[b]furanyl, benzo[b]thiophenyl, benzimidazolyl, benzo[2,1,3]thiadiazolyl, [1,2,3]-benzothiadiazolyl, [2,1,3]-benzoxadiazolyl, [1,2,3]-benzoxadiazolyl, benzoxazolyl, benzothiazolyl, benzisoxazolyl, benzisothiazolyl, imidazo[2,1-b]thiazolyl, 2H-chromenyl, indazolyl and quinazolinyl.

Suitable mono-, bi- or tricyclic heteroaryl radicals, which are condensed with an unsubstituted or at least mono-substituted saturated or unsaturated mono- or bicyclic ring system, may preferably be selected from the group consisting of [1,3]-benzodioxolyl, [1,4]-benzodioxanyl, [1,2,3,4]-tetrahydronaphthyl, (2,3)-dihydro-1H-cyclopenta[b]indolyl, [1,2,3,4]-tetrahydroquinolinyl, [1,2,3,4]-tetrahydroisoquinolinyl, [1,2,3,4]-tetrahydroquinazolinyl and [3,4]-dihydro-2H-benzo[1,4]oxazinyl.

Suitable monocyclic heteroaryl radicals, which may optionally be at least mono-substituted, may preferably be selected from the group consisting of pyridinyl, furyl(furanyl), thiophenyl(thiophenyl), pyrrolyl, oxazolyl, isoxazolyl, thiazolyl, isothiazolyl, imidazolyl, pyrazolyl, oxadiazolyl, thiadiazolyl, triazolyl, pyridazinyl, pyrimidinyl, pyrazinyl and pyranyl.

A mono- or bicyclic ring system according to the present invention—if not defined otherwise—means a mono- or bicyclic hydrocarbon ring system that may be saturated, unsaturated or aromatic. Each of its different rings may show a different degree of saturation, i.e. it may be saturated, unsaturated or aromatic. Optionally each of the rings of the mono- or bicyclic ring system may contain one or more, preferably 1, 2 or 3, heteroatom(s) as ring member(s), which may be identical or different and which can preferably be selected from the group consisting of N, O and S. The rings of the mono- or bicyclic ring system are preferably 5-, 6- or 7-membered.

Preferably a mono-or bicyclic ring system according to the present invention is a phenyl or naphthyl ring system.

The term “condensed” according to the present invention means that a ring or ring system is attached to another ring or ring system, whereby the terms “annulated” or “annelated” are also used by those skilled in the art to designate this kind of attachment.

Such a mono- or bicyclic ring system may—if not defined otherwise—be unsubstituted or substituted by one or more substituents, preferably unsubstituted or substituted with 1, 2, 3, 4 or 5 substituent(s). Said substituents may preferably be selected independently from the group consisting of C₁₋₅-alkyl, —O—C₁₋₅-alkyl, —S—C₁₋₅-alkyl, —C(═O)—OH, oxo (═O), thioxo (═S), —C(═O)—O—C₁₋₅-alkyl, —O—C(═O)—C₁₋₅-alkyl, F, Cl, Br, I, —CN, —CF₃, —OCF₃, —SCF₃, —OH, —SH, —NH₂, —NH(C₁₋₅-alkyl), —N(C₁₋₅-alkyl)₂, —NO₂, —CHO, —CF₂H, —CFH₂, —C(═O)—NH₂, —C(═O)—NH(C₁₋₅-alkyl), —C(═O)—N(C₁₋₅-alkyl)₂, —S(═O)₂—C₁₋₅-alkyl, —S(═O)₂-phenyl, phenyl, phenoxy and benzyl; whereby in each occurrence C₁₋₅-alkyl may be linear or branched and whereby said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

More preferably said substituents may be selected from the group consisting of oxo (═O), thioxo (═S), methyl, ethyl, n-propyl, isopropyl, n-butyl, tert-butyl, sec-butyl, isobutyl, n-pentyl, —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, —C(═O)—OH, —C(═O)—O—CH₃, —C(═O)—O—O₂H₅, —C(═O)—O—CH₃—CH₃—CH₃, —C(═O)—O—CH(CH₃)₂, —C(═O)—O—C(CH₃)₃, —C(═O)—CH₃, —C(═O)—O₂H₅, —C(═O)—CH₃—CH₃—CH₃, —C(═O)—CH(CH₃)₂, —C(═O)—C(CH₃)₃, F, Cl, Br, I, —CN, —CF₃, —OCF₃, —SCF₃, —OH, —SH, —NH₂, —NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂, —NO₂, —CHO, —CF₂H, —CFH₂, —C(═O)—NH₂, —C(═O)—NH—CH₃, —C(═O)—NH—C₂H₅, —C(═O)—N(CH₃)₂, —C(═O)—N(C₂H₅)₂, —S(═O)₂—CH₃, —S(═O)₂-phenyl, phenyl, phenoxy and benzyl; whereby in each occurrence said cyclic substituents may be unsubstituted or substituted by 1, 2 or 3 substituent(s) independently selected from the group consisting of methyl, ethyl, n-propyl, isopropyl, methoxy, ethoxy, F, Cl, Br, —CN, —CF₃, —OCF₃, —SCF₃, —OH, —SH, —NH₂ and —NO₂.

If any of the substituents in any of the above defined formulae represents a saturated or unsaturated aliphatic radical, i.e. an alkyl radical, preferably an C₁₋₁₀ alkyl radical; an alkenyl radical, preferably an C₂₋₁₀ alkenyl radical or an alkinyl radical, preferably an C₂₋₁₀ alkinyl radical; said aliphatic radical may—if not defined otherwise—be unsubstituted or substituted by one or more substituents, preferably unsubstituted or substituted with 1, 2, 3, 4 or 5 substituent(s). Said substituent(s) may preferably be selected independently from the group consisting of —O—C₁₋₅-alkyl, —S—C₁₋₅-alkyl, F, Cl, Br, I, —CN, —CF₃, —OCF₃, —SCF₃, —OH, —SH, —NH₂, —NH(C₁₋₅-alkyl) and —N(C₁₋₅-alkyl)₂, whereby in each occurrence C₁₋₅-alkyl may be linear or branched. More preferably said substituent(s) may preferably be selected independently from the group consisting of —O—CH₃, —O—C₂H₅, —O—CH₂—CH₂—CH₃, —O—CH(CH₃)₂, —O—C(CH₃)₃, —S—CH₃, —S—C₂H₅, —S—CH₂—CH₂—CH₃, —S—CH(CH₃)₂, —S—C(CH₃)₃, F, Cl, Br, I, —CN, —CF₃, —OCF₃, —SCF₃, —OH, —SH, —NH₂, NH—CH₃, —NH—C₂H₅, —NH—CH₂—CH₂—CH₃, —NH—CH(CH₃)₂, —NH—C(CH₃)₃, —N(CH₃)₂, —N(C₂H₅)₂.

An alkenyl radical comprises at least one carbon-carbon double bond, an alkinyl radical comprises at least one carbon-carbon triple bond.

Suitable alkyl radicals, which may be substituted by one or more substituents, may preferably be selected from the group consisting of methyl, ethyl, n-propyl, isopropyl, n-butyl, isobutyl, sec-butyl, tert-butyl, n-pentyl, n-hexyl, n-heptyl, n-octyl, n-nonyl and n-decyl.

Suitable alkenyl radicals, which may be substituted by one or more substituents, may preferably be selected from the group consisting of vinyl, 1-propenyl, 2-propenyl, 1-butenyl, 2-butenyl and 3-butenyl.

Suitable alkinyl radicals, which may be substituted by one or more substituents, may preferably be selected from the group consisting of ethinyl, 1-propinyl, 2-propinyl, 1-butinyl, 2-butinyl and 3-butinyl.

Also preferably a compound selected from the group consisting of 5-methoxy-tryptamine, bromocriptine, octoclothepin, clozapine, olanzapine, loxapine, chlorpromazine, fluphenazine, BVT-5182, EMDT, Ro 65-7199, Ro 04-6790, Ro 63-0563, MS-245, SB-271046, LY-483518, ALX-0440, WAY-181189, WAY-466, ALX-1175, ALX-1161, SB-271046, SB-258510, SB-357134, SB-214111, SB-399885, Ro 65-7674, SB-699929, Ro 43-68554, SB-742457, Ro 66-0074, [¹¹C]GSK-215083,
[HT1] N,N-dimethyl-2-(1-(phenylsulfonyl)-1H-indol-3-yl)ethanamine,
[HT2] 5-chloro-2-methyl-3-(1,2,3,6-tetrahydropyridin-4-yl)-1H-indole,
[HT3] 2-(1-(5-(phenylsulfonamido)-1H-indol-3-yl)ethylidene)hydrazinecarboximidamide,
[HT4] 5-fluoro-3-(3-fluorophenylsulfonyl)-1-(piperidin-3-yl)-1H-indole,
[HT5] 2-((1-(4-aminophenylsulfonyl)-1H-indol-3-yl)methylene)hydrazinecarboximidamide,
[HT6] 3-(phenylsulfonyl)-7-(2-(pyrrolidin-1-yl)ethoxy)-1H-indole,
[HT7] N,N-dimethyl-2-(1-(phenylsulfonyl)-1H-indol-4-yloxy)ethanamine,
[HT8] 1-(phenylsulfonyl)-3-(pyrrolidin-2-ylmethyl)-1H-indole,
[HT9] 2-(3-benzyl-1H-indol-1-yl)-N,N-dimethylethanamine,
[HT10] 4-(3-(1,2,3,6-tetrahydropyridin-4-yl)-1H-indol-1-ylsulfonyl)benzenamine,
[HT12] (6S,7S)-7-(dimethylamino)-2-tosyl-2,6,7,8-tetrahydrobenzo[cd]indol-6-ol,
[HT13] 2-(1-(phenylsulfonyl)-6,7-dihydro-1H-indol-4(5H)-ylidene)hydrazinecarboximidamide,
[HT14] 6-(phenylsulfonyl)-2,3,4,9-tetrahydro-1H-carbazol-3-amine,
[HT15] 3-(3-chlorophenylsulfonyl)-1-(pyrrolidin-2-ylmethyl)-1H-pyrrolo[2,3-b]pyridine,
[HT16] 4-(5,6,7,8-tetrahydrocarbazol-9-ylsulfonyl)benzenamine,
[HT17] 3-(2-aminoethyl)-1-(naphthalen-2-ylsulfonyl)-1H-imidazo[4,5-b]pyridin-2(3H)-one,
[HT18] 2-(4-(6-chloroimidazo[2,1-b]thiazol-5-ylsulfonyl)-4H-thieno[3,2-b]pyrrol-6-yl)-N,N-dimethylethanamine,
[HT19] N,N-dimethyl-3-(phenylsulfonyl)-6,7,8,9-tetrahydro-3H-benzo[e]indol-8-amine,
[HT20] N-(4-(piperidin-4-yl)-2H-chromen-6-yl)naphthalene-1-sulfonamide,
[HT21] 2-(3-benzyl-3H-inden-1-yl)-N,N-dimethylethanamine,
[HT22] N-(7-((3S,4S)-3-fluoropiperidin-4-yloxy)benzofuran-5-yl)-2-methoxy-5-methylbenzenesulfonamide,
[HT23] 2-Methoxy-10-(1-methylpyrrolidin-3-yl)-5-thia-4b-aza-indeno[2,1-a]indene 5,5-dioxide,
[HT24] 1-(6-(phenylsulfonyl)pyridin-3-yl)piperazine,
[HT25] 1-(2-methoxy-5-(naphthalen-1-ylsulfonyl)phenyl)piperazine,
[HT26] 1-(2-chloro-5-(phenylsulfonyl)phenyl)piperazine,
[HT27] 8-methoxy-1-(3-methoxy-5-(piperazin-1-yl)benzyl)-3,4-dihydroquinolin-2(1H)-one,
[HT28] 5-chloro-3-(3-chlorophenylsulfonyl)-7-(4-methylpiperazin-1-yl)-1H-indole,
[HT29] 1-(4-(phenylsulfonyl)naphthalen-1-yl)piperazine,
[HT30] 3-(phenylsulfonyl)-7-(piperazin-1-yl)-1H-pyrrolo[3,2-b]pyridine,
[HT31] 4-(2-chlorophenylsulfonyl)-6-methyl-8-(piperazin-1-yl)-3,4-dihydro-2H-benzo[b][1,4]oxazine,
[HT32] 5-(4-methylpiperazin-1-yl)-3-(naphthalen-1-ylsulfonyl)-1H-indazole,
[HT33] 4-(4-(4-(4,5-dihydro-1H-imidazol-2-yl)piperazin-1-yl)-1H-indol-1-ylsulfonyl)benzenamine,
[HT34] 5-(4-benzylpiperazin-1-yl)-3-(phenylsulfonyl)-1H-pyrrolo[3,2-b]pyridine,
[HT35] 2-(2,3-dichlorophenylsulfonyl)-5-(piperazin-1-yl)-1,2,3,4-tetrahydroisoquinoline,
[HT36] 6-fluoro-4-(2-fluorobenzyl)-8-(piperazin-1-yl)-2H-benzo[b][1,4]oxazin-3(4H)-one,
[HT37] 2-(phenylsulfonyl)-4-(piperazin-1-yl)-1H-indole,
[HT38] 4-(4-(piperazin-1-yl)naphthalen-1-ylsulfonyl)benzenamine,
[HT39] 8-(4-(4-fluorobenzyl)piperazin-1-yl)-3-(phenylsulfonyl)quinoline,
[HT40] 2,3-dichloro-N-(2,3,4,5-tetrahydro-1H-benzo[d]azepin-7-yl)benzenesulfonamide,
[HT41] 2-(methylthio)-3-(phenylsulfonyl)-4H-pyrido[1,2-a]pyrimidin-4-imine and
[HT42] 8-(4-(3-fluoro-5-(trifluoromethyl)benzyl)phenylsulfonyl)-N,N,3-trimethyl-2,3,4,5-tetrahydro-1H-benzo[d]azepin-7-amine;
   or salts, free bases, racemates or enantiomers thereof, is present as component (A).

The structure of these compounds is depicted below:

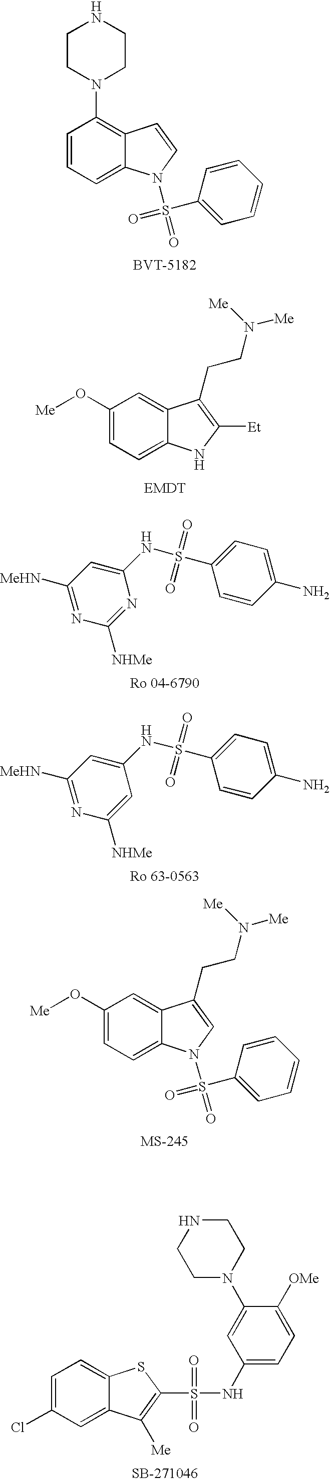

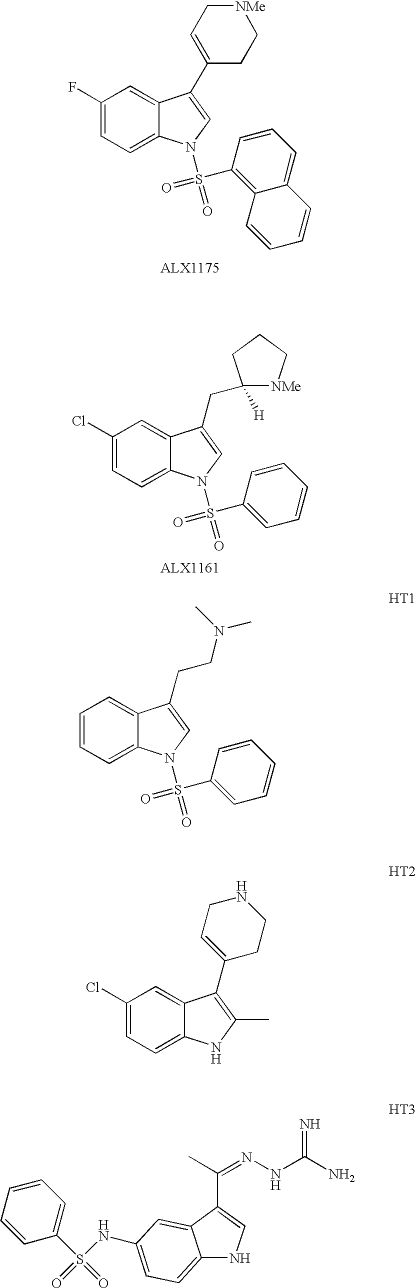

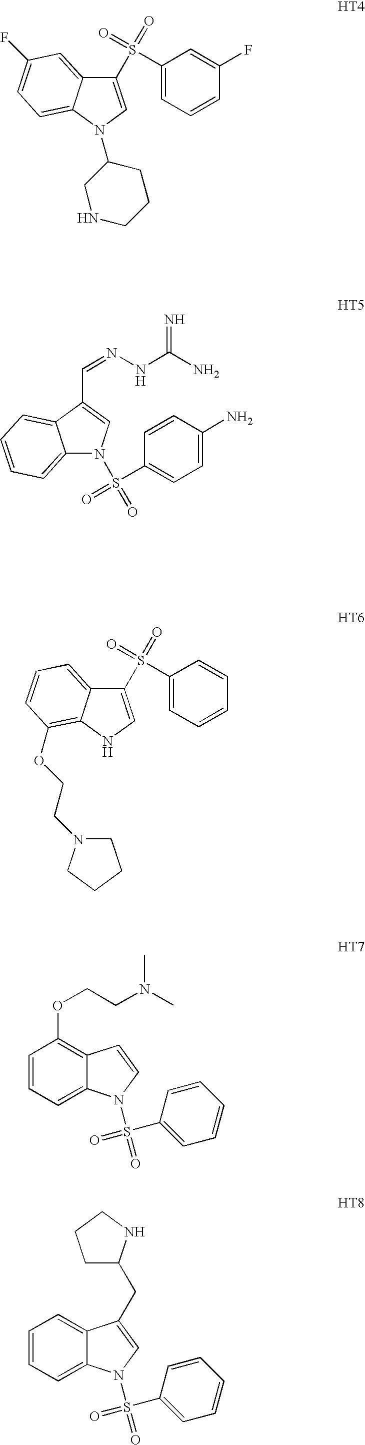

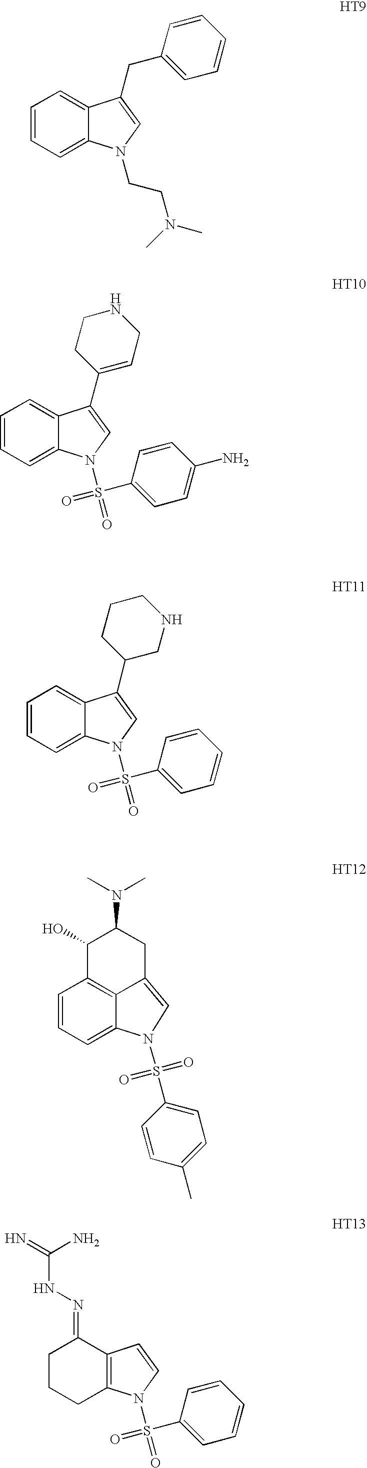

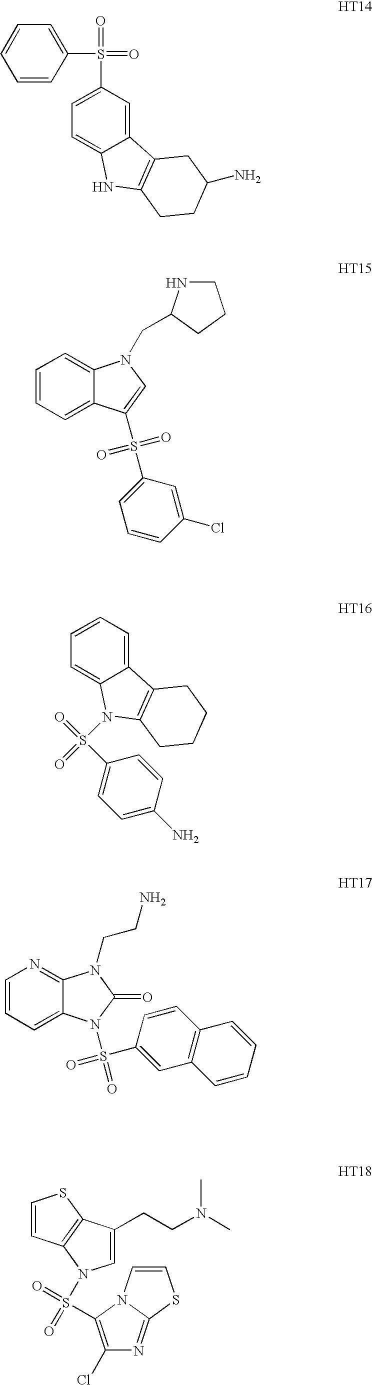

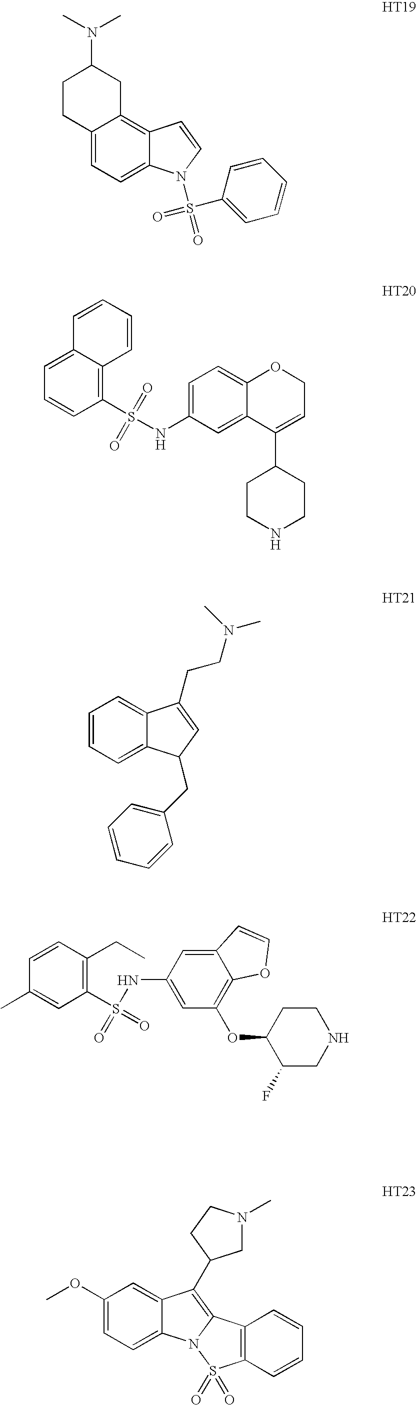

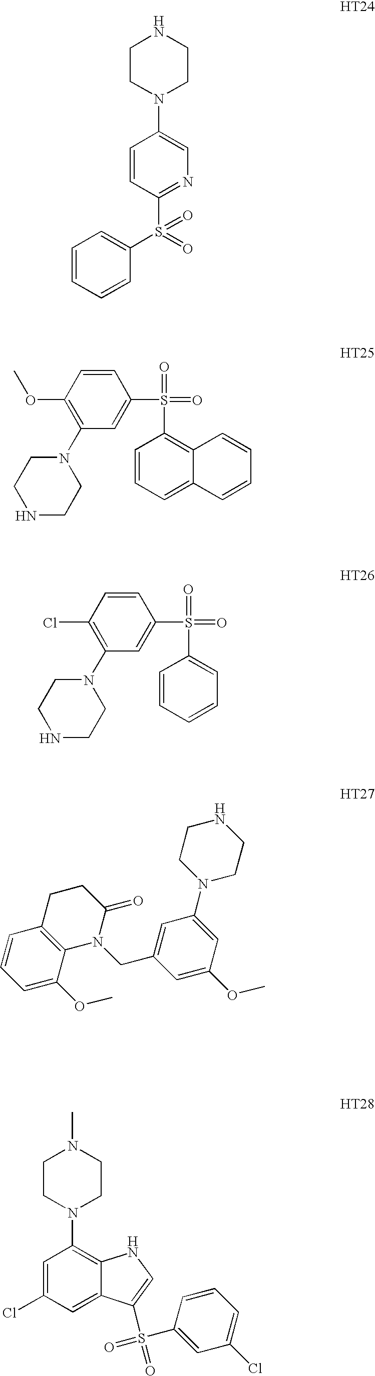

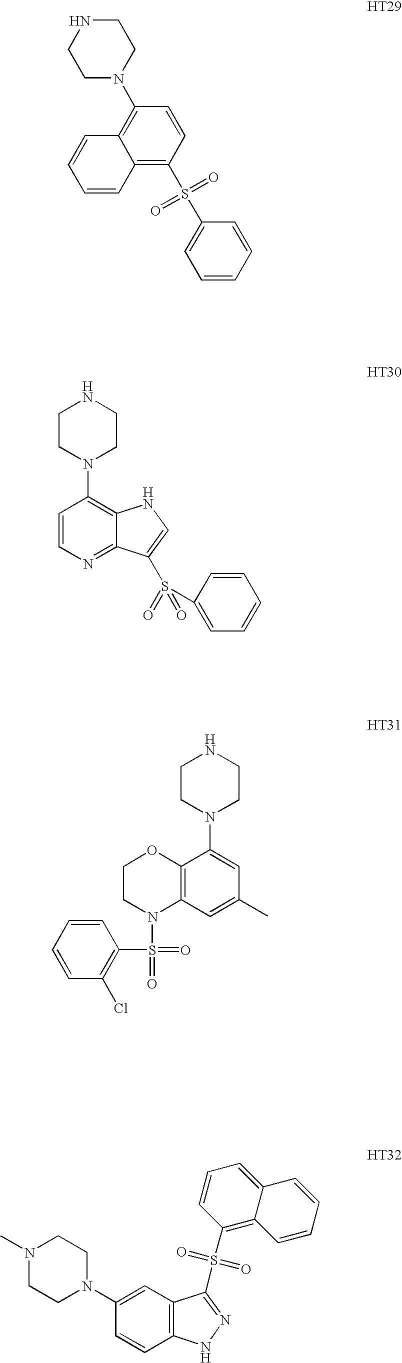

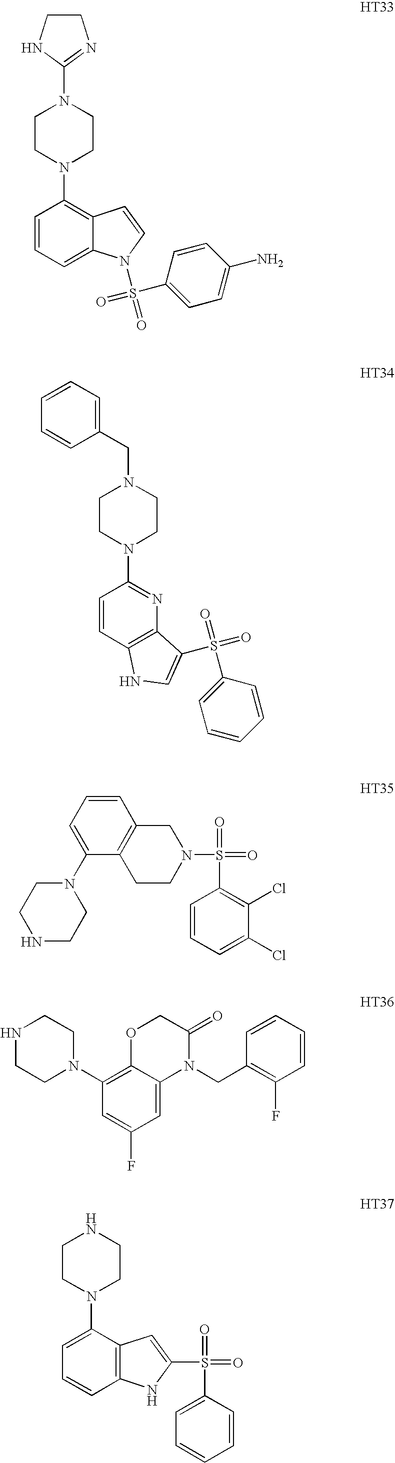

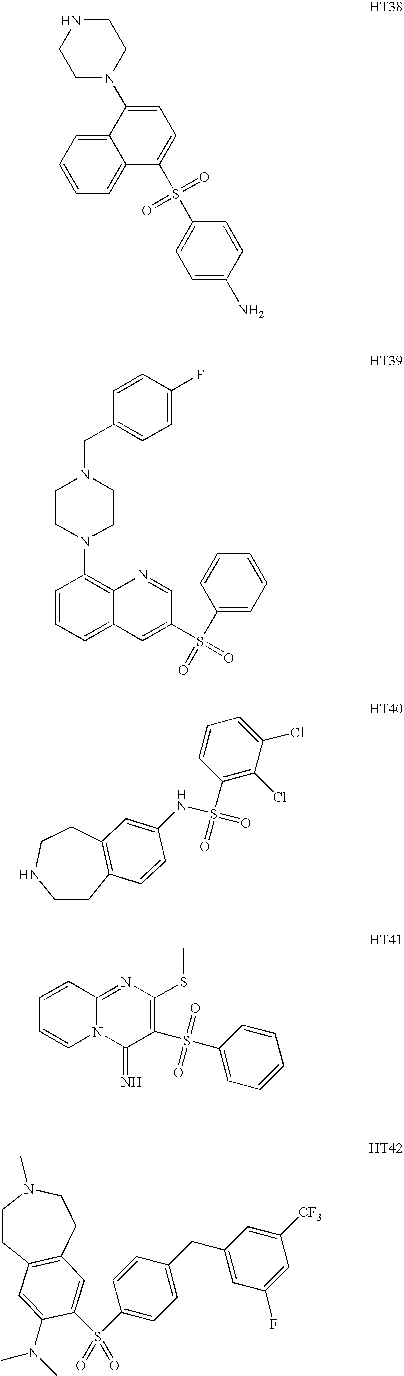

The compounds ALX-0440, ALX-1161 and ALX-1175 can prepared according to the disclosure of WO 2000/63203.

The compounds MS-245, HT12 and HT1 can be prepared according to the disclosure of Bioorg. Med. Chem. Lett. 2000, 10, 2295-2299 and J. Med. Chem. 2001, 44, 3881-3895.

The compound Ro 65-1799 can be prepared according to the disclosure of Eur. J. Med. Chem. 2001, 36, 165-178.

The compound HT2 can be prepared according to the disclosure of Bioorg. Med. Chem. Lett. 2005, 15, 4230-4234.

The compound LY-483518 or SGS518 can be prepared according to the disclosure of WO 2002/60871.

The compound WAY-181187 can be prepared according to the disclosure of WO 2003/53433 and WO 2006/2125.

The compound HT3 can be prepared according to the disclosure of US 2003/232843.

The compound HT4 can be prepared according to the disclosure of WO 2004/9548.

The compound HT5 can be prepared according to the disclosure of US 2004/2527.

The compound HT6 can be prepared according to the disclosure of WO 2004/50085.

The compound WAY-466 can be prepared according to the disclosure of J. Med. Chem. 2005, 48, 353-356.

The compound HT7 can be prepared according to the disclosure of Bioorg. Med. Chem. Lett. 2005, 15, 1393-1396.

The compound HT8 can be prepared according to the disclosure of Bioorg. Med. Chem. Lett. 2005, 15, 3510-3513.

The compounds HT9 and HT21 can be prepared according to the disclosure of Bioorg. Med. Chem. Lett. 2005, 15, 1987-1991.

The compound HT10 can be prepared according to the disclosure of Bioorg. Med. Chem. Lett. 2005, 15, 379-383.

The compound HT11 can be prepared according to the disclosure of Bioorg. Med. Chem. Lett. 2005, 15, 4780-4785.

The compound HT13 can be prepared according to the disclosure of WO 2003/68740.

The compound HT14 can be prepared according to the disclosure of WO 2003/30901.

The compound HT15 can be prepared according to the disclosure of WO 2004/9600.

The compound HT16 can be prepared according to the disclosure of Biorg. Med. Chem. Lett. 2004, 14, 1961-1964.

The compound HT17 can be prepared according to the disclosure of WO 2005/10003.

The compound HT18 can be prepared according to the disclosure of WO 2005/12311.

The compound HT19 can be prepared according to the disclosure of US 2005/101596.

The compound HT20 can be prepared according to the disclosure of WO 2005/37830.

The compound HT22 can be prepared according to the disclosure of WO 2005/58858.

The compound HT23 can be prepared according to the disclosure of WO 2005/66184.

The compounds SB-271040 and SB-258510 can be prepared according to the disclosure of J. Med. Chem. 1999, 42, 202-205.

The compound SB-357134 can be prepared according to the disclosure of Bioorg. Med. Chem. Lett. 2001, 11, 55-58.

The compound HT24 can be prepared according to the disclosure of WO 2003/72548.

The compound HT25 can be prepared according to the disclosure of WO 2003/14097.

The compound HT26 can be prepared according to the disclosure of WO 2004/80968.

The compound HT27 can be prepared according to the disclosure of WO 2004/80969.

The compounds SB-699929 and HT28 can be prepared according to the disclosure of WO 2002/41889.

The compound Ro 43-68554 can be prepared according to the disclosure of WO 2002/98857.

The compound HT29 can be prepared according to the disclosure of WO 2003/72558.

The compound HT30 can be prepared according to the disclosure of WO 2003/80608.

The compound BVT-5182 can be prepared according to the disclosure of WO 2002/102774.

The compound HT31 can be prepared according to the disclosure of WO 2003/95434.

The compound SB-742457 can be prepared according to the disclosure of WO 2003/80580.

The compound HT32 can be prepared according to the disclosure of US 2004/167122.

The compound HT33 can be prepared according to the disclosure of US 2004/192749.

The compound HT34 can be prepared according to the disclosure of WO 2004/74286.

The compound HT35 can be prepared according to the disclosure of WO 2004/78176.

The compound HT36 can be prepared according to the disclosure of US 2004/92512.

The compound HT37 can be prepared according to the disclosure of WO 2004/26831.

The compound HT38 can be prepared according to the disclosure of Bioorg. Med. Chem. Lett. 2005, 15, 1707-1711.

The compound HT39 can be prepared according to the disclosure of WO 2005/26125.

The compounds Ro 04-6790 and Ro 63-0563 can be prepared according to the disclosure of Br. J. Phamacol. 1998, 124, 556-562.

The compound HT40 can be prepared according to the disclosure of WO 2003/68751.

The compound Ro 66-0074 can be prepared according to the disclosure of J. Med. Chem. 2003, 46, 1273-1276.

The compound HT41 can be prepared according to the disclosure of US 2004/19064.

The compound HT42 can be prepared according to the disclosure of WO 2005/51398.

The respective parts of the literature are hereby incorporated by reference and form part of the present disclosure.

Those skilled in the art understand that according to the present invention any combination of compounds with 5-HT₆ receptor affinity may be present as component (A) in the active substance combination. Also any combination of cholinesterase inhibitors may be present as component (B) in the active substance combination.

Particularly preferably a compound selected from the group consisting of 6-chloro-N-(3-(2-(dimethylamino)ethyl)-1H-indol-5-yl)imidazo[2,1-b]thiazole-5-sulfonamide, 1-[1-(Naphthalene-1-sulfonyl)-piperidin-4-yl]-1,4-dihydro-benzo[d][1,3]oxazin-2-one, 5-chloro-3-methyl-N-(3-(1-methylpiperidin-4-yl)-1H-indol-5-yl)benzo[b]thiophene-2-sulfonamide, BVT-5182, EMDT, Ro 04-6790, Ro 63-0563, MS-245, SB-271046, ALX-1175 and ALX-1161, or salts, free bases, racemates or enantiomers thereof, is present as component (A).

More particularly preferably the compound [1140] 6-chloro-N-(3-(2-(dimethylamino)ethyl)-1H-indol-5-yl)imidazo[2,1-b]thiazole-5-sulfonamide (also known as E-6801) and/or 5-chloro-3-methyl-N-(3-(1-methylpiperidin-4-yl)-1H-indol-5-yl)benzo[b]thiophene-2-sulfonamide [1141] and/or 1-[1-(Naphthalene-1-sulfonyl)-piperidin-4-yl]-1,4-dihydro-benzo[d][1,3]oxazin-2-one [1b] as depicted below is present as component (A).

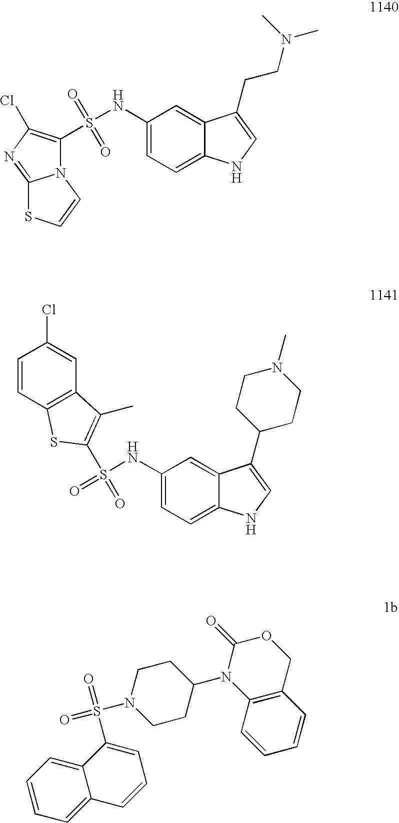

Examples of preferred acetylcholinesterase inhibitors according to the present invention include edrophonium, ganstigmine, demecarium, ambenonium, neostigmine bromide, dehydroevodiamine chloride, eseroline, imperatorin, scopoletin (SCT), huperizine A (Hup A), huperzine A, tacrine hydrochloride (CI-970, THA.HCl), 7-methoxytacrine (7-MEOTA), velnacrine maleate (HP-029, P83-6029A), rivastigmine tartrate (ENA-713, ENA-713D, ONO-2540, SDZ-212-713, SDZ-ENA-713), eptastigmine tartrate (L-693487), heptylstigmine tartrate (MF-201), donepezil hydrochloride (BNAG, E-2020), suronacrine maleate (HP-128), UCB-11056, SM-10888, stacofylline hydrochloride (S-9977, S-9977-2), berberine iodide, zifrosilone (MDL-73745), norpyridostigmine, quilostigmine (HP-290, NXX-066), E-2030, THB-013, F-3796, PD-142012, CI-1002, PD-142676, terestigmine tartrate (CHF-2060), thiacymserine, phenserine tartrate, galantamine hydrobromide (GP-37267), galantamine hydrobromide (R-113675), Ro-46-5934, P11012, MF-8615, MF-268 bitartrate, anseculin hydrochloride (KA-672.HCl), ensaculin hydrochloride, icopezil maleate (CP-118954, CP-118954-11), eserine salicylate, physostigmine salicylate, isovanihuperzine A (IVHA), JWS-USC-75IX, FR-152558, UR-1827, P11467, P-10358, bis(7)-tacrine, HMR-2420, T-82, CP-126998, TV-3279, MSF, THA-C8, subergorgic acid, suberogorgin, SPH-1286, huperzine B (Hup B), pyridostigmine bromide (Ro-1-5130), huprine X, ER-127528, tolserine tartrate, huprine Y, coronaridine, RS-1233, kobophenol A, bis(12)-huperine, BGC-20-1259, RS-1259, NP-7557, ZT-1, ITH-4012, TK-19, T-81, TH-171, TH-185, distigmine bromide (BC-51), (−)-9-dehydrogalanthaminium bromide, memoquin, NP-0361, scopoletin 7-O-beta-D-glucopyranoside (NSC-404560), scopolin (SCN), scopoloside, BW-284c51, SP-004, SP-04, withaferin A (NSC-101088), withaferine (NSC-273757), (+)-corynoline, corynoline, (S)-(−)-oxypeucedanin, oxypeucedanin, (−)-voacangine, carbomethoxyibogaine, voacangine, dieckol, phlorofucofuroeckol (PFF), phlorofucofuroeckol A, (−)-3-O-acetylspectaline hydrochloride and rhaphiasaponin 1, or salts, free bases, racemates or enantiomers thereof.

Exemplary reversible inhibitors include tacrine, donepezil, edrophonium and galantamine. Exemplary pseudo-irreversible inhibitors include physostigmine, eptastigmine, pyridostigmine, neostigmine, ganstigmine and rivastigmine. Pseudo-irreversible cholinesterase inhibitors also include carbamate insecticides, including carbaryl (Sevin), propoxur (Baygon), and aldicarb (Temik). Typically, pseudo-irreversible acetylcholinesterase inhibitors comprise a carbamate moiety, for example, rivastigmine, eptastigmine, physostigmine, neostigmine, demecarium, ambenonium, pyridostigmine, and ganstigmine. Additional cholinesterase inhibitors that can find use in the present invention include huperzine A, T-82, phenserine, quilostigmine, and TAK-147.

Huperzine A, a novel potent, reversible, and selective acetylcholinesterase (AchE) inhibitor has been expected to be superior to other AchE inhibitors now known for the treatment of memory deficits in patients with Alzheimer's disease. It has been shown to inhibit the enzyme that is responsible for the breakdown of acetylcholine, an important neurotransmitter, or brain chemical, which is critical in not only memory and/or learning but is critical in peripheral nervous system as well. This could have a beneficial effect in the disorder known as Myastenia Gravis. Huperzine is a naturally occurring compound that was originally isolated from the club moss Huperzine Serrata. It has been used in Chinese folk medicine and more recently in limited clinical trials conducted in China as a treatment for age-related memory disorders.

More preferably a cholinesteresterase inhibitor selected from the group consisting of huperzine A, rivastigmine, pyridostigmine, distigmine, neostigmine, physostigmine, tacrine, galantamine and donepezil is present as component (B).

Even more preferably donepezil is present as component (B).

Preferably the acetylcholinesterase inhibitor of the present also includes one or more of a cholinesterase inhibitory agent that binds to the acyl pocket of the active centre of AChE, the choline subsite of the active center of AChE, or the peripheral anionic site of AChE. For example, edrophonium and tacrine bind to the choline subsite in the vicinity of tryptophan 86 and glutamate 202 of AChE. Donepezil binds with higher affinity to the active center of AChE. Propidium and the peptide toxin fasciculin bind to the peripheral anionic site on AChE. This is reviewed in Goodman and Gilman's The Pharmacological Basis of Therapeutics, supra, at pages 175-89.

Preferred is an active substance combination that comprises at least one compound selected from the group consisting of 6-chloro-N-(3-(2-(dimethylamino)ethyl)-1H-indol-5-yl)imidazo[2,1-b]thiazole-5-sulfonamide, 1-[1-(Naphthalene-1 -sulfonyl)-piperidin-4-yl]-1,4-dihydro-benzo[d][1,3]oxazin-2-one, 5-chloro-3-methyl-N-(3-(1-methylpiperidin-4-yl)-1H-indol-5-yl)benzo[b]thiophene-2-sulfonamide, 5-methoxy-tryptamine, bromocriptine, octoclothepin, clozapine, olanzapine, loxapine, chlorpromazine, fluphenazine, BVT-5182, EMDT, Ro 65-7199, Ro 04-6790, Ro 63-0563, MS-245, SB-271046, LY-483518, ALX-0440, WAY-181189, WAY-466, ALX-1175, ALX-1161, SB-271046, SB-258510, SB-357134, SB-214111, SB-399885, Ro 65-7674, SB-699929, Ro 43-68554, SB-742457, Ro 66-0074, [¹¹C]GSK-215083,
[HT1] N,N-dimethyl-2-(1-(phenylsulfonyl)-1H-indol-3-yl)ethanamine,
[HT2] 5-chloro-2-methyl-3-(1,2,3,6-tetrahydropyridin-4-yl)-1H-indole,
[HT3] 2-(1-(5-(phenylsulfonamido)-1H-indol-3-yl)ethylidene)hydrazinecarboximidamide,
[HT4] 5-fluoro-3-(3-fluorophenylsulfonyl)-1-(piperidin-3-yl)-1H-indole,
[HT5] 2-((1-(4-aminophenylsulfonyl)-1H-indol-3-yl)methylene)hydrazinecarboximidamide,
[HT6] 3-(phenylsulfonyl)-7-(2-(pyrrolidin-1-yl)ethoxy)-1H-indole,
[HT7] N,N-dimethyl-2-(1-(phenylsulfonyl)-1H-indol-4-yloxy)ethanamin,
[HT8] 1-(phenylsulfonyl)-3-(pyrrolidin-2-ylmethyl)-1H-indole,
[HT9] 2-(3-benzyl-1H-indol-1-yl)-N,N-dimethylethanamine,
[HT10] 4-(3-(1,2,3,6-tetrahydropyridin-4-yl)-1H-indol-1-ylsulfonyl)benzenamine,
[HT12] (6S,7S)-7-(dimethylamino)-2-tosyl-2,6,7,8-tetrahydrobenzo[cd]indol-6-ol,
[HT13] 2-(1-(phenylsulfonyl)-6,7-dihydro-1H-indol-4(5H)-ylidene)hydrazinecarboximidamide,
[HT14] 6-(phenylsulfonyl)-2,3,4,9-tetrahydro-1H-carbazol-3-amine,
[HT15] 3-(3-chlorophenylsulfonyl)-1-(pyrrolidin-2-ylmethyl)-1H-pyrrolo[2,3-b]pyridine,
[HT16] 4-(5,6,7,8-tetrahydrocarbazol-9-ylsulfonyl)benzenamine,
[HT17] 3-(2-aminoethyl)-1-(naphthalen-2-ylsulfonyl)-1H-imidazo[4,5-b]pyridin-2(3H)-one,
[HT18] 2-(4-(6-chloroimidazo[2,1-b]thiazol-5-ylsulfonyl)-4H-thieno[3,2-b]pyrrol-6-yl)-N,N-dimethylethanamine,
[HT19] N,N-dimethyl-3-(phenylsulfonyl)-6,7,8,9-tetrahydro-3H-benzo[e]indol-8-amine,
[HT20] N-(4-(piperidin-4-yl)-2H-chromen-6-yl)naphthalene-1-sulfonamide,
[HT21] 2-(3-benzyl-3H-inden-1-yl)-N,N-dimethylethanamine,
[HT22] N-(7-((3S,4S)-3-fluoropiperidin-4-yloxy)benzofuran-5-yl)-2-methoxy-5-methylbenzenesulfonamide,
[HT23] 2-Methoxy-10-(1-methylpyrrolidin-3-yl)-5-thia-4b-aza-indeno[2,1-a]indene 5,5-dioxide,
[HT24] 1-(6-(phenylsulfonyl)pyridin-3-yl)piperazine,
[HT25] 1-(2-methoxy-5-(naphthalen-1-ylsulfonyl)phenyl)piperazine,
[HT26] 1-(2-chloro-5-(phenylsulfonyl)phenyl)piperazine,
[HT27] 8-methoxy-1-(3-methoxy-5-(piperazin-1-yl)benzyl)-3,4-dihydroquinolin-2(1H)-one,
[HT28] 5-chloro-3-(3-chlorophenylsulfonyl)-7-(4-methylpiperazin-1-yl)-1H-indole,
[HT29] 1-(4-(phenylsulfonyl)naphthalen-1-yl)piperazine,
[HT30] 3-(phenylsulfonyl)-7-(piperazin-1-yl)-1H-pyrrolo[3,2-b]pyridine,
[HT31] 4-(2-chlorophenylsulfonyl)-6-methyl-8-(piperazin-1-yl)-3,4-dihydro-2H-benzo[b][1,4]oxazine,
[HT32] 5-(4-methylpiperazin-1-yl)-3-(naphthalen-1-ylsulfonyl)-1H-indazole,
[HT33] 4-(4-(4-(4,5-dihydro-1H-imidazol-2-yl)piperazin-1-yl)-1H-indol-1-ylsulfonyl)benzenamine,
[HT34] 5-(4-benzylpiperazin-1-yl)-3-(phenylsulfonyl)-1H-pyrrolo[3,2-b]pyridine,
[HT35] 2-(2,3-dichlorophenylsulfonyl)-5-(piperazin-1-yl)-1,2,3,4-tetrahydroisoquinoline,
[HT36] 6-fluoro-4-(2-fluorobenzyl)-8-(piperazin-1-yl)-2H-benzo[b][1,4]oxazin-3(4H)-one,
[HT37] 2-(phenylsulfonyl)-4-(piperazin-1-yl)-1H-indole,
[HT38] 4-(4-(piperazin-1-yl)naphthalen-1-ylsulfonyl)benzenamine,
[HT39] 8-(4-(4-fluorobenzyl)piperazin-1-yl)-3-(phenylsulfonyl)quinoline,
[HT40] 2,3-dichloro-N-(2,3,4,5-tetrahydro-1H-benzo[d]azepin-7-yl)benzenesulfonamide,
[HT41] 2-(methylthio)-3-(phenylsulfonyl)-4H-pyrido[1,2-a]pyrimidin-4-imine and
[HT42] 8-(4-(3-fluoro-5-(trifluoromethyl)benzyl)phenylsulfonyl)-N,N,3-trimethyl-2,3,4,5-tetrahydro-1H-benzo[d]azepin-7-amine;
   or salts, free bases, racemates or enantiomers thereof,

as component (A);

and at least one compound selected from the group consisting of edrophonium, ganstigmine, demecarium, ambenonium, neostigmine bromide, dehydroevodiamine chloride, eseroline, imperatorin, scopoletin (SCT), huperizine A (Hup A), huperzine A, tacrine hydrochloride (CI-970, THA.HCl), 7-methoxytacrine (7-MEOTA), velnacrine maleate (HP-029, P83-6029A), rivastigmine tartrate (ENA-713, ENA-713D, ONO-2540, SDZ-212-713, SDZ-ENA-713), eptastigmine tartrate (L-693487), heptylstigmine tartrate (MF-201), donepezil hydrochloride (BNAG, E-2020), suronacrine maleate (HP-128), UCB-11056, SM-10888, stacofylline hydrochloride (S-9977, S-9977-2), berberine iodide, zifrosilone (MDL-73745), norpyridostigmine, quilostigmine (HP-290, NXX-066), E-2030, THB-013, F-3796, PD-142012, CI-1002, PD-142676, terestigmine tartrate (CHF-2060), thiacymserine, phenserine tartrate, galantamine hydrobromide (GP-37267), galantamine hydrobromide (R-113675), Ro-46-5934, P11012, MF-8615, MF-268 bitartrate, anseculin hydrochloride (KA-672.HCl), ensaculin hydrochloride, icopezil maleate (CP-118954, CP-118954-11), eserine salicylate, physostigmine salicylate, isovanihuperzine A (IVHA), JWS-USC-751X, FR-152558, UR-1827, P11467, P-10358, bis(7)-tacrine, HMR-2420, T-82, CP-126998, TV-3279, MSF, THA-C8, subergorgic acid, suberogorgin, SPH-1286, huperzine B (Hup B), pyridostigmine bromide (Ro-1-5130), huprine X, ER-127528, tolserine tartrate, huprine Y, coronaridine, RS-1233, kobophenol A, bis(12)-huperine, BGC-20-1259, RS-1259, NP-7557, ZT-1, ITH-4012, TK-19, T-81, TH-171, TH-185, distigmine bromide (BC-51), (−)-9-dehydrogalanthaminium bromide, memoquin, NP-0361, scopoletin 7-O-beta-D-glucopyranoside (NSC-404560), scopolin (SCN), scopoloside, BW-284c51, SP-004, SP-04, withaferin A (NSC-101088), withaferine (NSC-273757), (+)-corynoline, corynoline, (S)-(−)-oxypeucedanin, oxypeucedanin, (−)-voacangine, carbomethoxyibogaine, voacangine, dieckol, phlorofucofuroeckol (PFF), phlorofucofuroeckol A, (−)-3-O-acetylspectaline hydrochloride and rhaphiasaponin 1, or salts, free bases, racemates or enantiomers thereof,

as component (B).

Particularly preferred is an active substance combination that comprises 1140 as component (A) and donepezil as component (B).

Particularly preferred is an active substance combination that comprises 1140 as component (A) and huperzine A as component (B).

Particularly preferred is an active substance combination that comprises 1140 as component (A) and rivastigmine as component (B).

Particularly preferred is an active substance combination that comprises 1140 as component (A) and galantamine as component (B).

Particularly preferred is an active substance combination that comprises 1140 as component (A) and tacrine as component (B).

Particularly preferred is an active substance combination that comprises 1140 as component (A) and physostigmine as component (B).

Particularly preferred is an active substance combination that comprises 1140 as component (A) and pyridostigmine as component (B).

Particularly preferred is an active substance combination that comprises 1140 as component (A) and distigmine as component (B).

Particularly preferred is an active substance combination that comprises 1140 as component (A) and neostigmine as component (B).

“Obesity” is a condition in which there is an excess of body fat. The operational definition of obesity is based on the Body Mass Index(BMI), which is calculated as body weight per height in meters squared (kg/m²).“Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BMI) greater than or equal to 30 kg/m² or a subject with at least one co-morbidity with a BMI greater than or equal to 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index(BMI) in Asians. In Asian countries, including Japan, “obesity” refers to a condition whereby a subject with at least one obesity-induced or obesity-related co-morbidity, that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asia-Pacific, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus-type II (2), impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver; cerebral infarction, cerebral thrombosis, transient ischemic attack, orthopedic disorders, arthritis deformans, lumbodynia, emmeniopathy, and infertility.

In particular, co-morbidities include: hypertension, hyperlipidemia, dyslipidemia, glucose intolerance, cardiovascular disease, sleep apnea, diabetes mellitus, and other obesity-related conditions.

The term “cognitive disorder” indicates disruptions in performance including one or more of the following signs:

1) memory deficits (impaired ability to learn new information or recall previously learned information;

2) one (or more) of the following disturbances:

a) aphasia (language disturbance)

b) apraxia (impaired ability to carry out motor activities despite intact motor function)

c) agnosia (failure to recognize or identify objects despite in tact sensory function)

d) disturbance in executive functioning (i.e. planning, organizing, sequencing, abstracting);

3) memory disturbances causing significant impairment in social or occupational functioning, and representing a significant decline from a previous level of functioning; and

4) impairment in cognitive functioning as evidenced by neuropsychological testing or quantified clinical assessment, accompanied by objective evidence of a systemic general medical condition or central nervous system dysfunction.

Cognitive disorders may include Alzheimer's disease, learning disabilities caused by degenerative disorders, learning disabilities caused by non-degenerative disorders, memory or cognitive dysfunction such as mild cognitive impairment, age-related cognitive decline, cerebral senility, vascular dementia, AIDS-associated dementia, electric shock induced amnesia, memory impairment associated with depression or anxiety, cognitive defects in Parkinson's disease, Down's syndrome, stroke, traumatic brain injury, Huntington's disease, and attention deficit disorder.

“Treatment” (of obesity and obesity-related disorders) refers to the administration of the compounds or combinations of the present invention to reduce or maintain the body weight of an obese subject. One outcome of treatment may be reducing the body weight of an obese subject relative to that subject's body weight immediately before the administration of the compounds or combinations of the present invention. Another outcome of treatment may be preventing body weight regain of body weight previously lost as a result of diet, exercise, or pharmacotherapy.

Another outcome of treatment may be decreasing the occurrence of and/or the severity of obesity-related diseases. Another outcome of treatment may be to maintain weight loss. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

The term “Metabolic syndrome”, also known as syndrome X, is defined in the Third Report of the National Cholesterol Education Program Expert Panel on Detection, Evaluation and Treatment of High Blood Cholesterol in Adults (ATP-E). E. S. Ford et al., JAMA, Vol. 287 (3), Jan. 16, 2002, pp 356-359. Briefly, a person is defined as having Metabolic syndrome if the person has three or more of the following symptoms: abdominal obesity, hypertriglyceridemia, low HDL cholesterol, high blood pressure, and high fasting plasma glucose.

The terms “administration of and or “administering a” compound should be understood to mean providing a compound of the invention or a prodrug of a compound of the invention to a subject in need of treatment.

The instant pharmaceutical composition includes administration of a single pharmaceutical dosage formulation which contains both the compound with 5-HT₆ receptor affinity, and at least one cholinesterase inhibitor, as well as administration of each active agent in its own separate pharmaceutical dosage formulation. Where separate dosage formulations are used, the individual components of the composition can be administered at essentially the same time, i. e., concurrently, or at separately staggered times, i. e. sequentially prior to or subsequent to the administration of the other component of the composition. The instant pharmaceutical composition is therefore to be understood to include all such regimes of simultaneous or alternating treatment, and the terms “administration” and “administering” are to be interpreted accordingly.

Administration in these various ways are suitable for the present compositions as long as the beneficial pharmaceutical effect of the combination of the compound with 5-HT₆ receptor affinity, and at least one cholinesterase inhibitor, is realised by the patient at substantially the same time.

Such beneficial effect is preferably achieved when the target blood level concentrations of each active drug are maintained at substantially the same time. It is preferred that the combination of the compound with 5-HT₆ receptor affinity, and at least one cholinesterase inhibitor, be co-administered concurrently on a once-a-day dosing schedule; however, varying dosing schedules, such as the compound with 5-HT₆ receptor affinity once a day and the cholinesterase inhibitor once, twice or more times per day, is also encompassed herein. A single oral dosage formulation comprised of both a compound with 5-HT₆ receptor affinity and a cholinesterase inhibitor is preferred. A single dosage formulation will provide convenience for the patient, which is an important consideration especially for patients with diabetes or obese patients who may be in need of multiple medications.

The term “subject” as used herein refers to an animal, preferably a mammal, most preferably a human, who has been the object of treatment, observation or experiment.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgement.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. The novel methods of treatment of this invention are for disorders known to those skilled in the art.

The term “salt” as used herein is to be understood as meaning any form of the compounds in which they assume an ionic form or are charged and are coupled with a counter-ion (a cation or anion) or are in solution. By this are also to be understood complexes of the active compound with other molecules and ions, in particular complexes which are complexed via ionic interactions.

The term “physiologically acceptable salt” is understood in particular, in the context of this invention, as salt (as defined above) formed either with a physiologically tolerated acid, that is to say salts of the particular active compound with inorganic or organic acids which are physiologically tolerated—especially if used on humans and/or mammals—or with at least one, preferably inorganic, cation which are physiologically tolerated—especially if used on humans and/or mammals. Examples of physiologically tolerated salts of particular acids are salts of: hydrochloric acid, hydrobromic acid, sulfuric acid, hydrobromide, monohydrobromide, monohydrochloride or hydrochloride, salicylic acid, methiodide, methanesulfonic acid, formic acid, acetic acid, oxalic acid, succinic acid, malic acid, tartaric acid, mandelic acid, fumaric acid, lactic acid, citric acid, glutamic acid, hippuric acid, picric acid and/or aspartic acid. Examples of physiologically tolerated salts of particular bases are salts of alkali metals and alkaline earth metals and with NH₄.

Solvates, preferably hydrates, of the compounds of the present invention and in each case of corresponding stereoisomers may also be obtained by standard procedures known to those skilled in the art.

The term “solvate” according to this invention is to be understood as meaning any form of the compounds in which they have attached to it via non-covalent binding another molecule (most likely a polar solvent) especially including hydrates and alcoholates, e.g. methanolate.

The active substance combination according to this invention comprises preferably 1-99% by weight of component (A) and 99-1% by weight of component (B), more preferably 70 to 95% by weight of component (A) and 30 to 5% by weight of component (B), even more preferably 85 to 95% by weight of component (A) and 15 to 5% by weight of component (B), in each case referring to the total weight of both components (A) and (B).

Another aspect of the present invention is the active substance combination of the invention for its use as a medicament.

Another aspect of the present invention is the use of an inventive active substance combination as defined above for the manufacture of a medicament for simultaneous acetylcholinesterase inhibition and 5-HT₆-receptor regulation.

Another aspect of the present invention is the use of the inventive active substance combination for the manufacture of a medicament for the regulation of appetite, for maintenance, increase or reduction of body weight, for prophylaxis and/or treatment of disorders related to food ingestion, preferably for prophylaxis and/or treatment of obesity, anorexia, cachexia, bulimia, diabetes, preferably type II diabetes (non-insulin-dependent diabetes mellitus), or for prophylaxis and/or treatment of gastrointestinal tract disorders, preferably of the irritable bowel syndrome, for prophylaxis and/or treatment of Peripheral Nervous System Disorders, Central Nervous System Disorders, arthritis, epilepsy, anxiety, panic, depression, preferably bipolar disorders, cognitive disorders, memory disorders, cardiovascular diseases, senile dementia processes, neurodegenerative disorders, preferably Alzheimer's disease, Parkinson's diesease, Huntington's Disease and/or multiple sclerosis, schizophrenia, psychosis, infantile hyperkinesia (ADHD, attention deficit/hyperactivity disorder), pain, hypertensive syndrome, inflammatoric diseases, immunologic diseases or for improvement of cognition.

Particularly preferred is the use of the inventive active substance combination for the manufacture of a medicament for the regulation of appetite, for maintenance, increase or reduction of body weight, for prophylaxis and/or treatment of disorders related to food ingestion, preferably for prophylaxis and/or treatment of obesity, anorexia, cachexia, bulimia, diabetes, preferably type II diabetes (non-insulin-dependent diabetes mellitus), or for prophylaxis and/or treatment of gastrointestinal tract disorders, preferably of the irritable bowel syndrome.

Also particularly preferred is the use of the inventive active substance combination for the manufacture of a medicament for prophylaxis and/or treatment of cognitive disorders, memory disorders, or senile dementia processes, such as Alzheimer's, Parkinson's and/or Huntington's Disease.

More particularly preferred is the use of the inventive active substance combination for the manufacture of a medicament for prophylaxis and/or treatment of obesity, cognitive disorders, memory disorders, or senile dementia processes, such as Alzheimer's, Parkinson's and/or Huntington's Disease.

Also particularly preferred is the use of the active substance combination for the manufacture of a medicament for the prophylaxis and/or treatment of obesity-related disorders such as elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovary disease, craniopharyngioma, the Prader-Willi Syndrome and Frohlich's syndrome. Further examples of obesity-related disorders are reproductive hormone abnormalities, sexual and reproductive dysfunction, such as impaired fertility, infertility, hypogonadism in males and hirsutism in females, fetal defects associated with maternal obesity, gastrointestinal motility disorders, such as obesity-related gastro-esophageal reflux, respiratory disorders, such as obesity-related hypoventilation syndrome (Pickwickian syndrome), breathlessness, cardiovascular disorders, inflammation, such as systemic inflammation of the vasculature, arteriosclerosis, hypercholesterolemia, hyperuricaemia, lower back pain, gallbladder disease, gout, kidney cancer, and increased anesthetic risk.

Those skilled in the art understand that the components (A) and (B) of the active substance combination according to the present invention may be administered simultaneously or sequentially to one another, whereby in each case components (A) and (B) may be administered via the same or different administration pathways, e.g. orally or parentally. preferably both components (A) and (B) are administered simultaneously in one and the same administration form.

Yet another aspect of the present invention are pharmaceutical formulations in different pharmaceutical forms comprising an inventive active substance combination and optionally one or more pharmacologically acceptable adjuvants.

As well known to somebody skilled in the art the pharmaceutical formulations may—depending on their route of administration, also contain one or more auxiliary substances known to those skilled in the art.

The pharmaceutical formulations according to the present invention may be produced according to standard procedures known to those skilled in the art, e.g. from the tables of contents from “Pharmaceutics: the Science of Dosage Forms”, Second Edition, Aulton, M. E. (Ed.) Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modern Pharmaceutics”, Fourth Edition, Banker G. S. and Rhodes C. T. (Eds.) Marcel Dekker, Inc. New York 2002 and “The Theory and Practice of Industrial Pharmacy”, Lachman L., Lieberman H. and Kanig J. (Eds.), Lea & Febiger, Philadelphia (1986). The respective descriptions are incorporated by reference and are part of the disclosure.

Preferred pharmaceutical formulations are solid pharmaceutical forms, preferably tablets, chewing tablets, chewing gums, dragees, capsules, suppositories, powder preparations, transdermal therapeutic systems, transmucosal therapeutic systems, preferably tablets or capsules.

Preferred pharmaceutical formulations are also liquid and semi-liquid pharmaceutical forms such as drops or such as juice, sirup, solution, emulsion, suspension, preferably drops or solutions.

In an additional preferred embodiment, the pharmaceutical formulations are in the form of multiparticulates, preferably microtablets, microcapsules, microspheroids, granules, crystals or pellets, optionally compacted in a tablet, filled in a capsule or suspended in a suitable liquid.

The pharmaceutical formulations according to the present invention are particularly preferably suitable for oral, intravenous, intramuscular, subcutaneous, intrathecal, epidural, buccal, sublingual, pulmonal, rectal, transdermal, nasal or intracerebroventricular application, more particularly for oral, intravenous or intraperitoneal application.

In one embodiment of the present invention the pharmaceutical formulation comprises at least one of the components (A) and (B) of the active substance combination at least partially in a sustained-release form.

By incorporating one or both of these components (A) and (B) at least partially or completely in a sustained-release form it is possible to extend the duration of their effect, allowing for the beneficial effects of such a sustained-release form, e.g. the maintenance of even concentrations in the blood.

Suitable sustained-release forms as well as materials and methods for their preparation are known to those skilled in the art, e.g. from the tables of contents from “Modified-Release Drug Delivery Technology”, Rathbone, M. J. Hadgraft, J. and Roberts, M. S. (Eds.), Marcel Dekker, Inc., New York (2002); “Handbook of Pharmaceutical Controlled Release Technology”, Wise, D. L. (Ed.), Marcel Dekker, Inc. New York, (2000); “Controlled Drug Delivery”, Vol. I, Basic Concepts, Bruck, S. D. (Ed.), CRC Press Inc., Boca Raton (1983) and from Takada, K. and Yoshikawa, H., “Oral Drug delivery”, Encyclopedia of Controlled Drug Delivery, Mathiowitz, E. (Ed.), John Wiley & Sons, Inc., New York (1999), Vol. 2, 728-742; Fix, J., “Oral drug delivery, small intestine and colon”, Encylopedia of Controlled Drug Delivery, Mathiowitz, E. (Ed.), John Wiley & Sons, Inc., New York (1999), Vol. 2, 698-728. The respective descriptions are incorporated by reference and are part of the disclosure.

If the pharmaceutical formulation according to the present invention comprises at least one of the components (A) and (B) at least partially in a sustained-release form, said sustained release may preferably be achieved by the application of at least one coating or provision of a matrix comprising at least one sustained-release material.

The sustained-release material is preferably based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

The water-insoluble polymers used to produce a sustained-release material are preferably based on an acrylic resin, which is preferably selected from the group of poly(meth)acrylates, particularly preferably poly(C₁₋₄)alkyl(meth)acrylates, poly(C₁₋₄dialkylamino(C₁₋₄alkyl(meth)acrylates and/or copolymers or mixtures thereof, and very particularly preferably copolymers of ethyl acrylate and methyl methacrylate with a monomer molar ratio of 2:1 (Eudragit)NE30D®), copolymers of ethyl acrylate, methyl methacrylate and trimethylammonium ethyl methacrylate-chloride with a monomer molar ratio of 1:2:0.1 (Eudragit RS®), copolymers of ethyl acrylate, methyl methacrylate and trimethylammonium ethyl methacrylate-chloride with a monomer molar ratio of 1:2:0.2 (Eudragit RL®), or a mixture of at least two of the above-mentioned copolymers. These coating materials are commercially available as 30 wt. % aqueous latex dispersions, i.e. as Eudragit RS30D®, Eudragit NE30D® or Eudragit RL30D®, and may also be used as such for coating purposes.

In another embodiment, the sustained-release material is based on water-insoluble cellulose derivatives, preferably alkyl celluloses, particularly preferably ethyl cellulose, or cellulose esters, e.g. cellulose acetate. Aqueous ethyl cellulose dispersions are commercially available, for example, under the trademarks Aquacoat® or Surelease®.

As natural, semisynthetic or synthetic waxes, fats or fatty alcohols, the sustained-release material may be based on carnauba wax, beeswax, glycerol monostearate, glycerol monobehenate, glycerol ditripalmitostearate, microcrystalline wax, cetyl alcohol, cetylstearyl alcohol or a mixture of at least two of these components.

The afore mentioned polymers of the sustained-release material may also comprise a conventional, physiologically acceptable plasticizer in amounts known to those skilled in the art.

Examples of suitable plasticizers are lipophilic diesters of a C₆-C₄₀ aliphatic or aromatic dicarboxylic acid and a C₁-C₈ aliphatic alcohol, e.g. dibutyl phthalate, diethyl phthalate, dibutyl sebacate or diethyl sebacate, hydrophilic or lipophilic citric acid esters, e.g. triethyl citrate, tributyl citrate, acetyltributyl citrate or acetyltriethyl citrate, polyethylene glycols, propylene glycol, glycerol esters, e.g. triacetin, Myvacet® (acetylated mono- and diglycerides, C₂₃H₄₄O₅ to C₂₅H₄₇O₇), medium-chain triglycerides (Miglyol®), oleic acid or mixtures of at least two of said plasticizers.

Aqueous dispersions of Eudragit RS® and optionally Eudragit RL® preferably contain triethyl citrate. The sustained-release material may comprise one or more plasticisers in amounts of, for example, 5 to 50 wt. % based on the amount of polymer(s) used.

The sustained-release material may also contain other conventional auxiliary substances known to those skilled in the art, e.g. lubricants, coloured pigments or surfactants.

The pharmaceutical formulation of the present invention may also comprise at least one of the components (A) and (B) covered by an enteric coating form which dissolves as a function of pH. Because of this coating, part or all of the pharmaceutical formulation can pass through the stomach undissolved and the components (A) and/or (B) are only released in the intestinal tract. The enteric coating preferably dissolves at a pH of between 5 and 7.5.

The enteric coating may be based on any enteric material known to those skilled in the art, e.g. on methacrylic acid/methyl methacrylate copolymers with a monomer molar ratio of 1:1 (Eudragit L®), methacrylic acid/methyl methacrylate copolymers with a monomer molar ratio of 1:2 (Eudragit S®), methacrylic acid/ethyl acrylate copolymers with a monomer molar ratio of 1:1 (Eudragit L30D-55®), methacrylic acid/methyl acrylate/methyl methacrylate copolymers with a monomer molar ratio of 7:3:1 (Eudragit FS®), shellac, hydroxypropyl methyl cellulose acetate-succinates, cellulose acetate-phthalates or a mixture of at least two of these components, which can optionally also be used in combination with the above-mentioned water-insoluble poly(meth)acrylates, preferably in combination with Eudragit NE30D® and/or Eudragit RL® and/or Eudragit RS®.

The coatings of the pharmaceutical formulations of the present invention may be applied by the conventional processes known to those skilled in the art, e.g. from Johnson, J. L., “Pharmaceutical tablet coating”, Coatings Technology Handbook (Second Edition), Satas, D. and Tracton, A. A. (Eds), Marcel Dekker, Inc. New York, (2001), 863-866; Carstensen, T., “Coating Tablets in Advanced Pharmaceutical Solids”, Swarbrick, J. (Ed.), Marcel Dekker, Inc. New York (2001), 455-468; Leopold, C. S., “Coated dosage forms for colon-specific drug delivery”, Pharmaceutical Science & Technology Today, 2(5), 197-204 (1999), Rhodes, C. T. and Porter, S. C., Coatings, in Encyclopedia of Controlled Drug Delivery. Mathiowitz, E. (Ed.), John Wiley & Sons, Inc., New York (1999), Vol. 1, 299-311. The respective descriptions are incorporated by reference and are part of the disclosure.

In another embodiment, the pharmaceutical formulation of the present invention contains one or both of components (A) and (B) not only in sustained-release form, but also in non-sustained-release form. By combination with the immediately released form, a high initial dose can be achieved for the rapid onset of the beneficial effect. The slow release from the sustained-release form then prevents the beneficial effect from diminishing. Such a pharmaceutical formulation is particularly useful for the treatment of acute health problems.

This may be achieved, for example, by a pharmaceutical formulation having at least one immediate-release coating comprising at least one of the components (A) and (B) to provide for rapid onset of the beneficial effect after administration to the patient.

Administered dosages for acetylcholinesterase inhibitors and compounds with 5-HT₆ receptor affinity are in accordance with dosages and scheduling regimens practised by those of skill in the art. General guidance for appropriate dosages of all pharmacological agents used in the present methods is provided in Goodman and Gilman's The Pharmacological Basis of Therapeutics, 10^{th} Ed., Hardman, Limbird and Goodman-Gilman, Eds., McGraw-Hill (2001) and in a Physicians' Desk Reference (PDR), for instance, in the 57^{th} or 58^{th} Eds., Thomson PDR (2003 or 2004). Published dosages of acetylcholinesterase inhibitors and compounds for with 5-HT₆ receptor affinity are for indications distinct from treatment of obesity. Typically, efficacious dosages of acetylcholinesterase inhibitors and compounds with 5-HT₆ receptor affinity for practising the present invention can be equal to or less than (e.g., about 25, 50, 75 or 100%) the dosages published for other indications, such as for Alzheimer's disease and depression, respectively.

The present invention also relates to the treatment the aforementioned disorders and/or diseases with a combination of at least one compound with 5-HT₆ receptor affinity and at least one cholinesterase inhibitor which may be administered separately, therefore the invention also relates to combining separate pharmaceutical compositions into a kit form. The kit, according to this invention, comprises two separate pharmaceutical compositions: a first unit dosage form comprising a prophylactically or therapeutically effective amount of at least one cholinesterase inhibitor, or a pharmaceutically acceptable salt or ester thereof, and a pharmaceutically acceptable carrier or diluent in a first unit dosage form, and a second unit dosage form comprising a prophylactically or therapeutically effective amount of at least one compound with 5-HT₆ receptor affinity, or a pharmaceutically acceptable salt or ester thereof, and a pharmaceutically acceptable carrier or diluent in a second unit dosage form. The kit further comprises a container. Such kits are especially suited for the delivery of solid oral forms such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days or time in the treatment schedule in which the dosages can be administered.

### Examples

Pharmacological Methods:

I) Binding to Serotonin Receptor 5-HT₆

Cell membranes of HEK-293 cells expressing the 5HT₆-human recombinant receptor were supplied by Receptor Biology. In said membranes the receptor concentration is 2.18 pmol/mg protein and the protein concentration is 9.17 mg/ml. The experimental protocol follows the method of B. L. Roth et al. [B. L. Roth, S. C. Craigo, M. S. Choudhary, A. Uluer, F. J. Monsma, Y. Shen, H. Y. Meltzer, D. R. Sibley: Binding of Typical and Atypical Antipsychotic Agents to 5-Hydroxytryptamine-6 and Hydroxytryptamine-7 Receptors. The Journal of Pharmacology and Experimental Therapeutics, 1994, 268, 1403] with the following slight changes. The respective part of the literature description is hereby incorporated by reference and forms part of the disclosure.

The commercial membrane is diluted (1:40 dilution) with the binding buffer: 50 mM Tris-HCl, 10 mM MgCl₂, 0.5 mM EDTA (pH 7.4). The radioligand used is [³H]-LSD at a concentration of 2.7 nM with a final volume of 200 μl. Incubation is initiated by adding 100 μl of membrane suspension, (≈22.9 μg membrane protein), and is prolonged for 60 minutes at a temperature of 37° C. The incubation is ended by fast filtration in a Brandel Cell Harvester through fiber glass filters made by Schleicher & Schuell G F 3362 pretreated with a solution of polyethylenimine at 0.5%. The filters are washed three times with three milliliters of buffer Tris-HCl 50 mM pH 7.4. The filters are transferred to flasks and 5 ml of Ecoscint H liquid scintillation cocktail are added to each flask. The flasks are allowed to reach equilibrium for several hours before counting with a Wallac Winspectral 1414 scintillation counter. Non-specific binding is determined in the presence of 100 μM of serotonin. Tests were made in triplicate. The inhibition constants (Kᵢ, nM) were calculated by non-linear regression analysis using the program EBDA/LIGAND described in Munson and Rodbard, Analytical Biochemistry, 1980, 107, 220, the respective part of which is hereby incorporated by reference and forms part of the disclosure.

II) Behavioural Assessment of Cognitive Effects of Compounds with 5-HT₆ Receptor Affinity: Novel Object Discrimination (NOD)

Two separate groups of 12 adult male Lister hooded rats are tested in the NOD paradigm using 4 h inter-trial interval (ITI) delay between the familiarisation and choice trials which should ensure impaired discrimination and permit the pro-cognitive effects of the compounds to be evaluated.

In two separate groups of animals compound [1140] (6-chloro-N-(3-(2-(dimethylamino)ethyl)-1H-indol-5-yl)imidazo[2,1-b]thiazole-5-sulfonamide) and donepezil HCl (or vehicle) were administered on four separate occasions, at seven day intervals. The semi-randomised design for drug administration ensures that for each group, each animal receives one of the four treatment combinations once during the study: compound [1140] and donepezil HCl, compound [1140] and vehicle, donepezil HCl and vehicle, vehicle and vehicle. Hence, each animal will serve as its own control within each group to minimise inter-individual variability. Drug pre-treatment occurred 20 min prior to the first NOD trial to ensure optimal drug plasma levels during acquisition and consolidation phases.

To maximise the opportunity to see a synergistic effect, the animals received one dose of donepezil HCl i.p. (0.05 mg/kg for group 1 and 0.1 mg/kg for group 2) in combination with the selected dose of compound [1140] (1 mg/kg) or the vehicle (saline containing 0.5 methylcellulose, 2 ml/kg). The synergism between compound [1140] and donepezil HCl was studied at both 0.05 and 0.1 mg/kg i.p. At the end of the study, seven days after the last drug administration all rats were given donepezil HCl at 0.3 mg/kg i.p. to confirm reversal of natural forgetting and hence the animals were tested in the NOD paradigm at five occasions.

The exact experimental protocol is outlined in detail in:

M. V. King et al, 5-HT6 receptor antagonist reverse delay-dependent deficits in novel object discrimination by enhancing consolidation—an effect sensitive to NMDA receptor antagonism, Neuropharmacology 2004, 47, 195-204 and Woolley et al. Reversal of cholinergic-induced deficit in a rodent model of recognition memory by the selective 5-HT6 receptor antagonist, Ro 04-6790; Psychopharmacology 2003, 170, 358-367.

The respective parts of the literature are hereby incorporated by reference and form part of the present disclosure.

Pharmacological Data:

The behavioural assessment of cognitive effects of compounds with 5-HT₆ receptor affinity with the novel object discrimination (NOD) test was carried out as described above.

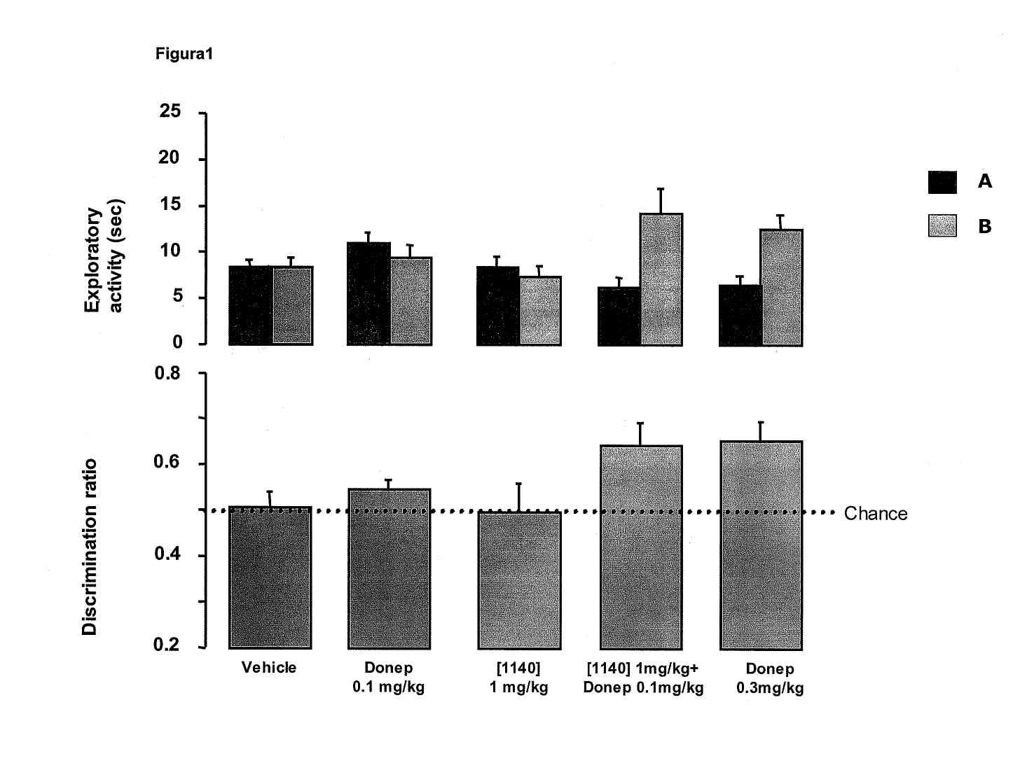

The results are depicted in FIG. 1. A denotes familiar and B denotes novel objects in this figure.

FIG. 1. shows that the acetylcholinesterase inhibitor donepezil potentiates the action of the compound with 5-HT₆ receptor affinity, compound [1140]. Thus, the discrimination ratio is significantly greater when compound [1140] (1 mg/kg) is administered in combination with donepezil hydrochloride [compound [1140] (1 mg/kg)+donepezil hydrochloride(0.1 mg/kg)]. FIG. 1. also shows that the combination of an amount of donepezil hydrochloride which does not have any effect in the NOD test by itself and an amount of compound [1140] which does not have any effect in the NOD test by itself can be combined to yield an active substance combination which shows a significant effect in the NOD test.

1. An active substance combination that comprises:
as component (A) at least one compound with 5-HT₆ receptor affinity, and
as component (B) at least one cholinesterase inhibitor,
wherein an active substance combination comprising as component (A) the 5-HT₆ antagonist SB271046 and as component (B) the cholinesterase inhibitor donepezil hydrochloride is excluded.

2. The combination according to claim 1, wherein component (A) has a Kᵢ value for binding to the 5-HT₆-receptor of below or equal to 1 μM.

3. The combination according to claim 1, wherein component (A) acts as agonist or inverse agonist of the 5-HT₆ receptor, preferably the compound with 5-HT₆ receptor affinity acts as agonist of the 5-HT₆ receptor.

4. The combination according to claims 1, wherein component (B) has an IC₅₀ for the inhibition of acetylcholinesterase in erythrocytes below or equal to 1 μM.

5. The combination according to claim 1, wherein the cholinesterase inhibitor is selected from the group consisting of reversible cholinesterase inhibitors and pseudo-reversible cholinesterase inhibitors.

6. The combination according to claim 1, wherein component (A) is selected from the group consisting of the benzoxazinone-derived sulfonamide compounds of general formula (Ia) 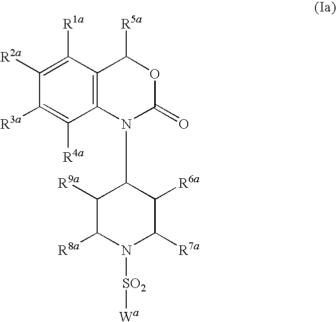
wherein
R^{1a}, R^{2a}, R^{3a} and R^{4a}, independently of one another, each represent a hydrogen atom; halogen; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl- or heteroaryl radical, which may be bonded via an unsubstituted or at least mono-substituted alkylene group and/or which may be condensed with an unsubstituted or at least mono-substituted mono- or bicyclic ringsystem; nitro; cyano; —O—R^{10a}; —O—(C═O)—R^{11a}; —(C═O)—OR^{11a}; —SR^{12a}; —SOR^{12a}; —SO₂R^{12a}; —NH—SO₂R^{12a}; —SO₂NH₃ or —NR^{13a}R^{14a};
R^{5a} represents a hydrogen atom; an unbranched or branched, saturated or unsaturated, unsubstituted or at least mono-substituted aliphatic radical or a saturated or unsaturated, unsubstituted or at least mono-substituted, optionally at least one heteroatom as ring member containing cycloaliphatic radical;
R^{6a}, R^{7a}, R^{8a}, R^{9a}, independetly of one another, each represent a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical; a cyano group or a —C(═O)—OR^{15a} moiety;
W^{a} represents an unbranched or branched, saturated or unsaturated, unsubstituted or at least mono-substituted aliphatic radical;
a saturated or unsaturated, unsubstituted or at least mono-substituted, optionally at least one heteroatom as ring member containing cycloaliphatic radical, which may be bonded via an optionally mono-substituted alkylene group and/or which may be condensed with an unsubstituted or at least mono-substituted mono- or bicyclic ring system;
an unsubstituted or at least mono-substituted aryl or heteroaryl radical, which may be bonded via an unsubstituted or at least mono-substituted alkylene or alkenylene group and/or which may be condensed with an unsubstituted or at least mono-substituted mono- or bicyclic ring system;
a —NR^{16a}R^{17a} moiety, or
a —C(═O)—R^{18a} moiety;
R^{10a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;
R^{11a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;
R^{12a} represents an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;
R^{13a} and R^{14a}, independently of one another, each represent a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical, which may be bonded via an unsubstituted or at least mono-substituted alkylene group and/or which may be condensed with an unsubstituted or at least mono-substituted mono- or bicyclic ring system; or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;
or R^{13a} and R^{14a} together with the bridging nitrogen atom form a saturated, unsaturated or aromatic heterocyclic ring, which is unsubstituted or at least mono-substituted and/or which may contain at least one further heteroatom as a ring member;
R^{15a} represents a hydrogen atom; an unbranched or branched, saturated or unsaturated, unsubstituted or at least mono-substituted aliphatic radical; a saturated or unsaturated, unsubstituted or at least mono-substituted, optionally at least one heteroatom as ring member containing cycloaliphatic radical or an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system;
R^{16a} represents an unbranched or branched, saturated or unsaturated, unsubstituted or at least mono-substituted aliphatic radical;
R^{17a} represents an unbranched or branched, saturated or unsaturated, unsubstituted or at least mono-substituted aliphatic radical, and
R^{18a} represents an unsubstituted or at least mono-substituted aryl radical;
optionally in form of one of its stereoisomers, preferably enantiomers or diastereomers, its racemate or in form of a mixture of at least two of its stereoisomers, preferably enantiomers or diastereomers, in any mixing ratio, or a physiologically acceptable salt thereof, or a solvate, respectively;
indole-derived sulfonamide compounds of general formula (Ib) 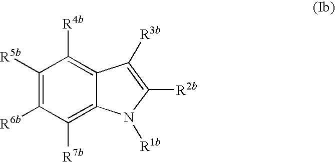
wherein
R^{1b} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical,
which may be bonded via a linear or branched alkylene group and/or which may be condensed with an unsubstituted or at least mono-substituted mono- or bicyclic ring system; an unsubstituted or at least mono-substituted aryl or heteroaryl radical, which may be bonded via a linear or branched alkylene group and/or which may be condensed with an unsubstituted or at least mono-substituted mono- or bicyclic ring system; a —(CH₂)_{mb}—NR^{13b}R^{14b} moiety with mb=0, 1, 2, 3, 4 or 5; a —C(═O)—R^{8b} moiety; a —S(═O)₂—R^{9b} moiety; or a —S(═O)₂—C(H)A^{b}B^{b} moiety;
R^{2b} represents a hydrogen atom; —F; —Cl; —Br; —I; —NO₂; —NH₂; —SH; —OH; —CN; —C(═O)—OH; —O—R^{10b}; —C(═O)—OR^{12b}; a linear or branched, saturated or unsaturated, unsubstituted or at least mono-substituted, optionally at least one heteroatom as a chain member containing aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R^{3b} represents a hydrogen atom; —F; —Cl; —Br; —I; —NO₂; —CN; —O—R^{10b}; —S—R^{11b}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; a —CH(OC₂H₅)—CH₂—NR^{13b}R^{14b} moiety or a —(CH₂)_{nb}—NR^{13b}R^{14b} moiety with nb=0, 1, 2, 3, 4 or 5; a —S(═O)₂—R^{9b} moiety; a —S(═O)₂—C(H)A^{b}B^{b} moiety; or a —C(═O)—(CH₂)_{pb}—C(═O)—N-D^{b}E^{b} moiety with pb=0, 1, 2, 3, 4 or 5;
R^{4b}; R^{5b}; R^{6b} and R^{7b}, independently of one another, each represent a hydrogen atom; —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{8b}; —S(═O)₂—R^{9b}; —O—R^{10b}; —S—R^{11b}; C(═O)—OR^{12b}; —N(R^{15b})—S(═O)₂R^{16b}; —NH—R^{17b}; —NR^{18b}R^{19b}; —C(═O)—NHR^{20b}, —C(═O)—NR^{21b}R^{22b}; —S(═O)₂—NHR^{23b}; —S(═O)₂—NR^{24b}R^{25b}; —O—C(═O)—R^{26b}; —NH—C(═O)—R^{27b}; —NR^{28b}—C(═O)—R^{29b}; NH—C(═O)—O—R^{30b}; NR^{31b}—C(═O)—O—R^{32b}; —S(═O)₂—O—R^{33b}; a halogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene group;
with the proviso that at least one of the substituents R^{4b}, R^{5b}, R^{6b} and R^{7b} represents a —N(R^{15b}—S(═O)₂—R^{16b} moiety;
R^{8b}, R^{12b}, R^{17b}, R^{18b}, R^{19b}, R^{20b}, R^{21b}, R^{22b}, R^{23b}, R^{24b}, R^{25b}, R^{26b}, R^{27b}, R^{28b}, R^{29b}, R^{30b}, R^{31b}, R^{32b} and R^{33b}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene, alkenylene or alkinylene group;
R^{9b} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{10b} and R^{11b}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{13b} and R^{14b}, independently of one another, each represent a hydrogen atom; or a linear or branched, saturated or unsaturated, unsubstituted or at least mono-substituted aliphatic radical;
R^{13b} and R^{14b} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{15b} represents a hydrogen atom; a linear or branched, saturated or unsaturated, unsubstituted or at least mono-substituted aliphatic radical or a —S(═O)₂—R^{16b} moiety;
R^{16b} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
A^{b} and B^{b} together with the bridging carbon form an unsubstituted or at least mono-substituted, saturated or unsaturated cycloaliphatic ring which may contain at least one further heteroatom as a ring member and/or which may be condensed with an unsubstituted or at least mono-substituted mono- or bicyclic ring system;
D^{b} and E^{b} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system; or
D^{b} and E^{b}, independently of one another, each represent a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively; indazolyl- and (2,3)-dihydro-indolyl-derived sulfonamide compounds of general formula (Ic) 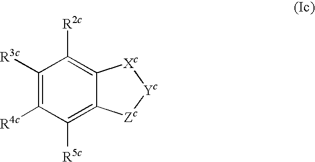
wherein
X^{c}—Y^{c} from left to right represents CR^{1c}═N and Z^{c} is N[(CH₂)_{nc}R^{6c}] or
X^{c}—Y^{c} from left to right represents CR^{7c}═N, Z^{c} is NH, R^{7c} represents the following moiety 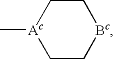
A^{c} represents CH or N and B^{c} represents NR^{8c}, O or S;
X^{c}—Y^{c} from left to right represents C[(CH₂)_{nc}R^{9c}═N and Z^{c} is NR^{10c} or
X^{c}—Y^{c} represents CH₂—CH₂ and Z^{c} is N[(CH₂)_{nc}R^{11c}];
nc is 0, 1, 2, 3 or 4;
R^{1c} represents a hydrogen atom; NO₂; —NH₂; —SH; —OH; —CN; —C(═O)—R^{12c}; —OR^{13c}; —SR^{14c}; —F; —Cl, —Br; —I; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{2c}, R^{3c}, R^{4c} and R^{5c}, independently of one another, each represent a hydrogen atom; —NO₂; —NH₂; —SH; —OH; —CN; —C(═O)—H; —C(═O)—R^{12c}; —OR^{13c}; —SR^{14c}; —N(R^{15c})—S(═O)₂—R^{16c}; —NH—R^{17c}; —NR^{18c}R^{19c}; —F; —Cl, —Br; —I; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
with the proviso that at least one of the substituents R^{2c}, R^{3c}, R^{4c} and R^{5c} represents a —N(R^{15c})—S(═O)₂—R^{16c} moiety;
R^{6c}, R^{9c} and R^{11c}, independently of one another, each represent a —NR^{20c}R^{21c} radical or
a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{8c} represents —C(═O)—R^{22c}; a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{10c} represents a linear or branched, saturated or unsaturated, unsubstituted or at least mono-substituted aliphatic radical-; or a —S(═O)₂—R^{23c} moiety;
R^{12c}, R^{13c}, R^{14c}, R^{17c}, R^{18c} and R^{19c}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{15c} represents a hydrogen atom; a linear or branched, saturated or unsaturated, unsubstituted or at least mono-substituted aliphatic radical; or a —S(═O)₂—R^{24c} moiety;
R^{16c} and R^{24c}, independently of one another, each represent an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{20c} and R^{21c}, independently of one another, each represent a hydrogen atom; or a linear or branched, saturated or unsaturated, unsubstituted or at least mono-substituted aliphatic radical;
or R^{20c} and R^{21c} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{22c} represents a linear or branched, saturated or unsaturated, unsubstituted or at least mono-substituted aliphatic radical; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system; and
R^{23c} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
phenyl-piperazine-derived compounds of general formula (Id) 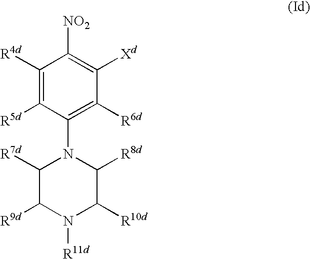
wherein
X^{d} represents a —NR^{1d}R^{2d} moiety t or a —OR^{3d} moiety;
R^{1d} represents a linear or branched, saturated or unsaturated, unsubstituted or at least mono-substituted aliphatic radical;
an unsubstituted or at least mono-substituted radical selected from the group consisting of adamantyl, bicyclo[2.2.1]heptyl and bicyclo[3.1.1]heptyl, which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);
a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);
or a —C(═O)—R^{12d} moiety;
R^{2d} represents a hydrogen atom or a linear or branched, saturated or unsaturated, unsubstituted or at least mono-substituted aliphatic radical; or
R^{1d} and R^{2d} together with the bridging nitrogen form an optionally at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{3d} represents or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
R^{4d}, R^{5d} and R^{6d}, independently of one another, each represent a hydrogen atom or a halogen atom; or
R^{4d} and R^{5d} together with the bridging carbon atoms form an unsubstituted 5- or 6-membered heterocyclic ring which contains 1, 2 or 3 heteroatom(s) independently selected from the group consisting of nitrogen, oxygen and sulfur as ring member(s) and which together with the phenyl ring which it is fused with forms a 9- or 10-membered bicyclic aromatic ring system;
R^{7d} and R^{8d}, independently of one another, each represent a hydrogen atom or a linear or branched, saturated or unsaturated, unsubstituted or at least mono-substituted aliphatic radical;
R^{9d} and R^{10d}, independently of one another, each represent a hydrogen atom or a linear or branched, saturated or unsaturated, unsubstituted or at least mono-substituted aliphatic radical;
R^{11d} represents a hydrogen atom; or a linear or branched, saturated or unsaturated, unsubstituted or at least mono-substituted aliphatic radical, which may contain 1, 2 or 3 heteroatom(s) independently selected from the group consisting of nitrogen, oxygen and sulfur as chain member(s);
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
-a-C(═O)—R^{13d} moiety or a —S(═O)₂—R^{14d} moiety;
R^{12d} represents a linear or branched, saturated or unsaturated, unsubstituted or at least mono-substituted aliphatic radical;
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group; and
R^{13d} and R^{14d}, independently of one another, each represent a linear or branched, saturated or unsaturated, unsubstituted or at least mono-substituted aliphatic radical; or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and phenyl-piperazine-derived compounds of general formula (Ie) 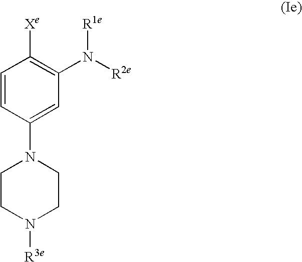
wherein
X^{e} represents —CN, —C(═O)—OH, —C(═O)—OR^{4e}, —O—R^{5e}, —NH₂, —NR^{6e}—C(═O)—R^{7e}, —NH—S(═O)₂—R^{8e} or —NH—R^{9e};
R¹ represents a hydrogen atom;
a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group which may contain 1, 2 or 3 heteroatom(s) independently selected from the group consisting of nitrogen, oxygen and sulfur as chain member(s);
R^{2e} represents a hydrogen atom or a —C(═O)—R^{10e} moiety; or
R^{1e} and R^{2e} together with the bridging nitrogen form a nitro (NO₂)-group or
an unsubstituted or at least mono-substituted 5- or 6-membered heteroaryl radical which may be condensed with an unsubstituted or at least mono-substituted mono- or bicyclic ring system;
R^{3e} represents a linear or branched, saturated or unsaturated, unsubstituted or at least mono-substituted aliphatic radical;
R^{4e} represents a linear or branched, saturated or unsaturated, unsubstituted or at least mono-substituted aliphatic radical;
R^{5e} represents a linear or branched, saturated or unsaturated, unsubstituted or at least mono-substituted aliphatic radical; or
an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
R^{6e} represents a hydrogen atom or
an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
R^{7e} represents a linear or branched, saturated or unsaturated, unsubstituted or at least mono-substituted aliphatic radical;
R^{8e} represents a linear or branched, saturated or unsaturated, unsubstituted or at least mono-substituted aliphatic radical;
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
R^{9e} represents a linear or branched, saturated or unsaturated, unsubstituted or at least mono-substituted aliphatic radical;
or an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group; and
R^{10e} represents a linear or branched, saturated or unsaturated, unsubstituted or at least mono-substituted aliphatic radical;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and tetrahydroisoquinoline-derived sulfonamide compounds of general formula (If) 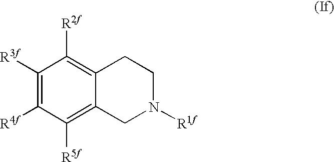
wherein
R^{1f} represents a hydrogen atom; a —C(═O)—OR^{37f} moiety;
a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{2f}, R^{3f}, R^{4f} and R^{5f}, independently of one another, each represent a hydrogen atom; F, Cl, Br, I, —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{6f}; —S(═O)—R^{7f}; —S(═O)₂—R^{7f}; —OR^{8f}; —SR^{9f}; —C(═O)—OR^{10f}; —N(R^{11f})—S(═O)₂—R^{12f}; —NR^{13f}R^{14f}; —NH—R^{15f}; —C(═O)—NR^{16f}R^{17f}; C(═O)—NHR^{18f};
a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;
with the proviso that at least one of the substituents R^{2f}, R^{3f}, R^{4f} and R^{5f} represents a —N(R^{11f})—S(═O)₂—R^{12f} moiety;
R^{6f}, R^{7f}, R^{8f}, R^{9f}, R^{10f}, R^{13f}, R^{14f}, R^{15f}, R^{16f}, R^{17f} and R^{18f},
independently of one another, each represent a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;
R^{11f} represents a hydrogen atom or a linear or branched, saturated or unsaturated, unsubstituted or at least mono-substituted aliphatic radical;
R^{12f}represents a phenyl radical of general formula (Af), 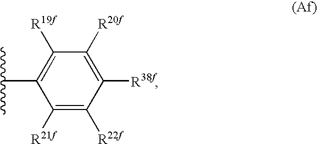
wherein
R^{19f}, R^{20f}, R^{21f}, R^{22f} and R^{38f}, independently of one another, each represent a hydrogen atom; F, Cl, Br, I, —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{23f}; —S(═O)—R^{24f}; —S(═O)₂—R^{24f}; —OR^{25f}; —SR^{26f}; —C(═O)—OR^{27f}; —N(R^{28f})—S(═O)₂—R^{29f}; —NH—S(═O)₂—R^{30f}; —NR^{31f}R^{32f}; —NH—R^{33f}; —C(═O)—NHR^{34f}; —C(═O)—NR^{35f}R^{36f}; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system;
an unsubstituted or at least mono-substituted aryl or heteroaryl radical, which may be condensed with an unsubstituted or at least mono-substituted mono- or bicyclic ring system and/or which may be bonded via a linear or branched, unsubstituted or at least mono-substituted alkylene, alkenylene or alkinylene group;
or a saturated or unsaturated, unsubstituted or at least mono-substituted, optionally at least one heteroatom as a ring member containing cycloaliphatic radical, which may be condensed with an unsubstituted or at least mono-substituted saturated, unsaturated or aromatic mono- or bicyclic ring system;
R^{23f}, R^{27f}, R^{28f}, R^{29f} and R^{30f}, independently of one another, each represent a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;
R^{24f}, R^{26f}, R^{31f}, R^{32f} and R^{33f}, each represent a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be condensed with an unsubstituted or at least mono-substituted saturated or unsaturated, but not aromatic, mono- or bicyclic ring system;
R^{25f}, R^{34f}, R^{35f} and R^{36f}, represents a linear or branched, saturated or unsaturated, unsubstituted or at least mono-substituted aliphatic radical;
and R^{37f} represents a linear or branched, saturated or unsaturated, unsubstituted or at least mono-substituted aliphatic radical;
a saturated or unsaturated, unsubstituted or at least mono-substituted, optionally at least one heteroatom as a ring member containing cycloaliphatic radical, which may be bonded via a linear or branched alkylene, alkenylene or alkinylene group and which may be condensed with an unsubstituted or at least mono-substituted saturated, unsaturated or aromatic mono- or bicyclic ring system;
or an unsubstituted or at least mono-substituted aryl or heteroaryl radical, which may be bonded via a linear or branched alkylene, alkenylene or alkinylene group and which may be condensed with an unsubstituted or at least mono-substituted saturated or unsaturated, but not aromatic, mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a salt thereof, or a corresponding solvate thereof.

7. The combination according to claim 6, wherein the indole-derived sulfonamide is selected from the group consisting of compounds of general formula (Ih) 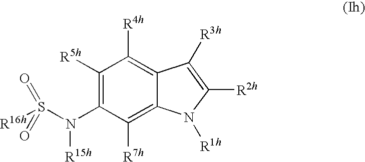
wherein
R^{1h} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₕ—NR^{13h}R^{14h} moiety with mh=0, 1, 2, 3, 4 or 5;
R^{2h} represents a hydrogen atom; —F; —Cl; —Br; —I; —NO₂; —CN; —O—R^{10h}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R^{3h} represents a hydrogen atom; —F; —Cl; —Br; —I; —NO₂; —CN; —O—R^{10h}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₕ—NR^{13h}R^{14h} moiety with nh=0, 1, 2, 3, 4 or 5;
R^{4h}, R^{5h} and R^{7h}, independently of one another, each represent a hydrogen atom; —NO₂; —CN; —O—R^{10h}; —C(═O)—OR^{12h}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{10h} represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{13h} and R^{14h}, independently of one another, each represent a hydrogen atom; or a linear or branched, saturated or unsaturated, unsubstituted or at least mono-substituted aliphatic radical; or
R^{13h} and R^{14h} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{15h} represents a hydrogen atom; a linear or branched, saturated or unsaturated, unsubstituted or at least mono-substituted aliphatic radical or a —S(═O)₂—R^{16h} moiety;
and R^{16h} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and compounds of general formula (Ik) 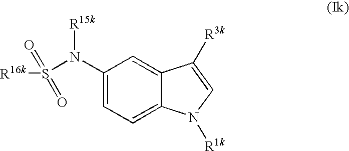
wherein
R^{1k} represents a hydrogen atom; a linear or branched, saturated or unsaturated, unsubstituted or at least mono-substituted aliphatic radical; an unsubstituted or at least mono-substituted phenyl radical or an unsubstituted or at least mono-substituted benzyl radical;
R^{3k} represents a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; or a —(CH₂)ₙₖ—NR^{13k}R^{14k} moiety with nk=0, 1, 2, 3, 4 or 5;
R^{13k} and R^{14k}, independently of one another, each represent a hydrogen atom; or a linear or branched, saturated or unsaturated, unsubstituted or at least mono-substituted aliphatic radical; or
R^{13k} and R^{14k} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{15k} represents a hydrogen atom; or a linear or branched, saturated or unsaturated, unsubstituted or at least mono-substituted aliphatic radical;
and R^{16k} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
compounds of general formula (Im) 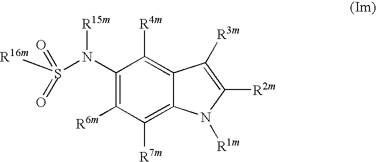
wherein
R^{1m} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₘ—NR^{13m}R^{14m} moiety with mm=0, 1, 2, 3, 4 or 5;
R^{2m} represents a hydrogen atom; —F; —Cl; —Br; —I; —NO₂; —CN; —O—R^{10m}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R^{3m} represents a hydrogen atom; —F; —Cl; —Br; —I; —NO₂; —CN; —O—R^{10m}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₘ—NR^{13m}R^{14m} moiety with nm=0, 1, 2, 3, 4 or 5;
R^{4m}, R^{6m} and R^{7m}, independently of one another, each represent a hydrogen atom; —NO₂; —CN; —O—R^{10m}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{10m} represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{13m} and R^{14m}, independently of one another, each represent a hydrogen atom; or a linear or branched, saturated or unsaturated, unsubstituted or at least mono-substituted aliphatic radical; or
R^{13m} and R^{14m} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{15m} represents a hydrogen atom; a linear or branched, saturated or unsaturated, unsubstituted or at least mono-substituted aliphatic radical or a —S(═O)₂—R^{16m} moiety;
and R^{16m} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and compounds of general formula (In) 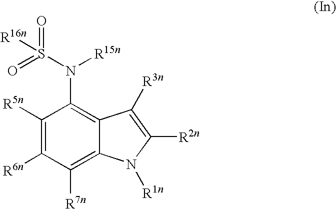
wherein
R¹ⁿ represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₙ—NR¹³ⁿR¹⁴ⁿ moiety with mn=0, 1, 2, 3, 4 or 5;
R²ⁿ represents a hydrogen atom; —F; —Cl; —Br; —I; —NO₂; —CN; —O—R¹⁰ⁿ; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R³ⁿ represents a hydrogen atom; —F; —Cl; —Br; —I; —NO₂; —CN; —O—R¹⁰ⁿ; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; or a —(CH₂)ₙₙ—NR¹³ⁿR¹⁴ⁿ moiety with nn=0, 1, 2, 3, 4 or 5;
R⁵ⁿ, R⁶ⁿ and R⁷ⁿ, independently of one another, each represent a hydrogen atom; —NO₂; —CN; —O—R¹⁰ⁿ; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R¹⁰ⁿ represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R¹³ⁿ and R¹⁴ⁿ, independently of one another, each represent a hydrogen atom; or a linear or branched, saturated or unsaturated, unsubstituted or at least mono-substituted aliphatic radical; or
R¹³ⁿ and R¹⁴ⁿ together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R¹⁵ⁿ represents a hydrogen atom; a linear or branched, saturated or unsaturated, unsubstituted or at least mono-substituted aliphatic radical or a —S(═O)₂—R¹⁶ⁿ moiety;
R¹⁶ⁿ represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and compounds of general formula (Io) 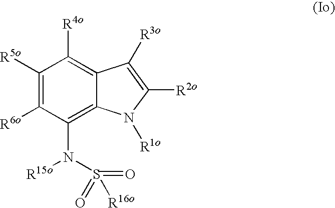
wherein
R^{1o} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or a —(CH₂)ₘₒ—NR^{13o}R^{14o} moiety with mo=0, 1, 2, 3, 4 or 5;
R^{2o} represents a hydrogen atom; —F; —Cl; —Br; —I; —NO₂; —CN; —O—R^{10o}; a linear or branched, saturated or unsaturated, unsubstituted or at least mono-substituted aliphatic radical; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R^{3o} represents a hydrogen atom; —F; —Cl; —Br; —I; —NO₂; —CN; —O—R^{10o}; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; an unsubstituted or at least mono-substituted aryl or heteroaryl radical, which may be bonded via an unsubstituted or at least mono-substituted alkylene group and/or which may be condensed with an unsubstituted or at least mono-substituted mono- or bicyclic ring system; a —CH(OC₂H₅)—CH₂—NR^{13o}R^{14o} moiety or a —(CH₂)ₙₒ—NR^{13o}R^{14o} moiety with no=0, 1, 2, 3, 4 or 5;
R^{4o}, R^{5o and R}^{6o}, independently of one another, each represent a hydrogen atom; —NO₂; —CN; —O—R^{10o}; a halogen atom; a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{10o} represents a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{13o} and R^{14o}, independently of one another, each represent a hydrogen atom; or a linear or branched, saturated or unsaturated, unsubstituted or at least mono-substituted aliphatic radical; or
R^{13o} and R^{14o} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
R^{15o} represents a hydrogen atom; a linear or branched, saturated or unsaturated, unsubstituted or at least mono-substituted aliphatic radical or a —S(═O)₂—R^{16o} moiety;
and R^{16o} represents an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
compounds of general formula (Ip) 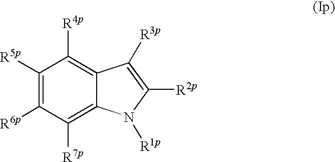
wherein
R^{1p} represents a —S(═O)₂—R^{9p} moiety or a —S(═O)₂—C(H)A^{p}B^{p} moiety;
R^{2p} represents a hydrogen atom; —F; —Cl; —Br; —I; —NO₂; —OH; —CN; —O—R^{10p}; —S—R^{11p}; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system;
R^{3p} represents a saturated or unsaturated, unsubstituted or at least mono-substituted, optionally at least one heteroatom as a ring member containing cycloaliphatic radical, which may be bonded via an unsubstituted or at least mono-substituted alkylene group and/or which may be condensed with an unsubstituted or at least mono-substituted mono- or bicyclic ring system; or a —(CH₂)ₙₚ—NR^{13p}R^{14p} moiety with np=0, 1, 2, 3, 4 or 5;
R^{4p}, R^{5p}, R^{6p}and R^{7p}, independently of one another, each represent a hydrogen atom; —NO₂; —NH₂; —OH; —CN; —C(═O)—R^{8p}; —O—R^{10p}; —S—R^{11p}; —NH—R^{17p}; —NR^{18p}R^{19p}; a halogen atom; a linear or branched, saturated or unsaturated, unsubstituted or at least mono-substituted aliphatic radical; or a saturated or unsaturated, unsubstituted or at least mono-substituted, optionally at least one heteroatom as a ring member containing cycloaliphatic radical, which may be bonded via a linear or branched alkylene group;
R^{8p} represents a hydrogen atom or a linear or branched, saturated or unsaturated, unsubstituted or at least mono-substituted aliphatic radical;
R^{8p}, R^{17p}, R^{18p}and R^{19p}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene, alkenylene or alkinylene group;
R^{9p} represents a linear or branched, saturated or unsaturated, unsubstituted or at least mono-substituted aliphatic radical;
R^{10p} and R^{11p}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{13p} and R^{14p}, independently of one another, each represent a hydrogen atom; or a linear or branched, saturated or unsaturated, unsubstituted or at least mono-substituted aliphatic radical; or
R^{13p} and R^{14p} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system;
A^{p} and B^{p} together with the bridging carbon form an unsubstituted or at least mono-substituted, saturated or unsaturated cycloaliphatic ring which may contain at least one further heteroatom as a ring member and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively;
and compounds of general formula (Iq) 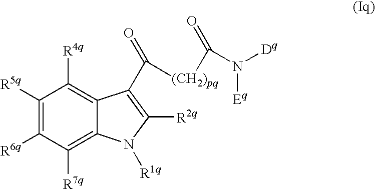
wherein
R^{1q} represents a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical,
which may be bonded via a linear or branched alkylene group and/or which may be condensed with an unsubstituted or at least mono-substituted mono- or bicyclic ring system; an unsubstituted or at least mono-substituted aryl or heteroaryl radical, which may be bonded via a linear or branched alkylene group and/or which may be condensed with an unsubstituted or at least mono-substituted mono- or bicyclic ring system; a —C(═O)—R^{8q} moiety; a —S(═O)₂—R^{9q} moiety;
R^{2q} represents a hydrogen atom; —F; —Cl; —Br; —I; —NO₂; —NH₂; —SH; —OH; —CN; a linear or branched, saturated or unsaturated, unsubstituted or at least mono-substituted, optionally at least one heteroatom as a chain member containing aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched alkylene group and/or which may be condensed with an unsubstituted or at least mono-substituted mono- or bicyclic ring system;
R^{4q}, R^{5q}, R^{6q} and R^{7q}, independently of one another, each represent a hydrogen atom; —NO₂; —NH₂; —SH; —OH; —CN; —C(═O)—OH; —C(═O)—H; —S(═O)₂—OH; —C(═O)—NH₂; —S(═O)₂—NH₂; —C(═O)—R^{8q}; —S(═O)₂—R^{9q}; —O—R^{10q}; —S—R¹¹q; —C(═O)—OR^{12q}; —N(R^{15q})—S(═O)₂—R^{16q}; —NH—R^{17q}; —NR^{18q}R^{19q}; —C(═O)—NHR^{20g}, —C(═O)—NR^{21q}R^{22q}; —S(═O)₂—NHR^{23q}; —S(═O)₂—NR^{24q}R^{25q}; —O—C(═O)—R^{26q}; —NH—C(═O)—R^{27q}; —NR^{28q}—C(═O)—R^{29q}; NH—C(═O)—O—R^{30q}; NR^{31q}—C(═O)—O—R^{32q}; —S(═O)₂—O—R^{33q}; a halogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene group;
with the proviso that at least one of the substituents R^{4q}, R^{5q}, R^{6q} and R^{7q} represents a —N(R^{15q})—S(═O)₂—R^{16q} moiety;
R^{8q}, R^{12q}, R^{17q}, R^{18q}, R^{19q}, R^{20q}, R^{21q}, R^{22q}, R^{23q}, R^{24q}, R^{25q}, R^{26q}, R^{27q}, R^{28q}, R^{29q}, R^{30q}, R^{31q}, R^{32q} and R^{33q}, independently of one another, each represent a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group; or an unsubstituted or at least mono-substituted aryl or heteroaryl radical, which may be bonded via a linear or branched alkylene, alkenylene or alkinylene group;
R^{9q} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
R^{10q} and R^{11q}, independently of one another, each represent a linear or branched, saturated or unsaturated, unsubstituted or at least mono-substituted aliphatic radical; or an unsubstituted or at least mono-substituted aryl or heteroaryl radical, which may be bonded via a linear or branched alkylene group;
R^{15q} represents a hydrogen atom; a linear or branched, saturated or unsaturated, unsubstituted or at least mono-substituted aliphatic radical or a —S(═O)₂—R^{16q} moiety;
R^{16q} represents a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched, unsubstituted or at least mono-substituted alkylene, alkenylene or alkinylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
D^{q} and E^{q} together with the bridging nitrogen form an unsubstituted or at least mono-substituted, saturated, unsaturated or aromatic heterocyclic ring which may contain at least one further heteroatom as a ring member and/or which may be condensed with an unsubstituted or at least mono-substituted mono- or bicyclic ring system; or
D^{q} and E^{q}, independently of one another, each represent a hydrogen atom; a linear or branched, saturated or unsaturated, unsubstituted or at least mono-substituted aliphatic radical; a saturated or unsaturated, unsubstituted or at least mono-substituted, optionally at least one heteroatom as a ring member containing cycloaliphatic radical, which may be bonded via a linear or branched alkylene group and/or which may be condensed with an unsubstituted or at least mono-substituted mono- or bicyclic ring system; or an unsubstituted or at least mono-substituted aryl or heteroaryl radical, which may be bonded via a linear or branched, unsubstituted or at least mono-substituted alkylene, alkenylene or alkinylene group and/or which may be condensed with an unsubstituted or at least mono-substituted mono- or bicyclic ring system;
optionally in form of one of its stereoisomers, preferably enantiomers or diasteromers, a racemate or in form of a mixture of at least two of its stereoisomers, preferably enantiomers and/or diastereomers, in any mixing ratio, or a physiologically acceptable salt thereof, or a corresponding solvate thereof, respectively.

8. The combination according to claim 1, wherein component (A) is at least one compound selected from the group consisting of BVT-5182, EMDT, Ro 65-7199, Ro 04-6790, Ro 63-0563, MS-245, SB-271046, LY-483518, ALX-0440, WAY-181189, WAY-466, ALX-1175, ALX-1161, SB-271046, SB-258510, SB-357134, SB-214111, SB-399885, Ro 65-7674, SB-699929, Ro 43-68554, SB-742457, Ro 66-0074, [¹¹C]GSK-215083,
[HT1] N,N-dimethyl-2-(1-(phenylsulfonyl)-1H-indol-3-yl)ethanamine,
[HT2] 5-chloro-2-methyl-3-(1,2,3,6-tetrahydropyridin-4-yl)-1H-indole,
[HT3] 2-(1-(5-(phenylsulfonamido)-1H-indol-3-yl)ethylidene)hydrazinecarboximidamide,
[HT4] 5-fluoro-3-(3-fluorophenylsulfonyl)-1-(piperidin-3-yl)-1H-indole,
[HT5] 2-((1-(4-aminophenylsulfonyl)-1H-indol-3-yl)methylene)hydrazinecarboximidamide,
[HT6] 3-(phenylsulfonyl)-7-(2-(pyrrolidin-1-yl)ethoxy)-1H-indole,
[HT7] N,N-dimethyl-2-(1-(phenylsulfonyl)-1H-indol-4-yloxy)ethanamin,
[HT8] 1-(phenylsulfonyl)-3-(pyrrolidin-2-ylmethyl)-1H-indole,
[HT9] 2-(3-benzyl-1H-indol-1-yl)-N,N-dimethylethanamine,
[HT10] 4-(3-(1,2,3,6-tetrahydropyridin-4-yl)-1H-indol-1-ylsulfonyl)benzenamine,
[HT12] (6S,7S)-7-(dimethylamino)-2-tosyl-2,6,7,8-tetrahydrobenzo[cd]indol-6-ol,
[HT13] 2-(1-(phenylsulfonyl)-6,7-dihydro-1H-indol-4(5H)-ylidene)hydrazinecarboximidamide,
[HT14] 6-(phenylsulfonyl)-2,3,4,9-tetrahydro-1H-carbazol-3-amine,
[HT15] 3-(3-chlorophenylsulfonyl)-1-(pyrrolidin-2-ylmethyl)-1H-pyrrolo[2,3-b]pyridine,
[HT16] 4-(5,6,7,8-tetrahydrocarbazol-9-ylsulfonyl)benzenamine,
[HT17] 3-(2-aminoethyl)-1-(naphthalen-2-ylsulfonyl)-1H-imidazo[4,5-b]pyridin-2(3H)-one,
[HT18] 2-(4-(6-chloroimidazo[2,1-b]thiazol-5-ylsulfonyl)-4H-thieno[3,2-b]pyrrol-6-yl)-N,N-dimethylethanamine,
[HT19] N,N-dimethyl-3-(phenylsulfonyl)-6,7,8,9-tetrahydro-3H-benzo[e]indol-8-amine,
[HT20] N-(4-(piperidin-4-yl)-2H-chromen-6-yl)naphthalene-1-sulfonamide,
[HT21] 2-(3-benzyl-3H-inden-1-yl)-N,N-dimethylethanamine,
[HT22] N-(7-((3S,4S)-3-fluoropiperidin-4-yloxy)benzofuran-5-yl)-2-methoxy-5-methylbenzenesulfonamide,
[HT23] 2-Methoxy-10-(1-methylpyrrolidin-3-yl)-5-thia-4b-aza-indeno[2,1-a]indene 5,5-dioxide,
[HT24] 1-(6-(phenylsulfonyl)pyridin-3-yl)piperazine,
[HT25] 1-(2-methoxy-5-(naphthalen-1-ylsulfonyl)phenyl)piperazine,
[HT26] 1-(2-chloro-5-(phenylsulfonyl)phenyl)piperazine,
[HT27] 8-methoxy-1-(3-methoxy-5-(piperazin-1-yl)benzyl)-3,4-dihydroquinolin-2(1H)-one,
[HT28] 5-chloro-3-(3-chlorophenylsulfonyl)-7-(4-methylpiperazin-1-yl)-1H-indole,
[HT29] 1-(4-(phenylsulfonypnaphthalen-1-yl)piperazine,
[HT30] 3-(phenylsulfonyl)-7-(piperazin-1-yl)-1H-pyrrolo[3,2-b]pyridine,
[HT31] 4-(2-chlorophenylsulfonyl)-6-methyl-8-(piperazin-1-yl)-3,4-dihydro-2H-benzo[b][1,4]oxazine,
[HT32] 5-(4-methylpiperazin-1-yl)-3-(naphthalen-1-ylsulfonyl)-1H-indazole,
[HT33] 4-(4-(4-(4,5-dihydro-1H-imidazol-2-yl)piperazin-1-yl)-1H-indol-1-ylsulfonyl)benzenamine,
[HT34] 5-(4-benzylpiperazin-1-yl)-3-(phenylsulfonyl)-1H-pyrrolo[3,2-b]pyridine,
[HT35] 2-(2,3-dichlorophenylsulfonyl)-5-(piperazin-1-yl)-1,2,3,4-tetrahydroisoquinoline,
[HT36] 6-fluoro-4-(2-fluorobenzyl)-8-(piperazin-1-yl)-2H-benzo[b][1,4]oxazin-3(4H)-one,
[HT37] 2-(phenylsulfonyl)-4-(piperazin-1-yl)-1H-indole,
[HT38] 4-(4-(piperazin-1-yl)naphthalen-1-ylsulfonyl)benzenamine,
[HT39] 8-(4-(4-fluorobenzyl)piperazin-1-yl)-3-(phenylsulfonyl)quinoline,
[HT40] 2,3-dichloro-N-(2,3,4,5-tetrahydro-1H-benzo[d]azepin-7-yl)benzenesulfonamide,
[HT41] 2-(methylthio)-3-(phenylsulfonyl)-4H-pyrido[1,2-a]pyrimidin-4-imine and
[HT42] 8-(4-(3-fluoro-5-(trifluoromethypbenzyl)phenylsulfonyl)-N,N,3-trimethyl-2,3,4,5-tetrahydro-1H-benzo[d]azepin-7-amine;
salts thereof, free bases thereof, racemates thereof, enantiomers thereof, and combinations thereof.

9. The combination according to claim 1, wherein component (B) is at least one compound selected from the group consisting of
edrophonium, ganstigmine, demecarium, ambenonium, neostigmine bromide, dehydroevodiamine chloride, eseroline, imperatorin, scopoletin (SCT), huperizine A (Hup A), huperzine A, tacrine hydrochloride (CI-970, THA.HCl), 7-methoxytacrine (7-MEOTA), velnacrine maleate (HP-029, P83-6029A), rivastigmine tartrate (ENA-713, ENA-713D, ONO-2540, SDZ-212-713, SDZ-ENA-713), eptastigmine tartrate (L-693487), heptylstigmine tartrate (MF-201), donepezil hydrochloride (BNAG, E-2020), suronacrine maleate (HP-128), UCB-11056, SM-10888, stacofylline hydrochloride (S-9977, S-9977-2), berberine iodide, zifrosilone (MDL-73745), norpyridostigmine, quilostigmine (HP-290, NXX-066), E-2030, THB-013, F-3796, PD-142012, CI-1002, PD-142676, terestigmine tartrate (CHF-2060), thiacymserine, phenserine tartrate, galantamine hydrobromide (GP-37267), galantamine hydrobromide (R-113675), Ro-46-5934, P11012, MF-8615, MF-268 bitartrate, anseculin hydrochloride (KA-672.HCl), ensaculin hydrochloride, icopezil maleate (CP-118954, CP-118954-11), eserine salicylate, physostigmine salicylate, isovanihuperzine A (IVHA), JWS-USC-751X, FR-152558, UR-1827, P11467, P-10358, bis(7)-tacrine, HMR-2420, T-82, CP-126998, TV-3279, MSF, THA-C8, subergorgic acid, suberogorgin, SPH-1286, huperzine B (Hup B), pyridostigmine bromide (Ro-1-5130), huprine X, ER-127528, tolserine tartrate, huprine Y, coronaridine, RS-1233, kobophenol A, bis(12)-huperine, BGC-20-1259, RS-1259, NP-7557, ZT-1, ITH-4012, TK-19, T-81, TH-171, TH-185, distigmine bromide (BC-51), (−)-9-dehydrogalanthaminium bromide, memoquin, NP-0361, scopoletin 7-O-beta-D-glucopyranoside (NSC-404560), scopolin (SCN), scopoloside, BW-284c51, SP-004, SP-04, withaferin A (NSC-101088), withaferine (NSC-273757), (+)-corynoline, corynoline, (S)-(−)-oxypeucedanin, oxypeucedanin, (−)-voacangine, carbomethoxyibogaine, voacangine, dieckol, phlorofucofuroeckol (PFF), phlorofucofuroeckol A, (−)-3-O-acetylspectaline hydrochloride and rhaphiasaponin 1, salts thereof, free bases thereof, racemates thereof, enantiomers thereof, and combinations thereof.

10. The combination according to claim 1, which comprises 1-99% by weight of component (A) and 99-1% by weight of component (B), preferably 70 to 95% by weight of component (A) and 30 to 5% by weight of component (B), more preferably 85 to 95% by weight of component (A) and 15 to 5% by weight of component (B), in each case referring to the total weight of both components (A) and (B).

11. An active substance combination as defined in claim 1 which is a pharmaceutical composition.

12. A method of administration of the combination of claim 1 for simultaneous acetylcholinesterase inhibition and 5-HT₆-receptor regulation.

13. A method of administration of the active substance combination of claim 1 for regulation of appetite, for maintenance, increase or reduction of body weight, for prophylaxis and/or treatment of disorders related to food ingestion, preferably for prophylaxis and/or treatment of obesity, anorexia, cachexia, bulimia, diabetes, preferably type II diabetes (non-insulin-dependent diabetes mellitus), or for prophylaxis and/or treatment of gastrointestinal tract disorders, preferably of the irritable bowel syndrome, for prophylaxis and/or treatment of Metabolic Syndrome, Peripheral Nervous System Disorders, Central Nervous System Disorders, arthritis, epilepsy, anxiety, panic, depression, cognitive disorders, memory disorders, cardiovascular diseases, senile dementia processes, such as Alzheimer's, Parkinson's and/or Huntington's Disease, schizophrenia, psychosis, infantile hyperkinesia (ADHD, attention deficit / hyperactivity disorder), pain, hypertensive syndrome, inflammatory diseases, immunologic diseases or for improvement of cognition.

14. A pharmaceutical formulation, characterized in that it comprises an active substance combination according to claim 1 and optionally one or more pharmacologically acceptable adjuvants.

15. The pharmaceutical formulation according to claim 14, which is in solid pharmaceutical forms selected from the group consisting of tablets, tablets, chewing tablets, chewing gums, dragées, capsules, suppositories, powder preparations, transdermal therapeutic systems, and transmucosal therapeutic systems.

16. The pharmaceutical formulation according to claim 14, which is in the form of multiple particles, microtablets, microcapsules, microspheroids, granules, crystals or pellets, and is optionally compacted in a tablet, filled in a capsule or suspended in a suitable liquid.

17. The pharmaceutical formulation according to claim 14, which is for oral, intravenous, intramuscular, subcutaneous, intrathecal, epidural, buccal, sublingual, pulmonal, rectal, transdeitual, nasal or intracerebroventricular application.

18. The pharmaceutical formulation according to claim 14, wherein at least one of the components of the active substance combination (A) or (B) is present at least partially in sustained-release form.

19. The pharmaceutical formulation according to claim 18, wherein the medicament has at least one coating or at least one matrix comprising at least one material, which sustains active substance release.

20. The pharmaceutical formulation according to claim 19, wherein the sustained-release material is based on optionally modified, water-insoluble, natural, semisynthetic or synthetic polymer, or a natural wax or fat or fatty alcohol or semisynthetic or synthetic fatty acid, or on a mixture of at least two of these afore mentioned components.

21. The pharmaceutical formulation according to claim 20, wherein the water-insoluble polymer is based on an acrylic resin, which is selected from the group consisting of poly(meth)acrylates, poly(C₁₋₄)dialkylamino(C₁₋₄)alkyl(meth)acrylates and/or copolymers thereof, mixtures thereof, and combinations thereof.

22. The pharmaceutical formulation according to claim 20, wherein the water-insoluble polymers are selected from the group consisting of cellulose derivatives, alkyl cellulose ethyl cellulose, and cellulose esters.

23. The pharmaceutical formulation according to claim 20, wherein the wax is selected from the group consiting of carnauba wax, beeswax, glycerol monostearate, glycerol monobehenate, glycerol ditripalmitostearate, microcrystalline wax a, mixtures thereof, and combinations thereof.

24. The pharmaceutical formulation according to claim 20, wherein the polymers are in combination with one or more plasticizers.

25. The pharmaceutical formulation according to claim 24, wherein that besides the sustained-release form, at least one of the active substance components (A) or (B) is present in a non-sustained-release form.

26. The combination according to claim 1, wherein component (A) has a Kᵢ value for binding to the 5-HT₆-receptor below or equal to 500 nM.

27. The combination according to claim 1, wherein component (A) has a Kᵢ value for binding to the 5-HT₆-receptor below or equal to 100 nM.

28. The combination according to claim 1, wherein component (A) has a Kᵢ value for binding to the 5-HT₆-receptor below or equal to 50 nM.

29. The combination according to claim 1, wherein component (A) has a Kᵢ value for binding to the 5-HT₆-receptor below or equal to 25 nM.

30. The combination according to claim 1 wherein component (B) has an IC₅₀ for the inhibition of acetylcholinesterase in erythrocytes below or equal to 500 nM.

31. The combination according to claim 1 wherein component (B) has an IC₅₀ for the inhibition of acetylcholinesterase in erythrocytes below or equal to 300 nM.

32. The combination according to claim 1 wherein component (B) has an IC₅₀ for the inhibition of acetylcholinesterase in erythrocytes below or equal to 200 nM.

33. The combination according to claim 1 wherein component (B) has an IC₅₀ for the inhibition of acetylcholinesterase in erythrocytes below or equal to 100 nM.

34. The pharmaceutical formulation according to claim 14, which is in liquid and semi-liquid pharmaceutical forms selected from the group consisting of drops, juice, sirup, solutions, emulsions, and suspensions.

35. The pharmaceutical formulation according to claim 34, which is in a form selected from the group consisting of tablets, capsules, drops or solution.
